(19) United States
(12) Patent Application Publication
(10) Pub. No.: US 2024/0190874 A1
(43) Pub. Date: Jun. 13, 2024

(54) SMALL MOLECULE DEGRADERS OF ANDROGEN RECEPTOR

(71) Applicant: THE REGENTS OF THE UNIVERSITY OF MICHIGAN, Ann Arbor, MI (US)

(72) Inventors: Shaomeng Wang, Superior Township, MI (US); Lijie Zhao, Ann Arbor, MI (US); Xin Han, Ann Arbor, MI (US); Weiguo Xiang, Ypsilanti, MI (US)

(21) Appl. No.: 18/279,229

(22) PCT Filed: Mar. 3, 2022
(86) PCT No.: PCT/US22/18599

§ 371 Date: Aug. 29, 2023

Related U.S. Application Data
(60) Provisional application No. 63/156,292, filed on Mar. 3, 2021.

(51) Int. Cl. C07D 471/10; A61K 31/438

(52) U.S. Cl. CPC C07D 471/10; A61K 31/438

(57) ABSTRACT

The present disclosure provides compounds of Formula (I): and the salts or solvates thereof, wherein A¹, B¹, L, and X² are as defined in the specification. The present disclosure also relates to uses of the compounds, e.g., as androgen receptor degraders useful for the treatment of diseases (e.g., cancer).

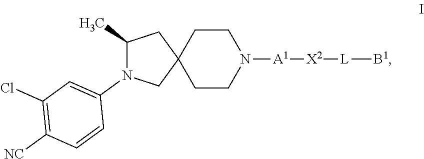

### RELATED APPLICATIONS

The application claims priority to, and the benefit of, U.S. Provisional Application No. 63/156,292, filed on Mar. 3, 2021, the content of which is incorporated herein by reference in its entirety.

### GOVERNMENT SUPPORT

This invention was made with government support under CA186786 awarded by the National Institutes of Health. The government has certain rights in the invention.

### BACKGROUND

Despite improvements in medical treatments over the past three decades, prostate cancer is significant cause of cancer-related death, and is second only to lung cancer among men in developed countries. Hamdy et al., N Engl J Med, 2016, 375, 1415-1424; Litwin and Tan, H. J. JAMA, 2017, 317, 2532-2542. In addition to surgery and radiotherapy, androgen deprivation therapies (ADT) are front-line treatments for prostate cancer patients with high-risk localized disease, and second-generation anti-androgens such as abiraterone and enzalutamide have been shown to benefit patients with advanced prostate cancer. Karantanos et al., Oncogene. 2013, 32, 5501-511; Harris et al., Nat Clin Pract Urol, 2009, 6, 76-85. Nevertheless, patients who progress to metastatic castration-resistant prostate cancer (mCRPC), a hormone-refractory form of the disease, face a high mortality rate and no cure is currently available. Narayanan et al., Oncoscience. 2017, 4, 175-177; Crowder et al., Endocrinology. 2018, 159, 980-993.

The androgen receptor (AR) and its downstream signaling play a critical role in the development and progression of both localized and metastatic prostate cancer. Previous strategies that successfully target AR signaling have focused on blocking androgen synthesis by drugs such as abiraterone and inhibition of AR function by AR antagonists such as enzalutamide and apalutamide (ARN-509). Watson et al., Nat Rev Cancer. 2015, 15, 701-711. However, such agents become ineffective in advanced prostate cancer with AR gene amplification, mutation, and alternate splicing. Balbas et al., Elife. 2013, 2, e00499; Lottrup et al., J Clin Endocrinol Metab. 2013, 98, 2223-2229. But in most patients with CRPC, the AR protein continues to be expressed and tumors are still dependent upon AR signaling. Consequently, AR is an attractive therapeutic target for mCRPC, see e.g., Zhu et al., Nat Commun. 2018, 9, 500; Munuganti et al., Chem Biol. 2014, 21, 1476-485, and other diseases. Student et al., European Journal of Pharmacology 866: 172783 (2020).

The Proteolysis Targeting Chimera (PROTAC) strategy has gained momentum with its promise in the discovery and development of completely new types of small molecule therapeutics by inducing targeted protein degradation. Raina et al., Proc Natl Acad Sci USA. 2016, 113, 7124-7129; Zhou et al., J. Med. Chem. 2018, 61, 462-481.

A PROTAC molecule is a heterobifunctional small molecule containing one ligand, which binds to the target protein of interest, and a second ligand for an E3 ligase system, tethered together by a chemical linker. Bondeson, D. P.; Crews, C. M. Targeted Protein Degradation by Small Molecules. Annu Rev Pharmacol Toxicol. 2017, 57, 107-123. Because AR protein plays a key role in CRPC, AR degraders designed based upon the PROTAC concept could be effective for the treatment of CRPC when the disease becomes resistant to AR antagonists or to androgen synthesis inhibitors. Salami et al., Commun Biol. 2018, 1, 100; Pal et al., Cancer. 2018, 124, 1216-1224; Wang et al., Clin Cancer Res. 2018, 24, 708-723; Gustafson et al., Angew. Chem. Int. Ed. 2015, 54, 9659-9662. Naito et al. have recently reported AR degraders designed based upon the PROTAC concept, which were named Specific and Nongenetic IAP-dependent Protein Erasers (SNIPERs). Shibata et al., J. Med. Chem. 2018, 61, 543-575.

While SNIPER AR degraders are effective in inducing partial degradation of the AR protein in cells, they also induce the auto-ubiquitylation and proteasomal degradation of the cIAP1 protein, the E3 ligase needed for induced degradation of AR protein, thus limiting their AR degradation efficiency and therapeutic efficacy.

(4R)-1-((S)-2-(2-(4-((4′-(3-(4-cyano-3-(trifluoromethyl)phenyl)-5,5-dimethyl-4-oxo-2-thioxoimidazolidin-1-yl)-[1,1′-biphenyl]-4-yl)oxy)butoxy)acetamido)-3,3-dimethyl-butanoyl)-4-hydroxy-N-(4-(4-methylthiazol-5-yl)benzyl)pyrrolidine-2-carboxamide ((ARCC-4) was recently reported as another PROTAC degrader, which was designed using enzalutamide as the AR antagonist and a von Hippel-Lindau (VHL) ligand. Salami et al., Commun Biol. 2018, 1, 100; US 20170327469. ARCC-4 was shown to be more potent and effective than enzalutamide at inducing apoptosis and inhibiting proliferation of AR-amplified prostate cancer cells. ARD-69 was also recently reported as a PROTAC AR degrader. Han et al., J. Med. Chem. 62:941-964 (2019).

There is a need in the art for additional AR degraders to treat prostate cancer and other diseases.

### SUMMARY

In some aspects, the present disclosure provides heterobifunctional small molecules represented by any one or more of Formulae I-VI, below, and the pharmaceutically acceptable salts and solvates, e.g., hydrates, thereof. These compounds are collectively referred to herein as “Compounds of the Disclosure.” Compounds of the Disclosure are androgen receptor (AR) degraders and are thus useful in treating diseases or conditions wherein degradation of the androgen receptor protein provides a therapeutic benefit to a subject.

In some aspects, the present disclosure provides methods of treating a condition or disease by administering a therapeutically effective amount of a Compound of the Disclosure to a subject, e.g., a human cancer patient, in need thereof. The disease or condition treatable by degradation of the androgen receptor is, for example, a cancer, e.g., prostate cancer, e.g., metastatic castration-resistant prostate cancer.

In some aspects, the present disclosure provides a method of degrading, e.g., reducing the level of, of androgen receptor protein in a subject in need thereof, comprising administering to the individual an effective amount of at least one Compound of the Disclosure.

In some aspects, the present disclosure provides a pharmaceutical composition comprising a Compound of the Disclosure and an excipient and/or pharmaceutically acceptable carrier.

In some aspects, the present disclosure provides a composition comprising a Compound of the Disclosure and an excipient and/or pharmaceutically acceptable carrier for use treating diseases or conditions wherein degradation of the androgen receptor provides a benefit, e.g., cancer.

In some aspects, the present disclosure provides a composition comprising: (a) a Compound of the Disclosure; (b) a second therapeutically active agent; and (c) optionally an excipient and/or pharmaceutically acceptable carrier.

In some aspects, the present disclosure provides a Compound of the Disclosure for use in treatment of a disease or condition of interest, e.g., cancer.

In some aspects, the present disclosure provides a use of a Compound of the Disclosure for the manufacture of a medicament for treating a disease or condition of interest, e.g., cancer.

In some aspects, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In some aspects, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

It is to be understood that both the foregoing summary and the following detailed description are exemplary and explanatory only, and are not restrictive of the invention as claimed.

### DETAILED DESCRIPTION

The present disclosure relates to compounds that may be heterobifunctional small molecules which could function as androgen receptor (AR) protein degraders. The present disclosure also relates to uses of the compounds, e.g., in therapeutic methods of treating diseases (e.g., cancer), wherein the degradation of AR proteins provides a benefit.

### Compounds of the Disclosure

It is understood that Compounds of the Disclosure may function as heterobifunctional AR degraders.

In some embodiments, Compounds of the Disclosure are compounds of Formula I.

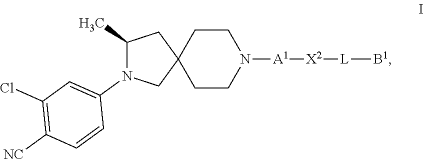

or a pharmaceutically acceptable salt or solvate thereof, wherein:
A¹ is selected from phenylenyl, 5-membered heteroarylenyl, and 6-membered heteroarylenyl;
X² is selected from —C(═O)—, —S(═O)₂—, —O—, and —CR^{4c}R^{4d}—; or X² is absent;
   R^{4c} and R^{4d} are independently selected from hydrogen and C₁-C₄ alkyl;
   L is -J¹-J²-J³-J⁴-J⁵-; wherein J¹ is attached to X²;
   J¹ is selected from cycloalkylenyl and heterocyclenyl; or J¹ is absent;
   J² is selected from —(CH₂)_{b}—, —C(═O)—, —CH═CH—, and —C≡C—;
   b is 0, 1, 2, or 3;
   J³ is selected from alkylenyl, heteroalkylenyl, cycloalkylenyl, heterocyclenyl, phenylenyl, and heteroarylenyl; or J³ is absent;
   J⁴ is selected from alkylenyl, cycloalkylenyl, and heterocyclenyl; or J⁴ is absent;
   J⁵ is selected from —(CH₂)_{c}—, —O—, —N(R⁵)—, and —C(═O)—;
   c is 0, 1, 2, or 3;
   R⁵ is selected from hydrogen and C₁-C₄ alkyl;
B¹ is selected from the group consisting of:

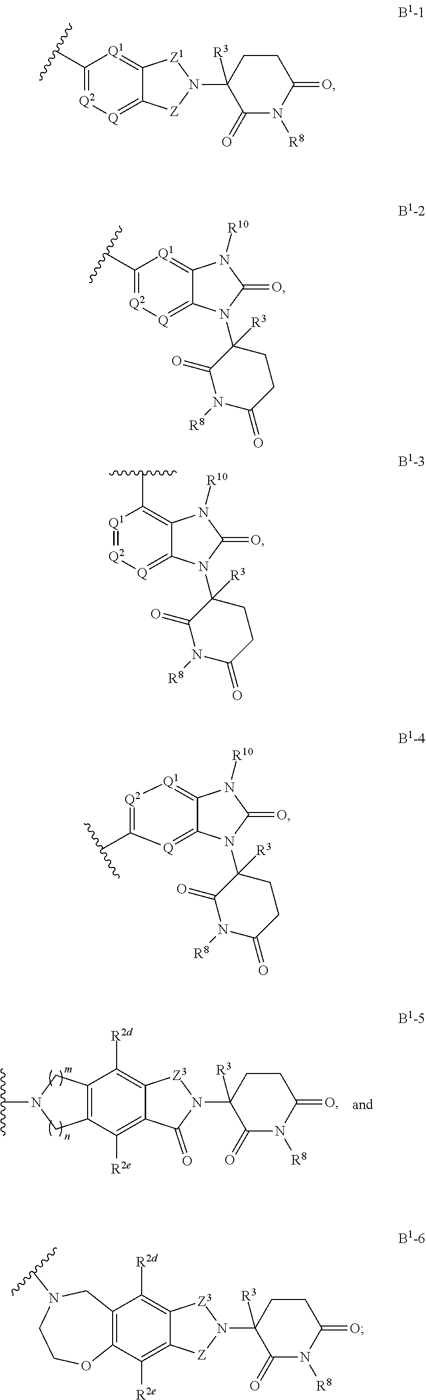

Q¹ is selected from —CR^{2a}═ and —N═;
Q² is selected from —CR^{2b}═ and —N═;
Q is selected from —CR^{2c}═ and —N═;
R^{2a}, R^{2b}, R^{2c}, R^{2d}, and R^{2e} are independently selected from hydrogen, halo, amino, C₁-C₃ alkyl, and C₁-C₃ alkoxy;
R³ is selected from hydrogen, deuterium, fluoro, and C₁-C₄ alkyl;
m is 1, 2, or 3;
n is 1, 2, or 3;
Z and Z¹ are —C(═O)—; or
Z is —C(═O)— and Z¹ is —CR^{6a}R^{6b}—; or
Z is —CR^{6a}R^{6b}— and Z¹ is —C(═O)—;
Z³ is selected from —CR^{6c}R^{6d}— and —C(═O)—;
R^{6a} and R^{6b} are independently selected from hydrogen and C₁-C₃ alkyl; or
R^{6c} and R^{6d} taken together with the carbon to which they are attached from a C₃-C₆ cycloalkyl;
R⁸ is selected from hydrogen and C₁-C₃ alkyl; and
R¹⁰ is selected from hydrogen and C₁-C₄ alkyl.

In some embodiments, A¹ is phenylenyl.

In some embodiments, A¹ is 5-membered heteroarylenyl.

In some embodiments, A¹ is 6-membered heteroarylenyl.

In some embodiments, X² is —C(═O)—.

In some embodiments, X² is —S(═O)₂—.

In some embodiments, X² is —O—.

In some embodiments, X² is —CR^{4c}R^{4d}—.

In some embodiments, X² is absent.

In some embodiments, R^{4c} is hydrogen.

In some embodiments, R^{4c} is C₁-C₄ alkyl (e.g., methyl, ethyl, or propyl).

In some embodiments, R^{4d} is hydrogen.

In some embodiments, R^{4d} is C₁-C₄ alkyl (e.g., methyl, ethyl, or propyl).

In some embodiments, J¹ is cycloalkylenyl.

In some embodiments, J¹ is heterocyclenyl.

In some embodiments, J¹ is absent.

In some embodiments, J² is —(CH₂)_{b}—.

In some embodiments, J² is —C(═O)—.

In some embodiments, J² is —CH═CH—.

In some embodiments, J² is —C≡C—.

In some embodiments, b is 0.

In some embodiments, b is 1.

In some embodiments, b is 2.

In some embodiments, b is 3.

In some embodiments, J³ is alkylenyl.

In some embodiments, J³ is heteroalkylenyl.

In some embodiments, J³ is cycloalkylenyl.

In some embodiments, J³ is heterocyclenyl.

In some embodiments, J³ is phenylenyl.

In some embodiments, J³ is heteroarylenyl.

In some embodiments, J³ is absent.

In some embodiments, J⁴ is alkylenyl.

In some embodiments, J⁴ is cycloalkylenyl.

In some embodiments, J⁴ is heterocyclenyl.

In some embodiments, J⁴ is absent.

In some embodiments, J⁵ is —(CH₂)_{c}—.

In some embodiments, J⁵ is —O—.

In some embodiments, J⁵ is —N(R⁵)—.

In some embodiments, J⁵ is —C(═O)—.

In some embodiments, c is 0.

In some embodiments, c is 1.

In some embodiments, c is 2.

In some embodiments, c is 3.

In some embodiments, R⁵ is hydrogen.

In some embodiments, R⁵ is C₁-C₄ alkyl (e.g., methyl, ethyl, or propyl).

In some embodiments, B¹ is B¹-1.

In some embodiments, B¹ is B¹-2.

In some embodiments, B¹ is B¹-3.

In some embodiments, B¹ is B¹-4.

In some embodiments, B¹ is B¹-5.

In some embodiments, B¹ is B¹-6.

In some embodiments, Q¹ is —CR^{2a}.

In some embodiments, Q¹ is —N═.

In some embodiments, Q² is —CR^{2b}.

In some embodiments, Q² is —N═.

In some embodiments, Q is —CR^{2c}.

In some embodiments, Q is —N═.

In some embodiments, R^{2a} is hydrogen.

In some embodiments, R^{2a} is halo (e.g., F, Cl, or Br).

In some embodiments, R^{2a} is amino.

In some embodiments, R^{2a} is C₁-C₃ alkyl (e.g., methyl, ethyl, or propyl).

In some embodiments, R^{2a} is C₁-C₃ alkoxy.

In some embodiments, R^{2b} is hydrogen.

In some embodiments, R^{2b} is halo (e.g., F, Cl, or Br).

In some embodiments, R^{2b} is amino.

In some embodiments, R^{2b} is C₁-C₃ alkyl (e.g., methyl, ethyl, or propyl).

In some embodiments, R^{2b} is C₁-C₃ alkoxy.

In some embodiments, R^{2c} is hydrogen.

In some embodiments, R^{2d} is halo (e.g., F, Cl, or Br).

In some embodiments, R^{2c} is amino.

In some embodiments, R^{2d} is C₁-C₃ alkyl (e.g., methyl, ethyl, or propyl).

In some embodiments, R^{2d} is C₁-C₃ alkoxy.

In some embodiments, R^{2d} is hydrogen.

In some embodiments, R^{2d} is halo (e.g., F, Cl, or Br).

In some embodiments, R^{2d} is amino.

In some embodiments, R^{2d} is C₁-C₃ alkyl (e.g., methyl, ethyl, or propyl).

In some embodiments, R^{2d} is C₁-C₃ alkoxy.

In some embodiments, R^{2e} is hydrogen.

In some embodiments, R^{2e} is halo (e.g., F, Cl, or Br).

In some embodiments, R^{2e} is amino.

In some embodiments, R^{2e} is C₁-C₃ alkyl (e.g., methyl, ethyl, or propyl).

In some embodiments, R^{2e} is C₁-C₃ alkoxy.

In some embodiments, R³ is hydrogen.

In some embodiments, R³ is deuterium.

In some embodiments, R³ is fluoro.

In some embodiments, R³ is C₁-C₄ alkyl (e.g., methyl, ethyl, or propyl).

In some embodiments, m is 1.

In some embodiments, m is 2.

In some embodiments, m is 3.

In some embodiments, n is 1.

In some embodiments, n is 2.

In some embodiments, n is 3.

In some embodiments, Z and Z¹ are —C(═O)—.

In some embodiments, Z is —C(═O)— and Z¹ is —CR^{6a}R^{6b}—.

In some embodiments, Z is —CR^{6a}R^{6b} and Z¹ is —C(═O)—.

In some embodiments, Z³ is —CR^{6c}R^{6d}—.

In some embodiments, Z³ is —C(═O)—.

In some embodiments, R^{6a} is hydrogen.

In some embodiments, R^{6a} is C₁-C₃ alkyl (e.g., methyl, ethyl, or propyl).

In some embodiments, R^{6b} is hydrogen.

In some embodiments, R^{6b} is C₁-C₃ alkyl (e.g., methyl, ethyl, or propyl).

In some embodiments, R^{6c} and R^{6d} taken together with the carbon to which they are attached from a C₃-C₆ cycloalkyl.

In some embodiments, R⁸ is hydrogen.

In some embodiments, R⁸ is C₁-C₃ alkyl (e.g., methyl, ethyl, or propyl).

In some embodiments, R¹⁰ is hydrogen.

In some embodiments, R¹⁰ is C₁-C₄ alkyl (e.g., methyl, ethyl, or propyl).

In some embodiments, Compounds of the Disclosure are compounds of Formula II:

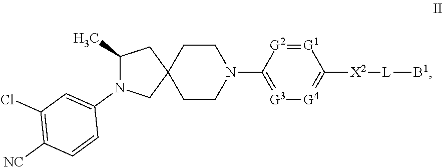

or a pharmaceutically acceptable salt or solvate thereof, wherein:
G¹ is selected from CR^{5a}═ and —N═;
G² is selected from —CR^{5a}═ and —N═;
G³ is selected from —CR^{5c}═ and —N═;
G⁴ is selected from —CR^{5d}═ and —N═;
R^{5a}, R^{5b}, R^{5c}, and R^{5d} are independently selected from hydrogen, halo, C₁-C₃ alkyl, and C₁-C₃ alkoxy; and
X², L, and B¹ are as defined in connection with Formula I.

In some embodiments, Compounds of the Disclosure are compounds of Formula II, or a pharmaceutically acceptable salt or solvate thereof, wherein G¹ is —CR^{5a}═. In some embodiments, R^{5a} is hydrogen.

In some embodiments, Compounds of the Disclosure are compounds of Formula II, or a pharmaceutically acceptable salt or solvate thereof, wherein G¹ is —N═.

In some embodiments, Compounds of the Disclosure are compounds of Formula II, or a pharmaceutically acceptable salt or solvate thereof, wherein G² is —CR^{5b}═. In some embodiments, R^{5b} is hydrogen.

In some embodiments, Compounds of the Disclosure are compounds of Formula II, or a pharmaceutically acceptable salt or solvate thereof, wherein G² is —N═.

In some embodiments, Compounds of the Disclosure are compounds of Formula II, or a pharmaceutically acceptable salt or solvate thereof, wherein G³ is —CR^{5c}═. In some embodiments, R^{5c} is hydrogen.

In some embodiments, Compounds of the Disclosure are compounds of Formula II, or a pharmaceutically acceptable salt or solvate thereof, wherein G³ is —N═.

In some embodiments, Compounds of the Disclosure are compounds of Formula II, or a pharmaceutically acceptable salt or solvate thereof, wherein G⁴ is —CR^{5d}═. In some embodiments, R^{5d} is hydrogen.

In some embodiments, Compounds of the Disclosure are compounds of Formula II, or a pharmaceutically acceptable salt or solvate thereof, wherein G⁴ is —N═.

In some embodiments, Compounds of the Disclosure are compounds of Formula III:

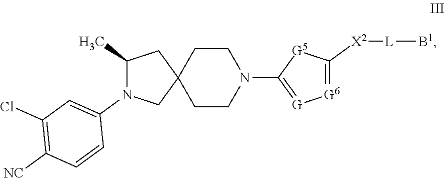

or a pharmaceutically acceptable salt or solvate thereof, wherein:
G is selected from —CH═ and —N═;
G⁵ is selected from —O—, —S—, and —NR⁹—;
G⁶ is selected from —CH═ and —N═;
R⁹ is selected from hydrogen and C₁-C₃ alkyl; and
X², L, and B¹ are as defined in connection with Formula I.

In some embodiments, Compounds of the Disclosure are compounds of Formula IV:

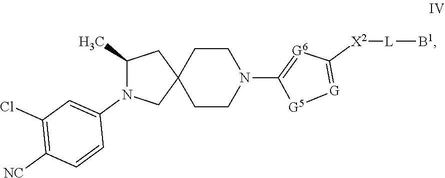

or a pharmaceutically acceptable salt or solvate thereof, wherein:
G is selected from —CH═ and —N═;
G⁵ is selected from —O—, —S—, and —NR⁹—;
G⁶ is selected from —CH═ and —N═;
R⁹ is selected from hydrogen and C₁-C₃ alkyl; and
X², L, and B¹ are as defined in connection with Formula I.

In some embodiments, Compounds of the Disclosure are compounds of Formula V:

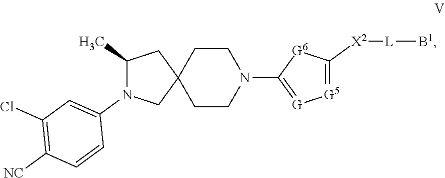

or a pharmaceutically acceptable salt or solvate thereof, wherein:
G is selected from —CH═ and —N═;
G⁵ is selected from —O—, —S—, and —NR⁹—;
G⁶ is selected from —CH═ and —N═;
R⁹ is selected from hydrogen and C₁-C₃ alkyl; and
X², L, and B¹ are as defined in connection with Formula I.

In some embodiments, Compounds of the Disclosure are compounds of any one of Formulae III-V, or a pharmaceutically acceptable salt or solvate thereof, wherein G⁵ is —S—.

In some embodiments, Compounds of the Disclosure are compounds of any one of Formulae III-V, or a pharmaceutically acceptable salt or solvate thereof, wherein G⁵ is —O—.

In some embodiments, Compounds of the Disclosure are compounds of any one of Formulae III-V, or a pharmaceutically acceptable salt or solvate thereof, wherein G⁵ is —NR⁹—.

In some embodiments, Compounds of the Disclosure are compounds of any one of Formulae III-V, or a pharmaceutically acceptable salt or solvate thereof, wherein G is —CH═.

In some embodiments, Compounds of the Disclosure are compounds of any one of Formulae III-V, or a pharmaceutically acceptable salt or solvate thereof, wherein G is —N═.

In some embodiments, Compounds of the Disclosure are compounds of any one of Formulae III-V, or a pharmaceutically acceptable salt or solvate thereof, wherein G⁶ is —CH═.

In some embodiments, Compounds of the Disclosure are compounds of any one of Formulae III-V, or a pharmaceutically acceptable salt or solvate thereof, wherein G⁶ is —N═.

In some embodiments, Compounds of the Disclosure are compounds of Formula VI:

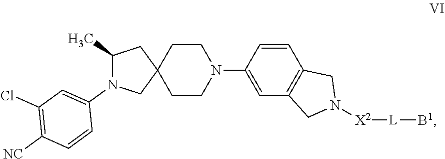

or a pharmaceutically acceptable salt or solvate thereof, wherein X², L, and B¹ are as defined in connection with Formula I.

In some embodiments, Compounds of the Disclosure are compounds of any one of Formulae I-VI, or a pharmaceutically acceptable salt or solvate thereof, wherein X² is —C(═O)—

In some embodiments, Compounds of the Disclosure are compounds of any one of Formulae I-VI, or a pharmaceutically acceptable salt or solvate thereof, wherein X² is —S(═O)₂—

In some embodiments, Compounds of the Disclosure are compounds of any one of Formulae I-VI, or a pharmaceutically acceptable salt or solvate thereof, wherein X² is —O—.

In some embodiments, Compounds of the Disclosure are compounds of any one of Formulae I-VI, or a pharmaceutically acceptable salt or solvate thereof, wherein X² is —CR^{4c}R^{4d}—. In some embodiments, R^{4c} and R^{4d} are hydrogen.

In some embodiments, Compounds of the Disclosure are compounds of any one of Formulae I-VI, or a pharmaceutically acceptable salt or solvate thereof, wherein X² is absent.

In some embodiments, Compounds of the Disclosure are compounds of any one of Formulae I-VI, or a pharmaceutically acceptable salt or solvate thereof, wherein J¹ is cycloalkylenyl.

In some embodiments, Compounds of the Disclosure are compounds of any one of Formulae I-VI, or a pharmaceutically acceptable salt or solvate thereof, J¹ is heterocyclenyl. In some embodiments, J¹ is selected from the group consisting of:

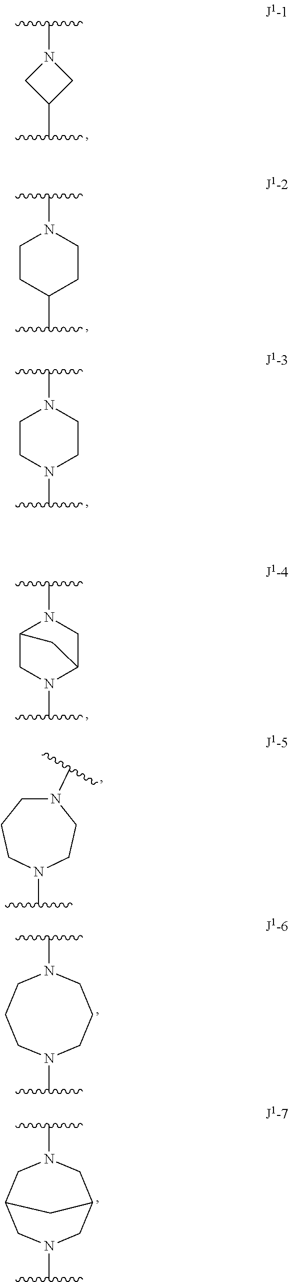

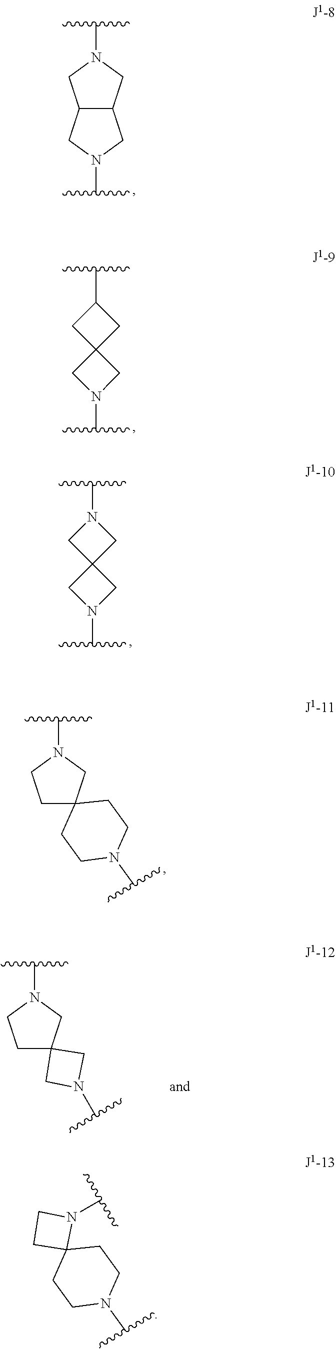 [SMILES: *N1CC2CN(*)CC2C1 ; *C1CC2(C1)CN(*)C2 ; *N1CC2(C1)CN(*)C2 ; *N1CCC2(CC1)CCN(*)C2 ; *N1CCC2(C1)CN(*)C2 ; *N1CCC2(CC1)CCN2*]

In some embodiments, J¹ is J¹-1. In some embodiments, J¹ is J¹-2. In some embodiments, J¹ is J¹-3. In some embodiments, J¹ is J¹-4. In some embodiments, J¹ is J¹-5. In some embodiments, J¹ is J¹-6. In some embodiments, J¹ is J¹-7. In some embodiments, J¹ is J¹-8. In some embodiments, J¹ is J¹-9. In some embodiments, J¹ is J¹-10. In some embodiments, J¹ is J¹-11. In some embodiments, J¹ is J¹-12. In some embodiments, J¹ is J¹-13.

In some embodiments, Compounds of the Disclosure are compounds of any one of Formulae I-VI, or a pharmaceutically acceptable salt or solvate thereof, wherein J¹ is absent.

In some embodiments, Compounds of the Disclosure are compounds of any one of Formulae I-VI, or a pharmaceutically acceptable salt or solvate thereof, wherein J² is selected from —(CH₂)_{b}— and —C≡C—; and b is 0, 1, or 2. In some embodiments, J² is —(CH₂)_{b}—; and b is 0. In some embodiments, J² is —(CH₂)_{b}—; and b is 1. In some embodiments, J² is —C≡C—.

In some embodiments, Compounds of the Disclosure are compounds of any one of Formulae I-VI, or a pharmaceutically acceptable salt or solvate thereof, wherein J³ is selected from cycloalkylenyl and heterocyclenyl.

In some embodiments, Compounds of the Disclosure are compounds of any one of Formulae I-VI, or a pharmaceutically acceptable salt or solvate thereof, wherein J³ is absent.

In some embodiments, Compounds of the Disclosure are compounds of any one of Formulae I-VI, or a pharmaceutically acceptable salt or solvate thereof, wherein J⁴ is selected from alkylenyl, cycloalkylenyl, and heterocyclenyl.

In some embodiments, Compounds of the Disclosure are compounds of any one of Formulae I-VI, or a pharmaceutically acceptable salt or solvate thereof, wherein J⁴ is absent.

In some embodiments, Compounds of the Disclosure are compounds of any one of Formulae I-VI, or a pharmaceutically acceptable salt or solvate thereof, wherein:
J⁵ is selected from —O— and —N(H)—; and
B¹ is selected from B¹-1, B¹-2, and B¹-3.

In some embodiments, Compounds of the Disclosure are compounds of any one of Formulae I-VI, or a pharmaceutically acceptable salt or solvate thereof, wherein:
J⁵ is selected from —(CH₂)_{c}— and —O—;
c is 0;
J⁴ is selected from the group consisting of:

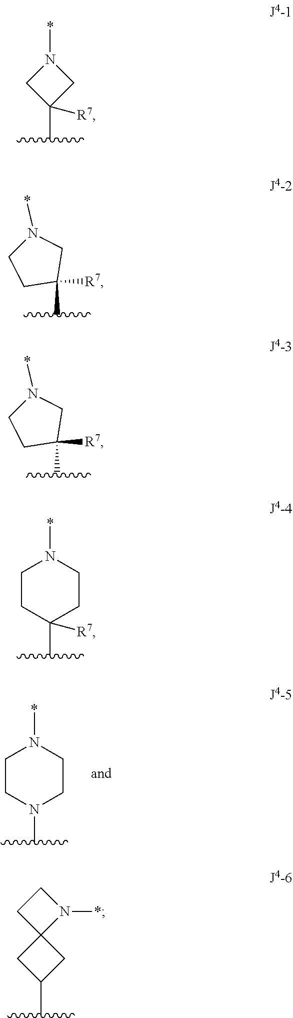

wherein the bond designated with an “*” is attached to B¹;
R⁷ is selected from hydrogen, halo, cyano, hydroxy, C₁-C₃ alkyl, and C₁-C₃ alkoxy; and
B¹ is selected from B¹-1, B¹-2, B¹-3, and B¹-4. In some embodiments, R⁷ is hydrogen. In some embodiments, J⁴ is J⁴-1, J⁴-4, or J⁴-5.

In some embodiments, Compounds of the Disclosure are compounds of any one of Formulae I-VI, or a pharmaceutically acceptable salt or solvate thereof, wherein B1 is B¹-1. In some embodiments, Z and Z¹ are —C(═O)—. In some embodiments, Z is —C(═O)— and Z¹ is —CR^{6a}R^{6b}—. In some embodiments, Z is —CR^{6a}R^{6b}— and Z¹ is —C(═O)—. In some embodiments, R^{6a} and R^{6b} are hydrogen.

In some embodiments, Compounds of the Disclosure are compounds of any one of Formulae I-VI, or a pharmaceutically acceptable salt or solvate thereof, wherein B¹-1 is B¹-1-B:

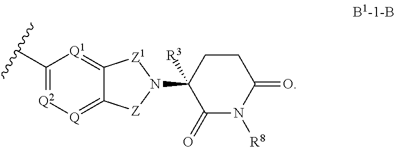

In some embodiments, Z and Z¹ are —C(═O)—. In some embodiments, Z is —C(═O)— and Z¹ is —CR^{6a}R^{6b}. In some embodiments, Z is —CR^{6a}R^{6b}— and Z¹ is —C(═O)—. In some embodiments, R^{6a} and R^{6b} are hydrogen

In some embodiments, Compounds of the Disclosure are compounds of any one of Formulae I-VI, or a pharmaceutically acceptable salt or solvate thereof, wherein B¹-1 is B¹-1-C:

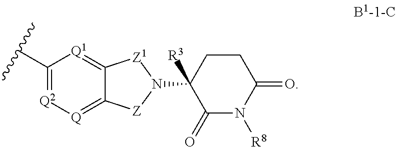

In some embodiments, Z and Z¹ are —C(═O)—. In some embodiments, Z is —C(═O)— and Z¹ is —CR^{6a}R^{6b}—. In some embodiments, Z is —CR^{6a}R^{6b}— and Z¹ is —C(═O)—. In some embodiments, R^{6a} and R^{6b} are hydrogen

In some embodiments, Compounds of the Disclosure are compounds of any one of Formulae I-VI, or a pharmaceutically acceptable salt or solvate thereof, wherein B¹ is B¹-2. In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-2 is B¹-2-B:

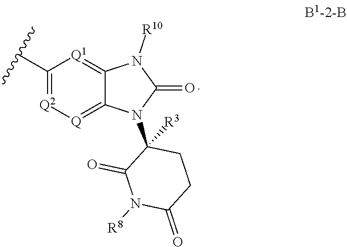

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-2 is B¹-2-C:

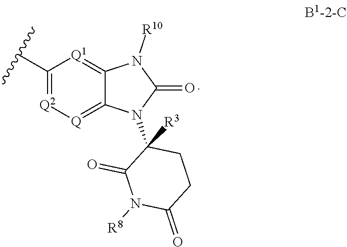

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹ is B¹-3. In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-3 is B¹-3-B:

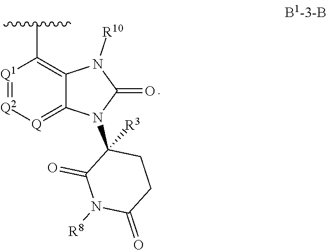

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-3 is B¹-3-C:

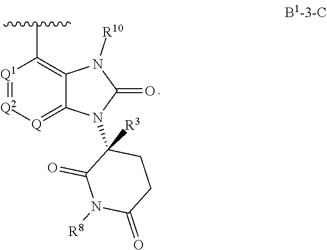

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹ is B¹-4. In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-4 is B¹-4-B:

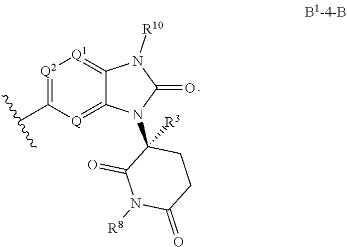

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹-4 is B¹-4-C:

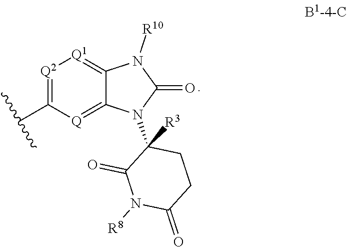

In some embodiments, R¹⁰ is C₁-C₃ alkyl. In some embodiments, R¹⁰ is methyl.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q¹ is —N═.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q¹ is —CR^{2a}. In some embodiments, R^{2a} is selected from hydrogen and halo. In some embodiments, R^{2a} is fluoro. In some embodiments, R^{2a} is hydrogen.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q² is —N═.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q² is —CR^{2b}. In some embodiments, R^{2b} is selected from hydrogen and halo. In some embodiments, R^{2b} is fluoro. In some embodiments, R^{2b} is hydrogen.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q is —N═.

In some embodiments, Compounds of the Disclosure are compounds of any one of Formulae I-VI, or a pharmaceutically acceptable salt or solvate thereof, wherein B¹ is B¹-1, B¹-1-B, B¹-1-C, B¹-2, B¹-2-B, B¹-2-C, B¹-3, B¹-3-B, B¹-3-C, B¹-4, B¹-4-B, or B¹-4-C; and Q is —CR^{2c}. In some embodiments, R^{2c} is selected from hydrogen and halo. In some embodiments, R², is fluoro. In some embodiments, R^{2b} is hydrogen.

In some embodiments, Compounds of the Disclosure are compounds of any one of Formulae I-VI, or a pharmaceutically acceptable salt or solvate thereof, wherein:
J⁵ is selected from —(CH₂)_{c}— and —C(═O)—;
c is 0, 1, 2, or 3; and

B¹ is selected from B¹-5 and B¹-6, or a pharmaceutically acceptable salt or solvate thereof.

In some embodiments, Compounds of the Disclosure are compounds of any one of Formulae I-VI, or a pharmaceutically acceptable salt or solvate thereof, wherein B¹ is B¹-5. In some embodiments, m is 1. In some embodiments, m is 2. In some embodiments, n is 1. In some embodiments, n is 2. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen In some embodiments, Z³ is —C(═O)—. In some embodiments, Z³ is —CR^{6a}R^{6b}—. In some embodiments, R^{6a} and R^{6b} are hydrogen.

In some embodiments, Compounds of the Disclosure are compounds of any one of Formulae I-VI, or a pharmaceutically acceptable salt or solvate thereof, wherein B¹-5 is B¹-5-B.

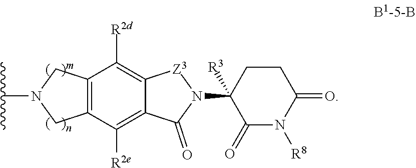

In some embodiments, m is 1. In some embodiments, m is 2. In some embodiments, n is 1. In some embodiments, n is 2. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen In some embodiments, Z³ is —C(═O)—. In some embodiments, Z³ is —CR^{6a}R^{6b}—. In some embodiments, R^{6a} and R^{6b} are hydrogen.

In some embodiments, Compounds of the Disclosure are compounds of any one of Formulae I-VI, or a pharmaceutically acceptable salt or solvate thereof, wherein B¹-5 is B¹-5-C:

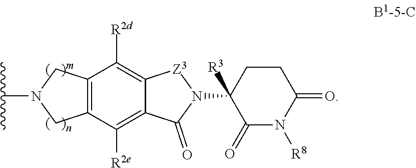

In some embodiments, m is 1. In some embodiments, m is 2. In some embodiments, n is 1. In some embodiments, n is 2. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen In some embodiments, Z³ is —C(═O)—. In some embodiments, Z³ is —CR^{6a}R^{6b}—. In some embodiments, R^{6a} and R^{6b} are hydrogen.

In some embodiments, Compounds of the Disclosure are compounds of any one of Formulae I-VI, or a pharmaceutically acceptable salt or solvate thereof, wherein B¹ is B¹-6. In some embodiments, Z and Z¹ are —C(═O)—. In some embodiments, Z is —C(═O)— and Z¹ is —CR^{6a}R^{6b}. In some embodiments, Z is —CR^{6a}R^{6b}— and Z¹ is —C(═O)—. In some embodiments, R^{6a} and R^{6b} are hydrogen. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen.

In some embodiments, Compounds of the Disclosure are compounds of any one of Formulae I-VI, or a pharmaceutically acceptable salt or solvate thereof, wherein B¹-6 is B¹-6-B:

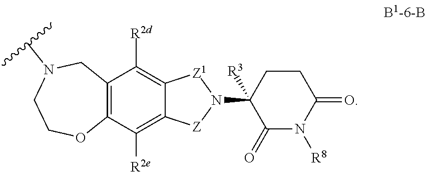

In some embodiments, Z and Z¹ are —C(═O)—. In some embodiments, Z is —C(═O)— and Z¹ is —CR^{6a}R^{6b}. In some embodiments, Z is —CR^{6a}R^{6b}— and Z¹ is —C(═O)—. In some embodiments, R^{6a} and R^{6b} are hydrogen. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen.

In some embodiments, Compounds of the Disclosure are compounds of any one of Formulae I-VI, or a pharmaceutically acceptable salt or solvate thereof, wherein B¹-6 is B¹-6-C.

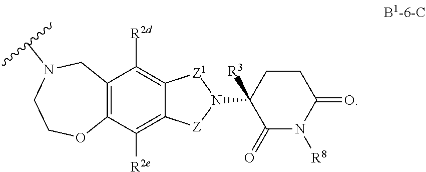

In some embodiments, Z and Z¹ are —C(═O)—. In some embodiments, Z is —C(═O)— and Z¹ is —CR^{6a}R^{6b}. In some embodiments, Z is —CR^{6a}R^{6b}— and Z¹ is —C(═O)—. In some embodiments, R^{6a} and R^{6b} are hydrogen. In some embodiments, R^{2d} and R^{2d} are independently selected from hydrogen and halo. In some embodiments, R^{2d} is fluoro. In some embodiments, R^{2d} is hydrogen. In some embodiments, R^{2e} is fluoro. In some embodiments, R^{2e} is hydrogen.

In some embodiments, Compounds of the Disclosure are compounds of any one of Formulae I-VI, or a pharmaceutically acceptable salt or solvate thereof, wherein R³ is hydrogen.

In some embodiments, Compounds of the Disclosure are compounds of any one of Formulae I-VI, or a pharmaceutically acceptable salt or solvate thereof, wherein R⁸ is hydrogen.

In some embodiments, Compounds of the Disclosure are compounds of any one of Formulae I-VI, or a pharmaceutically acceptable salt or solvate thereof, wherein B¹ is selected from the group consisting of:

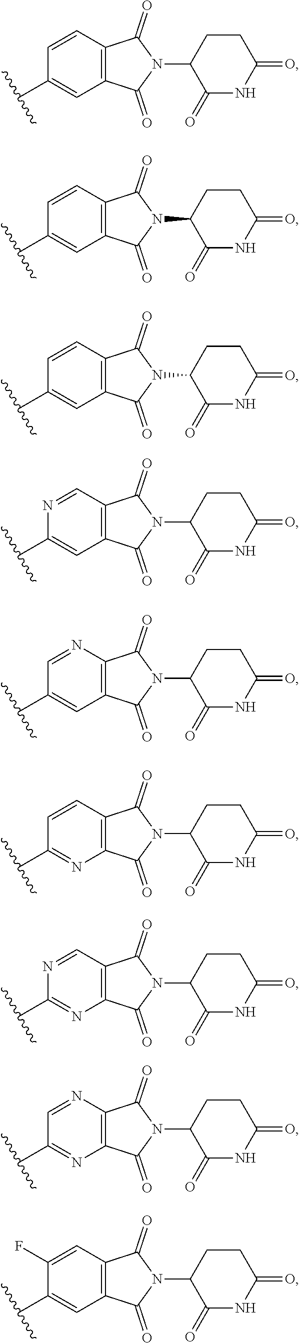

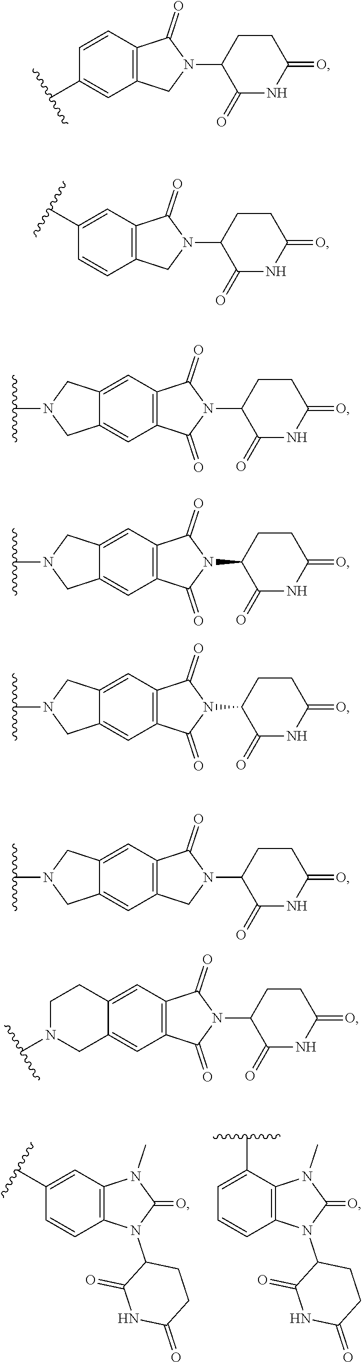

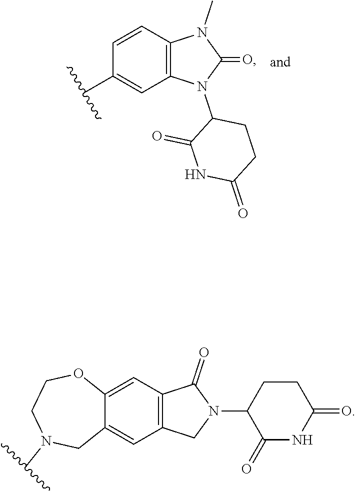 [SMILES: *c1ccc2c(c1)n(C1CCC(=O)NC1=O)c(=O)n2C ; *N1CCOc2cc3c(cc2C1)CN(C1CCC(=O)NC1=O)C3=O]

In some embodiments, Compounds of the Disclosure are compounds of any one of Formulae I-VI, or a pharmaceutically acceptable salt or solvate thereof wherein B¹ is:

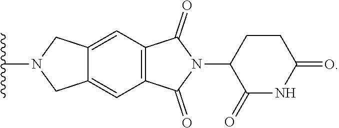 [SMILES: *N1Cc2cc3c(cc2C1)C(=O)N(C1CCC(=O)NC1=O)C3=O]

In some embodiments, Compounds of the Disclosure are compounds of any one of Formulae I-VI, or a pharmaceutically acceptable salt or solvate thereof, wherein B¹ is:

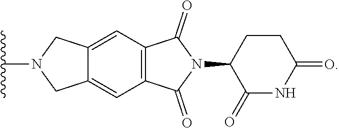 [SMILES: *N1Cc2cc3c(cc2C1)C(=O)N([C@H]1CCC(=O)NC1=O)C3=O]

In some embodiments, Compounds of the Disclosure are compounds of any one of Formulae I-VI, or a pharmaceutically acceptable salt or solvate thereof, wherein B¹ is:

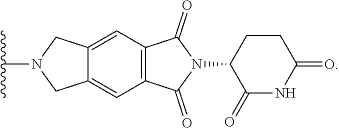 [SMILES: *N1Cc2cc3c(cc2C1)C(=O)N([C@@H]1CCC(=O)NC1=O)C3=O]

In some embodiments, Compounds of the Disclosure are compounds of any one of Formulae I-VI, or a pharmaceutically acceptable salt or solvate thereof, wherein B¹ is:

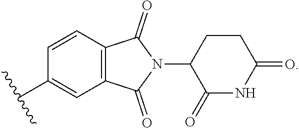 [SMILES: *c1ccc2c(c1)C(=O)N(C1CCC(=O)NC1=O)C2=O]

In some embodiments, Compounds of the Disclosure are compounds of any one of Formulae I-VI, or a pharmaceutically acceptable salt or solvate thereof, wherein B¹ is:

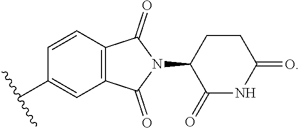 [SMILES: *c1ccc2c(c1)C(=O)N([C@H]1CCC(=O)NC1=O)C2=O]

In some embodiments, Compounds of the Disclosure are compounds of any one of Formulae I-VI, or a pharmaceutically acceptable salt or solvate thereof, wherein B¹ is:

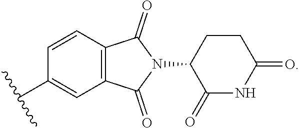 [SMILES: *c1ccc2c(c1)C(=O)N([C@@H]1CCC(=O)NC1=O)C2=O]

In some embodiments, Compounds of the Disclosure are selected from the compounds of Table 1, and pharmaceutically acceptable salts and solvates thereof.

In some embodiments, Compounds of the Disclosure are selected from the compounds of Table 1, and pharmaceutically acceptable salts thereof.

In some embodiments, Compounds of the Disclosure are selected from the compounds of Table 1.

TABLE 1

| Cpd. No. | Structure |
|---|---|
| 1 | 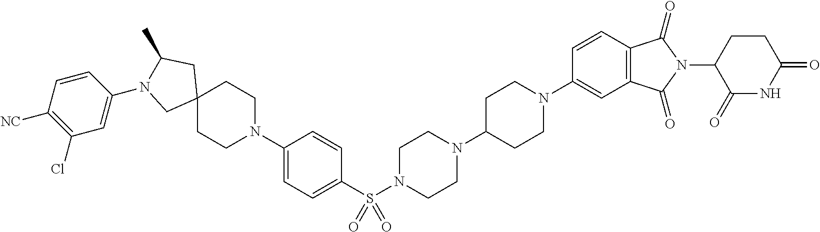 [SMILES: C[C@H]1CC2(CCN(c3ccc(S(=O)(=O)N4CCN(C5CCN(c6ccc7c(c6)C(=O)N(C6CCC(=O)NC6=O)C7=O)CC5)CC4)cc3)CC2)CN1c1ccc(C#N)c(Cl)c1] |
| 2 | 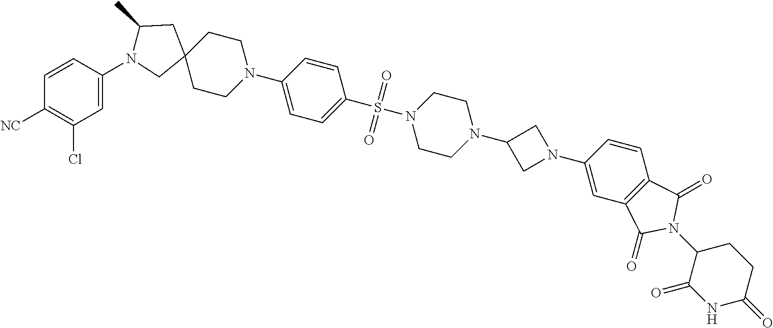 [SMILES: C[C@H]1CC2(CCN(c3ccc(S(=O)(=O)N4CCN(C5CN(c6ccc7c(c6)C(=O)N(C6CCC(=O)NC6=O)C7=O)C5)CC4)cc3)CC2)CN1c1ccc(C#N)c(Cl)c1] |
| 3 | 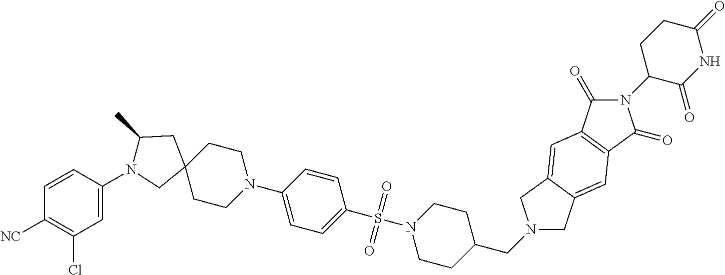 [SMILES: C[C@H]1CC2(CCN(c3ccc(S(=O)(=O)N4CCC(CN5Cc6cc7c(cc6C5)C(=O)N(C5CCC(=O)NC5=O)C7=O)CC4)cc3)CC2)CN1c1ccc(C#N)c(Cl)c1] |
| 4 | 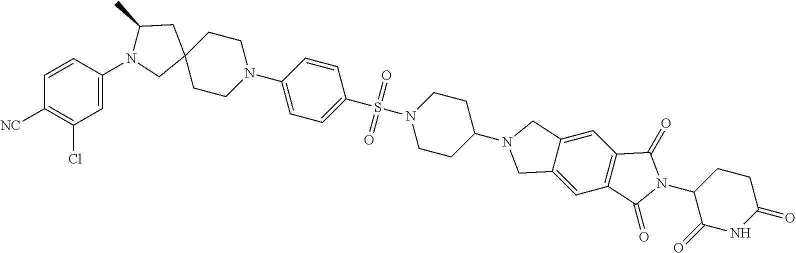 [SMILES: C[C@H]1CC2(CCN(c3ccc(S(=O)(=O)N4CCC(N5Cc6cc7c(cc6C5)C(=O)N(C5CCC(=O)NC5=O)C7=O)CC4)cc3)CC2)CN1c1ccc(C#N)c(Cl)c1] |
| 5 | 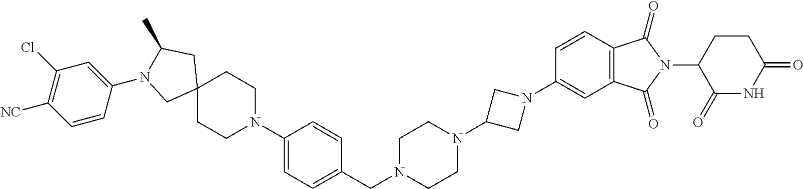 [SMILES: C[C@H]1CC2(CCN(c3ccc(CN4CCN(C5CN(c6ccc7c(c6)C(=O)N(C6CCC(=O)NC6=O)C7=O)C5)CC4)cc3)CC2)CN1c1ccc(C#N)c(Cl)c1] |
| 6 | 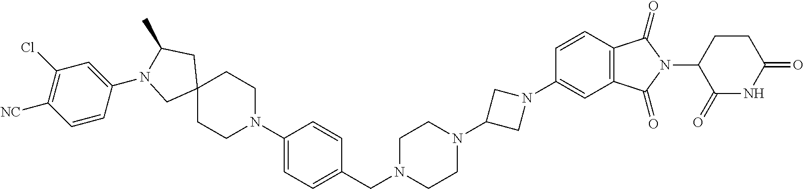 [SMILES: C[C@H]1CC2(CCN(c3ccc(CN4CCN(C5CN(c6ccc7c(c6)C(=O)N(C6CCC(=O)NC6=O)C7=O)C5)CC4)cc3)CC2)CN1c1ccc(C#N)c(Cl)c1] |
| 7 | 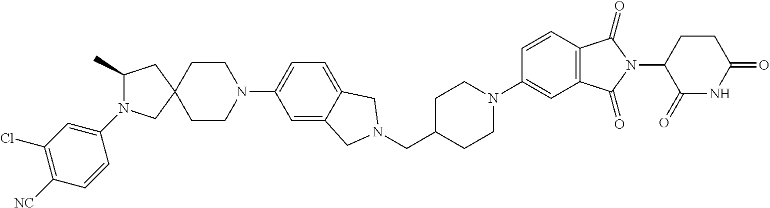 [SMILES: C[C@H]1CC2(CCN(c3ccc4c(c3)CN(CC3CCN(c5ccc6c(c5)C(=O)N(C5CCC(=O)NC5=O)C6=O)CC3)C4)CC2)CN1c1ccc(C#N)c(Cl)c1] |
| 8 | 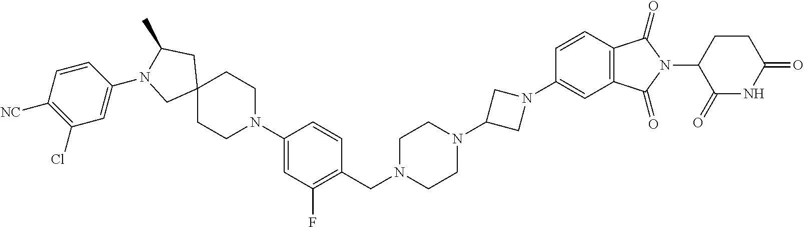 [SMILES: C[C@H]1CC2(CCN(c3ccc(CN4CCN(C5CN(c6ccc7c(c6)C(=O)N(C6CCC(=O)NC6=O)C7=O)C5)CC4)c(F)c3)CC2)CN1c1ccc(C#N)c(Cl)c1] |
| 9 | 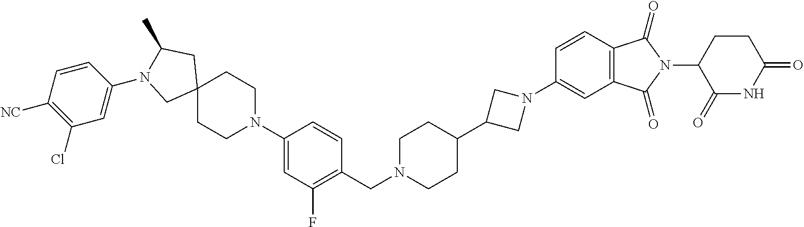 [SMILES: C[C@H]1CC2(CCN(c3ccc(CN4CCC(C5CN(c6ccc7c(c6)C(=O)N(C6CCC(=O)NC6=O)C7=O)C5)CC4)c(F)c3)CC2)CN1c1ccc(C#N)c(Cl)c1] |
| 10 | 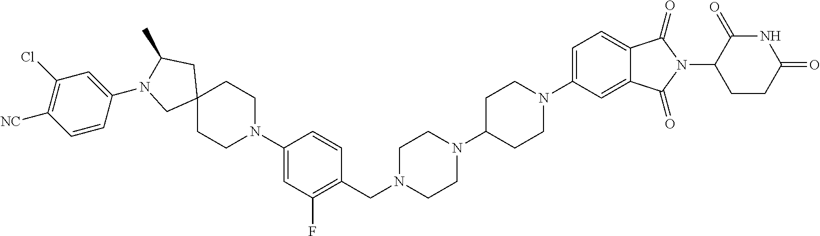 [SMILES: C[C@H]1CC2(CCN(c3ccc(CN4CCN(C5CCN(c6ccc7c(c6)C(=O)N(C6CCC(=O)NC6=O)C7=O)CC5)CC4)c(F)c3)CC2)CN1c1ccc(C#N)c(Cl)c1] |
| 11 | 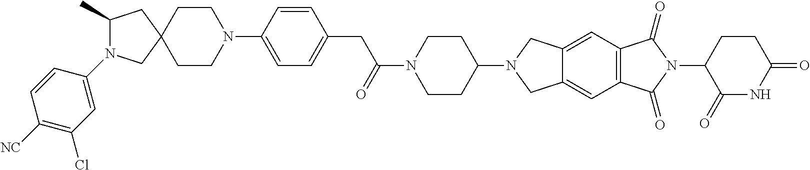 [SMILES: C[C@H]1CC2(CCN(c3ccc(CC(=O)N4CCC(N5Cc6cc7c(cc6C5)C(=O)N(C5CCC(=O)NC5=O)C7=O)CC4)cc3)CC2)CN1c1ccc(C#N)c(Cl)c1] |
| 12 | 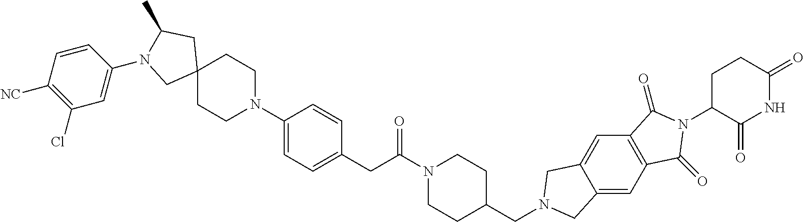 [SMILES: C[C@H]1CC2(CCN(c3ccc(CC(=O)N4CCC(CN5Cc6cc7c(cc6C5)C(=O)N(C5CCC(=O)NC5=O)C7=O)CC4)cc3)CC2)CN1c1ccc(C#N)c(Cl)c1] |
| 13 | 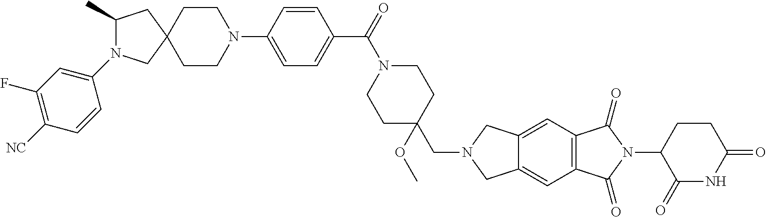 [SMILES: COC1(CN2Cc3cc4c(cc3C2)C(=O)N(C2CCC(=O)NC2=O)C4=O)CCN(C(=O)c2ccc(N3CCC4(CC3)C[C@H](C)N(c3ccc(C#N)c(F)c3)C4)cc2)CC1] |
| 14 | 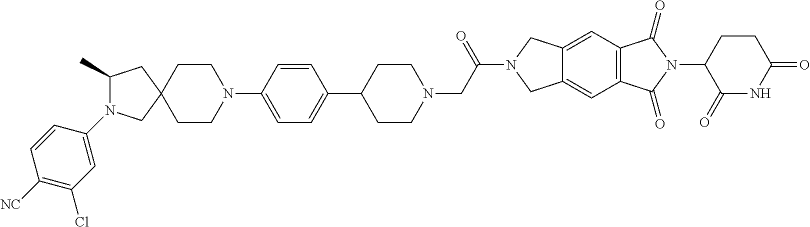 |
| 15 | 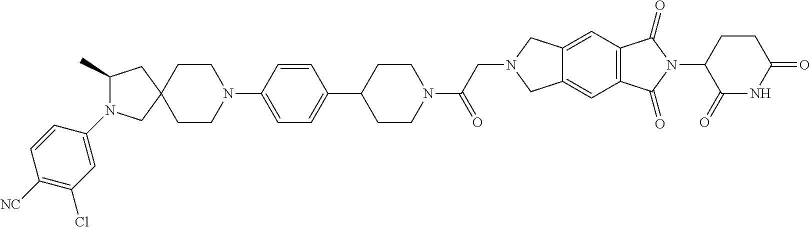 [SMILES: C[C@H]1CC2(CCN(c3ccc(C4CCN(C(=O)CN5Cc6cc7c(cc6C5)C(=O)N(C5CCC(=O)NC5=O)C7=O)CC4)cc3)CC2)CN1c1ccc(C#N)c(Cl)c1] |
| 16 | 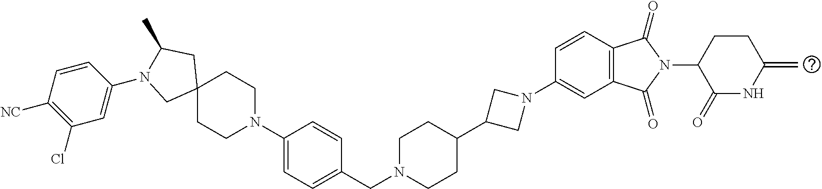 [SMILES: C=C1CCC(N2C(=O)c3ccc(N4CC(C5CCN(Cc6ccc(N7CCC8(CC7)C[C@H](C)N(c7ccc(C#N)c(Cl)c7)C8)cc6)CC5)C4)cc3C2=O)C(=O)N1] |
| 17 | 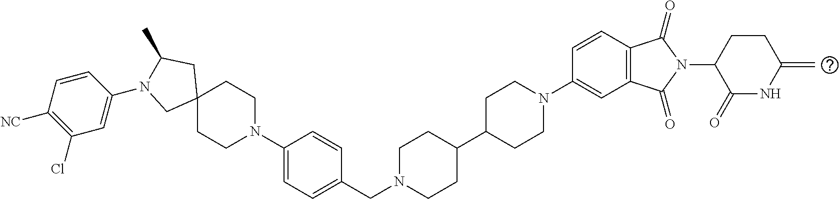 [SMILES: C=C1CCC(N2C(=O)c3ccc(N4CCC(C5CCN(Cc6ccc(N7CCC8(CC7)C[C@H](C)N(c7ccc(C#N)c(Cl)c7)C8)cc6)CC5)CC4)cc3C2=O)C(=O)N1] |
| 18 | 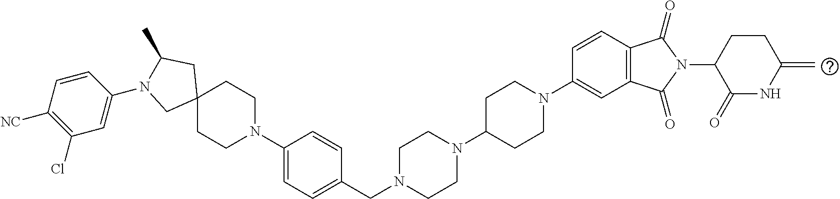 [SMILES: C=C1CCC(N2C(=O)c3ccc(N4CCC(N5CCN(Cc6ccc(N7CCC8(CC7)C[C@H](C)N(c7ccc(C#N)c(Cl)c7)C8)cc6)CC5)CC4)cc3C2=O)C(=O)N1] |
| 19 | 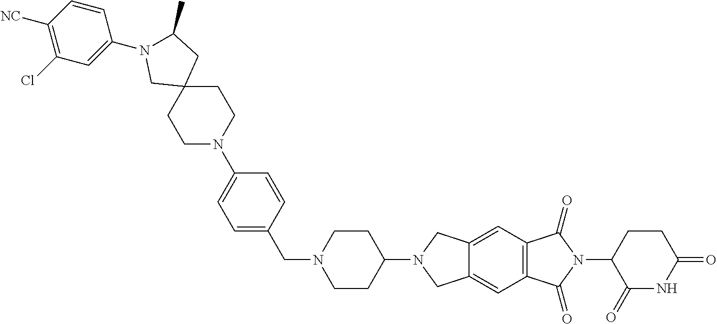 [SMILES: C[C@H]1CC2(CCN(c3ccc(CN4CCC(N5Cc6cc7c(cc6C5)C(=O)N(C5CCC(=O)NC5=O)C7=O)CC4)cc3)CC2)CN1c1ccc(C#N)c(Cl)c1] |
| 20 | 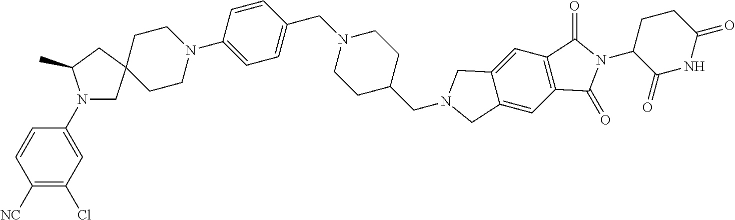 [SMILES: C[C@H]1CC2(CCN(c3ccc(CN4CCC(CN5Cc6cc7c(cc6C5)C(=O)N(C5CCC(=O)NC5=O)C7=O)CC4)cc3)CC2)CN1c1ccc(C#N)c(Cl)c1] |
| 21 | 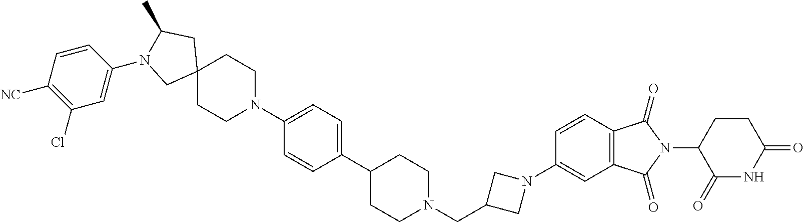 [SMILES: C[C@H]1CC2(CCN(c3ccc(C4CCN(CC5CN(c6ccc7c(c6)C(=O)N(C6CCC(=O)NC6=O)C7=O)C5)CC4)cc3)CC2)CN1c1ccc(C#N)c(Cl)c1] |
| 22 | 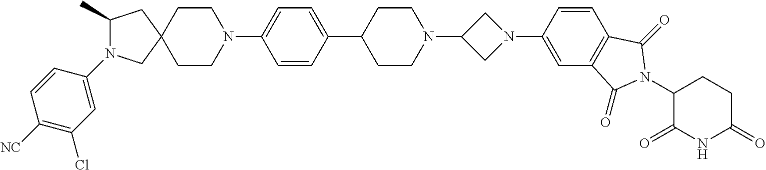 [SMILES: C[C@H]1CC2(CCN(c3ccc(C4CCN(C5CN(c6ccc7c(c6)C(=O)N(C6CCC(=O)NC6=O)C7=O)C5)CC4)cc3)CC2)CN1c1ccc(C#N)c(Cl)c1] |
| 23 | 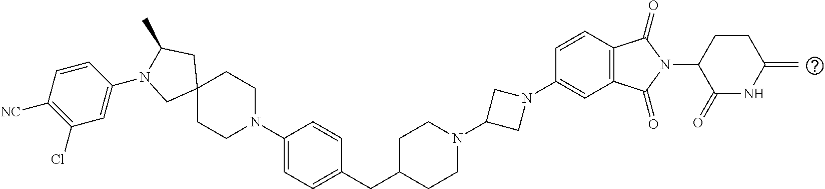 [SMILES: C=C1CCC(N2C(=O)c3ccc(N4CC(N5CCC(Cc6ccc(N7CCC8(CC7)C[C@H](C)N(c7ccc(C#N)c(Cl)c7)C8)cc6)CC5)C4)cc3C2=O)C(=O)N1] |
| 24 | 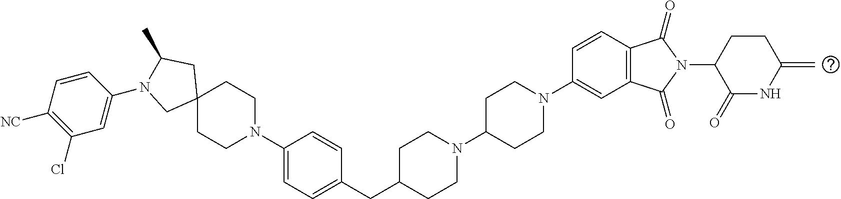 [SMILES: C=C1CCC(N2C(=O)c3ccc(N4CCC(N5CCC(Cc6ccc(N7CCC8(CC7)C[C@H](C)N(c7ccc(C#N)c(Cl)c7)C8)cc6)CC5)CC4)cc3C2=O)C(=O)N1] |
| 25 | 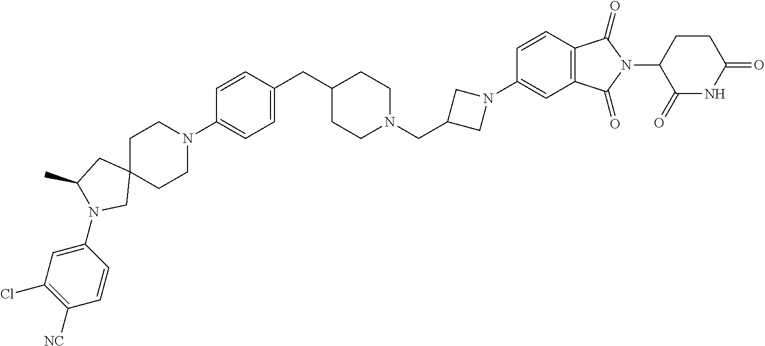 [SMILES: C[C@H]1CC2(CCN(c3ccc(CC4CCN(CC5CN(c6ccc7c(c6)C(=O)N(C6CCC(=O)NC6=O)C7=O)C5)CC4)cc3)CC2)CN1c1ccc(C#N)c(Cl)c1] |
| 26 | 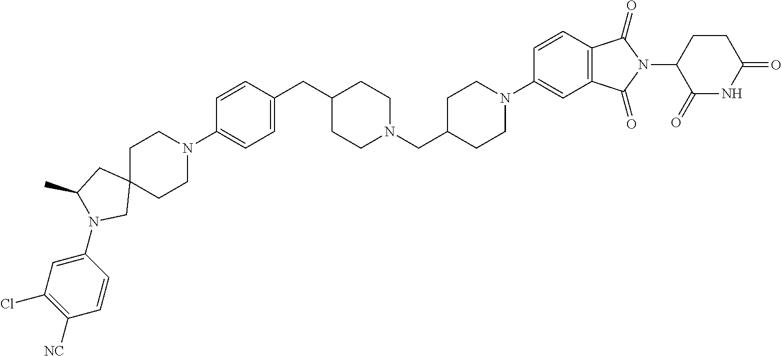 [SMILES: C[C@H]1CC2(CCN(c3ccc(CC4CCN(CC5CCN(c6ccc7c(c6)C(=O)N(C6CCC(=O)NC6=O)C7=O)CC5)CC4)cc3)CC2)CN1c1ccc(C#N)c(Cl)c1] |
| 27 | 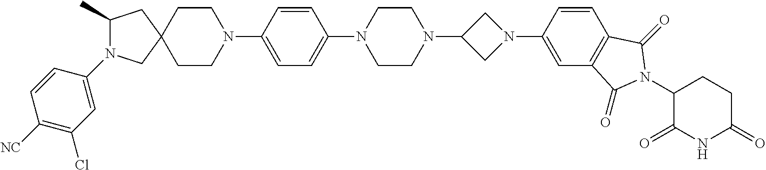 [SMILES: C[C@H]1CC2(CCN(c3ccc(N4CCN(C5CN(c6ccc7c(c6)C(=O)N(C6CCC(=O)NC6=O)C7=O)C5)CC4)cc3)CC2)CN1c1ccc(C#N)c(Cl)c1] |
| 28 | 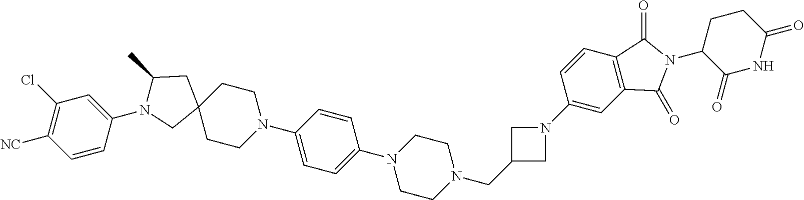 [SMILES: C[C@H]1CC2(CCN(c3ccc(N4CCN(CC5CN(c6ccc7c(c6)C(=O)N(C6CCC(=O)NC6=O)C7=O)C5)CC4)cc3)CC2)CN1c1ccc(C#N)c(Cl)c1] |
| 29 | 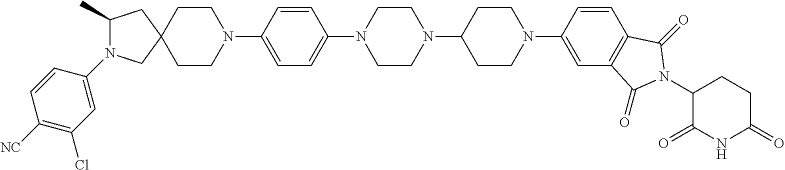 [SMILES: C[C@H]1CC2(CCN(c3ccc(N4CCN(C5CCN(c6ccc7c(c6)C(=O)N(C6CCC(=O)NC6=O)C7=O)CC5)CC4)cc3)CC2)CN1c1ccc(C#N)c(Cl)c1] |
| 30 | 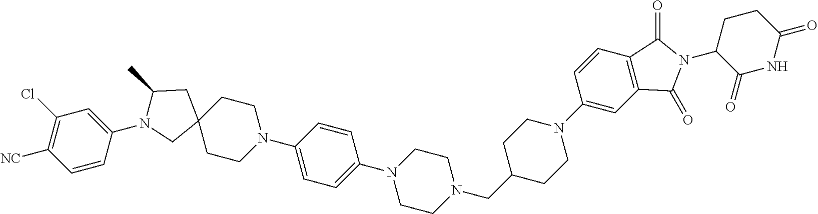 [SMILES: C[C@H]1CC2(CCN(c3ccc(N4CCN(CC5CCN(c6ccc7c(c6)C(=O)N(C6CCC(=O)NC6=O)C7=O)CC5)CC4)cc3)CC2)CN1c1ccc(C#N)c(Cl)c1] |
| 31 | 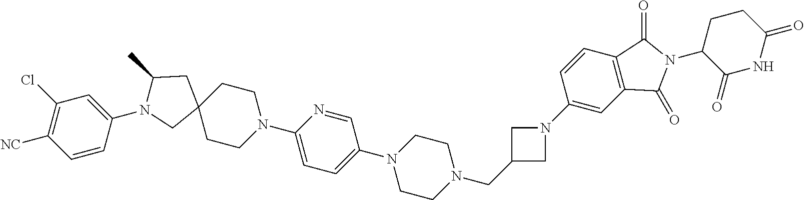 [SMILES: C[C@H]1CC2(CCN(c3ccc(N4CCN(CC5CN(c6ccc7c(c6)C(=O)N(C6CCC(=O)NC6=O)C7=O)C5)CC4)cn3)CC2)CN1c1ccc(C#N)c(Cl)c1] |
| 32 | 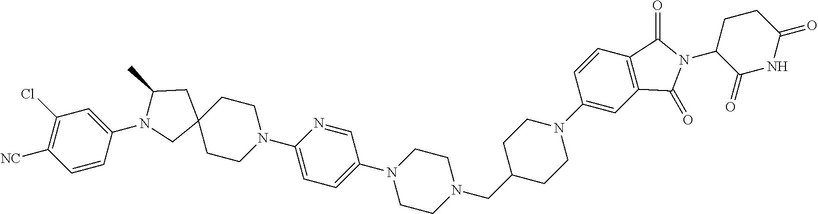 [SMILES: C[C@H]1CC2(CCN(c3ccc(N4CCN(CC5CCN(c6ccc7c(c6)C(=O)N(C6CCC(=O)NC6=O)C7=O)CC5)CC4)cn3)CC2)CN1c1ccc(C#N)c(Cl)c1] |
| 33 | 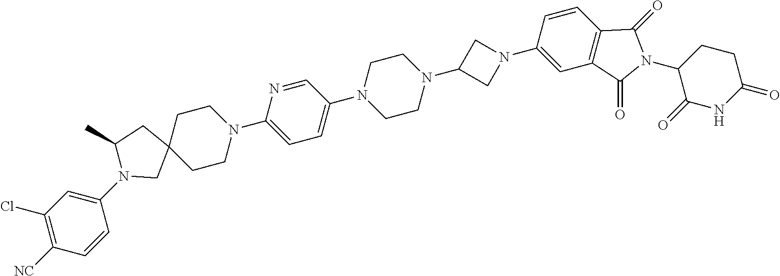 [SMILES: C[C@H]1CC2(CCN(c3ccc(N4CCN(C5CN(c6ccc7c(c6)C(=O)N(C6CCC(=O)NC6=O)C7=O)C5)CC4)cn3)CC2)CN1c1ccc(C#N)c(Cl)c1] |
| 34 | 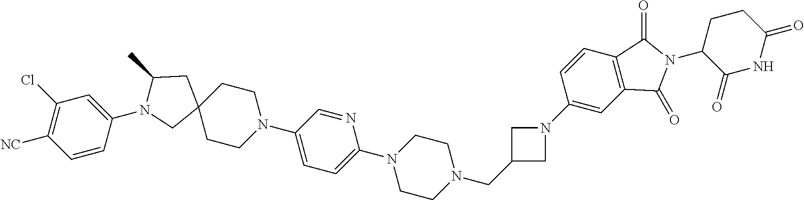 [SMILES: C[C@H]1CC2(CCN(c3ccc(N4CCN(CC5CN(c6ccc7c(c6)C(=O)N(C6CCC(=O)NC6=O)C7=O)C5)CC4)nc3)CC2)CN1c1ccc(C#N)c(Cl)c1] |
| 35 | 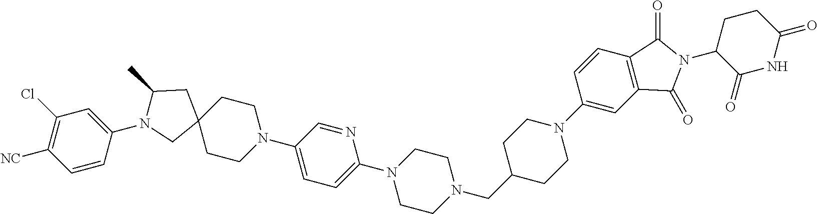 [SMILES: C[C@H]1CC2(CCN(c3ccc(N4CCN(CC5CCN(c6ccc7c(c6)C(=O)N(C6CCC(=O)NC6=O)C7=O)CC5)CC4)nc3)CC2)CN1c1ccc(C#N)c(Cl)c1] |
| 36 | 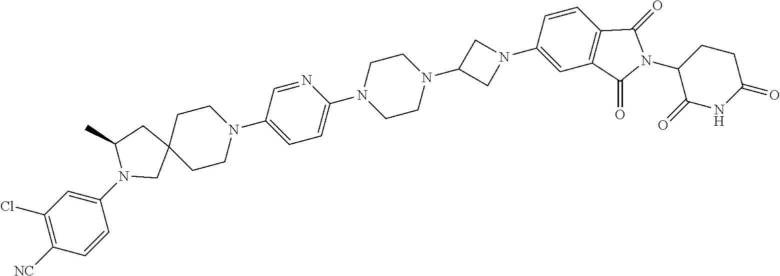 [SMILES: C[C@H]1CC2(CCN(c3ccc(N4CCN(C5CN(c6ccc7c(c6)C(=O)N(C6CCC(=O)NC6=O)C7=O)C5)CC4)nc3)CC2)CN1c1ccc(C#N)c(Cl)c1] |
| 37 | 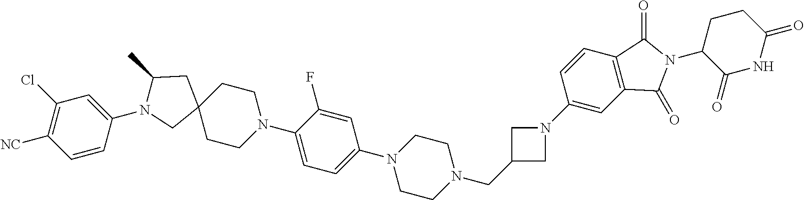 [SMILES: C[C@H]1CC2(CCN(c3ccc(N4CCN(CC5CN(c6ccc7c(c6)C(=O)N(C6CCC(=O)NC6=O)C7=O)C5)CC4)cc3F)CC2)CN1c1ccc(C#N)c(Cl)c1] |
| 38 | 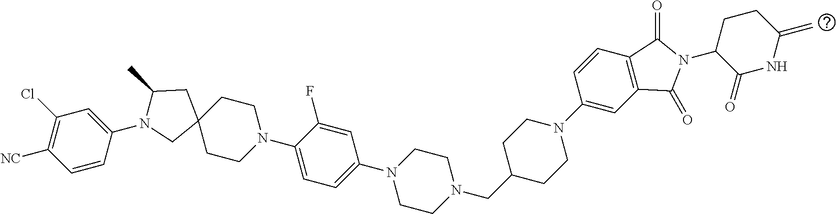 [SMILES: C=C1CCC(N2C(=O)c3ccc(N4CCC(CN5CCN(c6ccc(N7CCC8(CC7)C[C@H](C)N(c7ccc(C#N)c(Cl)c7)C8)c(F)c6)CC5)CC4)cc3C2=O)C(=O)N1] |
| 39 | 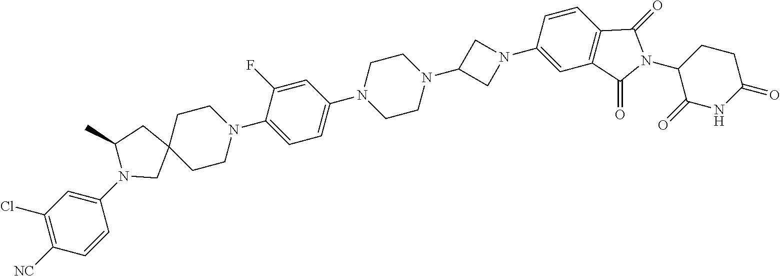 [SMILES: C[C@H]1CC2(CCN(c3ccc(N4CCN(C5CN(c6ccc7c(c6)C(=O)N(C6CCC(=O)NC6=O)C7=O)C5)CC4)cc3F)CC2)CN1c1ccc(C#N)c(Cl)c1] |
| 40 | 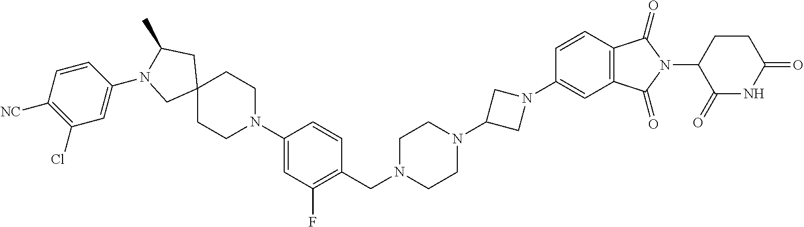 [SMILES: C[C@H]1CC2(CCN(c3ccc(CN4CCN(C5CN(c6ccc7c(c6)C(=O)N(C6CCC(=O)NC6=O)C7=O)C5)CC4)c(F)c3)CC2)CN1c1ccc(C#N)c(Cl)c1] |
| 41 | 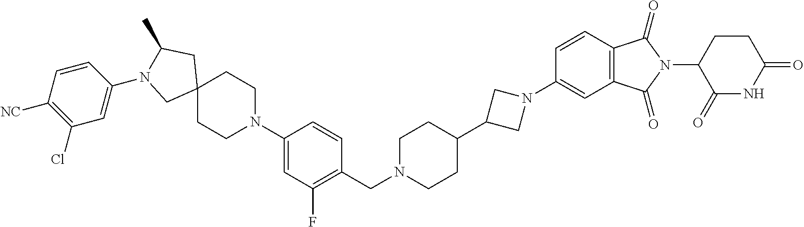 [SMILES: C[C@H]1CC2(CCN(c3ccc(CN4CCC(C5CN(c6ccc7c(c6)C(=O)N(C6CCC(=O)NC6=O)C7=O)C5)CC4)c(F)c3)CC2)CN1c1ccc(C#N)c(Cl)c1] |
| 42 | 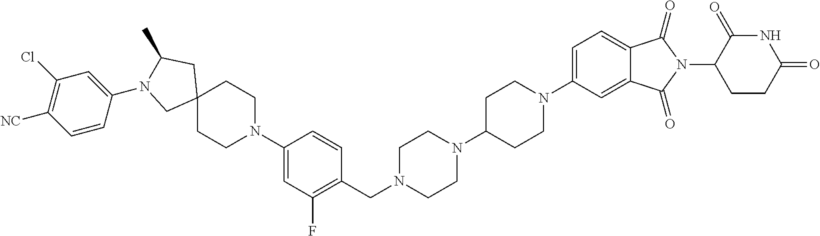 [SMILES: C[C@H]1CC2(CCN(c3ccc(CN4CCN(C5CCN(c6ccc7c(c6)C(=O)N(C6CCC(=O)NC6=O)C7=O)CC5)CC4)c(F)c3)CC2)CN1c1ccc(C#N)c(Cl)c1] |
| 43 | 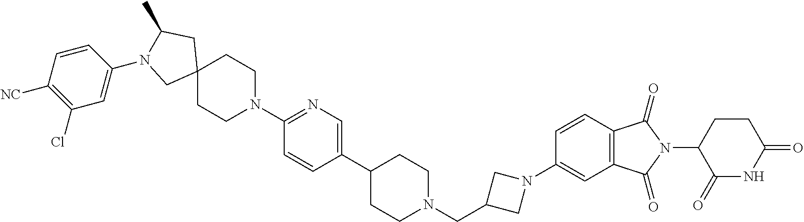 [SMILES: C[C@H]1CC2(CCN(c3ccc(C4CCN(CC5CN(c6ccc7c(c6)C(=O)N(C6CCC(=O)NC6=O)C7=O)C5)CC4)cn3)CC2)CN1c1ccc(C#N)c(Cl)c1] |
| 44 | 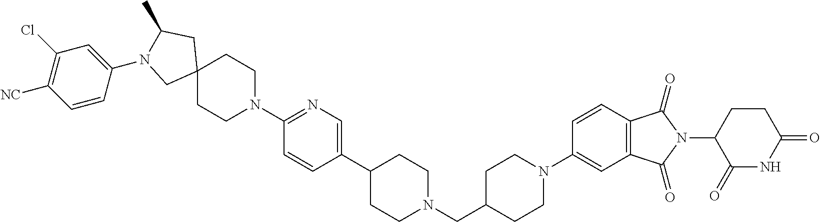 [SMILES: C[C@H]1CC2(CCN(c3ccc(C4CCN(CC5CCN(c6ccc7c(c6)C(=O)N(C6CCC(=O)NC6=O)C7=O)CC5)CC4)cn3)CC2)CN1c1ccc(C#N)c(Cl)c1] |
| 45 | 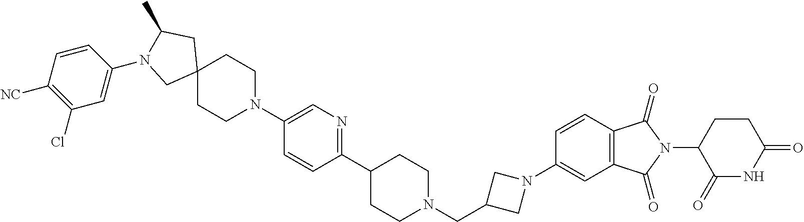 [SMILES: C[C@H]1CC2(CCN(c3ccc(C4CCN(CC5CN(c6ccc7c(c6)C(=O)N(C6CCC(=O)NC6=O)C7=O)C5)CC4)nc3)CC2)CN1c1ccc(C#N)c(Cl)c1] |
| 46 | 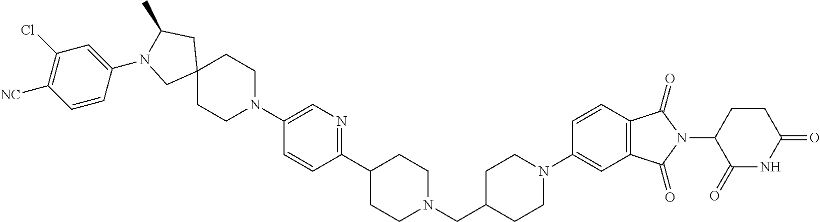 [SMILES: C[C@H]1CC2(CCN(c3ccc(C4CCN(CC5CCN(c6ccc7c(c6)C(=O)N(C6CCC(=O)NC6=O)C7=O)CC5)CC4)nc3)CC2)CN1c1ccc(C#N)c(Cl)c1] |
| 47 | 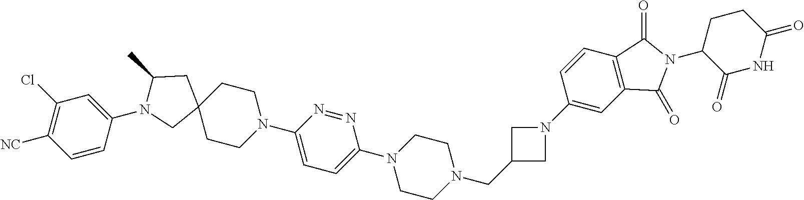 [SMILES: C[C@H]1CC2(CCN(c3ccc(N4CCN(CC5CN(c6ccc7c(c6)C(=O)N(C6CCC(=O)NC6=O)C7=O)C5)CC4)nn3)CC2)CN1c1ccc(C#N)c(Cl)c1] |
| 48 | 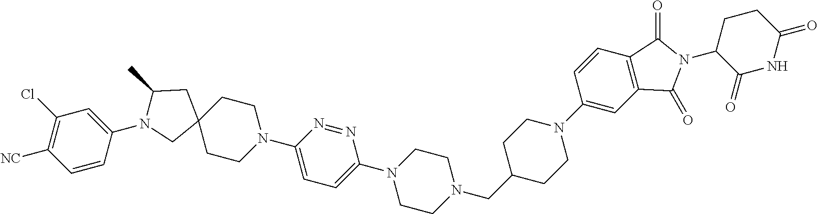 [SMILES: C[C@H]1CC2(CCN(c3ccc(N4CCN(CC5CCN(c6ccc7c(c6)C(=O)N(C6CCC(=O)NC6=O)C7=O)CC5)CC4)nn3)CC2)CN1c1ccc(C#N)c(Cl)c1] |
| 49 | 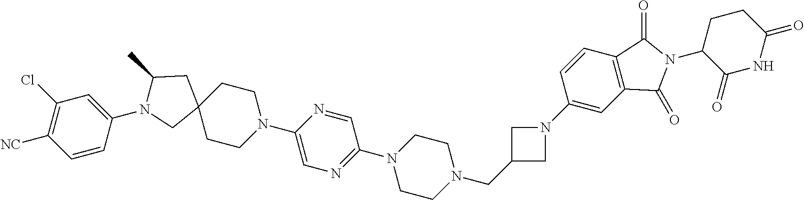 [SMILES: C[C@H]1CC2(CCN(c3cnc(N4CCN(CC5CN(c6ccc7c(c6)C(=O)N(C6CCC(=O)NC6=O)C7=O)C5)CC4)cn3)CC2)CN1c1ccc(C#N)c(Cl)c1] |
| 50 | 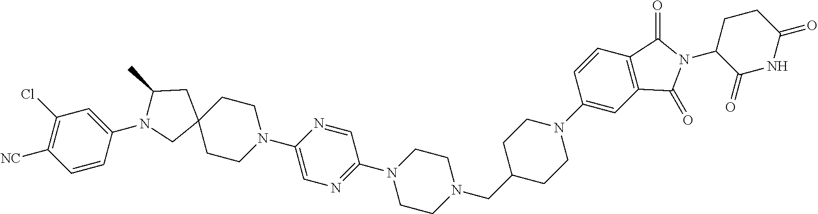 [SMILES: C[C@H]1CC2(CCN(c3cnc(N4CCN(CC5CCN(c6ccc7c(c6)C(=O)N(C6CCC(=O)NC6=O)C7=O)CC5)CC4)cn3)CC2)CN1c1ccc(C#N)c(Cl)c1] |
| 51 | 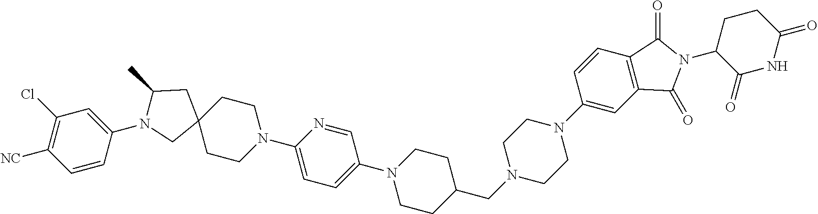 [SMILES: C[C@H]1CC2(CCN(c3ccc(N4CCC(CN5CCN(c6ccc7c(c6)C(=O)N(C6CCC(=O)NC6=O)C7=O)CC5)CC4)cn3)CC2)CN1c1ccc(C#N)c(Cl)c1] |
| 52 | 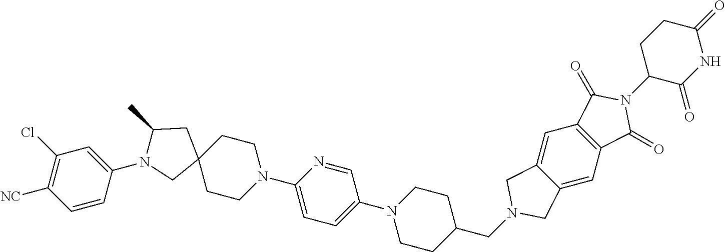 [SMILES: C[C@H]1CC2(CCN(c3ccc(N4CCC(CN5Cc6cc7c(cc6C5)C(=O)N(C5CCC(=O)NC5=O)C7=O)CC4)cn3)CC2)CN1c1ccc(C#N)c(Cl)c1] |
| 53 | 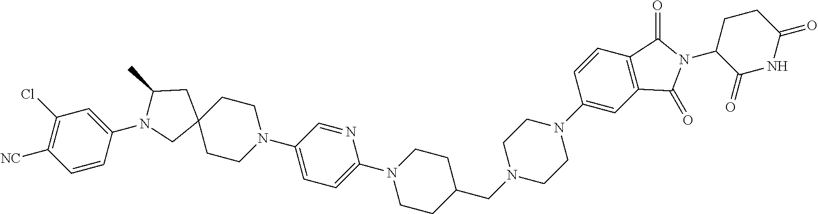 [SMILES: C[C@H]1CC2(CCN(c3ccc(N4CCC(CN5CCN(c6ccc7c(c6)C(=O)N(C6CCC(=O)NC6=O)C7=O)CC5)CC4)nc3)CC2)CN1c1ccc(C#N)c(Cl)c1] |
| 54 | 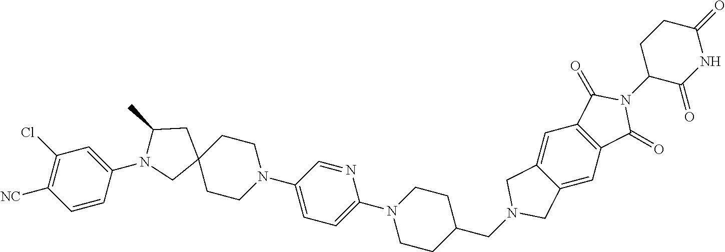 [SMILES: C[C@H]1CC2(CCN(c3ccc(N4CCC(CN5Cc6cc7c(cc6C5)C(=O)N(C5CCC(=O)NC5=O)C7=O)CC4)nc3)CC2)CN1c1ccc(C#N)c(Cl)c1] |
| 55 | 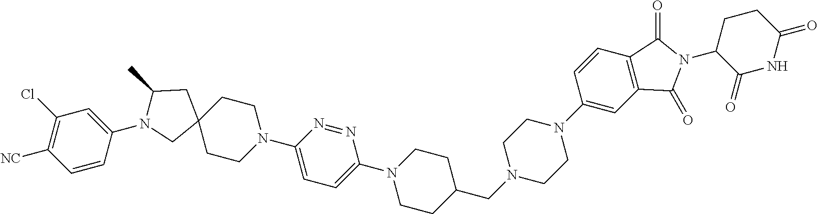 [SMILES: C[C@H]1CC2(CCN(c3ccc(N4CCC(CN5CCN(c6ccc7c(c6)C(=O)N(C6CCC(=O)NC6=O)C7=O)CC5)CC4)nn3)CC2)CN1c1ccc(C#N)c(Cl)c1] |
| 56 | 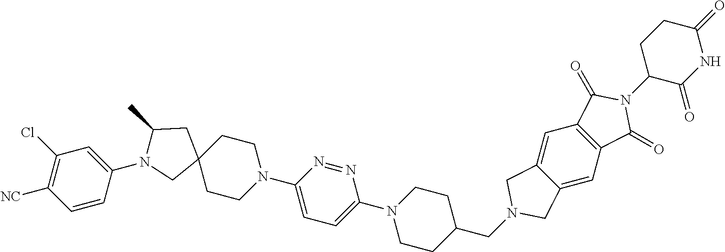 [SMILES: C[C@H]1CC2(CCN(c3ccc(N4CCC(CN5Cc6cc7c(cc6C5)C(=O)N(C5CCC(=O)NC5=O)C7=O)CC4)nn3)CC2)CN1c1ccc(C#N)c(Cl)c1] |
| 57 | 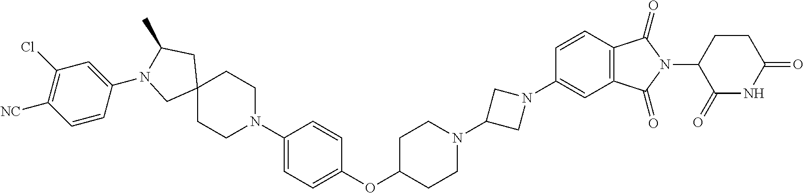 [SMILES: C[C@H]1CC2(CCN(c3ccc(OC4CCN(C5CN(c6ccc7c(c6)C(=O)N(C6CCC(=O)NC6=O)C7=O)C5)CC4)cc3)CC2)CN1c1ccc(C#N)c(Cl)c1] |
| 58 | 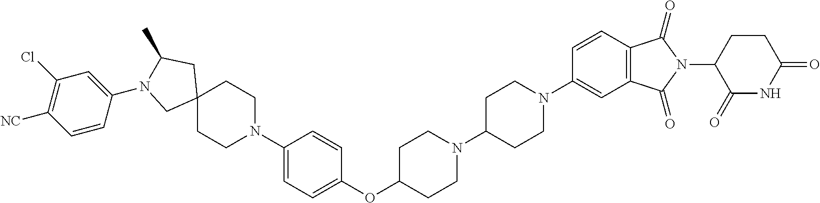 [SMILES: C[C@H]1CC2(CCN(c3ccc(OC4CCN(C5CCN(c6ccc7c(c6)C(=O)N(C6CCC(=O)NC6=O)C7=O)CC5)CC4)cc3)CC2)CN1c1ccc(C#N)c(Cl)c1] |
| 59 | 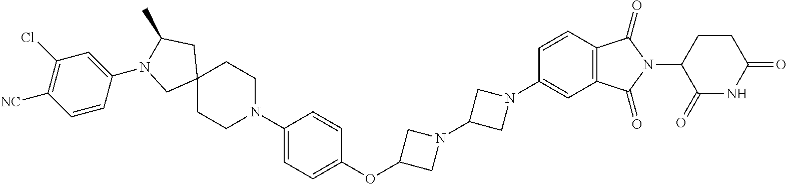 [SMILES: C[C@H]1CC2(CCN(c3ccc(OC4CN(C5CN(c6ccc7c(c6)C(=O)N(C6CCC(=O)NC6=O)C7=O)C5)C4)cc3)CC2)CN1c1ccc(C#N)c(Cl)c1] |
| 60 | 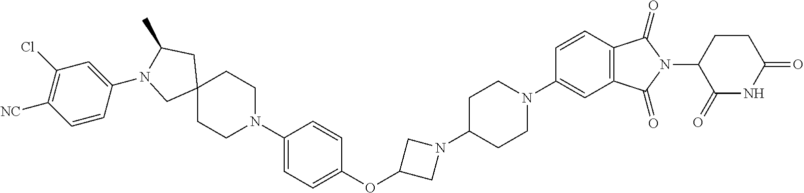 [SMILES: C[C@H]1CC2(CCN(c3ccc(OC4CN(C5CCN(c6ccc7c(c6)C(=O)N(C6CCC(=O)NC6=O)C7=O)CC5)C4)cc3)CC2)CN1c1ccc(C#N)c(Cl)c1] |
| 61 | 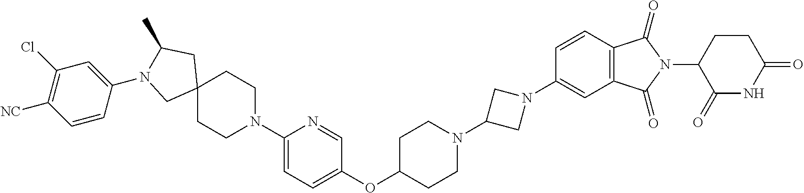 [SMILES: C[C@H]1CC2(CCN(c3ccc(OC4CCN(C5CN(c6ccc7c(c6)C(=O)N(C6CCC(=O)NC6=O)C7=O)C5)CC4)cn3)CC2)CN1c1ccc(C#N)c(Cl)c1] |
| 62 | 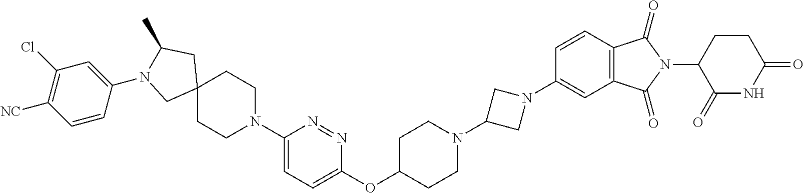 [SMILES: C[C@H]1CC2(CCN(c3ccc(OC4CCN(C5CN(c6ccc7c(c6)C(=O)N(C6CCC(=O)NC6=O)C7=O)C5)CC4)nn3)CC2)CN1c1ccc(C#N)c(Cl)c1] |
| 63 | 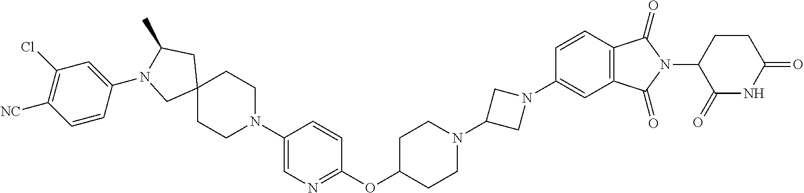 [SMILES: C[C@H]1CC2(CCN(c3ccc(OC4CCN(C5CN(c6ccc7c(c6)C(=O)N(C6CCC(=O)NC6=O)C7=O)C5)CC4)nc3)CC2)CN1c1ccc(C#N)c(Cl)c1] |
| 64 | 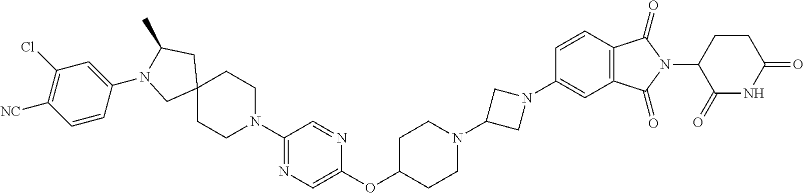 [SMILES: C[C@H]1CC2(CCN(c3cnc(OC4CCN(C5CN(c6ccc7c(c6)C(=O)N(C6CCC(=O)NC6=O)C7=O)C5)CC4)cn3)CC2)CN1c1ccc(C#N)c(Cl)c1] |
| 65 | 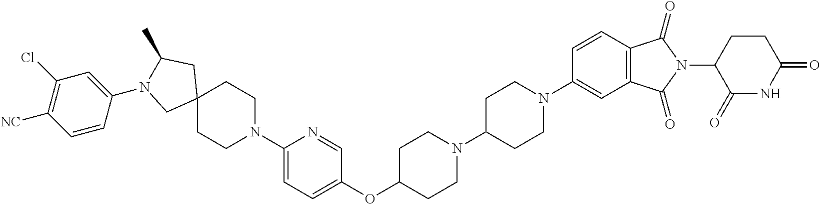 [SMILES: C[C@H]1CC2(CCN(c3ccc(OC4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| 66 | 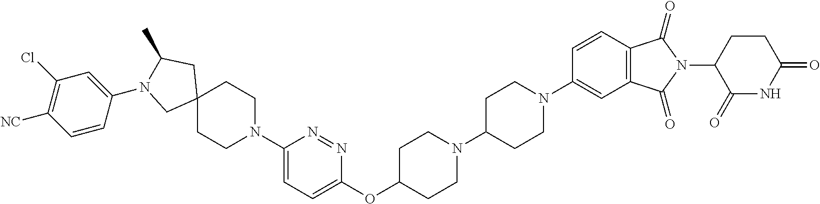 [SMILES: C[C@H]1CC2(CCN(c3ccc(OC4CCN(C5CCN(c6ccc7c(c6)C(=O)N(C6CCC(=O)NC6=O)C7=O)CC5)CC4)nn3)CC2)CN1c1ccc(C#N)c(Cl)c1] |
| 67 | 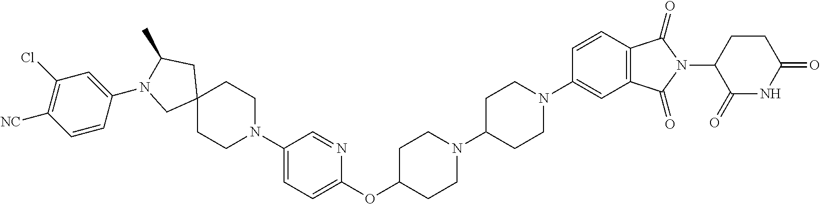 [SMILES: C[C@H]1CC2(CCN(c3ccc(OC4CCN(C5CCN(c6ccc7c(c6)C(=O)N(C6CCC(=O)NC6=O)C7=O)CC5)CC4)nc3)CC2)CN1c1ccc(C#N)c(Cl)c1] |
| 68 | 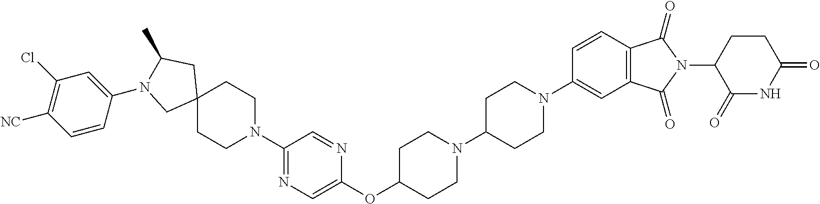 [SMILES: C[C@H]1CC2(CCN(c3cnc(OC4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| 69 | 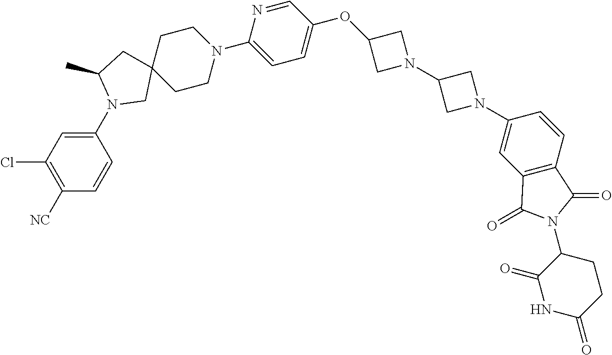 [SMILES: C[C@H]1CC2(CCN(c3ccc(OC4CN(C5CN(c6ccc7c(c6)C(=O)N(C6CCC(=O)NC6=O)C7=O)C5)C4)cn3)CC2)CN1c1ccc(C#N)c(Cl)c1] |
| 70 | 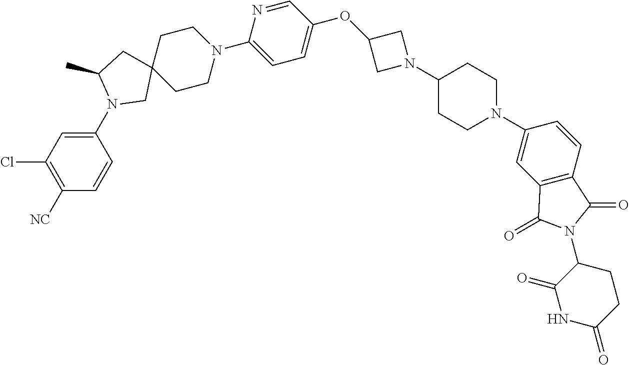 [SMILES: C[C@H]1CC2(CCN(c3ccc(OC4CN(C5CCN(c6ccc7c(c6)C(=O)N(C6CCC(=O)NC6=O)C7=O)CC5)C4)cn3)CC2)CN1c1ccc(C#N)c(Cl)c1] |
| 71 | 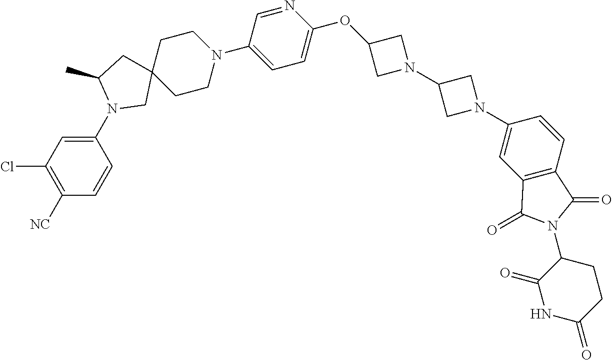 [SMILES: C[C@H]1CC2(CCN(c3ccc(OC4CN(C5CN(c6ccc7c(c6)C(=O)N(C6CCC(=O)NC6=O)C7=O)C5)C4)nc3)CC2)CN1c1ccc(C#N)c(Cl)c1] |
| 72 | 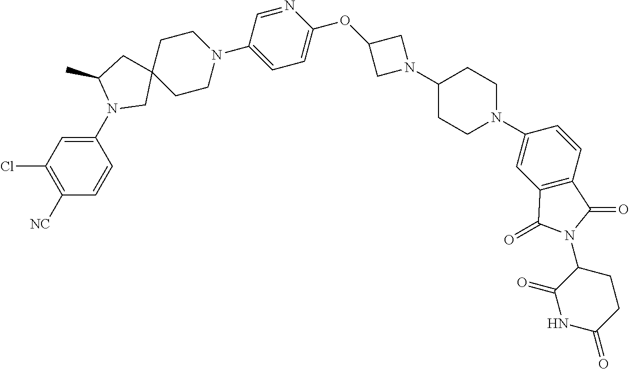 [SMILES: C[C@H]1CC2(CCN(c3ccc(OC4CN(C5CCN(c6ccc7c(c6)C(=O)N(C6CCC(=O)NC6=O)C7=O)CC5)C4)nc3)CC2)CN1c1ccc(C#N)c(Cl)c1] |
| 73 | 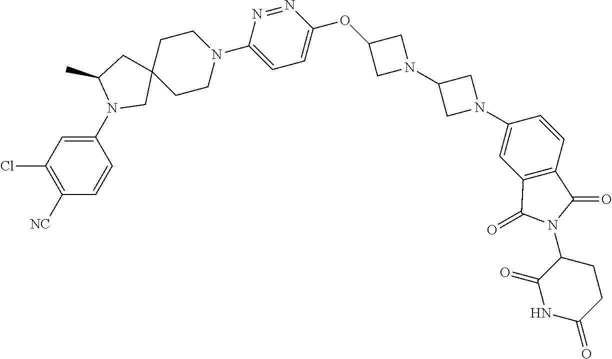 [SMILES: C[C@H]1CC2(CCN(c3ccc(OC4CN(C5CN(c6ccc7c(c6)C(=O)N(C6CCC(=O)NC6=O)C7=O)C5)C4)nn3)CC2)CN1c1ccc(C#N)c(Cl)c1] |
| 74 | 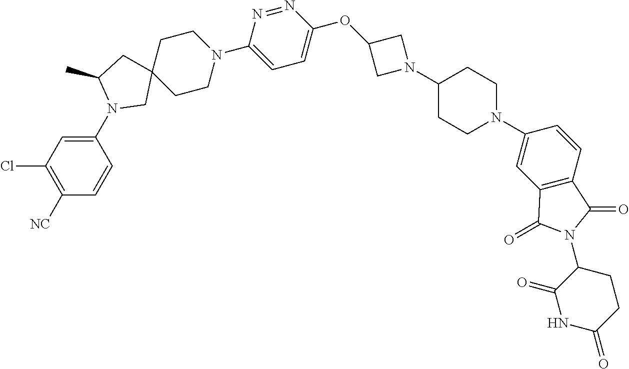 [SMILES: C[C@H]1CC2(CCN(c3ccc(OC4CN(C5CCN(c6ccc7c(c6)C(=O)N(C6CCC(=O)NC6=O)C7=O)CC5)C4)nn3)CC2)CN1c1ccc(C#N)c(Cl)c1] |
| 75 | 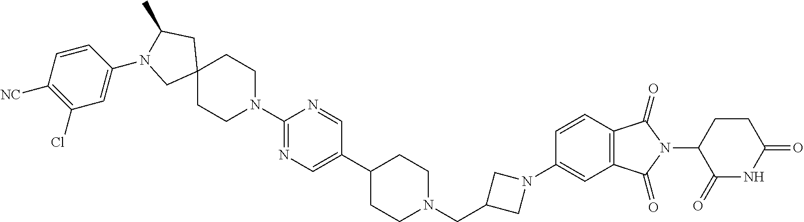 [SMILES: C[C@H]1CC2(CCN(c3ncc(C4CCN(CC5CN(c6ccc7c(c6)C(=O)N(C6CCC(=O)NC6=O)C7=O)C5)CC4)cn3)CC2)CN1c1ccc(C#N)c(Cl)c1] |
| 76 | 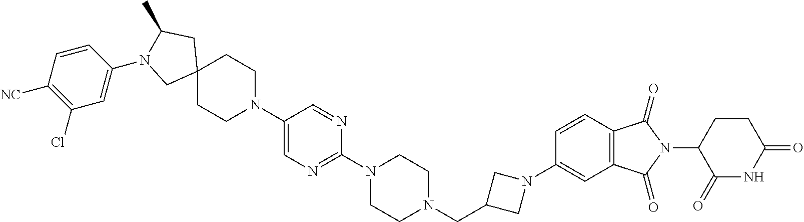 [SMILES: C[C@H]1CC2(CCN(c3cnc(N4CCN(CC5CN(c6ccc7c(c6)C(=O)N(C6CCC(=O)NC6=O)C7=O)C5)CC4)nc3)CC2)CN1c1ccc(C#N)c(Cl)c1] |
| 77 | 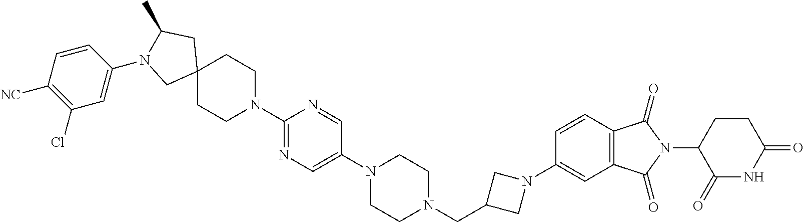 [SMILES: C[C@H]1CC2(CCN(c3ncc(N4CCN(CC5CN(c6ccc7c(c6)C(=O)N(C6CCC(=O)NC6=O)C7=O)C5)CC4)cn3)CC2)CN1c1ccc(C#N)c(Cl)c1] |
| 78 | 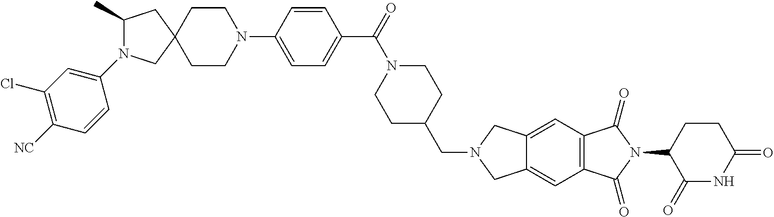 |
| 79 | 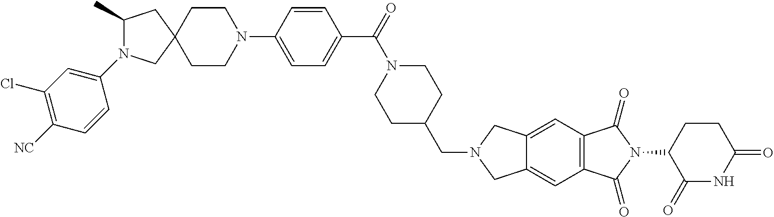 |
| 80 | 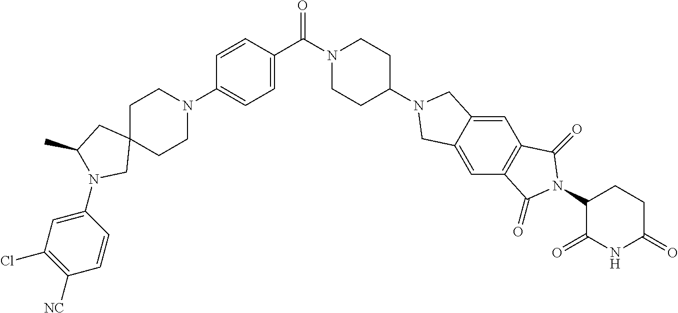 |
| 81 | 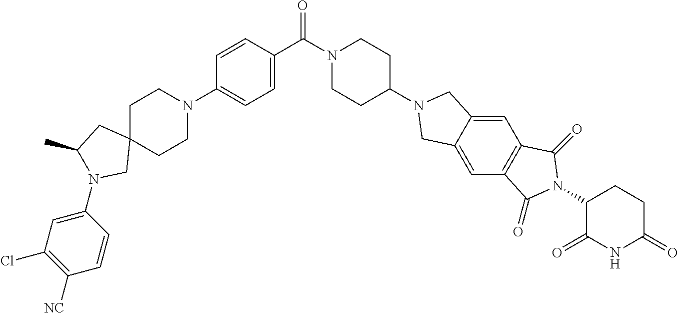 |
| 82 | 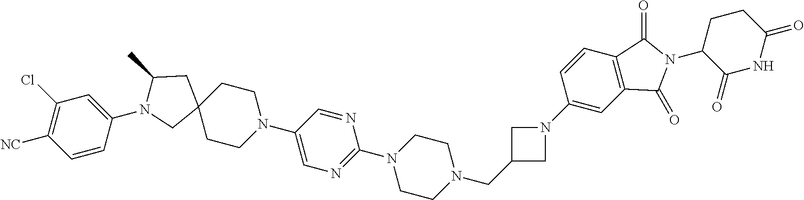 [SMILES: C[C@H]1CC2(CCN(c3cnc(N4CCN(CC5CN(c6ccc7c(c6)C(=O)N(C6CCC(=O)NC6=O)C7=O)C5)CC4)nc3)CC2)CN1c1ccc(C#N)c(Cl)c1] |
| 83 | 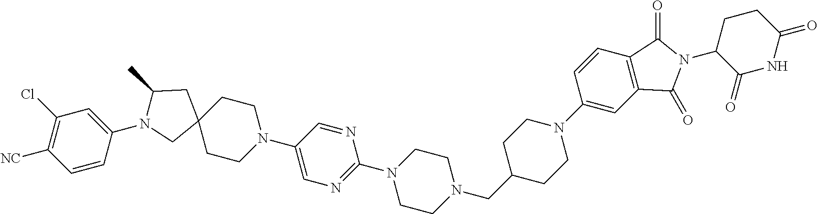 [SMILES: C[C@H]1CC2(CCN(c3cnc(N4CCN(CC5CCN(c6ccc7c(c6)C(=O)N(C6CCC(=O)NC6=O)C7=O)CC5)CC4)nc3)CC2)CN1c1ccc(C#N)c(Cl)c1] |
| 84 | 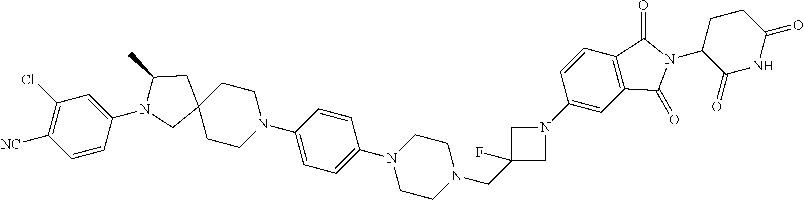 [SMILES: C[C@H]1CC2(CCN(c3ccc(N4CCN(CC5(F)CN(c6ccc7c(c6)C(=O)N(C6CCC(=O)NC6=O)C7=O)C5)CC4)cc3)CC2)CN1c1ccc(C#N)c(Cl)c1] |
| 85 | 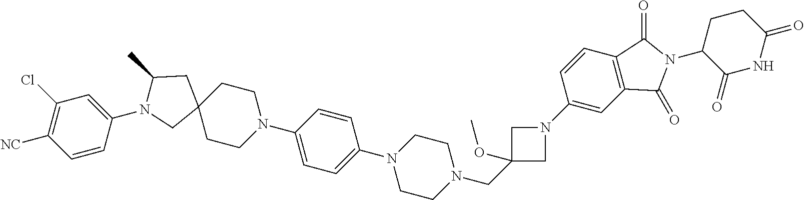 [SMILES: COC1(CN2CCN(c3ccc(N4CCC5(CC4)C[C@H](C)N(c4ccc(C#N)c(Cl)c4)C5)cc3)CC2)CN(c2ccc3c(c2)C(=O)N(C2CCC(=O)NC2=O)C3=O)C1] |
| 86 | 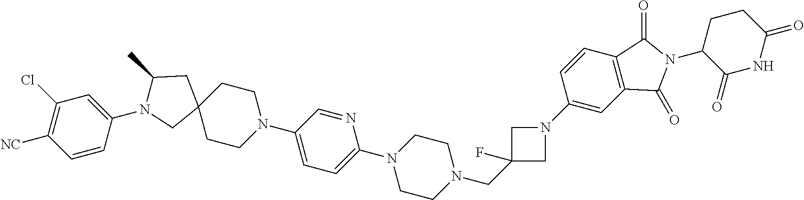 [SMILES: C[C@H]1CC2(CCN(c3ccc(N4CCN(CC5(F)CN(c6ccc7c(c6)C(=O)N(C6CCC(=O)NC6=O)C7=O)C5)CC4)nc3)CC2)CN1c1ccc(C#N)c(Cl)c1] |
| 87 | 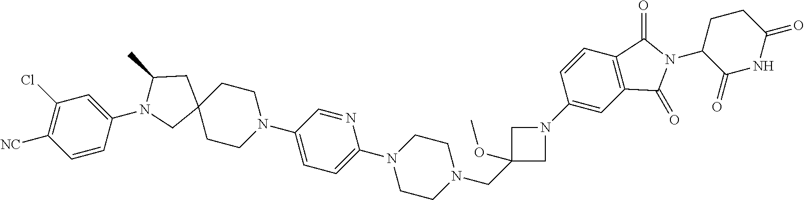 [SMILES: COC1(CN2CCN(c3ccc(N4CCC5(CC4)C[C@H](C)N(c4ccc(C#N)c(Cl)c4)C5)cn3)CC2)CN(c2ccc3c(c2)C(=O)N(C2CCC(=O)NC2=O)C3=O)C1] |
| 88 | 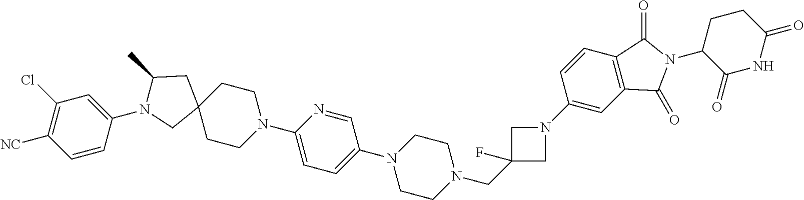 [SMILES: C[C@H]1CC2(CCN(c3ccc(N4CCN(CC5(F)CN(c6ccc7c(c6)C(=O)N(C6CCC(=O)NC6=O)C7=O)C5)CC4)cn3)CC2)CN1c1ccc(C#N)c(Cl)c1] |
| 89 | 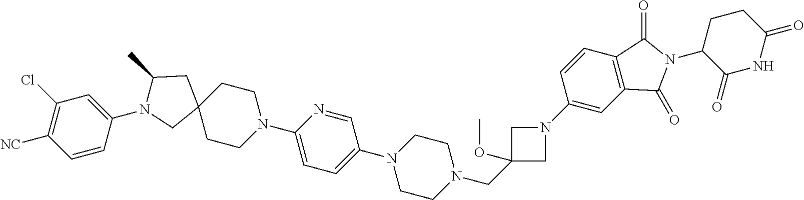 [SMILES: COC1(CN2CCN(c3ccc(N4CCC5(CC4)C[C@H](C)N(c4ccc(C#N)c(Cl)c4)C5)nc3)CC2)CN(c2ccc3c(c2)C(=O)N(C2CCC(=O)NC2=O)C3=O)C1] |
| 90 | 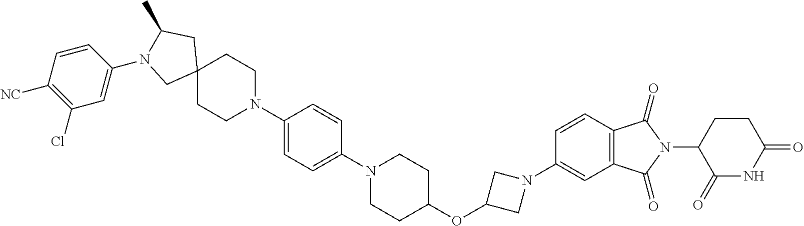 [SMILES: C[C@H]1CC2(CCN(c3ccc(N4CCC(OC5CN(c6ccc7c(c6)C(=O)N(C6CCC(=O)NC6=O)C7=O)C5)CC4)cc3)CC2)CN1c1ccc(C#N)c(Cl)c1] |
| 91 | 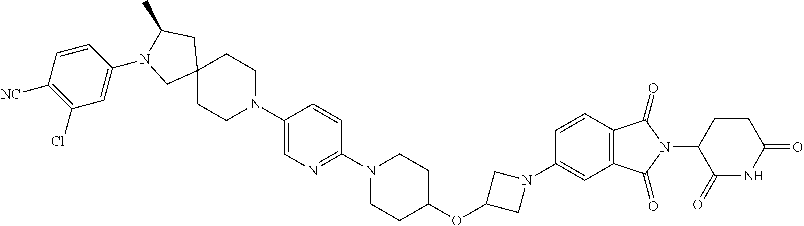 [SMILES: C[C@H]1CC2(CCN(c3ccc(N4CCC(OC5CN(c6ccc7c(c6)C(=O)N(C6CCC(=O)NC6=O)C7=O)C5)CC4)nc3)CC2)CN1c1ccc(C#N)c(Cl)c1] |
| 92 | 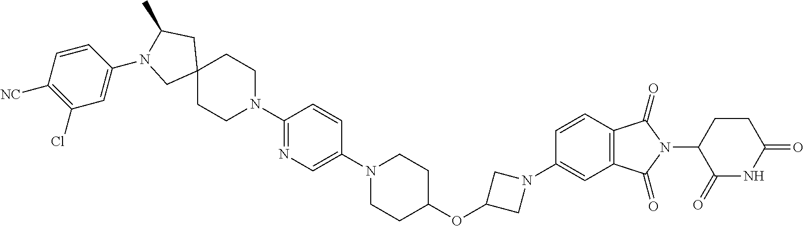 [SMILES: C[C@H]1CC2(CCN(c3ccc(N4CCC(OC5CN(c6ccc7c(c6)C(=O)N(C6CCC(=O)NC6=O)C7=O)C5)CC4)cn3)CC2)CN1c1ccc(C#N)c(Cl)c1] |
| 93 | 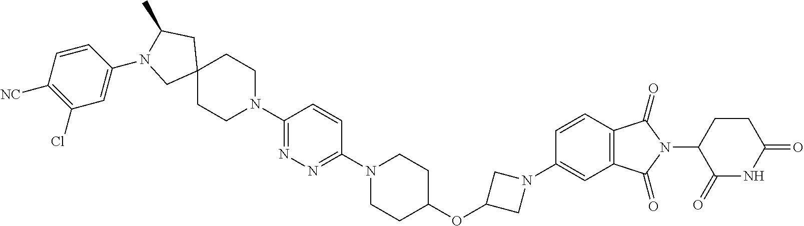 [SMILES: C[C@H]1CC2(CCN(c3ccc(N4CCC(OC5CN(c6ccc7c(c6)C(=O)N(C6CCC(=O)NC6=O)C7=O)C5)CC4)nn3)CC2)CN1c1ccc(C#N)c(Cl)c1] |
| 94 | 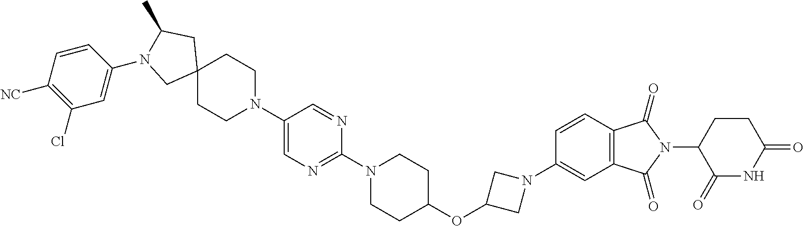 [SMILES: C[C@H]1CC2(CCN(c3cnc(N4CCC(OC5CN(c6ccc7c(c6)C(=O)N(C6CCC(=O)NC6=O)C7=O)C5)CC4)nc3)CC2)CN1c1ccc(C#N)c(Cl)c1] |
| 95 | 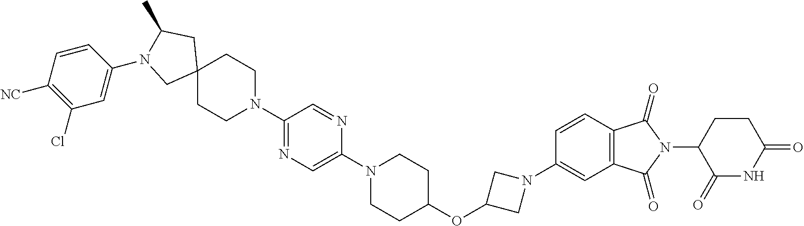 [SMILES: C[C@H]1CC2(CCN(c3cnc(N4CCC(OC5CN(c6ccc7c(c6)C(=O)N(C6CCC(=O)NC6=O)C7=O)C5)CC4)cn3)CC2)CN1c1ccc(C#N)c(Cl)c1] |
| 96 | 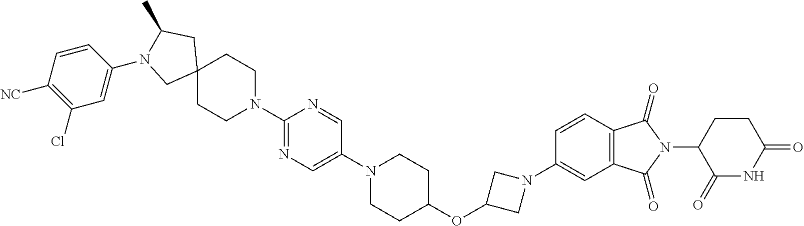 [SMILES: C[C@H]1CC2(CCN(c3ncc(N4CCC(OC5CN(c6ccc7c(c6)C(=O)N(C6CCC(=O)NC6=O)C7=O)C5)CC4)cn3)CC2)CN1c1ccc(C#N)c(Cl)c1] |
| 97 | 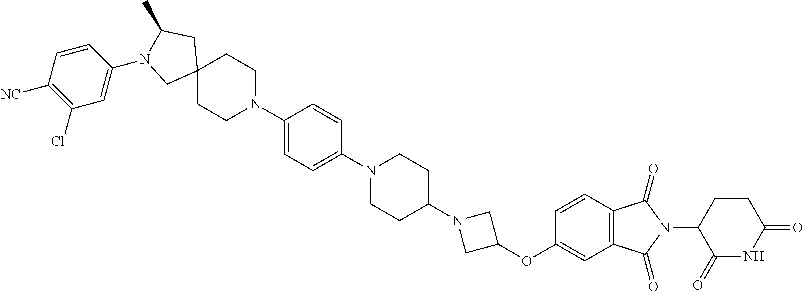 [SMILES: C[C@H]1CC2(CCN(c3ccc(N4CCC(N5CC(Oc6ccc7c(c6)C(=O)N(C6CCC(=O)NC6=O)C7=O)C5)CC4)cc3)CC2)CN1c1ccc(C#N)c(Cl)c1] |
| 98 | 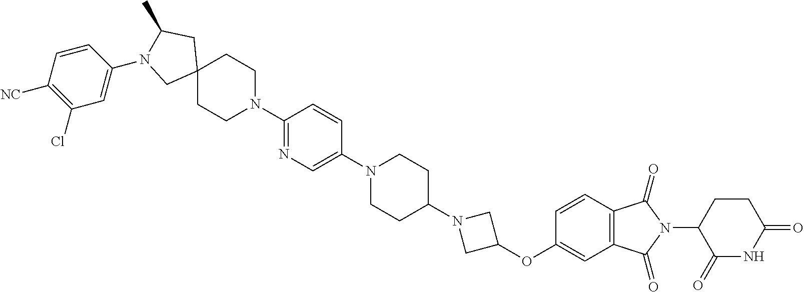 [SMILES: C[C@H]1CC2(CCN(c3ccc(N4CCC(N5CC(Oc6ccc7c(c6)C(=O)N(C6CCC(=O)NC6=O)C7=O)C5)CC4)cn3)CC2)CN1c1ccc(C#N)c(Cl)c1] |
| 99 | 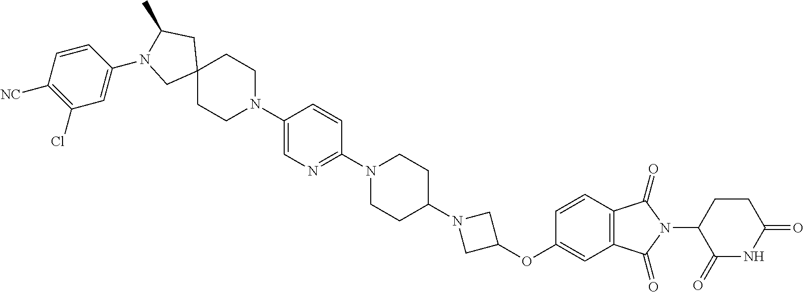 [SMILES: C[C@H]1CC2(CCN(c3ccc(N4CCC(N5CC(Oc6ccc7c(c6)C(=O)N(C6CCC(=O)NC6=O)C7=O)C5)CC4)nc3)CC2)CN1c1ccc(C#N)c(Cl)c1] |
| 100 | 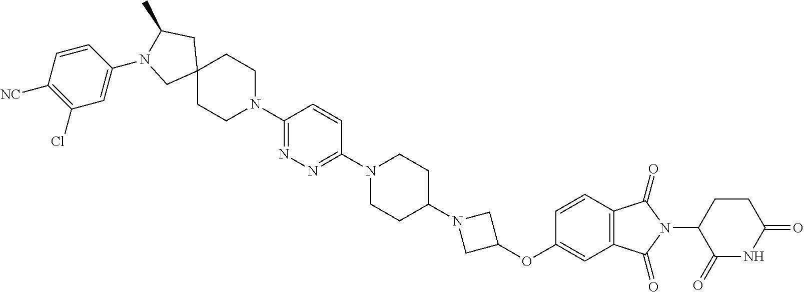 [SMILES: C[C@H]1CC2(CCN(c3ccc(N4CCC(N5CC(Oc6ccc7c(c6)C(=O)N(C6CCC(=O)NC6=O)C7=O)C5)CC4)nn3)CC2)CN1c1ccc(C#N)c(Cl)c1] |
| 101 | 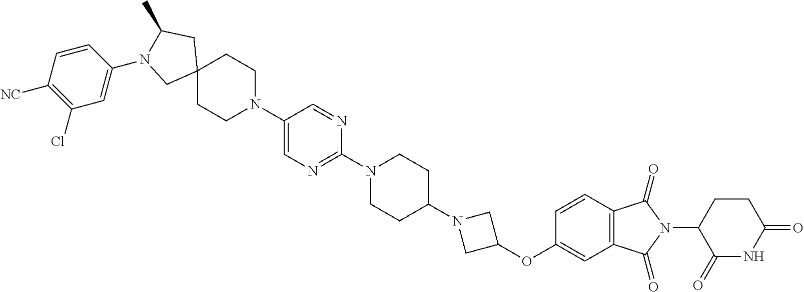 [SMILES: C[C@H]1CC2(CCN(c3cnc(N4CCC(N5CC(Oc6ccc7c(c6)C(=O)N(C6CCC(=O)NC6=O)C7=O)C5)CC4)nc3)CC2)CN1c1ccc(C#N)c(Cl)c1] |
| 102 | 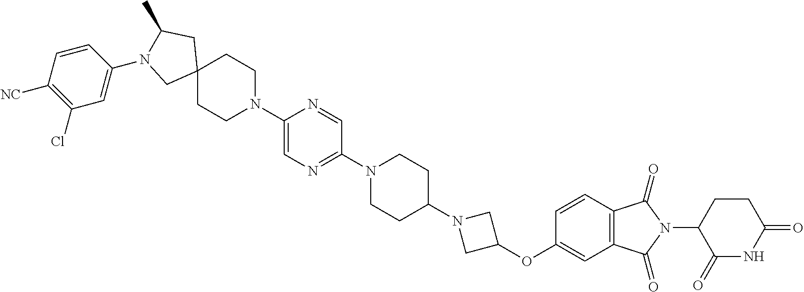 [SMILES: C[C@H]1CC2(CCN(c3cnc(N4CCC(N5CC(Oc6ccc7c(c6)C(=O)N(C6CCC(=O)NC6=O)C7=O)C5)CC4)cn3)CC2)CN1c1ccc(C#N)c(Cl)c1] |
| 103 | 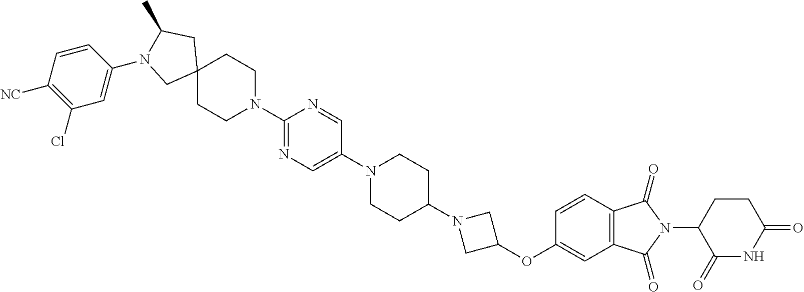 [SMILES: C[C@H]1CC2(CCN(c3ncc(N4CCC(N5CC(Oc6ccc7c(c6)C(=O)N(C6CCC(=O)NC6=O)C7=O)C5)CC4)cn3)CC2)CN1c1ccc(C#N)c(Cl)c1] |
| 104 | 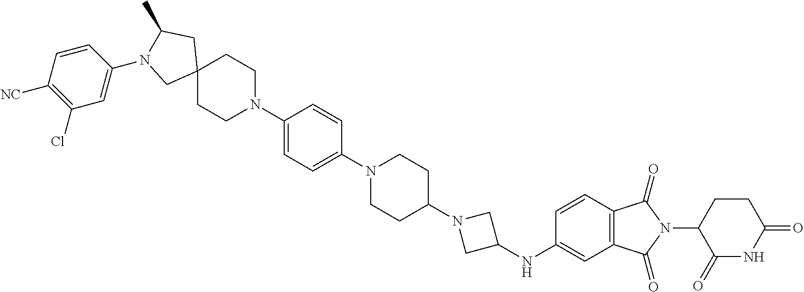 [SMILES: C[C@H]1CC2(CCN(c3ccc(N4CCC(N5CC(Nc6ccc7c(c6)C(=O)N(C6CCC(=O)NC6=O)C7=O)C5)CC4)cc3)CC2)CN1c1ccc(C#N)c(Cl)c1] |
| 105 | 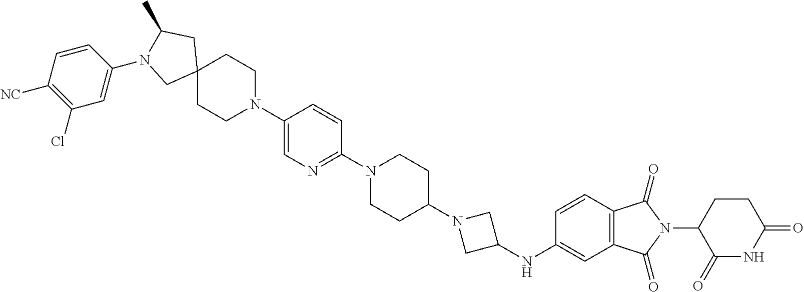 [SMILES: C[C@H]1CC2(CCN(c3ccc(N4CCC(N5CC(Nc6ccc7c(c6)C(=O)N(C6CCC(=O)NC6=O)C7=O)C5)CC4)nc3)CC2)CN1c1ccc(C#N)c(Cl)c1] |
| 106 | 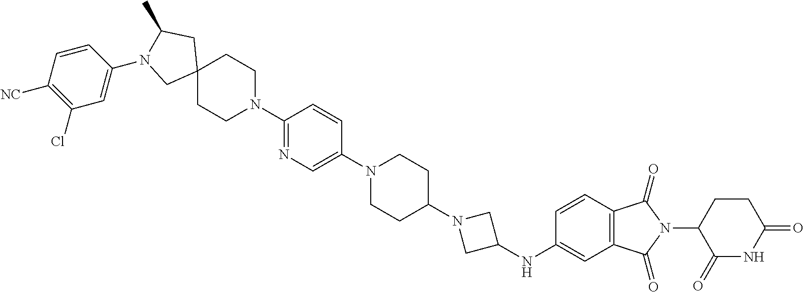 [SMILES: C[C@H]1CC2(CCN(c3ccc(N4CCC(N5CC(Nc6ccc7c(c6)C(=O)N(C6CCC(=O)NC6=O)C7=O)C5)CC4)cn3)CC2)CN1c1ccc(C#N)c(Cl)c1] |
| 107 | 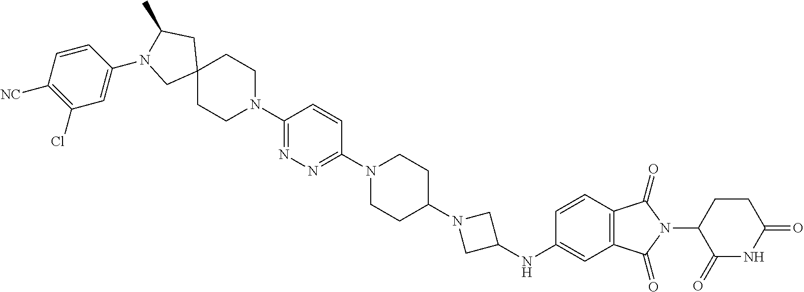 [SMILES: C[C@H]1CC2(CCN(c3ccc(N4CCC(N5CC(Nc6ccc7c(c6)C(=O)N(C6CCC(=O)NC6=O)C7=O)C5)CC4)nn3)CC2)CN1c1ccc(C#N)c(Cl)c1] |
| 108 | 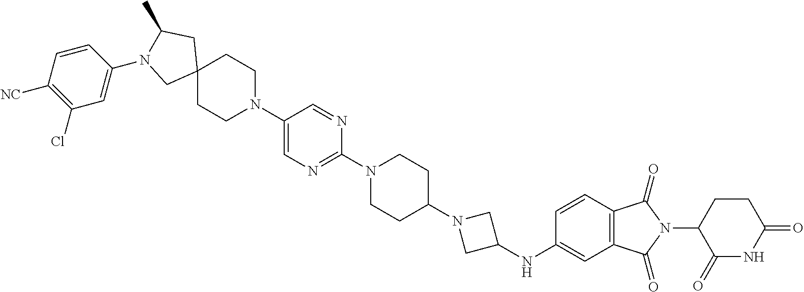 [SMILES: C[C@H]1CC2(CCN(c3cnc(N4CCC(N5CC(Nc6ccc7c(c6)C(=O)N(C6CCC(=O)NC6=O)C7=O)C5)CC4)nc3)CC2)CN1c1ccc(C#N)c(Cl)c1] |
| 109 | 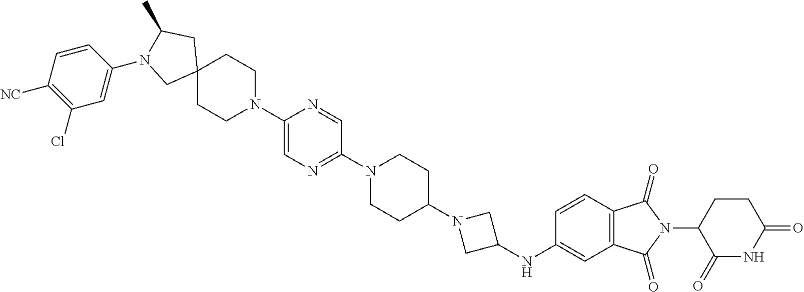 [SMILES: C[C@H]1CC2(CCN(c3cnc(N4CCC(N5CC(Nc6ccc7c(c6)C(=O)N(C6CCC(=O)NC6=O)C7=O)C5)CC4)cn3)CC2)CN1c1ccc(C#N)c(Cl)c1] |
| 110 | 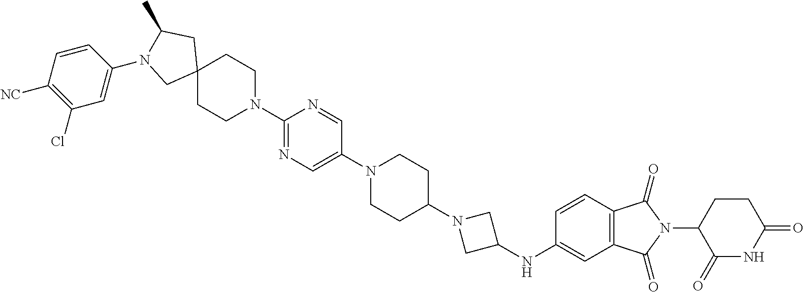 [SMILES: C[C@H]1CC2(CCN(c3ncc(N4CCC(N5CC(Nc6ccc7c(c6)C(=O)N(C6CCC(=O)NC6=O)C7=O)C5)CC4)cn3)CC2)CN1c1ccc(C#N)c(Cl)c1] |
| 111 | 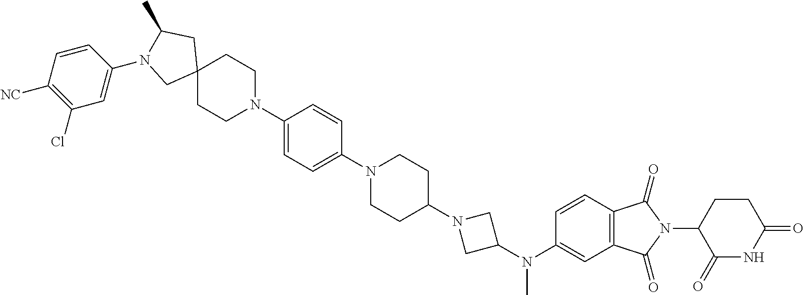 [SMILES: C[C@H]1CC2(CCN(c3ccc(N4CCC(N5CC(N(C)c6ccc7c(c6)C(=O)N(C6CCC(=O)NC6=O)C7=O)C5)CC4)cc3)CC2)CN1c1ccc(C#N)c(Cl)c1] |
| 112 | 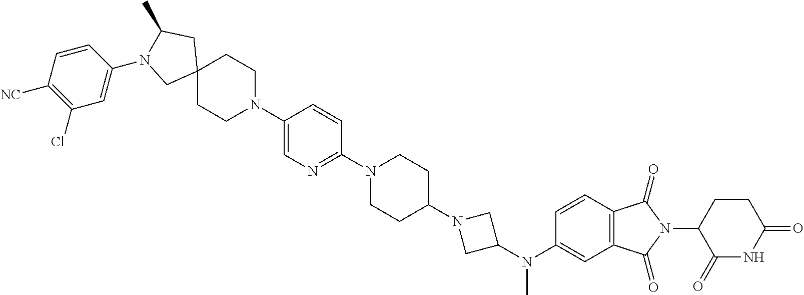 [SMILES: C[C@H]1CC2(CCN(c3ccc(N4CCC(N5CC(N(C)c6ccc7c(c6)C(=O)N(C6CCC(=O)NC6=O)C7=O)C5)CC4)nc3)CC2)CN1c1ccc(C#N)c(Cl)c1] |
| 113 | 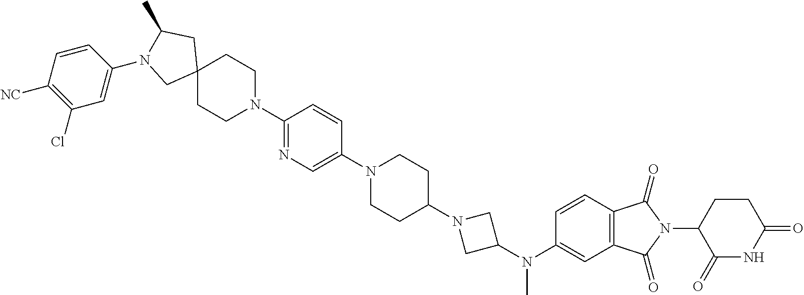 [SMILES: C[C@H]1CC2(CCN(c3ccc(N4CCC(N5CC(N(C)c6ccc7c(c6)C(=O)N(C6CCC(=O)NC6=O)C7=O)C5)CC4)cn3)CC2)CN1c1ccc(C#N)c(Cl)c1] |
| 114 | 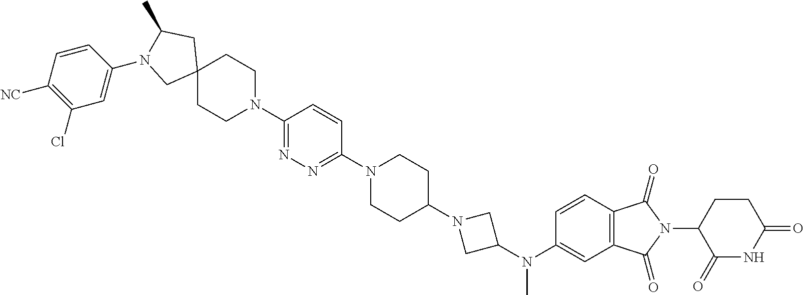 [SMILES: C[C@H]1CC2(CCN(c3ccc(N4CCC(N5CC(N(C)c6ccc7c(c6)C(=O)N(C6CCC(=O)NC6=O)C7=O)C5)CC4)nn3)CC2)CN1c1ccc(C#N)c(Cl)c1] |
| 115 | 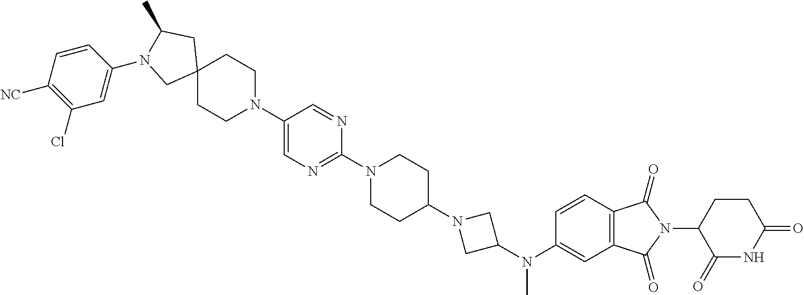 [SMILES: C[C@H]1CC2(CCN(c3cnc(N4CCC(N5CC(N(C)c6ccc7c(c6)C(=O)N(C6CCC(=O)NC6=O)C7=O)C5)CC4)nc3)CC2)CN1c1ccc(C#N)c(Cl)c1] |
| 116 | 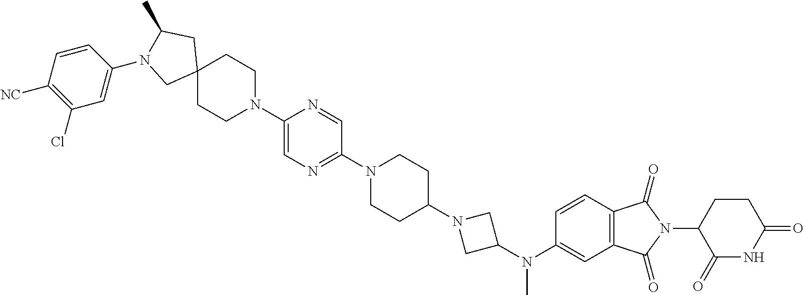 [SMILES: C[C@H]1CC2(CCN(c3cnc(N4CCC(N5CC(N(C)c6ccc7c(c6)C(=O)N(C6CCC(=O)NC6=O)C7=O)C5)CC4)cn3)CC2)CN1c1ccc(C#N)c(Cl)c1] |
| 117 | 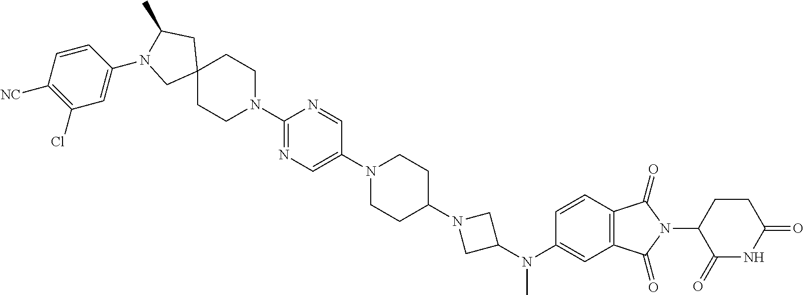 [SMILES: C[C@H]1CC2(CCN(c3ncc(N4CCC(N5CC(N(C)c6ccc7c(c6)C(=O)N(C6CCC(=O)NC6=O)C7=O)C5)CC4)cn3)CC2)CN1c1ccc(C#N)c(Cl)c1] |
| 118 | 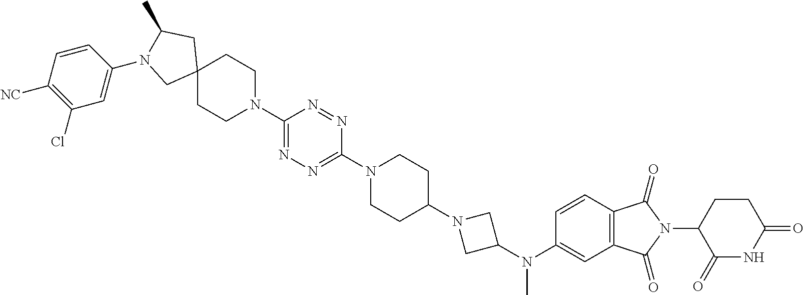 [SMILES: C[C@H]1CC2(CCN(c3nnc(N4CCC(N5CC(N(C)c6ccc7c(c6)C(=O)N(C6CCC(=O)NC6=O)C7=O)C5)CC4)nn3)CC2)CN1c1ccc(C#N)c(Cl)c1] |
| 119 | 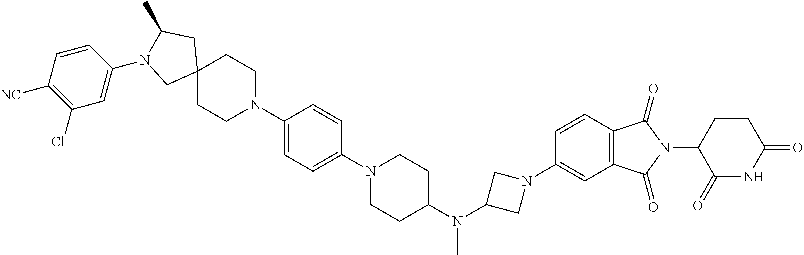 [SMILES: C[C@H]1CC2(CCN(c3ccc(N4CCC(N(C)C5CN(c6ccc7c(c6)C(=O)N(C6CCC(=O)NC6=O)C7=O)C5)CC4)cc3)CC2)CN1c1ccc(C#N)c(Cl)c1] |
| 120 | 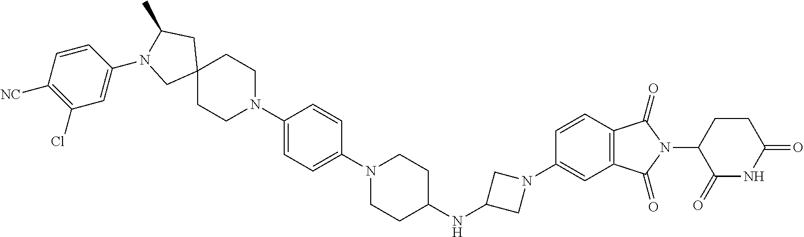 [SMILES: C[C@H]1CC2(CCN(c3ccc(N4CCC(NC5CN(c6ccc7c(c6)C(=O)N(C6CCC(=O)NC6=O)C7=O)C5)CC4)cc3)CC2)CN1c1ccc(C#N)c(Cl)c1] |
| 121 | 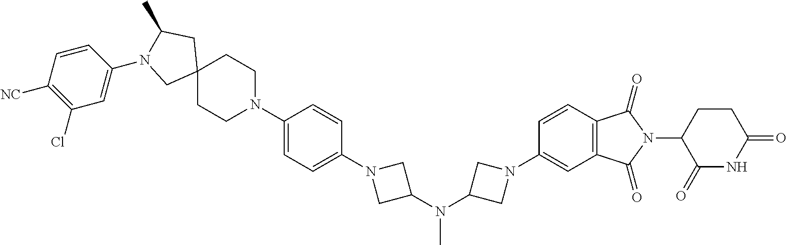 [SMILES: C[C@H]1CC2(CCN(c3ccc(N4CC(N(C)C5CN(c6ccc7c(c6)C(=O)N(C6CCC(=O)NC6=O)C7=O)C5)C4)cc3)CC2)CN1c1ccc(C#N)c(Cl)c1] |
| 123 | 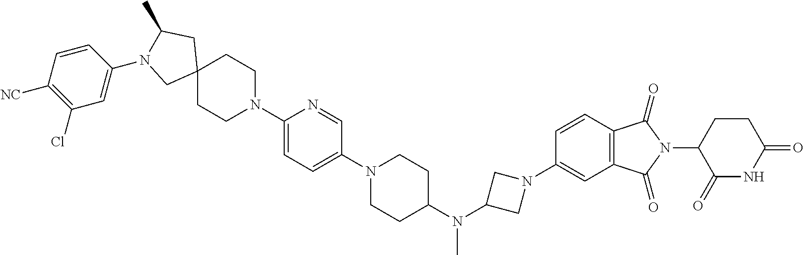 [SMILES: C[C@H]1CC2(CCN(c3ccc(N4CCC(N(C)C5CN(c6ccc7c(c6)C(=O)N(C6CCC(=O)NC6=O)C7=O)C5)CC4)cn3)CC2)CN1c1ccc(C#N)c(Cl)c1] |
| 124 | 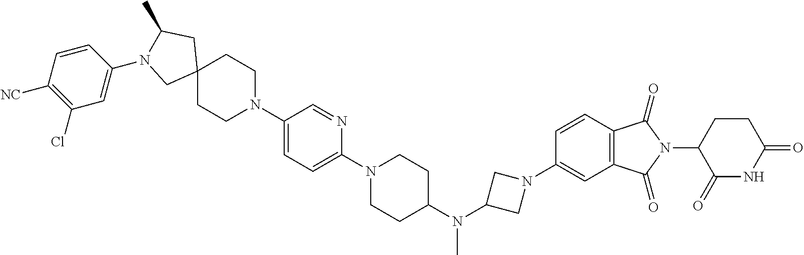 [SMILES: C[C@H]1CC2(CCN(c3ccc(N4CCC(N(C)C5CN(c6ccc7c(c6)C(=O)N(C6CCC(=O)NC6=O)C7=O)C5)CC4)nc3)CC2)CN1c1ccc(C#N)c(Cl)c1] |
| 125 | 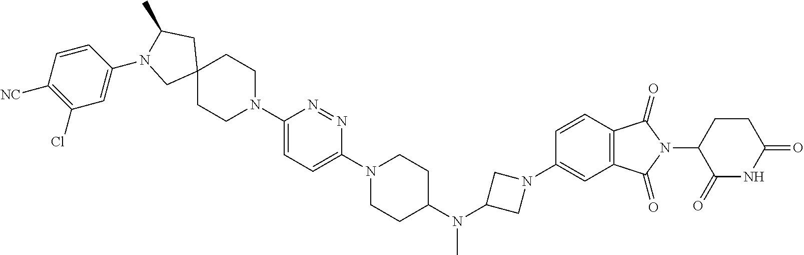 [SMILES: C[C@H]1CC2(CCN(c3ccc(N4CCC(N(C)C5CN(c6ccc7c(c6)C(=O)N(C6CCC(=O)NC6=O)C7=O)C5)CC4)nn3)CC2)CN1c1ccc(C#N)c(Cl)c1] |
| 126 | 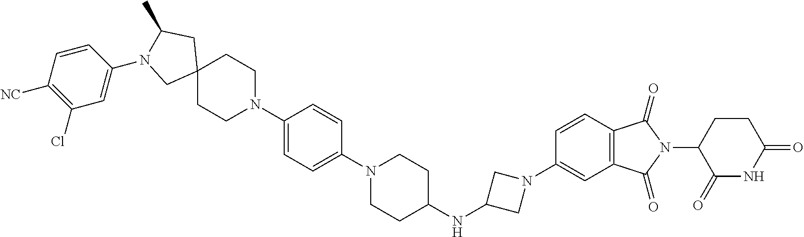 [SMILES: C[C@H]1CC2(CCN(c3ccc(N4CCC(NC5CN(c6ccc7c(c6)C(=O)N(C6CCC(=O)NC6=O)C7=O)C5)CC4)cc3)CC2)CN1c1ccc(C#N)c(Cl)c1] |
| 127 | 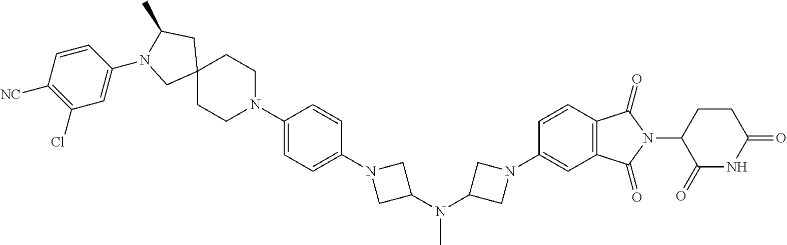 [SMILES: C[C@H]1CC2(CCN(c3ccc(N4CC(N(C)C5CN(c6ccc7c(c6)C(=O)N(C6CCC(=O)NC6=O)C7=O)C5)C4)cc3)CC2)CN1c1ccc(C#N)c(Cl)c1] |
| 128 | 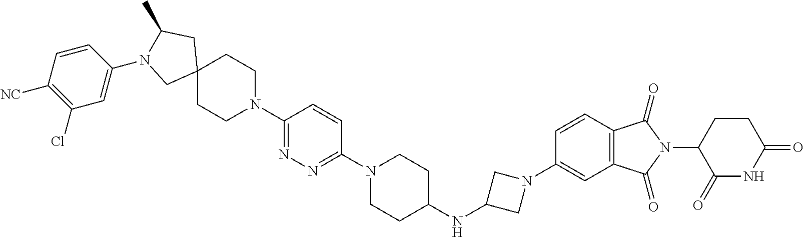 [SMILES: C[C@H]1CC2(CCN(c3ccc(N4CCC(NC5CN(c6ccc7c(c6)C(=O)N(C6CCC(=O)NC6=O)C7=O)C5)CC4)nn3)CC2)CN1c1ccc(C#N)c(Cl)c1] |
| 129 | 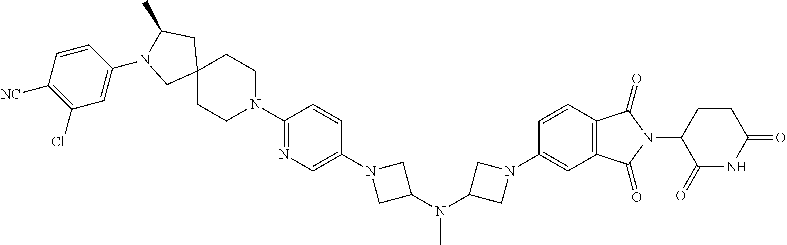 [SMILES: C[C@H]1CC2(CCN(c3ccc(N4CC(N(C)C5CN(c6ccc7c(c6)C(=O)N(C6CCC(=O)NC6=O)C7=O)C5)C4)cn3)CC2)CN1c1ccc(C#N)c(Cl)c1] |
| 130 | 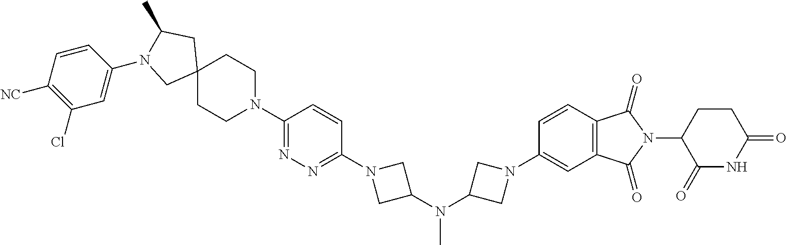 [SMILES: C[C@H]1CC2(CCN(c3ccc(N4CC(N(C)C5CN(c6ccc7c(c6)C(=O)N(C6CCC(=O)NC6=O)C7=O)C5)C4)nn3)CC2)CN1c1ccc(C#N)c(Cl)c1] |
| 131 | 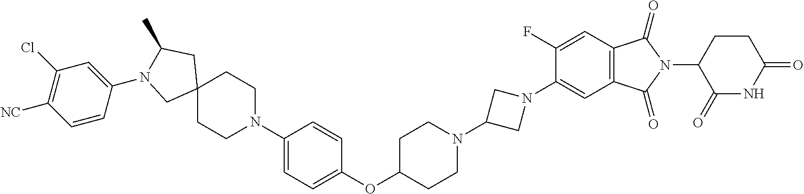 [SMILES: C[C@H]1CC2(CCN(c3ccc(OC4CCN(C5CN(c6cc7c(cc6F)C(=O)N(C6CCC(=O)NC6=O)C7=O)C5)CC4)cc3)CC2)CN1c1ccc(C#N)c(Cl)c1] |
| 132 | 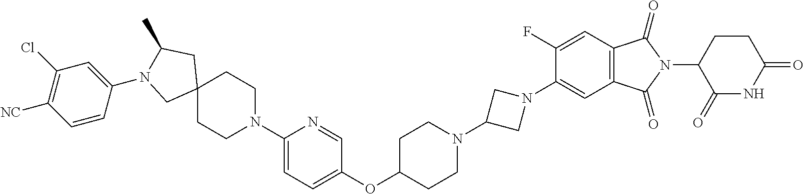 [SMILES: C[C@H]1CC2(CCN(c3ccc(OC4CCN(C5CN(c6cc7c(cc6F)C(=O)N(C6CCC(=O)NC6=O)C7=O)C5)CC4)cn3)CC2)CN1c1ccc(C#N)c(Cl)c1] |
| 133 | 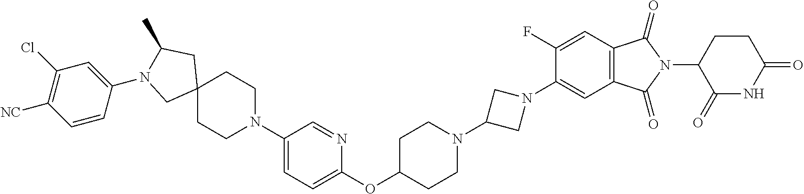 [SMILES: C[C@H]1CC2(CCN(c3ccc(OC4CCN(C5CN(c6cc7c(cc6F)C(=O)N(C6CCC(=O)NC6=O)C7=O)C5)CC4)nc3)CC2)CN1c1ccc(C#N)c(Cl)c1] |
| 134 | 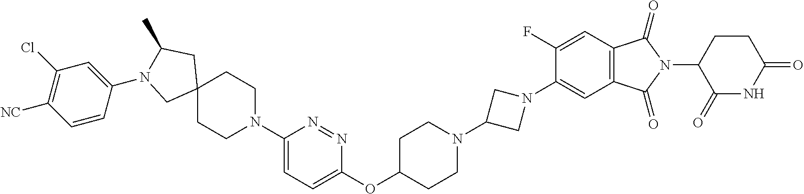 [SMILES: C[C@H]1CC2(CCN(c3ccc(OC4CCN(C5CN(c6cc7c(cc6F)C(=O)N(C6CCC(=O)NC6=O)C7=O)C5)CC4)nn3)CC2)CN1c1ccc(C#N)c(Cl)c1] |
| 135 | 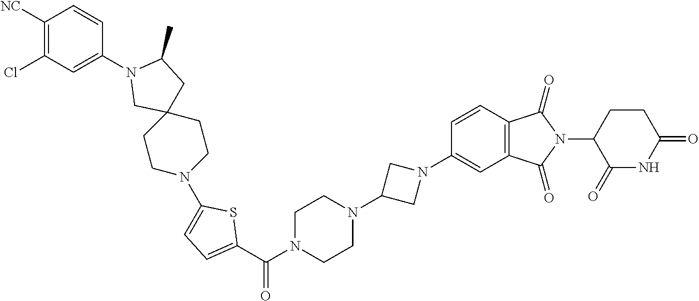 [SMILES: C[C@H]1CC2(CCN(c3ccc(C(=O)N4CCN(C5CN(c6ccc7c(c6)C(=O)N(C6CCC(=O)NC6=O)C7=O)C5)CC4)s3)CC2)CN1c1ccc(C#N)c(Cl)c1] |
| 136 | 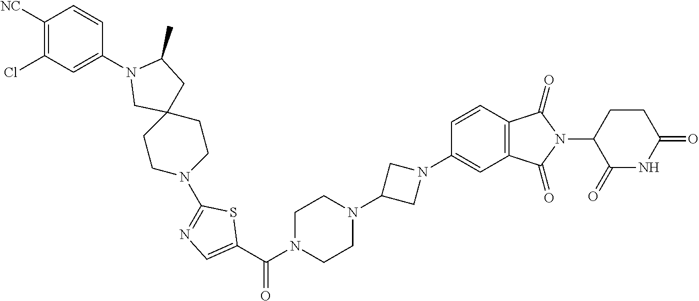 [SMILES: C[C@H]1CC2(CCN(c3ncc(C(=O)N4CCN(C5CN(c6ccc7c(c6)C(=O)N(C6CCC(=O)NC6=O)C7=O)C5)CC4)s3)CC2)CN1c1ccc(C#N)c(Cl)c1] |
| 137 | 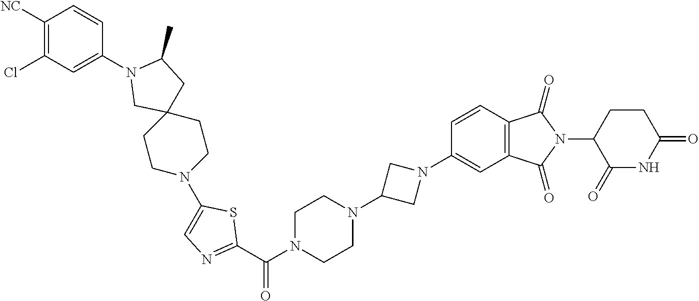 [SMILES: C[C@H]1CC2(CCN(c3cnc(C(=O)N4CCN(C5CN(c6ccc7c(c6)C(=O)N(C6CCC(=O)NC6=O)C7=O)C5)CC4)s3)CC2)CN1c1ccc(C#N)c(Cl)c1] |
| 138 | 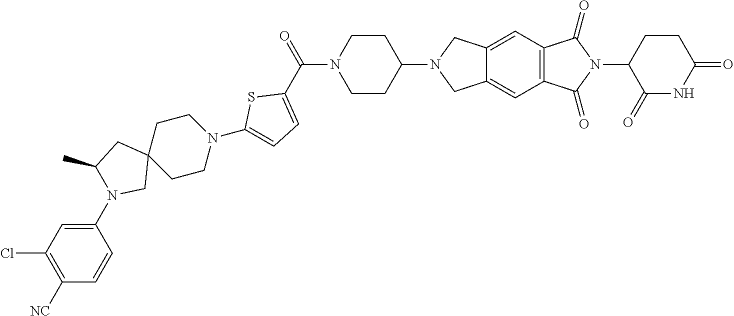 [SMILES: C[C@H]1CC2(CCN(c3ccc(C(=O)N4CCC(N5Cc6cc7c(cc6C5)C(=O)N(C5CCC(=O)NC5=O)C7=O)CC4)s3)CC2)CN1c1ccc(C#N)c(Cl)c1] |
| 139 | 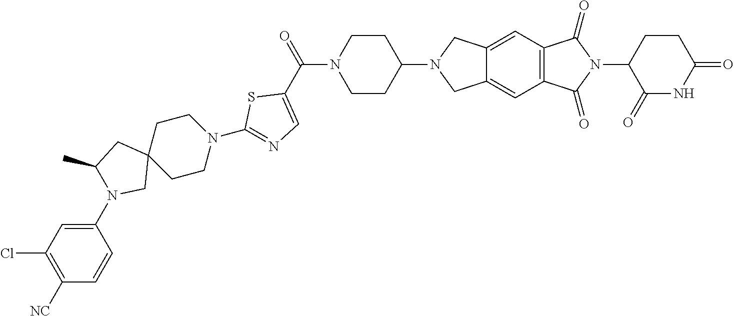 [SMILES: C[C@H]1CC2(CCN(c3ncc(C(=O)N4CCC(N5Cc6cc7c(cc6C5)C(=O)N(C5CCC(=O)NC5=O)C7=O)CC4)s3)CC2)CN1c1ccc(C#N)c(Cl)c1] |
| 140 | 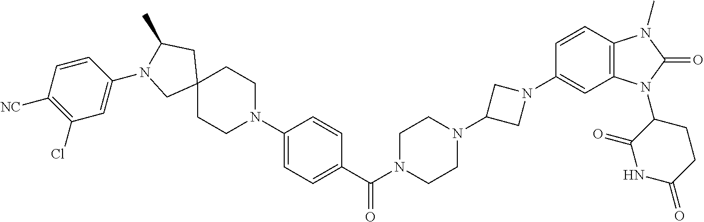 [SMILES: C[C@H]1CC2(CCN(c3ccc(C(=O)N4CCN(C5CN(c6ccc7c(c6)n(C6CCC(=O)NC6=O)c(=O)n7C)C5)CC4)cc3)CC2)CN1c1ccc(C#N)c(Cl)c1] |
| 141 | 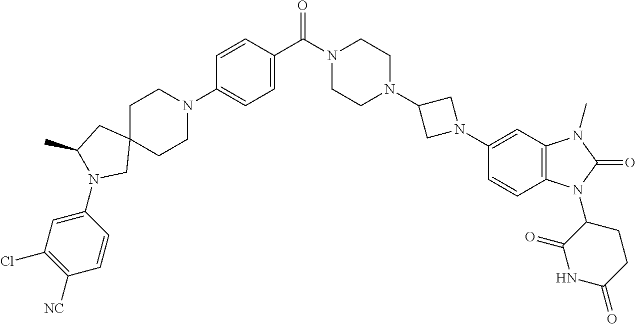 [SMILES: C[C@H]1CC2(CCN(c3ccc(C(=O)N4CCN(C5CN(c6ccc7c(c6)n(C)c(=O)n7C6CCC(=O)NC6=O)C5)CC4)cc3)CC2)CN1c1ccc(C#N)c(Cl)c1] |
| 142 | 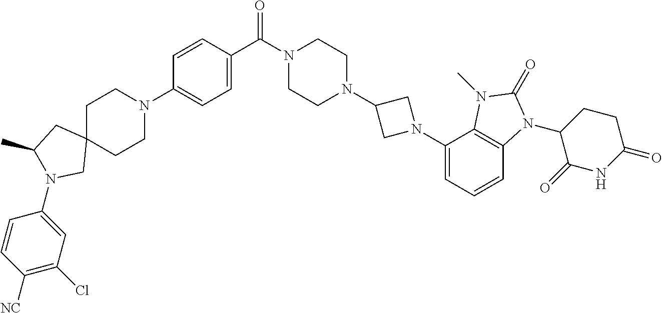 [SMILES: C[C@H]1CC2(CCN(c3ccc(C(=O)N4CCN(C5CN(c6cccc7c6n(C)c(=O)n7C6CCC(=O)NC6=O)C5)CC4)cc3)CC2)CN1c1ccc(C#N)c(Cl)c1] |
| 143 | 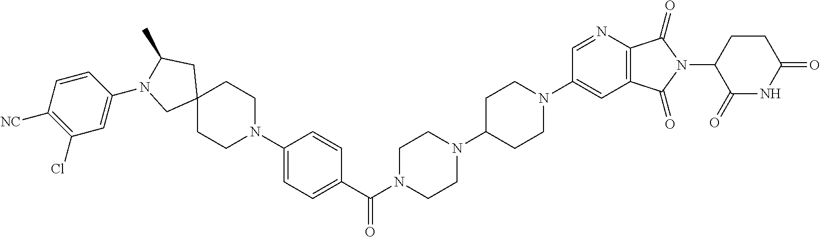 [SMILES: C[C@H]1CC2(CCN(c3ccc(C(=O)N4CCN(C5CCN(c6cnc7c(c6)C(=O)N(C6CCC(=O)NC6=O)C7=O)CC5)CC4)cc3)CC2)CN1c1ccc(C#N)c(Cl)c1] |
| 144 | 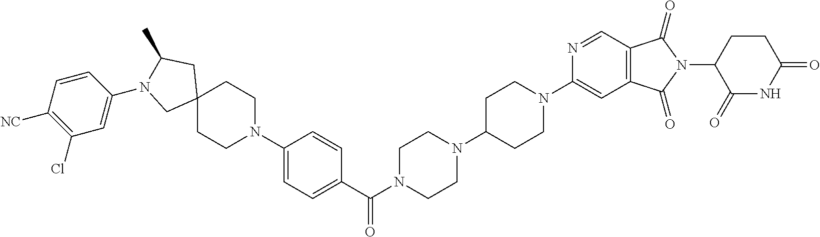 [SMILES: C[C@H]1CC2(CCN(c3ccc(C(=O)N4CCN(C5CCN(c6cc7c(cn6)C(=O)N(C6CCC(=O)NC6=O)C7=O)CC5)CC4)cc3)CC2)CN1c1ccc(C#N)c(Cl)c1] |
| 145 | 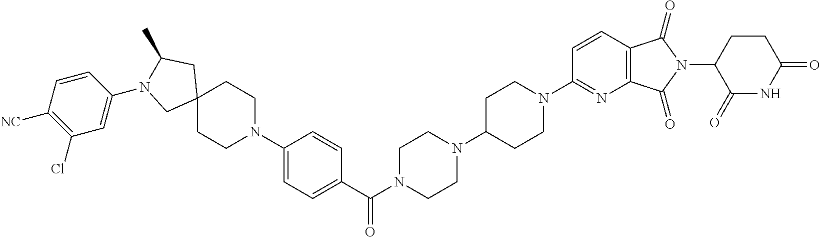 [SMILES: C[C@H]1CC2(CCN(c3ccc(C(=O)N4CCN(C5CCN(c6ccc7c(n6)C(=O)N(C6CCC(=O)NC6=O)C7=O)CC5)CC4)cc3)CC2)CN1c1ccc(C#N)c(Cl)c1] |
| 146 | 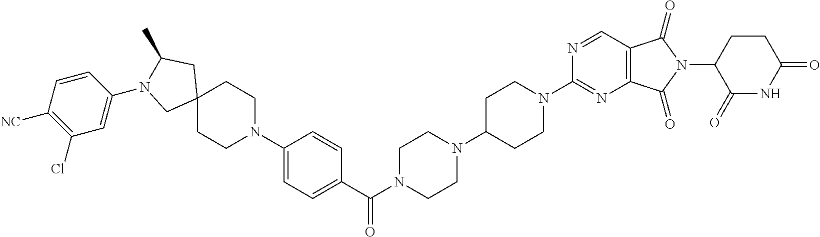 [SMILES: C[C@H]1CC2(CCN(c3ccc(C(=O)N4CCN(C5CCN(c6ncc7c(n6)C(=O)N(C6CCC(=O)NC6=O)C7=O)CC5)CC4)cc3)CC2)CN1c1ccc(C#N)c(Cl)c1] |
| 147 | 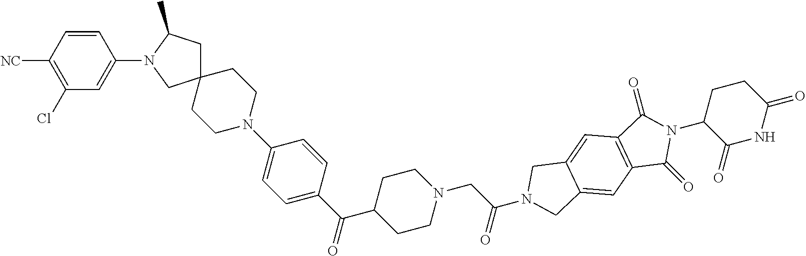 [SMILES: C[C@H]1CC2(CCN(c3ccc(C(=O)C4CCN(CC(=O)N5Cc6cc7c(cc6C5)C(=O)N(C5CCC(=O)NC5=O)C7=O)CC4)cc3)CC2)CN1c1ccc(C#N)c(Cl)c1] |
| 148 | 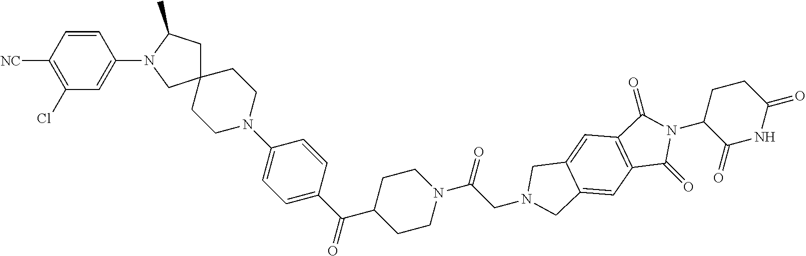 [SMILES: C[C@H]1CC2(CCN(c3ccc(C(=O)C4CCN(C(=O)CN5Cc6cc7c(cc6C5)C(=O)N(C5CCC(=O)NC5=O)C7=O)CC4)cc3)CC2)CN1c1ccc(C#N)c(Cl)c1] |
| 149 | 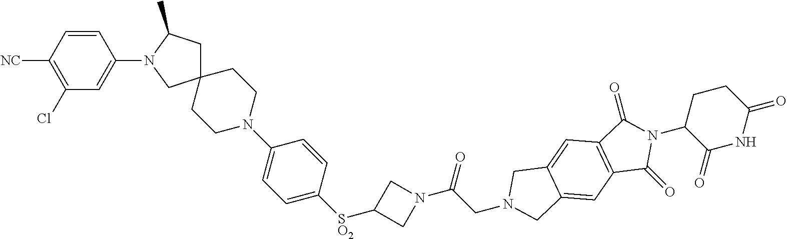 [SMILES: C[C@H]1CC2(CCN(c3ccc(S(=O)(=O)C4CN(C(=O)CN5Cc6cc7c(cc6C5)C(=O)N(C5CCC(=O)NC5=O)C7=O)C4)cc3)CC2)CN1c1ccc(C#N)c(Cl)c1] |
| 150 | 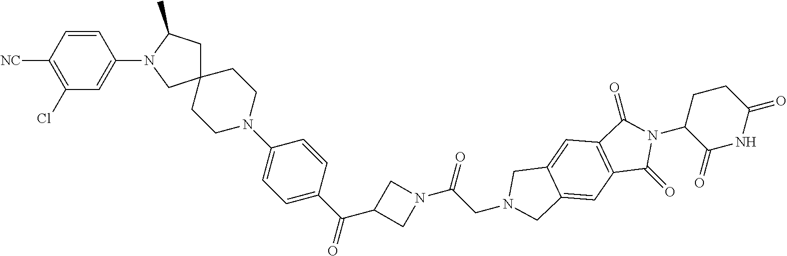 [SMILES: C[C@H]1CC2(CCN(c3ccc(C(=O)C4CN(C(=O)CN5Cc6cc7c(cc6C5)C(=O)N(C5CCC(=O)NC5=O)C7=O)C4)cc3)CC2)CN1c1ccc(C#N)c(Cl)c1] |
| 151 | 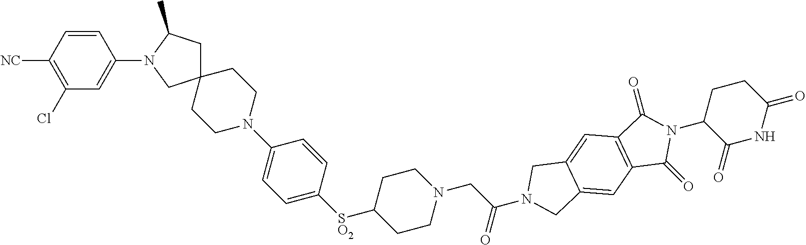 [SMILES: C[C@H]1CC2(CCN(c3ccc(S(=O)(=O)C4CCN(CC(=O)N5Cc6cc7c(cc6C5)C(=O)N(C5CCC(=O)NC5=O)C7=O)CC4)cc3)CC2)CN1c1ccc(C#N)c(Cl)c1] |
| 152 | 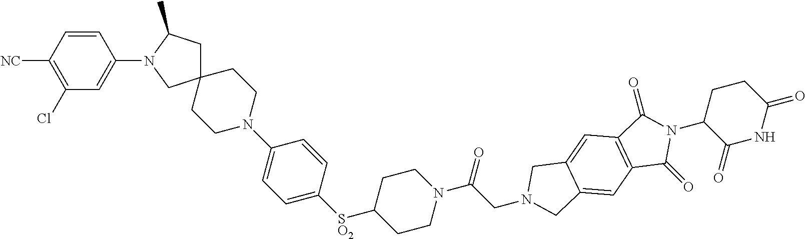 [SMILES: C[C@H]1CC2(CCN(c3ccc(S(=O)(=O)C4CCN(C(=O)CN5Cc6cc7c(cc6C5)C(=O)N(C5CCC(=O)NC5=O)C7=O)CC4)cc3)CC2)CN1c1ccc(C#N)c(Cl)c1] |
| 153 | 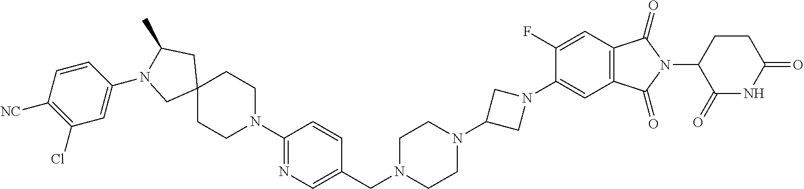 [SMILES: C[C@H]1CC2(CCN(c3ccc(CN4CCN(C5CN(c6cc7c(cc6F)C(=O)N(C6CCC(=O)NC6=O)C7=O)C5)CC4)cn3)CC2)CN1c1ccc(C#N)c(Cl)c1] |
| 154 | 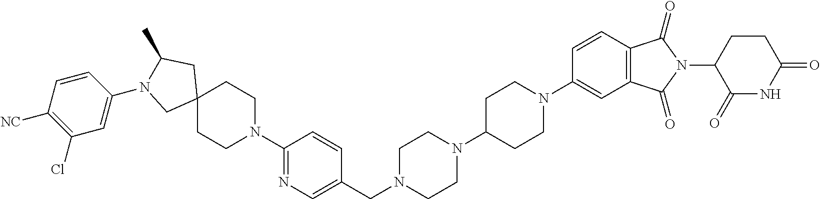 [SMILES: C[C@H]1CC2(CCN(c3ccc(CN4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| 155 | 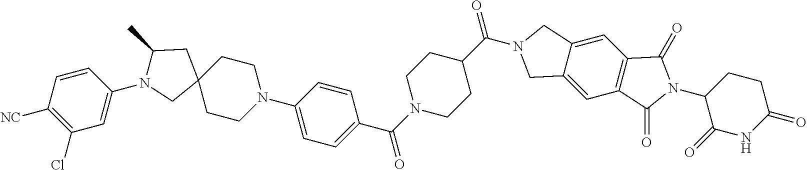 [SMILES: C[C@H]1CC2(CCN(c3ccc(C(=O)N4CCC(C(=O)N5Cc6cc7c(cc6C5)C(=O)N(C5CCC(=O)NC5=O)C7=O)CC4)cc3)CC2)CN1c1ccc(C#N)c(Cl)c1] |
| 156 | 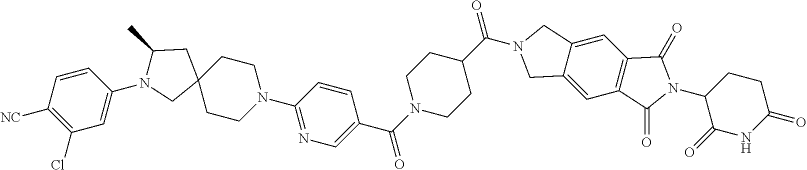 [SMILES: C[C@H]1CC2(CCN(c3ccc(C(=O)N4CCC(C(=O)N5Cc6cc7c(cc6C5)C(=O)N(C5CCC(=O)NC5=O)C7=O)CC4)cn3)CC2)CN1c1ccc(C#N)c(Cl)c1] |
| 157 | 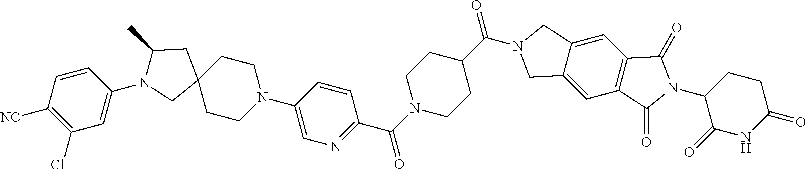 [SMILES: C[C@H]1CC2(CCN(c3ccc(C(=O)N4CCC(C(=O)N5Cc6cc7c(cc6C5)C(=O)N(C5CCC(=O)NC5=O)C7=O)CC4)nc3)CC2)CN1c1ccc(C#N)c(Cl)c1] |
| 158 | 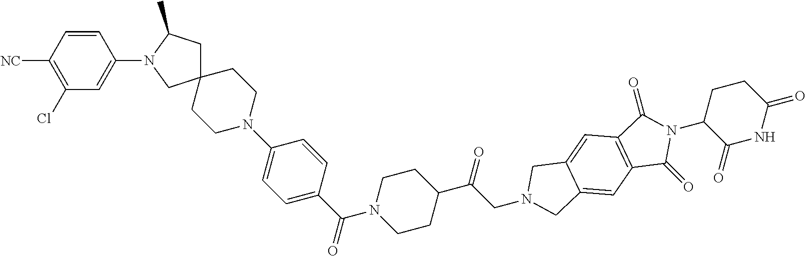 [SMILES: C[C@H]1CC2(CCN(c3ccc(C(=O)N4CCC(C(=O)CN5Cc6cc7c(cc6C5)C(=O)N(C5CCC(=O)NC5=O)C7=O)CC4)cc3)CC2)CN1c1ccc(C#N)c(Cl)c1] |
| 159 | 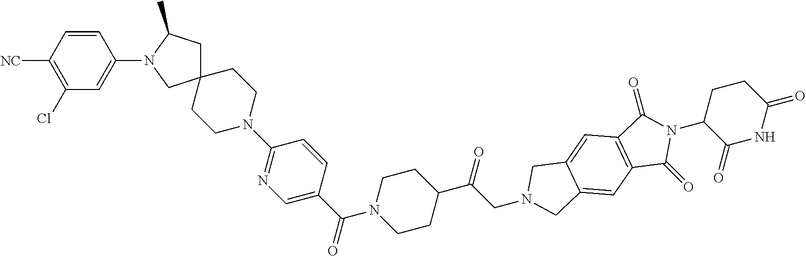 [SMILES: C[C@H]1CC2(CCN(c3ccc(C(=O)N4CCC(C(=O)CN5Cc6cc7c(cc6C5)C(=O)N(C5CCC(=O)NC5=O)C7=O)CC4)cn3)CC2)CN1c1ccc(C#N)c(Cl)c1] |
| 160 | 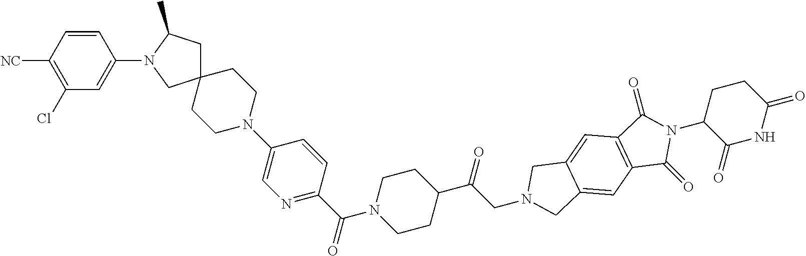 [SMILES: C[C@H]1CC2(CCN(c3ccc(C(=O)N4CCC(C(=O)CN5Cc6cc7c(cc6C5)C(=O)N(C5CCC(=O)NC5=O)C7=O)CC4)nc3)CC2)CN1c1ccc(C#N)c(Cl)c1] |
| 161 | 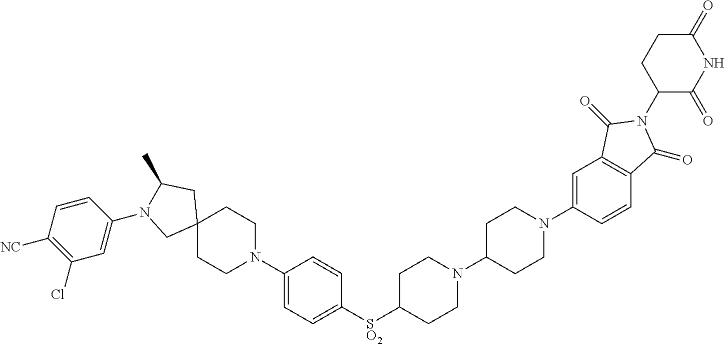 [SMILES: C[C@H]1CC2(CCN(c3ccc(S(=O)(=O)C4CCN(C5CCN(c6ccc7c(c6)C(=O)N(C6CCC(=O)NC6=O)C7=O)CC5)CC4)cc3)CC2)CN1c1ccc(C#N)c(Cl)c1] |
| 162 | 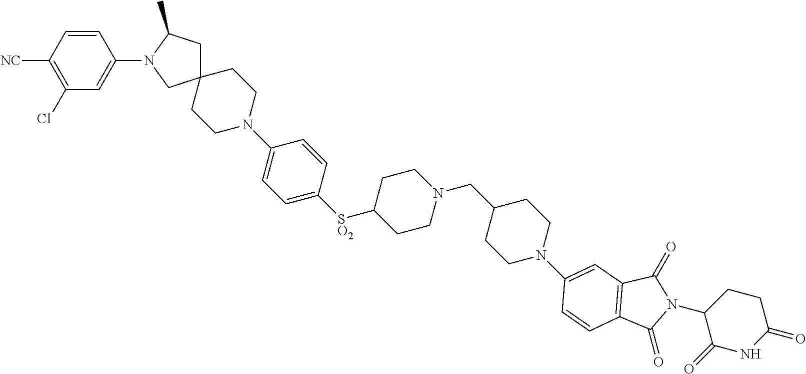 [SMILES: C[C@H]1CC2(CCN(c3ccc(S(=O)(=O)C4CCN(CC5CCN(c6ccc7c(c6)C(=O)N(C6CCC(=O)NC6=O)C7=O)CC5)CC4)cc3)CC2)CN1c1ccc(C#N)c(Cl)c1] |
| 163 | 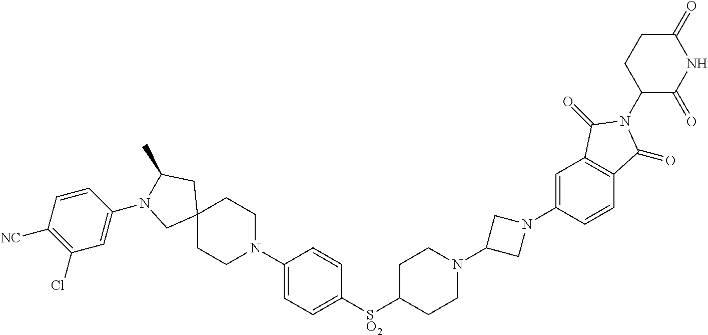 [SMILES: C[C@H]1CC2(CCN(c3ccc(S(=O)(=O)C4CCN(C5CN(c6ccc7c(c6)C(=O)N(C6CCC(=O)NC6=O)C7=O)C5)CC4)cc3)CC2)CN1c1ccc(C#N)c(Cl)c1] |
| 164 | 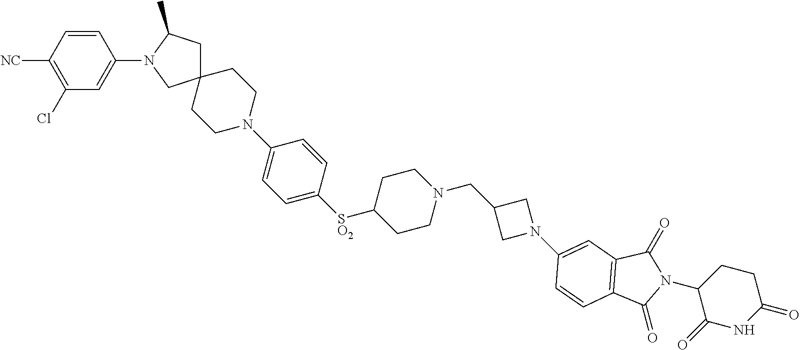 [SMILES: C[C@H]1CC2(CCN(c3ccc(S(=O)(=O)C4CCN(CC5CN(c6ccc7c(c6)C(=O)N(C6CCC(=O)NC6=O)C7=O)C5)CC4)cc3)CC2)CN1c1ccc(C#N)c(Cl)c1] |
| 165 | 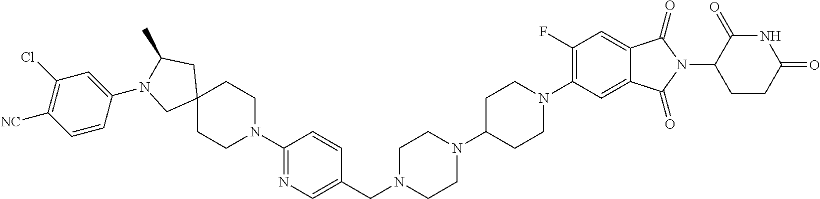 [SMILES: C[C@H]1CC2(CCN(c3ccc(CN4CCN(C5CCN(c6cc7c(cc6F)C(=O)N(C6CCC(=O)NC6=O)C7=O)CC5)CC4)cn3)CC2)CN1c1ccc(C#N)c(Cl)c1] |
| 166 | 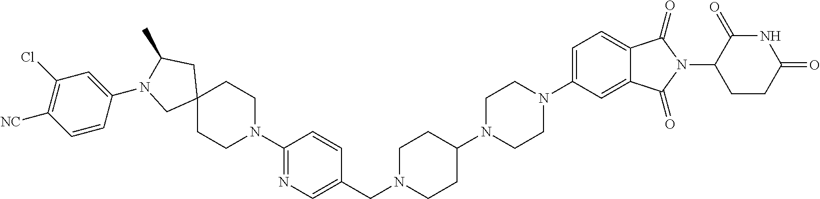 [SMILES: C[C@H]1CC2(CCN(c3ccc(CN4CCC(N5CCN(c6ccc7c(c6)C(=O)N(C6CCC(=O)NC6=O)C7=O)CC5)CC4)cn3)CC2)CN1c1ccc(C#N)c(Cl)c1] |
| 167 | 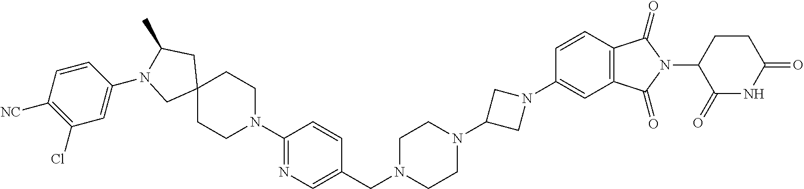 [SMILES: C[C@H]1CC2(CCN(c3ccc(CN4CCN(C5CN(c6ccc7c(c6)C(=O)N(C6CCC(=O)NC6=O)C7=O)C5)CC4)cn3)CC2)CN1c1ccc(C#N)c(Cl)c1] |
| 168 | 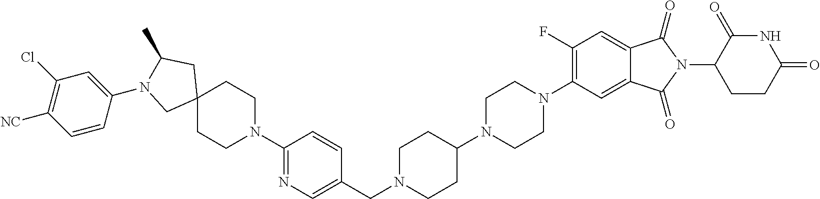 [SMILES: C[C@H]1CC2(CCN(c3ccc(CN4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| 169 | 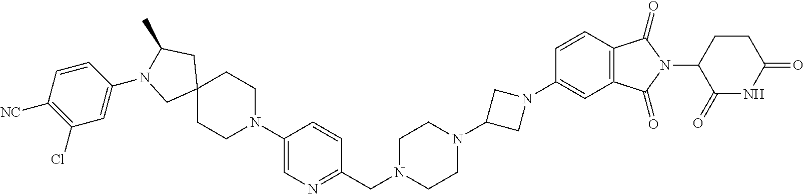 [SMILES: C[C@H]1CC2(CCN(c3ccc(CN4CCN(C5CN(c6ccc7c(c6)C(=O)N(C6CCC(=O)NC6=O)C7=O)C5)CC4)nc3)CC2)CN1c1ccc(C#N)c(Cl)c1] |
| 170 | 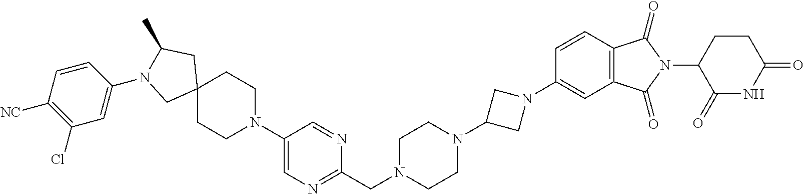 [SMILES: C[C@H]1CC2(CCN(c3cnc(CN4CCN(C5CN(c6ccc7c(c6)C(=O)N(C6CCC(=O)NC6=O)C7=O)C5)CC4)nc3)CC2)CN1c1ccc(C#N)c(Cl)c1] |
| 171 | 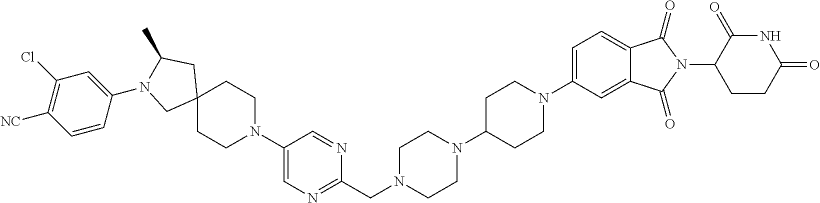 [SMILES: C[C@H]1CC2(CCN(c3cnc(CN4CCN(C5CCN(c6ccc7c(c6)C(=O)N(C6CCC(=O)NC6=O)C7=O)CC5)CC4)nc3)CC2)CN1c1ccc(C#N)c(Cl)c1] |
| 172 | 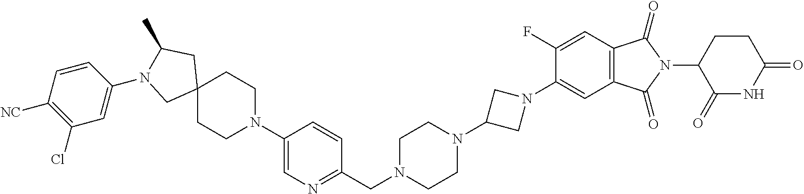 [SMILES: C[C@H]1CC2(CCN(c3ccc(CN4CCN(C5CN(c6cc7c(cc6F)C(=O)N(C6CCC(=O)NC6=O)C7=O)C5)CC4)nc3)CC2)CN1c1ccc(C#N)c(Cl)c1] |
| 173 | 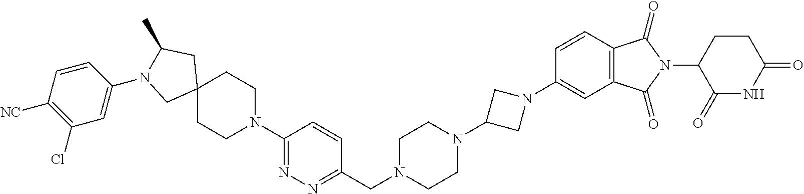 [SMILES: C[C@H]1CC2(CCN(c3ccc(CN4CCN(C5CN(c6ccc7c(c6)C(=O)N(C6CCC(=O)NC6=O)C7=O)C5)CC4)nn3)CC2)CN1c1ccc(C#N)c(Cl)c1] |
| 174 | 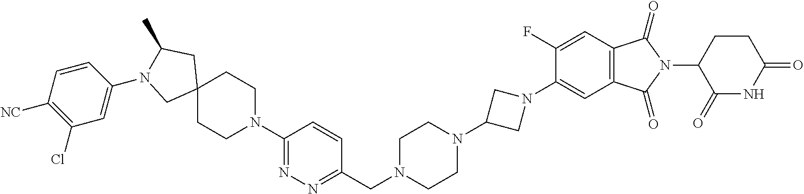 [SMILES: C[C@H]1CC2(CCN(c3ccc(CN4CCN(C5CN(c6cc7c(cc6F)C(=O)N(C6CCC(=O)NC6=O)C7=O)C5)CC4)nn3)CC2)CN1c1ccc(C#N)c(Cl)c1] |
| 175 | 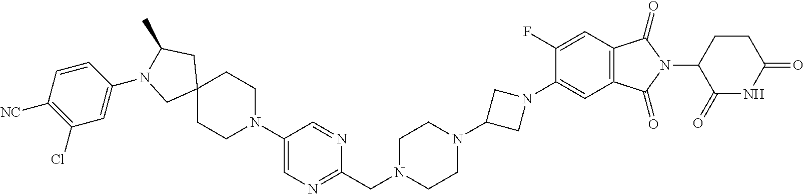 [SMILES: C[C@H]1CC2(CCN(c3cnc(CN4CCN(C5CN(c6cc7c(cc6F)C(=O)N(C6CCC(=O)NC6=O)C7=O)C5)CC4)nc3)CC2)CN1c1ccc(C#N)c(Cl)c1] |
| 176 | 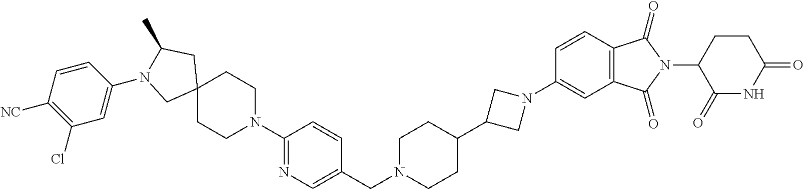 [SMILES: C[C@H]1CC2(CCN(c3ccc(CN4CCC(C5CN(c6ccc7c(c6)C(=O)N(C6CCC(=O)NC6=O)C7=O)C5)CC4)cn3)CC2)CN1c1ccc(C#N)c(Cl)c1] |
| 177 | 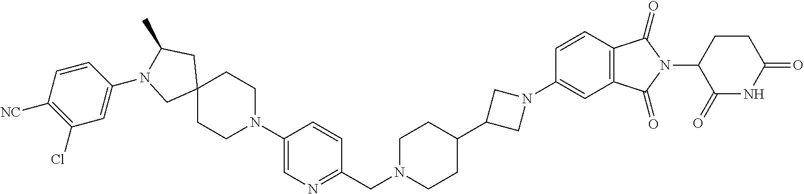 [SMILES: C[C@H]1CC2(CCN(c3ccc(CN4CCC(C5CN(c6ccc7c(c6)C(=O)N(C6CCC(=O)NC6=O)C7=O)C5)CC4)nc3)CC2)CN1c1ccc(C#N)c(Cl)c1] |
| 178 | 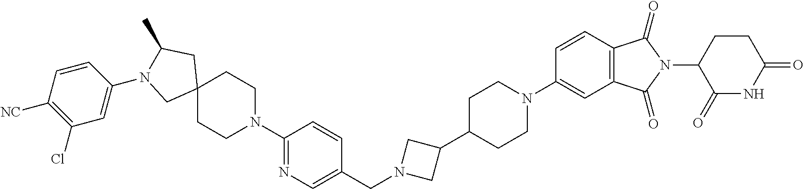 [SMILES: C[C@H]1CC2(CCN(c3ccc(CN4CC(C5CCN(c6ccc7c(c6)C(=O)N(C6CCC(=O)NC6=O)C7=O)CC5)C4)cn3)CC2)CN1c1ccc(C#N)c(Cl)c1] |
| 179 | 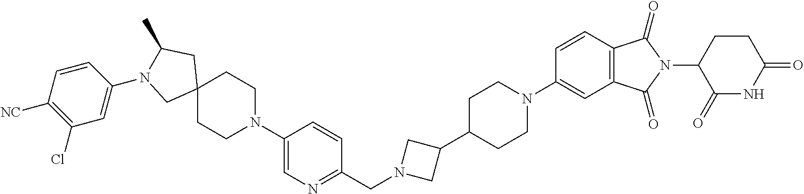 [SMILES: C[C@H]1CC2(CCN(c3ccc(CN4CC(C5CCN(c6ccc7c(c6)C(=O)N(C6CCC(=O)NC6=O)C7=O)CC5)C4)nc3)CC2)CN1c1ccc(C#N)c(Cl)c1] |
| 180 | 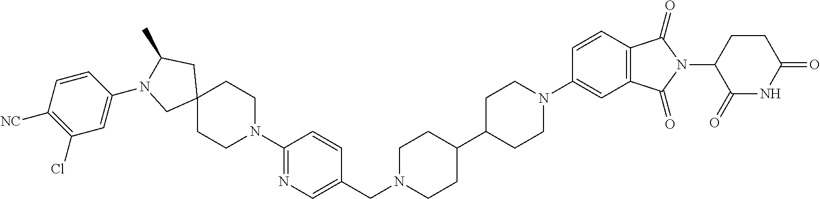 [SMILES: C[C@H]1CC2(CCN(c3ccc(CN4CCC(C5CCN(c6ccc7c(c6)C(=O)N(C6CCC(=O)NC6=O)C7=O)CC5)CC4)cn3)CC2)CN1c1ccc(C#N)c(Cl)c1] |
| 181 | 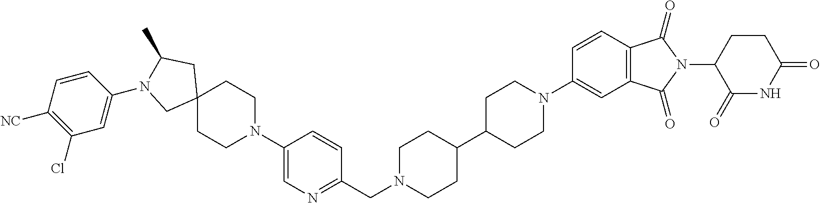 [SMILES: C[C@H]1CC2(CCN(c3ccc(CN4CCC(C5CCN(c6ccc7c(c6)C(=O)N(C6CCC(=O)NC6=O)C7=O)CC5)CC4)nc3)CC2)CN1c1ccc(C#N)c(Cl)c1] |
| 182 | 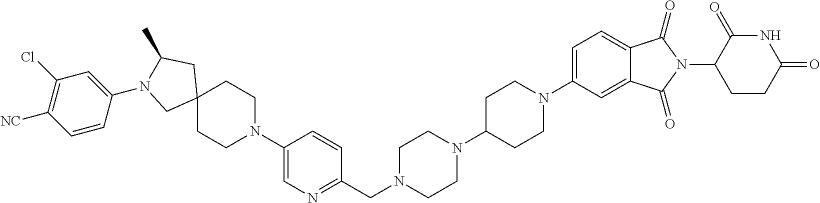 [SMILES: C[C@H]1CC2(CCN(c3ccc(CN4CCN(C5CCN(c6ccc7c(c6)C(=O)N(C6CCC(=O)NC6=O)C7=O)CC5)CC4)nc3)CC2)CN1c1ccc(C#N)c(Cl)c1] |
| 183 | 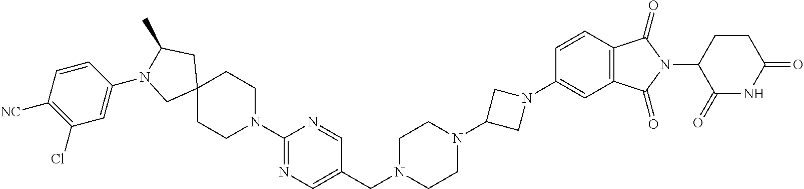 [SMILES: C[C@H]1CC2(CCN(c3ncc(CN4CCN(C5CN(c6ccc7c(c6)C(=O)N(C6CCC(=O)NC6=O)C7=O)C5)CC4)cn3)CC2)CN1c1ccc(C#N)c(Cl)c1] |
| 184 | 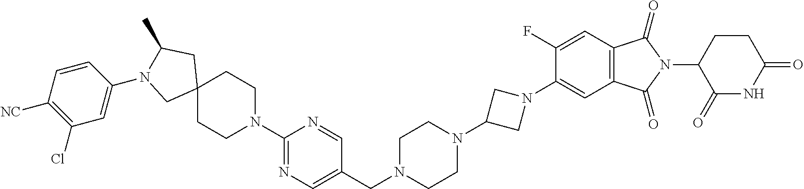 [SMILES: C[C@H]1CC2(CCN(c3ncc(CN4CCN(C5CN(c6cc7c(cc6F)C(=O)N(C6CCC(=O)NC6=O)C7=O)C5)CC4)cn3)CC2)CN1c1ccc(C#N)c(Cl)c1] |
| 185 | 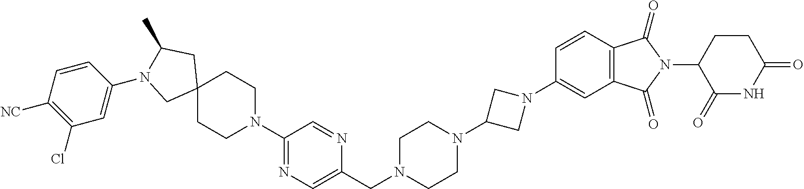 [SMILES: C[C@H]1CC2(CCN(c3cnc(CN4CCN(C5CN(c6ccc7c(c6)C(=O)N(C6CCC(=O)NC6=O)C7=O)C5)CC4)cn3)CC2)CN1c1ccc(C#N)c(Cl)c1] |
| 186 | 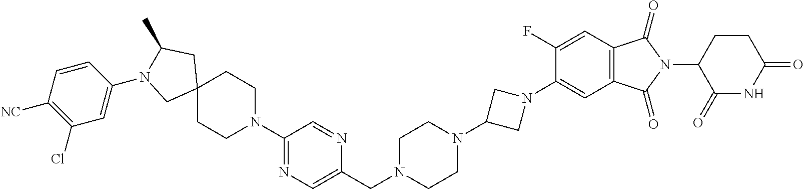 [SMILES: C[C@H]1CC2(CCN(c3cnc(CN4CCN(C5CN(c6cc7c(cc6F)C(=O)N(C6CCC(=O)NC6=O)C7=O)C5)CC4)cn3)CC2)CN1c1ccc(C#N)c(Cl)c1] |
| 187 | 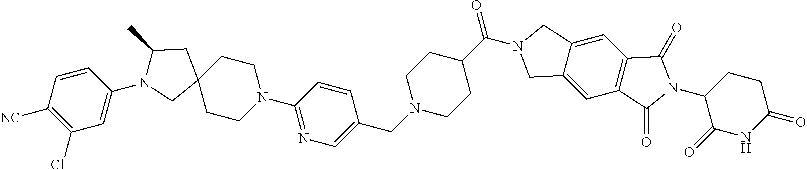 [SMILES: C[C@H]1CC2(CCN(c3ccc(CN4CCC(C(=O)N5Cc6cc7c(cc6C5)C(=O)N(C5CCC(=O)NC5=O)C7=O)CC4)cn3)CC2)CN1c1ccc(C#N)c(Cl)c1] |
| 188 | 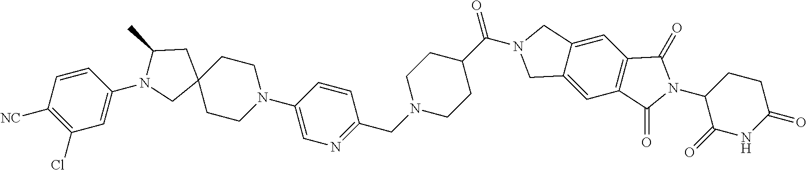 [SMILES: C[C@H]1CC2(CCN(c3ccc(CN4CCC(C(=O)N5Cc6cc7c(cc6C5)C(=O)N(C5CCC(=O)NC5=O)C7=O)CC4)nc3)CC2)CN1c1ccc(C#N)c(Cl)c1] |
| 189 | 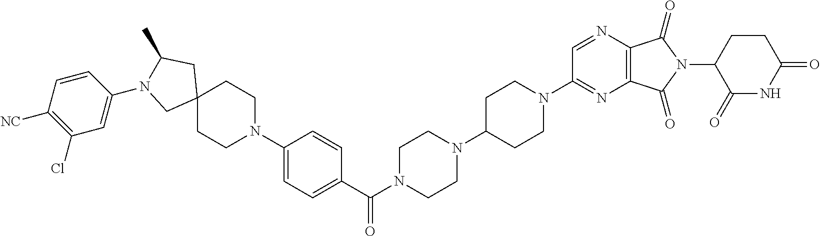 [SMILES: C[C@H]1CC2(CCN(c3ccc(C(=O)N4CCN(C5CCN(c6cnc7c(n6)C(=O)N(C6CCC(=O)NC6=O)C7=O)CC5)CC4)cc3)CC2)CN1c1ccc(C#N)c(Cl)c1] |
| 190 | 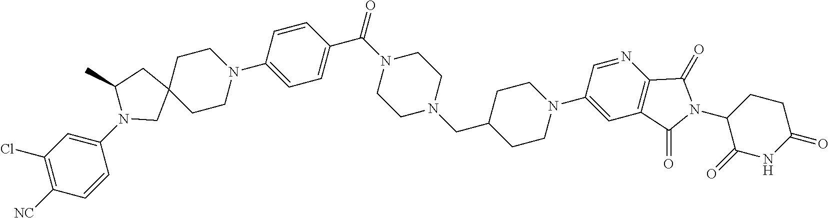 [SMILES: C[C@H]1CC2(CCN(c3ccc(C(=O)N4CCN(CC5CCN(c6cnc7c(c6)C(=O)N(C6CCC(=O)NC6=O)C7=O)CC5)CC4)cc3)CC2)CN1c1ccc(C#N)c(Cl)c1] |
| 191 | 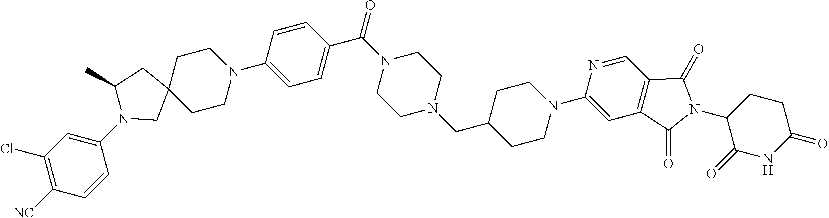 [SMILES: C[C@H]1CC2(CCN(c3ccc(C(=O)N4CCN(CC5CCN(c6cc7c(cn6)C(=O)N(C6CCC(=O)NC6=O)C7=O)CC5)CC4)cc3)CC2)CN1c1ccc(C#N)c(Cl)c1] |
| 192 | 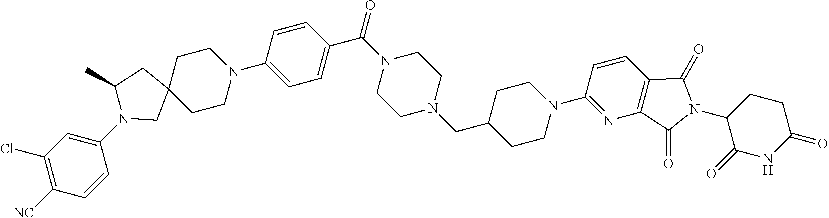 [SMILES: C[C@H]1CC2(CCN(c3ccc(C(=O)N4CCN(CC5CCN(c6ccc7c(n6)C(=O)N(C6CCC(=O)NC6=O)C7=O)CC5)CC4)cc3)CC2)CN1c1ccc(C#N)c(Cl)c1] |
| 193 | 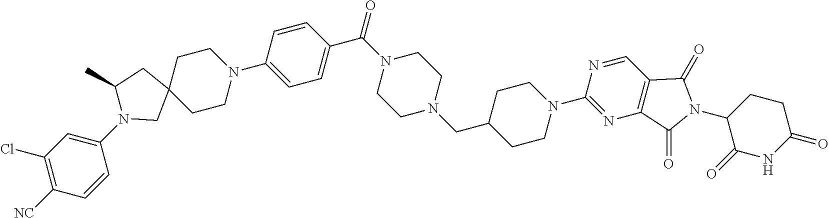 [SMILES: C[C@H]1CC2(CCN(c3ccc(C(=O)N4CCN(CC5CCN(c6ncc7c(n6)C(=O)N(C6CCC(=O)NC6=O)C7=O)CC5)CC4)cc3)CC2)CN1c1ccc(C#N)c(Cl)c1] |
| 194 | 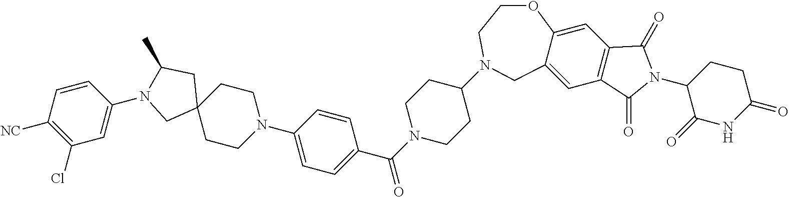 [SMILES: C[C@H]1CC2(CCN(c3ccc(C(=O)N4CCC(N5CCOc6cc7c(cc6C5)C(=O)N(C5CCC(=O)NC5=O)C7=O)CC4)cc3)CC2)CN1c1ccc(C#N)c(Cl)c1] |
| 195 | 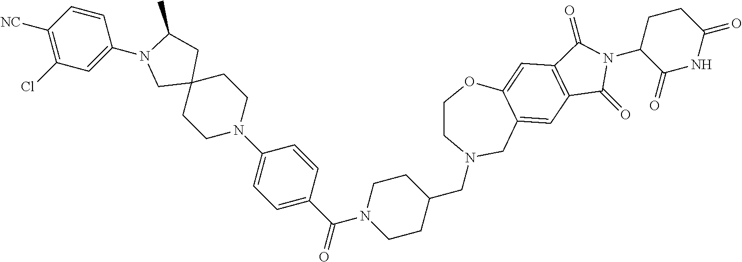 [SMILES: C[C@H]1CC2(CCN(c3ccc(C(=O)N4CCC(CN5CCOc6cc7c(cc6C5)C(=O)N(C5CCC(=O)NC5=O)C7=O)CC4)cc3)CC2)CN1c1ccc(C#N)c(Cl)c1] |
| 196 | 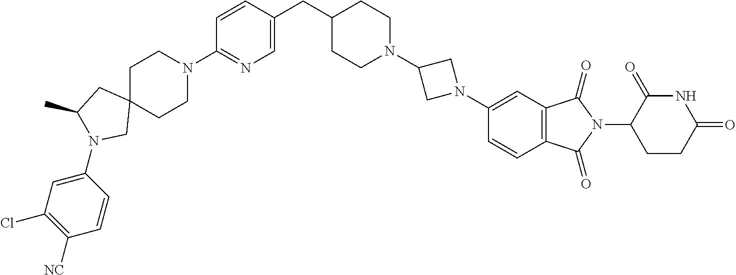 [SMILES: C[C@H]1CC2(CCN(c3ccc(CC4CCN(C5CN(c6ccc7c(c6)C(=O)N(C6CCC(=O)NC6=O)C7=O)C5)CC4)cn3)CC2)CN1c1ccc(C#N)c(Cl)c1] |
| 197 | 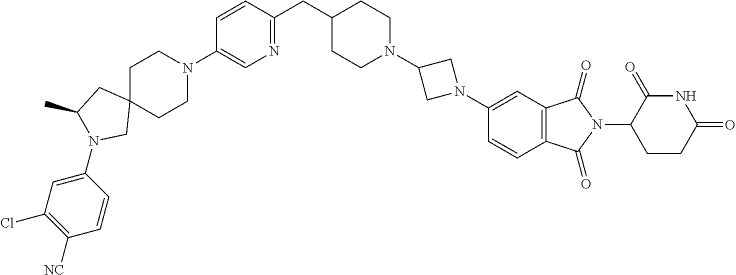 [SMILES: C[C@H]1CC2(CCN(c3ccc(CC4CCN(C5CN(c6ccc7c(c6)C(=O)N(C6CCC(=O)NC6=O)C7=O)C5)CC4)nc3)CC2)CN1c1ccc(C#N)c(Cl)c1] |
| 198 | 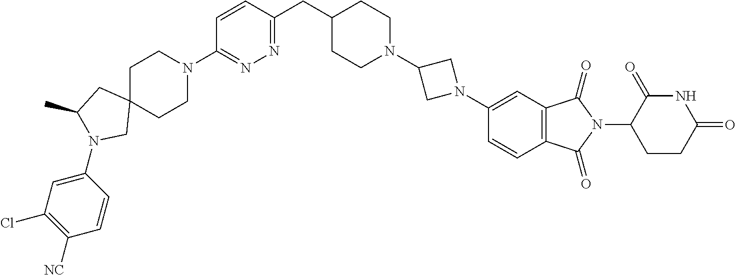 [SMILES: C[C@H]1CC2(CCN(c3ccc(CC4CCN(C5CN(c6ccc7c(c6)C(=O)N(C6CCC(=O)NC6=O)C7=O)C5)CC4)nn3)CC2)CN1c1ccc(C#N)c(Cl)c1] |
| 199 | 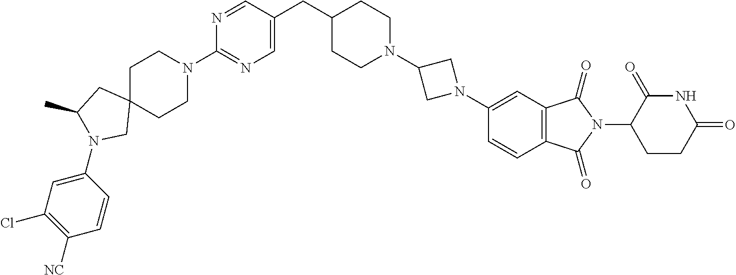 [SMILES: C[C@H]1CC2(CCN(c3ncc(CC4CCN(C5CN(c6ccc7c(c6)C(=O)N(C6CCC(=O)NC6=O)C7=O)C5)CC4)cn3)CC2)CN1c1ccc(C#N)c(Cl)c1] |
| 200 | 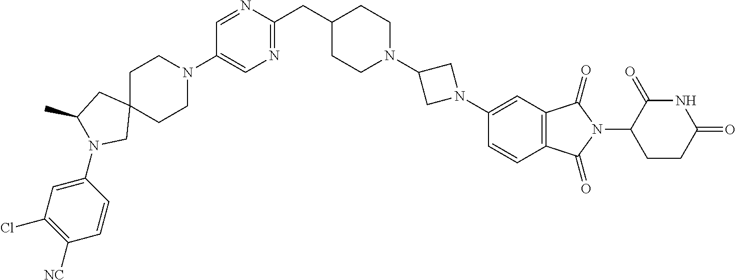 [SMILES: C[C@H]1CC2(CCN(c3cnc(CC4CCN(C5CN(c6ccc7c(c6)C(=O)N(C6CCC(=O)NC6=O)C7=O)C5)CC4)nc3)CC2)CN1c1ccc(C#N)c(Cl)c1] |
| 201 | 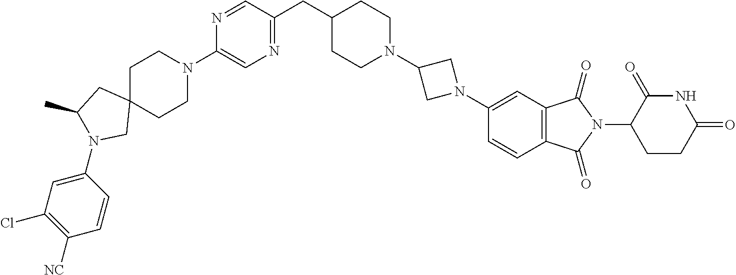 [SMILES: C[C@H]1CC2(CCN(c3cnc(CC4CCN(C5CN(c6ccc7c(c6)C(=O)N(C6CCC(=O)NC6=O)C7=O)C5)CC4)cn3)CC2)CN1c1ccc(C#N)c(Cl)c1] |
| 202 | 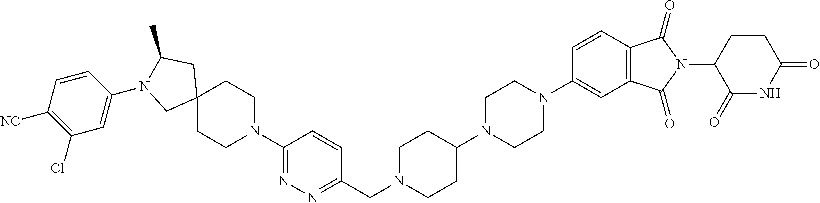 [SMILES: C[C@H]1CC2(CCN(c3ccc(CN4CCC(N5CCN(c6ccc7c(c6)C(=O)N(C6CCC(=O)NC6=O)C7=O)CC5)CC4)nn3)CC2)CN1c1ccc(C#N)c(Cl)c1] |
| 203 | 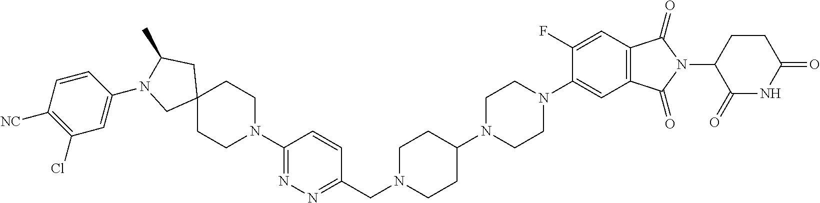 [SMILES: C[C@H]1CC2(CCN(c3ccc(CN4CCC(N5CCN(c6cc7c(cc6F)C(=O)N(C6CCC(=O)NC6=O)C7=O)CC5)CC4)nn3)CC2)CN1c1ccc(C#N)c(Cl)c1] |
| 204 | 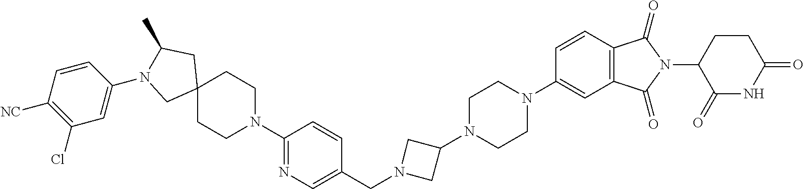 [SMILES: C[C@H]1CC2(CCN(c3ccc(CN4CC(N5CCN(c6ccc7c(c6)C(=O)N(C6CCC(=O)NC6=O)C7=O)CC5)C4)cn3)CC2)CN1c1ccc(C#N)c(Cl)c1] |
| 205 | 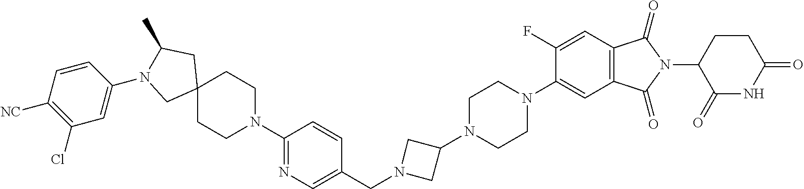 [SMILES: C[C@H]1CC2(CCN(c3ccc(CN4CC(N5CCN(c6cc7c(cc6F)C(=O)N(C6CCC(=O)NC6=O)C7=O)CC5)C4)cn3)CC2)CN1c1ccc(C#N)c(Cl)c1] |
| 206 | 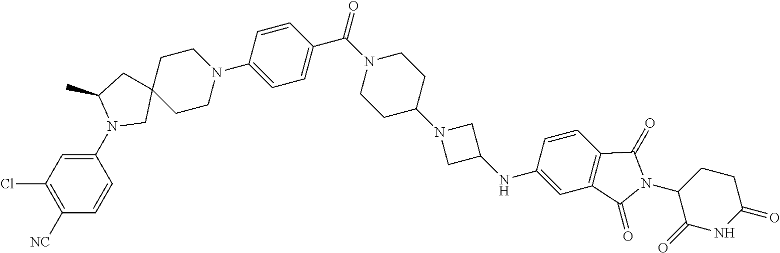 [SMILES: C[C@H]1CC2(CCN(c3ccc(C(=O)N4CCC(N5CC(Nc6ccc7c(c6)C(=O)N(C6CCC(=O)NC6=O)C7=O)C5)CC4)cc3)CC2)CN1c1ccc(C#N)c(Cl)c1] |
| 207 | 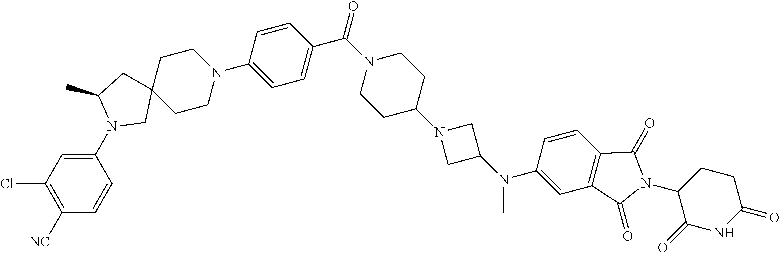 [SMILES: C[C@H]1CC2(CCN(c3ccc(C(=O)N4CCC(N5CC(N(C)c6ccc7c(c6)C(=O)N(C6CCC(=O)NC6=O)C7=O)C5)CC4)cc3)CC2)CN1c1ccc(C#N)c(Cl)c1] |
| 208 | 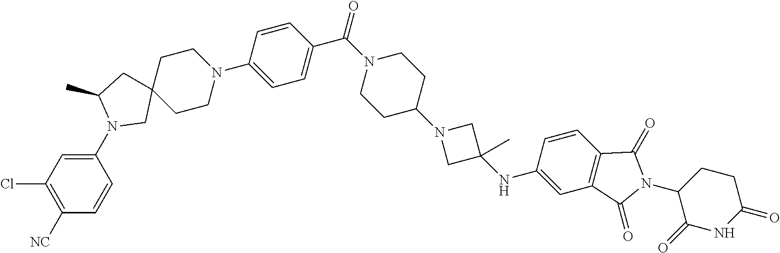 [SMILES: C[C@H]1CC2(CCN(c3ccc(C(=O)N4CCC(N5CC(C)(Nc6ccc7c(c6)C(=O)N(C6CCC(=O)NC6=O)C7=O)C5)CC4)cc3)CC2)CN1c1ccc(C#N)c(Cl)c1] |
| 209 | 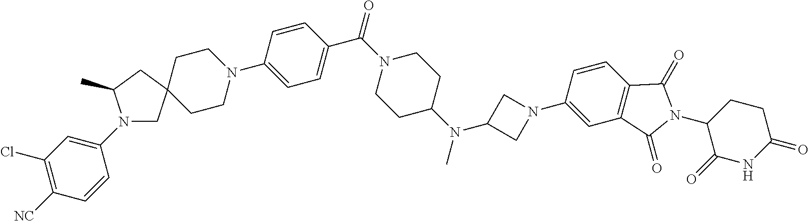 [SMILES: C[C@H]1CC2(CCN(c3ccc(C(=O)N4CCC(N(C)C5CN(c6ccc7c(c6)C(=O)N(C6CCC(=O)NC6=O)C7=O)C5)CC4)cc3)CC2)CN1c1ccc(C#N)c(Cl)c1] |
| 210 | 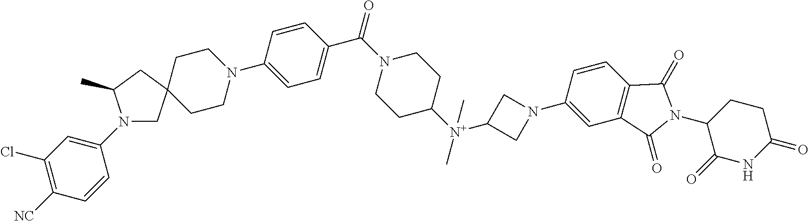 [SMILES: C[C@H]1CC2(CCN(c3ccc(C(=O)N4CCC([N+](C)(C)C5CN(c6ccc7c(c6)C(=O)N(C6CCC(=O)NC6=O)C7=O)C5)CC4)cc3)CC2)CN1c1ccc(C#N)c(Cl)c1] |
| 211 | 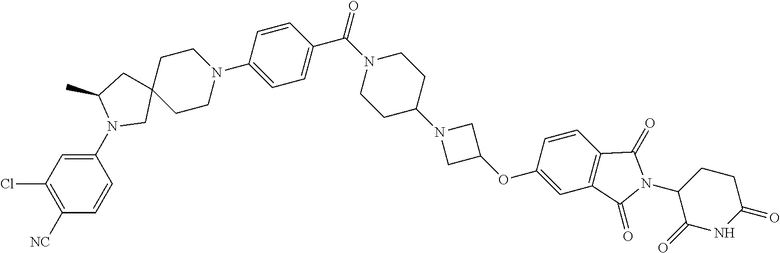 [SMILES: C[C@H]1CC2(CCN(c3ccc(C(=O)N4CCC(N5CC(Oc6ccc7c(c6)C(=O)N(C6CCC(=O)NC6=O)C7=O)C5)CC4)cc3)CC2)CN1c1ccc(C#N)c(Cl)c1] |
| 212 | 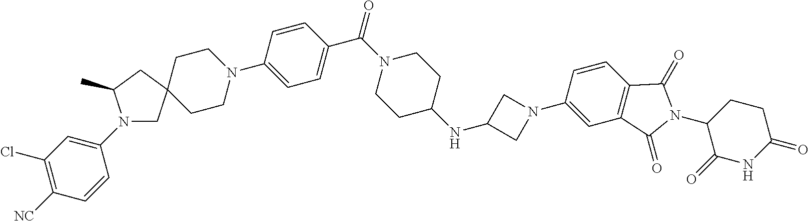 [SMILES: C[C@H]1CC2(CCN(c3ccc(C(=O)N4CCC(NC5CN(c6ccc7c(c6)C(=O)N(C6CCC(=O)NC6=O)C7=O)C5)CC4)cc3)CC2)CN1c1ccc(C#N)c(Cl)c1] |
| 213 | 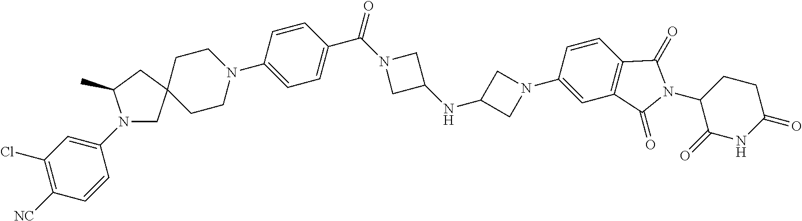 [SMILES: C[C@H]1CC2(CCN(c3ccc(C(=O)N4CC(NC5CN(c6ccc7c(c6)C(=O)N(C6CCC(=O)NC6=O)C7=O)C5)C4)cc3)CC2)CN1c1ccc(C#N)c(Cl)c1] |
| 214 | 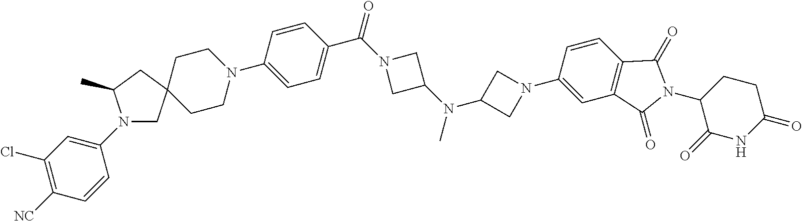 [SMILES: C[C@H]1CC2(CCN(c3ccc(C(=O)N4CC(N(C)C5CN(c6ccc7c(c6)C(=O)N(C6CCC(=O)NC6=O)C7=O)C5)C4)cc3)CC2)CN1c1ccc(C#N)c(Cl)c1] |
| 215 | 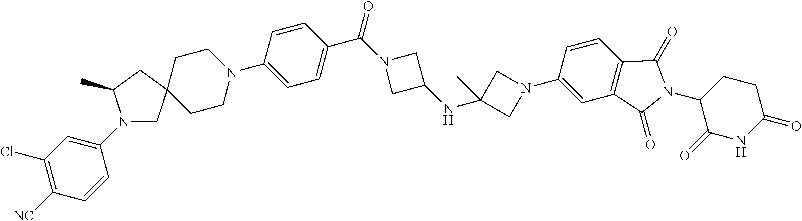 [SMILES: C[C@H]1CC2(CCN(c3ccc(C(=O)N4CC(NC5(C)CN(c6ccc7c(c6)C(=O)N(C6CCC(=O)NC6=O)C7=O)C5)C4)cc3)CC2)CN1c1ccc(C#N)c(Cl)c1] |
| 216 | 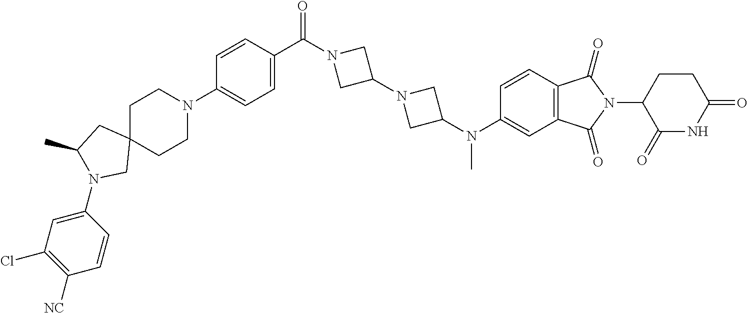 [SMILES: C[C@H]1CC2(CCN(c3ccc(C(=O)N4CC(N5CC(N(C)c6ccc7c(c6)C(=O)N(C6CCC(=O)NC6=O)C7=O)C5)C4)cc3)CC2)CN1c1ccc(C#N)c(Cl)c1] |
| 217 | 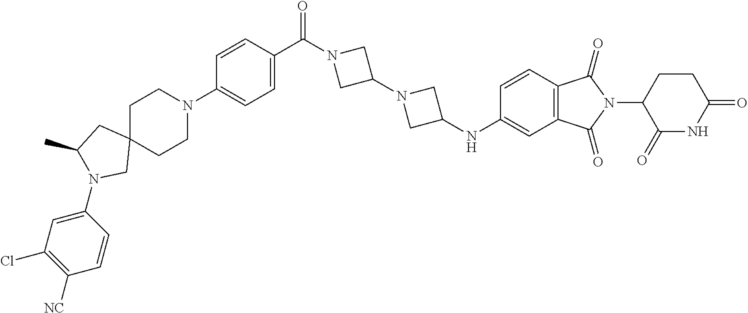 [SMILES: C[C@H]1CC2(CCN(c3ccc(C(=O)N4CC(N5CC(Nc6ccc7c(c6)C(=O)N(C6CCC(=O)NC6=O)C7=O)C5)C4)cc3)CC2)CN1c1ccc(C#N)c(Cl)c1] |
| 218 | 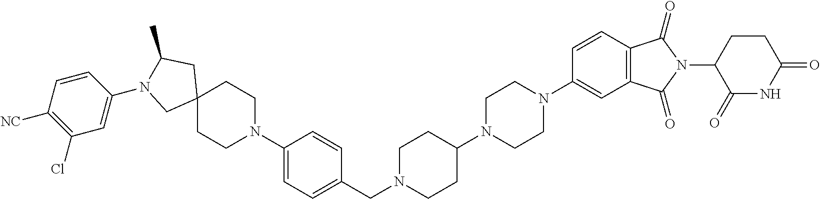 [SMILES: C[C@H]1CC2(CCN(c3ccc(CN4CCC(N5CCN(c6ccc7c(c6)C(=O)N(C6CCC(=O)NC6=O)C7=O)CC5)CC4)cc3)CC2)CN1c1ccc(C#N)c(Cl)c1] |
| 219 | 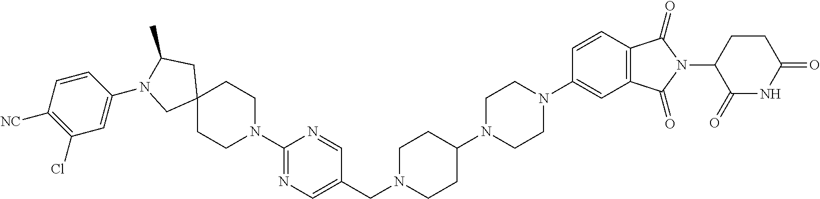 [SMILES: C[C@H]1CC2(CCN(c3ncc(CN4CCC(N5CCN(c6ccc7c(c6)C(=O)N(C6CCC(=O)NC6=O)C7=O)CC5)CC4)cn3)CC2)CN1c1ccc(C#N)c(Cl)c1] |
| 220 | 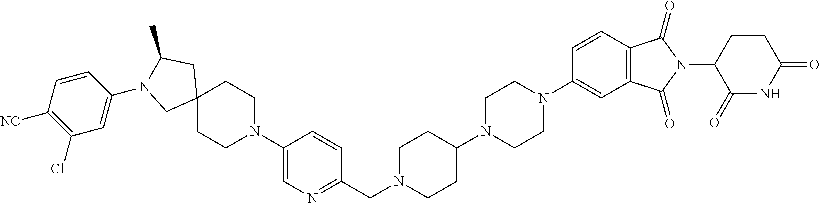 [SMILES: C[C@H]1CC2(CCN(c3ccc(CN4CCC(N5CCN(c6ccc7c(c6)C(=O)N(C6CCC(=O)NC6=O)C7=O)CC5)CC4)nc3)CC2)CN1c1ccc(C#N)c(Cl)c1] |
| 221 | 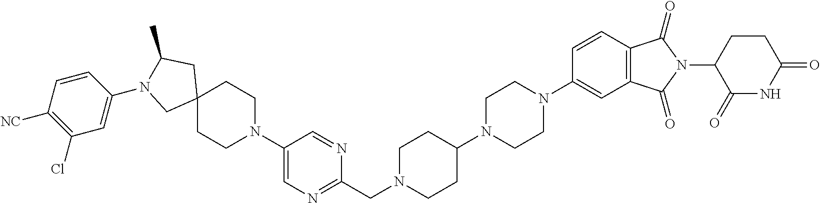 [SMILES: C[C@H]1CC2(CCN(c3cnc(CN4CCC(N5CCN(c6ccc7c(c6)C(=O)N(C6CCC(=O)NC6=O)C7=O)CC5)CC4)nc3)CC2)CN1c1ccc(C#N)c(Cl)c1] |
| 222 | 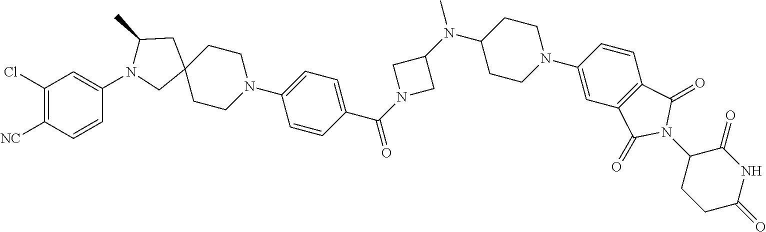 [SMILES: C[C@H]1CC2(CCN(c3ccc(C(=O)N4CC(N(C)C5CCN(c6ccc7c(c6)C(=O)N(C6CCC(=O)NC6=O)C7=O)CC5)C4)cc3)CC2)CN1c1ccc(C#N)c(Cl)c1] |
| 223 | 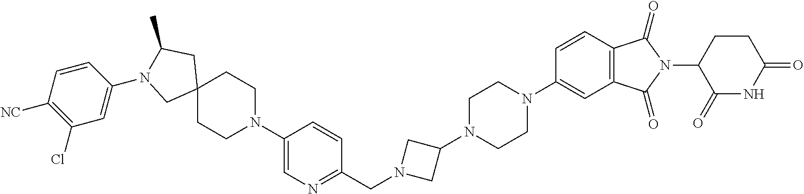 [SMILES: C[C@H]1CC2(CCN(c3ccc(CN4CC(N5CCN(c6ccc7c(c6)C(=O)N(C6CCC(=O)NC6=O)C7=O)CC5)C4)nc3)CC2)CN1c1ccc(C#N)c(Cl)c1] |
| 224 | 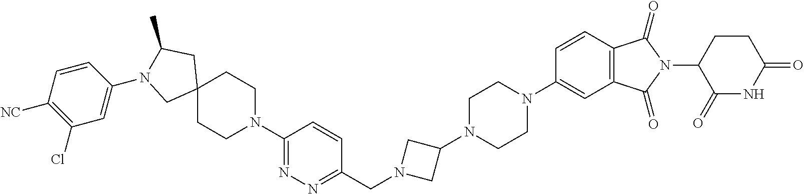 [SMILES: C[C@H]1CC2(CCN(c3ccc(CN4CC(N5CCN(c6ccc7c(c6)C(=O)N(C6CCC(=O)NC6=O)C7=O)CC5)C4)nn3)CC2)CN1c1ccc(C#N)c(Cl)c1] |
| 225 | 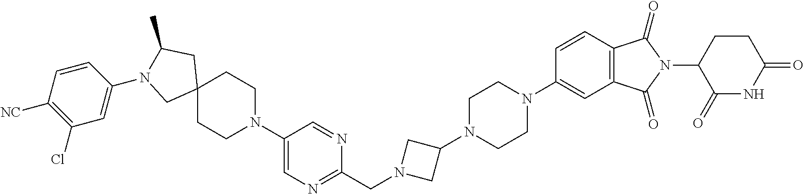 [SMILES: C[C@H]1CC2(CCN(c3cnc(CN4CC(N5CCN(c6ccc7c(c6)C(=O)N(C6CCC(=O)NC6=O)C7=O)CC5)C4)nc3)CC2)CN1c1ccc(C#N)c(Cl)c1] |
| 226 | 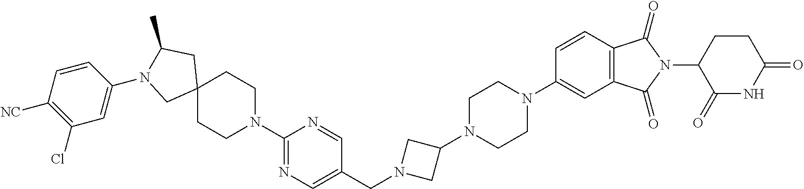 [SMILES: C[C@H]1CC2(CCN(c3ncc(CN4CC(N5CCN(c6ccc7c(c6)C(=O)N(C6CCC(=O)NC6=O)C7=O)CC5)C4)cn3)CC2)CN1c1ccc(C#N)c(Cl)c1] |
| 227 | 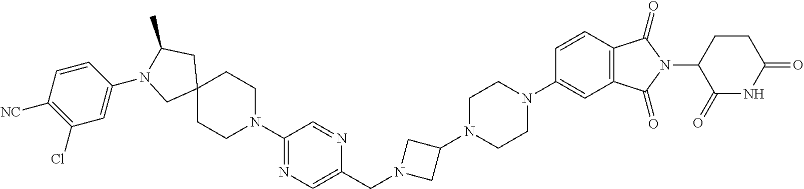 [SMILES: C[C@H]1CC2(CCN(c3cnc(CN4CC(N5CCN(c6ccc7c(c6)C(=O)N(C6CCC(=O)NC6=O)C7=O)CC5)C4)cn3)CC2)CN1c1ccc(C#N)c(Cl)c1] |
| 228 | 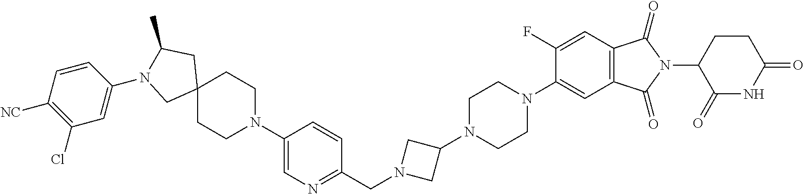 [SMILES: C[C@H]1CC2(CCN(c3ccc(CN4CC(N5CCN(c6cc7c(cc6F)C(=O)N(C6CCC(=O)NC6=O)C7=O)CC5)C4)nc3)CC2)CN1c1ccc(C#N)c(Cl)c1] |
| 229 | 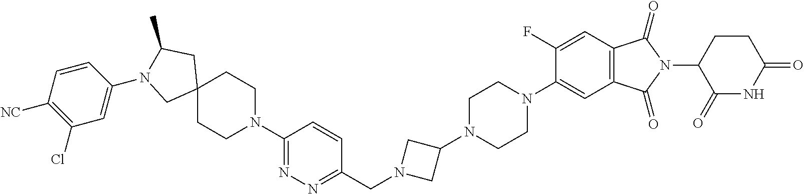 [SMILES: C[C@H]1CC2(CCN(c3ccc(CN4CC(N5CCN(c6cc7c(cc6F)C(=O)N(C6CCC(=O)NC6=O)C7=O)CC5)C4)nn3)CC2)CN1c1ccc(C#N)c(Cl)c1] |
| 230 | 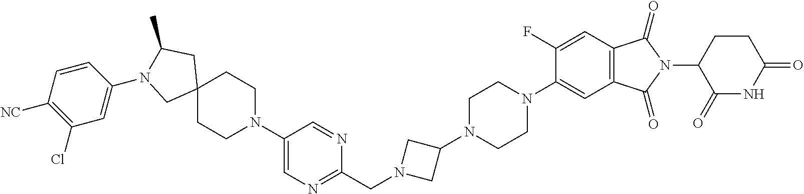 [SMILES: C[C@H]1CC2(CCN(c3cnc(CN4CC(N5CCN(c6cc7c(cc6F)C(=O)N(C6CCC(=O)NC6=O)C7=O)CC5)C4)nc3)CC2)CN1c1ccc(C#N)c(Cl)c1] |
| 231 | 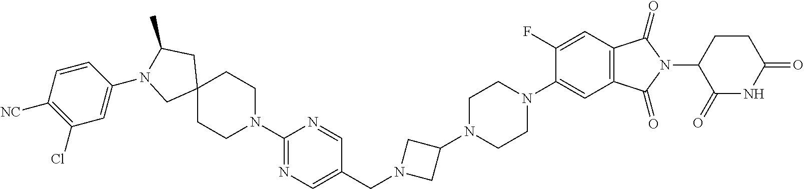 [SMILES: C[C@H]1CC2(CCN(c3ncc(CN4CC(N5CCN(c6cc7c(cc6F)C(=O)N(C6CCC(=O)NC6=O)C7=O)CC5)C4)cn3)CC2)CN1c1ccc(C#N)c(Cl)c1] |
| 232 | 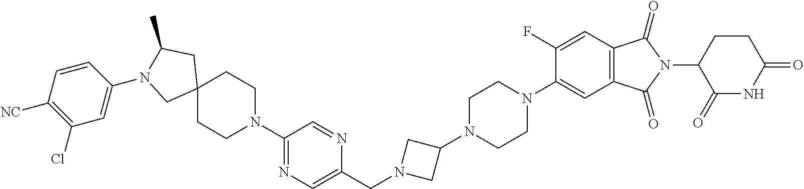 [SMILES: C[C@H]1CC2(CCN(c3cnc(CN4CC(N5CCN(c6cc7c(cc6F)C(=O)N(C6CCC(=O)NC6=O)C7=O)CC5)C4)cn3)CC2)CN1c1ccc(C#N)c(Cl)c1] |
| 233 | 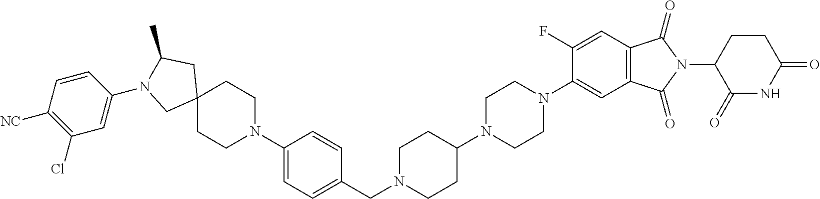 [SMILES: C[C@H]1CC2(CCN(c3ccc(CN4CCC(N5CCN(c6cc7c(cc6F)C(=O)N(C6CCC(=O)NC6=O)C7=O)CC5)CC4)cc3)CC2)CN1c1ccc(C#N)c(Cl)c1] |
| 234 | 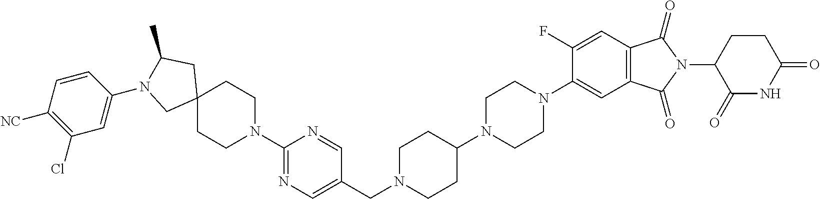 [SMILES: C[C@H]1CC2(CCN(c3ncc(CN4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| 235 | 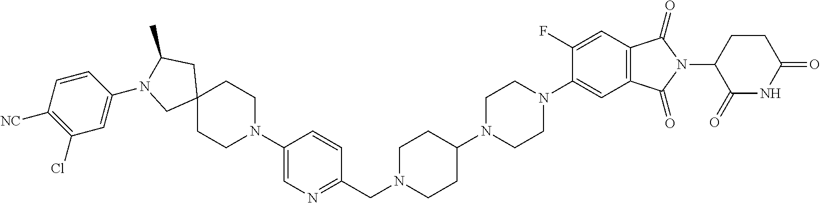 [SMILES: C[C@H]1CC2(CCN(c3ccc(CN4CCC(N5CCN(c6cc7c(cc6F)C(=O)N(C6CCC(=O)NC6=O)C7=O)CC5)CC4)nc3)CC2)CN1c1ccc(C#N)c(Cl)c1] |
| 236 | 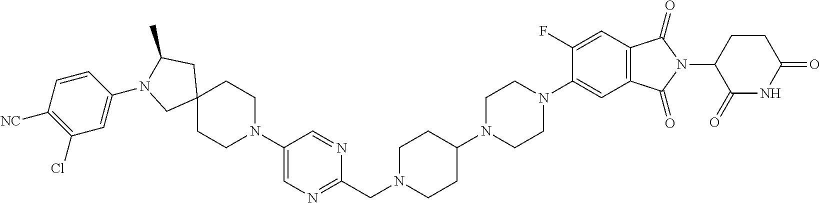 [SMILES: C[C@H]1CC2(CCN(c3cnc(CN4CCC(N5CCN(c6cc7c(cc6F)C(=O)N(C6CCC(=O)NC6=O)C7=O)CC5)CC4)nc3)CC2)CN1c1ccc(C#N)c(Cl)c1] |
| 237 | 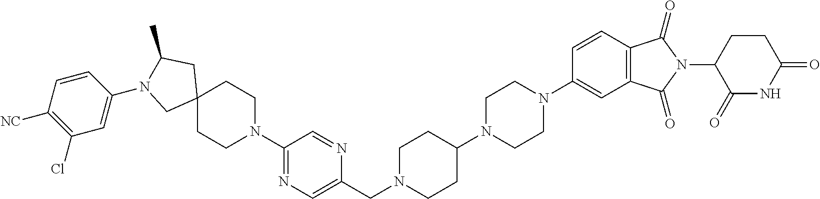 [SMILES: C[C@H]1CC2(CCN(c3cnc(CN4CCC(N5CCN(c6ccc7c(c6)C(=O)N(C6CCC(=O)NC6=O)C7=O)CC5)CC4)cn3)CC2)CN1c1ccc(C#N)c(Cl)c1] |
| 238 | 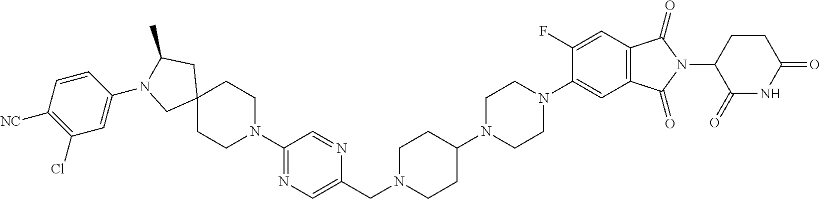 [SMILES: C[C@H]1CC2(CCN(c3cnc(CN4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| 239 | 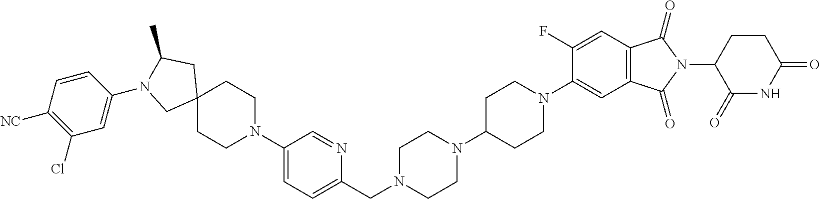 [SMILES: C[C@H]1CC2(CCN(c3ccc(CN4CCN(C5CCN(c6cc7c(cc6F)C(=O)N(C6CCC(=O)NC6=O)C7=O)CC5)CC4)nc3)CC2)CN1c1ccc(C#N)c(Cl)c1] |
| 240 | 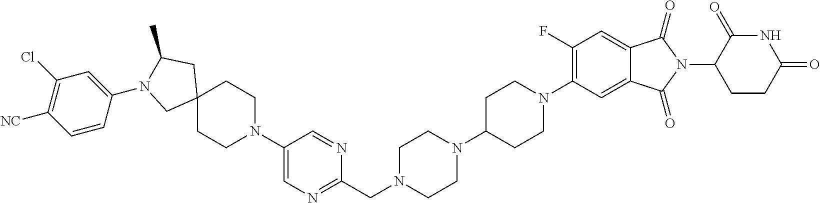 [SMILES: C[C@H]1CC2(CCN(c3cnc(CN4CCN(C5CCN(c6cc7c(cc6F)C(=O)N(C6CCC(=O)NC6=O)C7=O)CC5)CC4)nc3)CC2)CN1c1ccc(C#N)c(Cl)c1] |
| 241 | 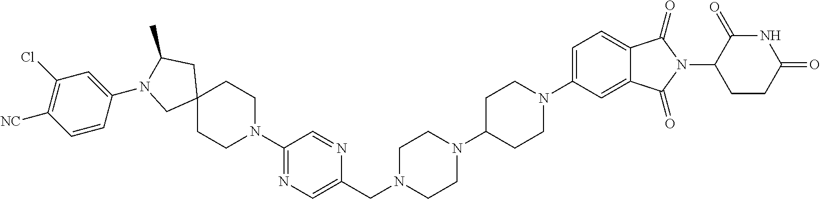 [SMILES: C[C@H]1CC2(CCN(c3cnc(CN4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| 242 | 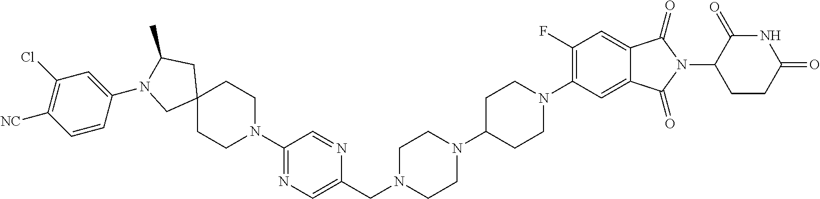 [SMILES: C[C@H]1CC2(CCN(c3cnc(CN4CCN(C5CCN(c6cc7c(cc6F)C(=O)N(C6CCC(=O)NC6=O)C7=O)CC5)CC4)cn3)CC2)CN1c1ccc(C#N)c(Cl)c1] |
| 243 | 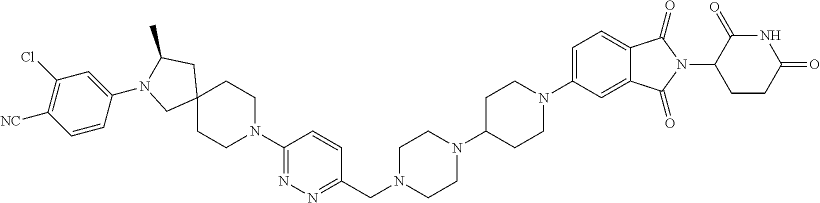 [SMILES: C[C@H]1CC2(CCN(c3ccc(CN4CCN(C5CCN(c6ccc7c(c6)C(=O)N(C6CCC(=O)NC6=O)C7=O)CC5)CC4)nn3)CC2)CN1c1ccc(C#N)c(Cl)c1] |
| 244 | 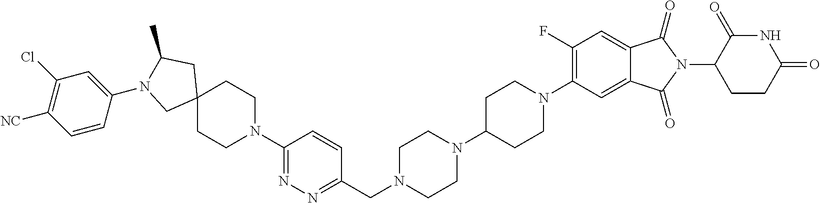 [SMILES: C[C@H]1CC2(CCN(c3ccc(CN4CCN(C5CCN(c6cc7c(cc6F)C(=O)N(C6CCC(=O)NC6=O)C7=O)CC5)CC4)nn3)CC2)CN1c1ccc(C#N)c(Cl)c1] |
| 245 | 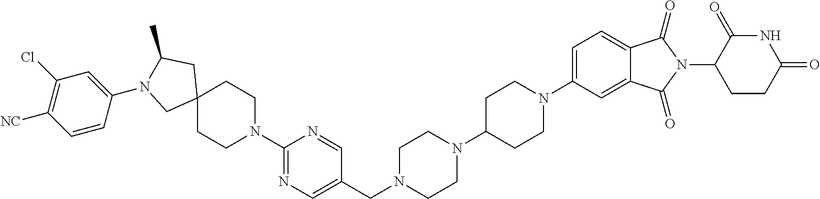 [SMILES: C[C@H]1CC2(CCN(c3ncc(CN4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| 246 | 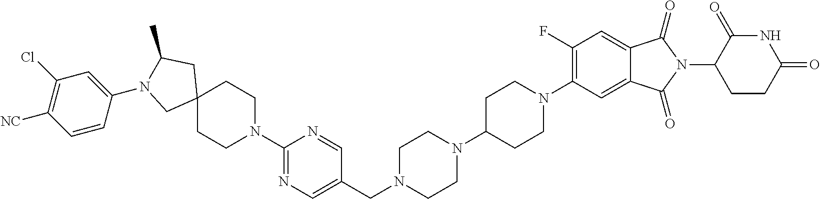 [SMILES: C[C@H]1CC2(CCN(c3ncc(CN4CCN(C5CCN(c6cc7c(cc6F)C(=O)N(C6CCC(=O)NC6=O)C7=O)CC5)CC4)cn3)CC2)CN1c1ccc(C#N)c(Cl)c1] |
| 247 | 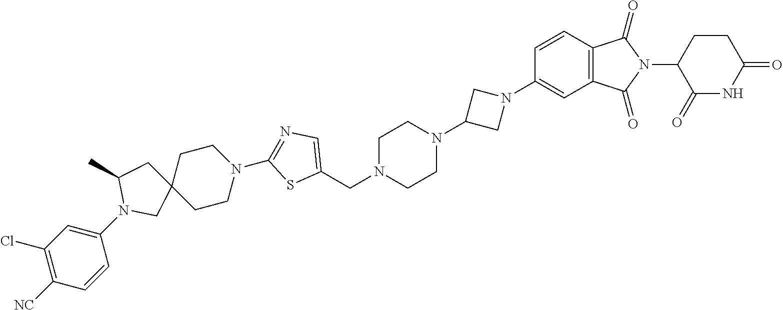 [SMILES: C[C@H]1CC2(CCN(c3ncc(CN4CCN(C5CN(c6ccc7c(c6)C(=O)N(C6CCC(=O)NC6=O)C7=O)C5)CC4)s3)CC2)CN1c1ccc(C#N)c(Cl)c1] |
| 248 | 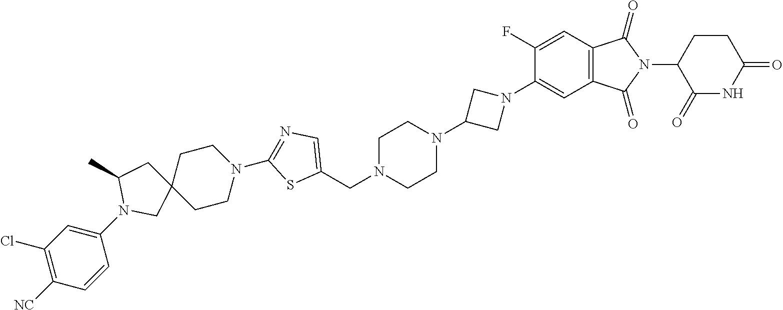 [SMILES: C[C@H]1CC2(CCN(c3ncc(CN4CCN(C5CN(c6cc7c(cc6F)C(=O)N(C6CCC(=O)NC6=O)C7=O)C5)CC4)s3)CC2)CN1c1ccc(C#N)c(Cl)c1] |
| 249 | 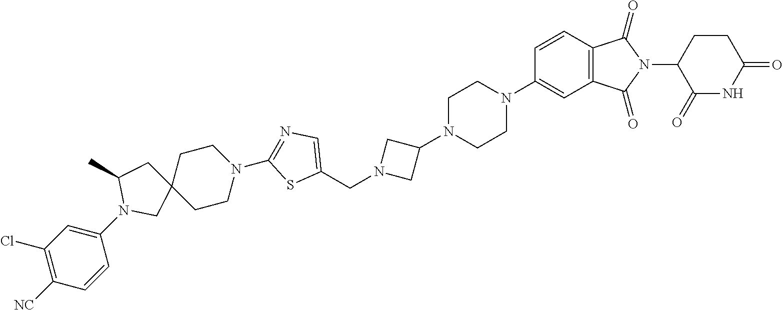 [SMILES: C[C@H]1CC2(CCN(c3ncc(CN4CC(N5CCN(c6ccc7c(c6)C(=O)N(C6CCC(=O)NC6=O)C7=O)CC5)C4)s3)CC2)CN1c1ccc(C#N)c(Cl)c1] |
| 250 | 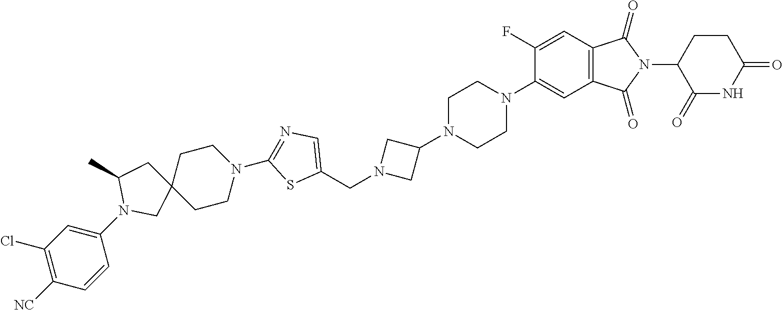 [SMILES: C[C@H]1CC2(CCN(c3ncc(CN4CC(N5CCN(c6cc7c(cc6F)C(=O)N(C6CCC(=O)NC6=O)C7=O)CC5)C4)s3)CC2)CN1c1ccc(C#N)c(Cl)c1] |
| 251 | 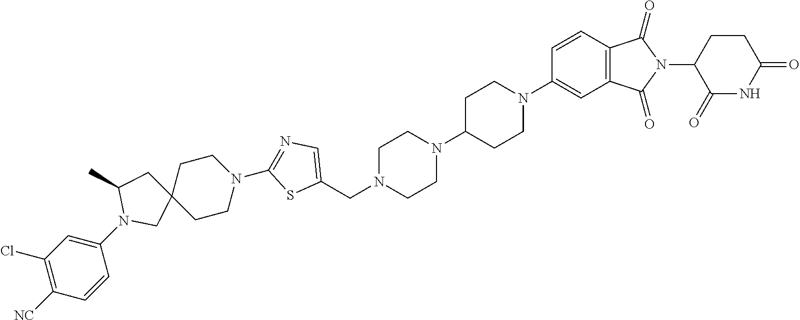 [SMILES: C[C@H]1CC2(CCN(c3ncc(CN4CCN(C5CCN(c6ccc7c(c6)C(=O)N(C6CCC(=O)NC6=O)C7=O)CC5)CC4)s3)CC2)CN1c1ccc(C#N)c(Cl)c1] |
| 252 | 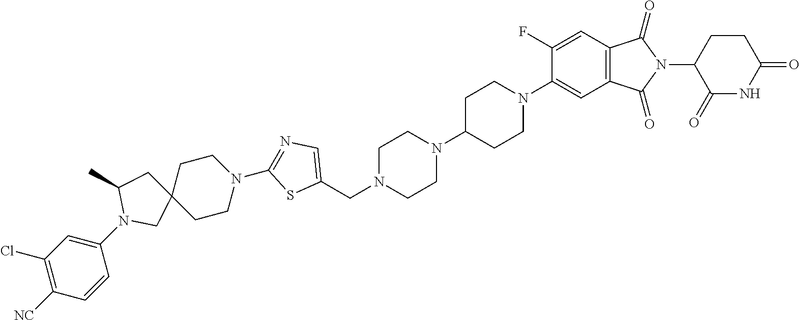 [SMILES: C[C@H]1CC2(CCN(c3ncc(CN4CCN(C5CCN(c6cc7c(cc6F)C(=O)N(C6CCC(=O)NC6=O)C7=O)CC5)CC4)s3)CC2)CN1c1ccc(C#N)c(Cl)c1] |
| 253 | 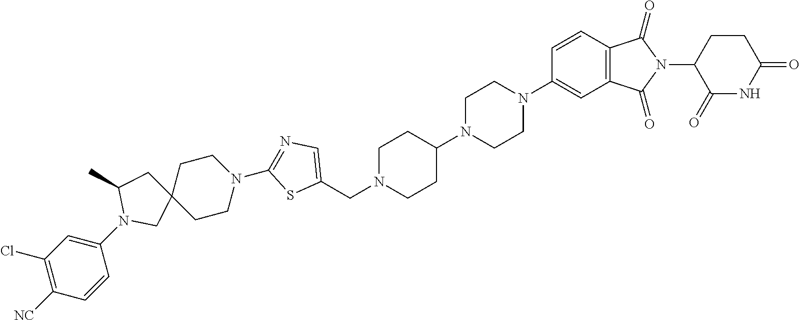 [SMILES: C[C@H]1CC2(CCN(c3ncc(CN4CCC(N5CCN(c6ccc7c(c6)C(=O)N(C6CCC(=O)NC6=O)C7=O)CC5)CC4)s3)CC2)CN1c1ccc(C#N)c(Cl)c1] |
| 254 | 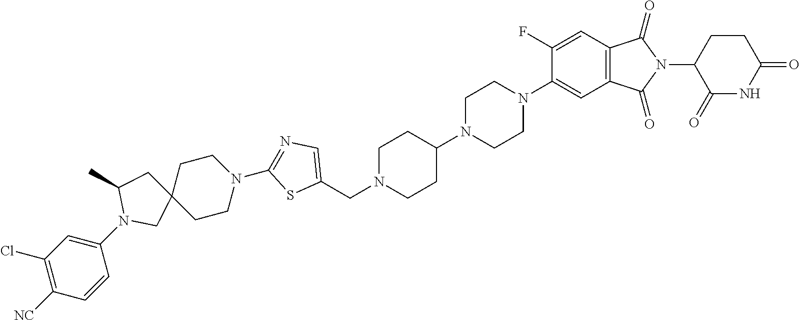 [SMILES: C[C@H]1CC2(CCN(c3ncc(CN4CCC(N5CCN(c6cc7c(cc6F)C(=O)N(C6CCC(=O)NC6=O)C7=O)CC5)CC4)s3)CC2)CN1c1ccc(C#N)c(Cl)c1] |

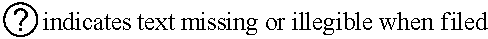

In some embodiments, Compounds of the Disclosure are not any of the compounds of Table IA, or a pharmaceutically acceptable salt or solvate thereof.

TABLE 1A

| Cmpd. | Structure |
|---|---|
| No. | |
| A1 | 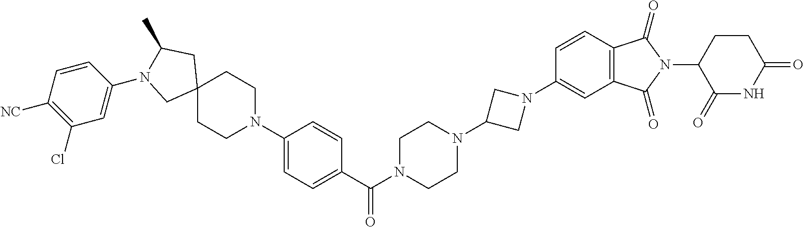 [SMILES: C[C@H]1CC2(CCN(c3ccc(C(=O)N4CCN(C5CN(c6ccc7c(c6)C(=O)N(C6CCC(=O)NC6=O)C7=O)C5)CC4)cc3)CC2)CN1c1ccc(C#N)c(Cl)c1] |
| A2 | 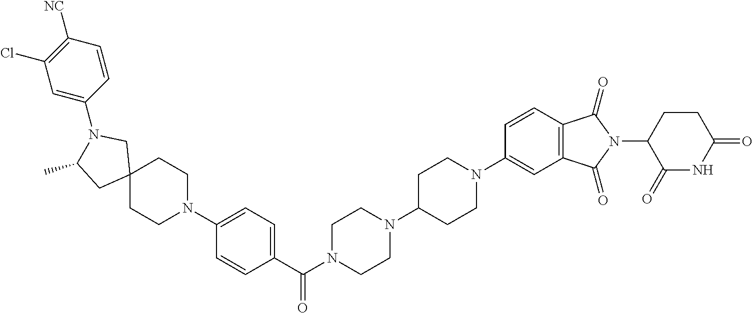 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3)CC2)CN1c1ccc(C#N)c(Cl)c1] |
| A3 | 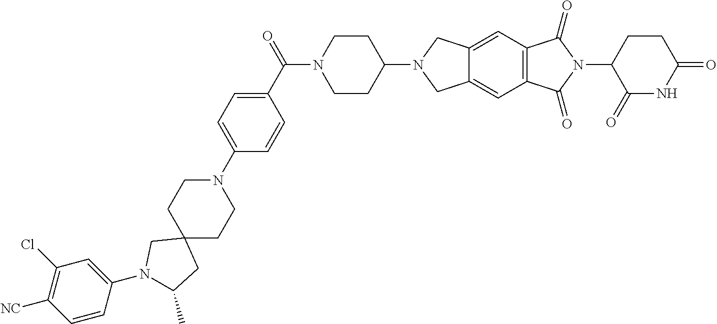 [SMILES: C[C@H]1CC2(CCN(c3ccc(C(=O)N4CCC(N5Cc6cc7c(cc6C5)C(=O)N(C5CCC(=O)NC5=O)C7=O)CC4)cc3)CC2)CN1c1ccc(C#N)c(Cl)c1] |
| A4 | 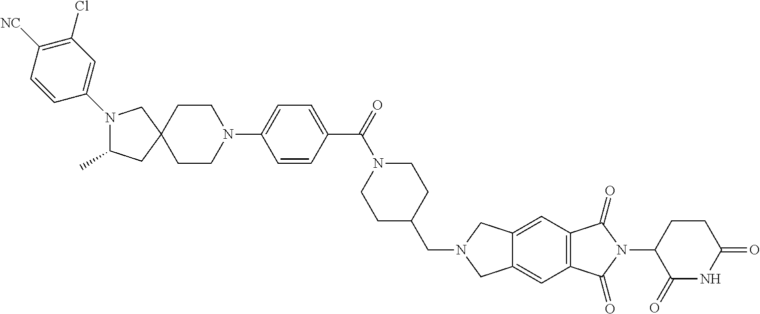 [SMILES: C[C@H]1CC2(CCN(c3ccc(C(=O)N4CCC(CN5Cc6cc7c(cc6C5)C(=O)N(C5CCC(=O)NC5=O)C7=O)CC4)cc3)CC2)CN1c1ccc(C#N)c(Cl)c1] |
| A5 | 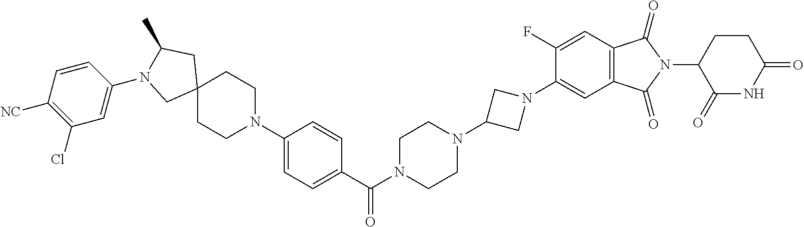 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cc3)CC2)CN1c1ccc(C#N)c(Cl)c1] |
| A6 | 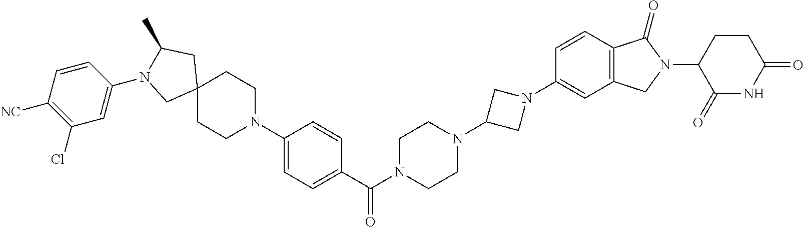 [SMILES: C[C@H]1CC2(CCN(c3ccc(C(=O)N4CCN(C5CN(c6ccc7c(c6)CN(C6CCC(=O)NC6=O)C7=O)C5)CC4)cc3)CC2)CN1c1ccc(C#N)c(Cl)c1] |
| A7 | 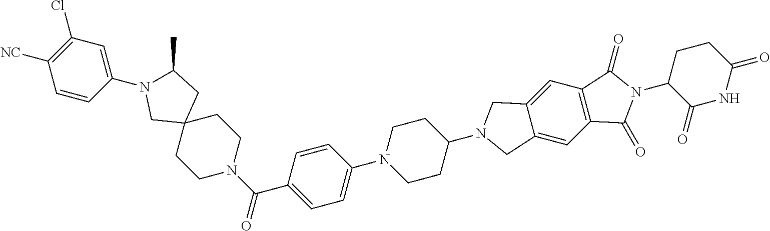 [SMILES: C[C@H]1CC2(CCN(C(=O)c3ccc(N4CCC(N5Cc6cc7c(cc6C5)C(=O)N(C5CCC(=O)NC5=O)C7=O)CC4)cc3)CC2)CN1c1ccc(C#N)c(Cl)c1] |
| A8 | 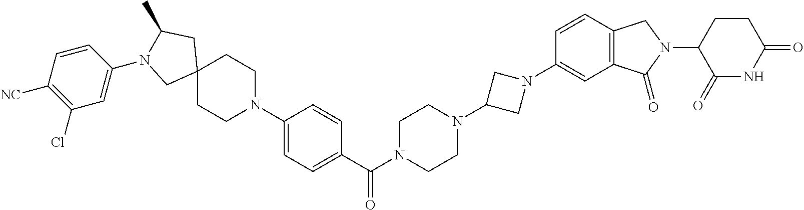 [SMILES: C[C@H]1CC2(CCN(c3ccc(C(=O)N4CCN(C5CN(c6ccc7c(c6)C(=O)N(C6CCC(=O)NC6=O)C7)C5)CC4)cc3)CC2)CN1c1ccc(C#N)c(Cl)c1] |
| A9 | 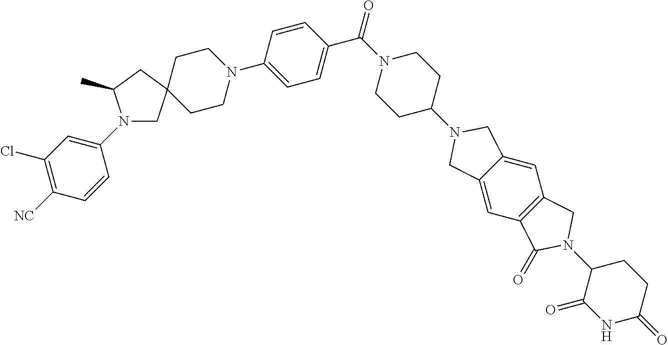 |
| A10 | 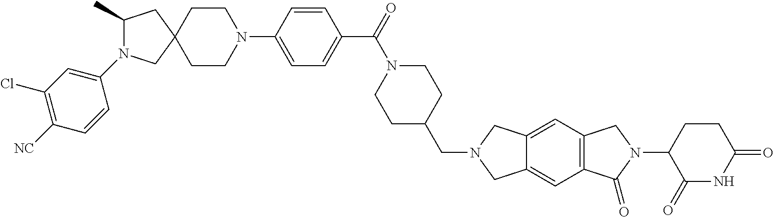 [SMILES: C[C@H]1CC2(CCN(c3ccc(C(=O)N4CCC(CN5Cc6cc7c(cc6C5)C(=O)N(C5CCC(=O)NC5=O)C7)CC4)cc3)CC2)CN1c1ccc(C#N)c(Cl)c1] |
| A11 | 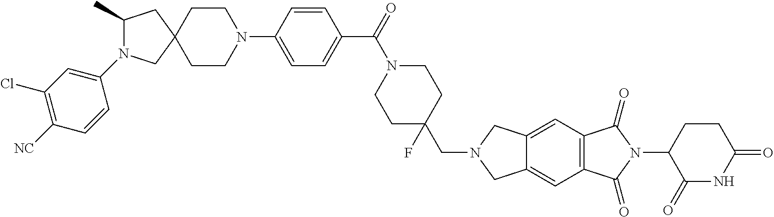 [SMILES: C[C@H]1CC2(CCN(c3ccc(C(=O)N4CCC(F)(CN5Cc6cc7c(cc6C5)C(=O)N(C5CCC(=O)NC5=O)C7=O)CC4)cc3)CC2)CN1c1ccc(C#N)c(Cl)c1] |
| A12 | 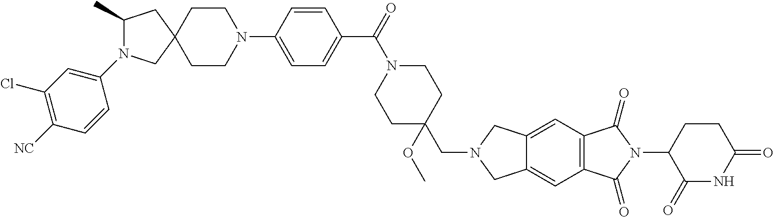 [SMILES: COC1(CN2Cc3cc4c(cc3C2)C(=O)N(C2CCC(=O)NC2=O)C4=O)CCN(C(=O)c2ccc(N3CCC4(CC3)C[C@H](C)N(c3ccc(C#N)c(Cl)c3)C4)cc2)CC1] |
| A13 | 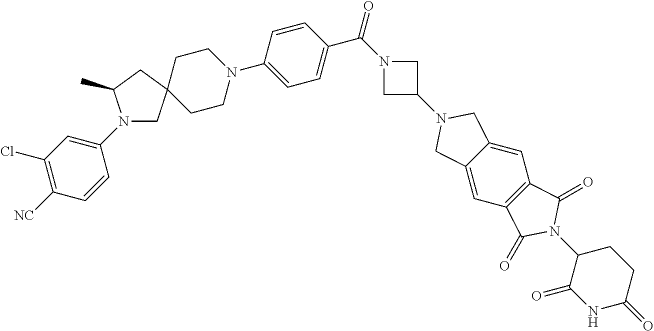 [SMILES: C[C@H]1CC2(CCN(c3ccc(C(=O)N4CC(N5Cc6cc7c(cc6C5)C(=O)N(C5CCC(=O)NC5=O)C7=O)C4)cc3)CC2)CN1c1ccc(C#N)c(Cl)c1] |
| A14 | 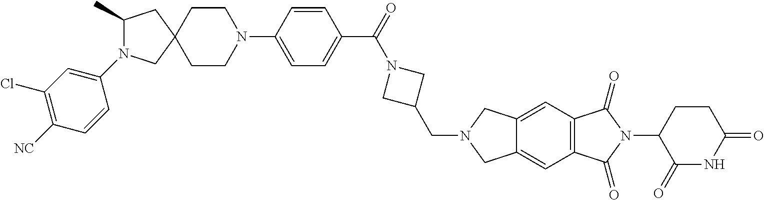 [SMILES: C[C@H]1CC2(CCN(c3ccc(C(=O)N4CC(CN5Cc6cc7c(cc6C5)C(=O)N(C5CCC(=O)NC5=O)C7=O)C4)cc3)CC2)CN1c1ccc(C#N)c(Cl)c1] |
| A15 | 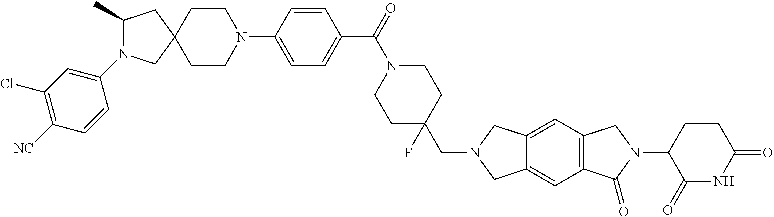 [SMILES: C[C@H]1CC2(CCN(c3ccc(C(=O)N4CCC(F)(CN5Cc6cc7c(cc6C5)C(=O)N(C5CCC(=O)NC5=O)C7)CC4)cc3)CC2)CN1c1ccc(C#N)c(Cl)c1] |
| A16 | 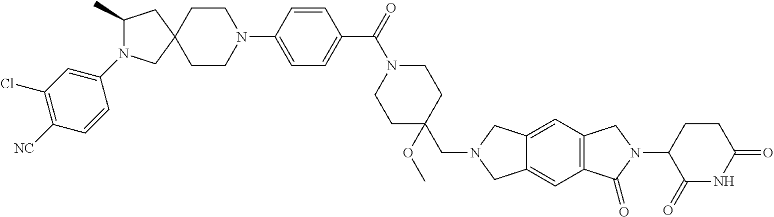 [SMILES: COC1(CN2Cc3cc4c(cc3C2)C(=O)N(C2CCC(=O)NC2=O)C4)CCN(C(=O)c2ccc(N3CCC4(CC3)C[C@H](C)N(c3ccc(C#N)c(Cl)c3)C4)cc2)CC1] |
| A17 | 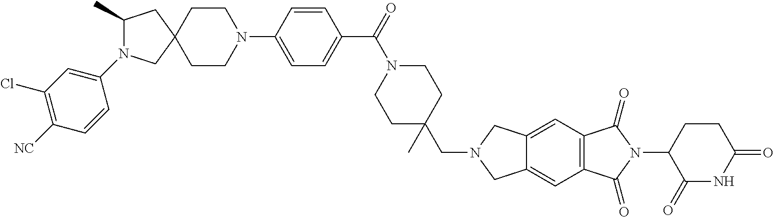 [SMILES: C[C@H]1CC2(CCN(c3ccc(C(=O)N4CCC(C)(CN5Cc6cc7c(cc6C5)C(=O)N(C5CCC(=O)NC5=O)C7=O)CC4)cc3)CC2)CN1c1ccc(C#N)c(Cl)c1] |
| A18 | 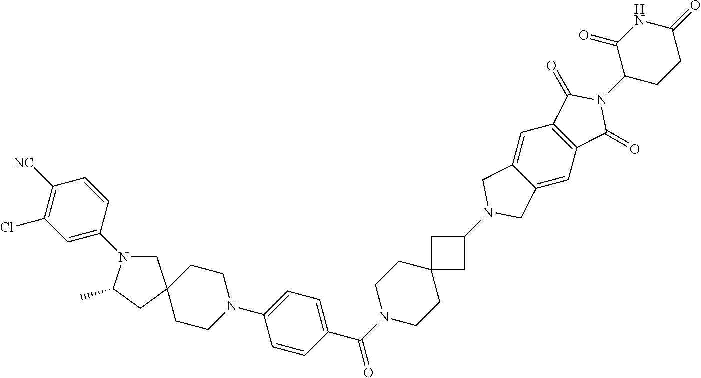 [SMILES: C[C@H]1CC2(CCN(c3ccc(C(=O)N4CCC5(CC4)CC(N4Cc6cc7c(cc6C4)C(=O)N(C4CCC(=O)NC4=O)C7=O)C5)cc3)CC2)CN1c1ccc(C#N)c(Cl)c1] |
| A19 | 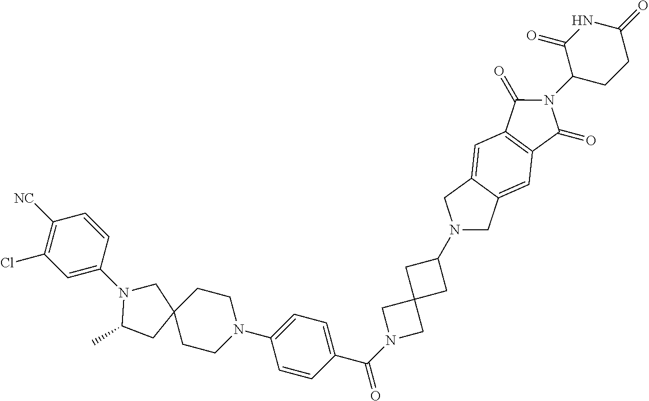 [SMILES: C[C@H]1CC2(CCN(c3ccc(C(=O)N4CC5(CC(N6Cc7cc8c(cc7C6)C(=O)N(C6CCC(=O)NC6=O)C8=O)C5)C4)cc3)CC2)CN1c1ccc(C#N)c(Cl)c1] |
| A20 | 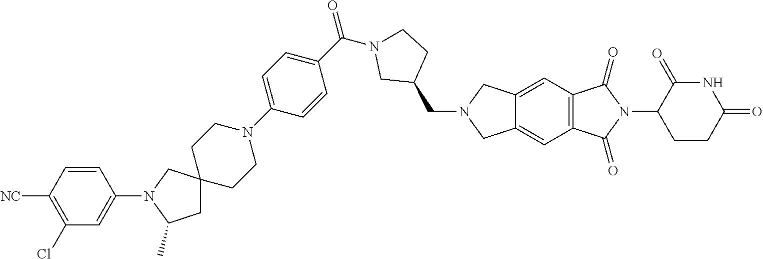 [SMILES: C[C@H]1CC2(CCN(c3ccc(C(=O)N4CC[C@@H](CN5Cc6cc7c(cc6C5)C(=O)N(C5CCC(=O)NC5=O)C7=O)C4)cc3)CC2)CN1c1ccc(C#N)c(Cl)c1] |
| A21 | 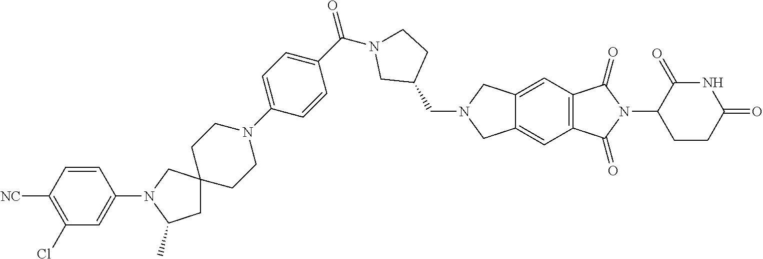 [SMILES: C[C@H]1CC2(CCN(c3ccc(C(=O)N4CC[C@H](CN5Cc6cc7c(cc6C5)C(=O)N(C5CCC(=O)NC5=O)C7=O)C4)cc3)CC2)CN1c1ccc(C#N)c(Cl)c1] |
| A22 | 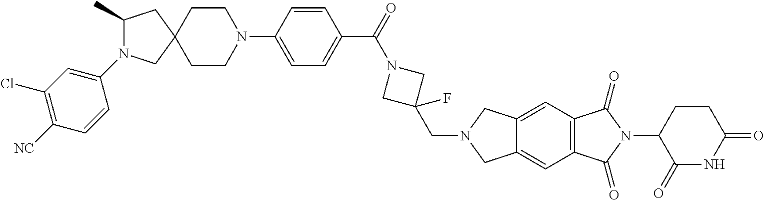 [SMILES: C[C@H]1CC2(CCN(c3ccc(C(=O)N4CC(F)(CN5Cc6cc7c(cc6C5)C(=O)N(C5CCC(=O)NC5=O)C7=O)C4)cc3)CC2)CN1c1ccc(C#N)c(Cl)c1] |
| A23 | 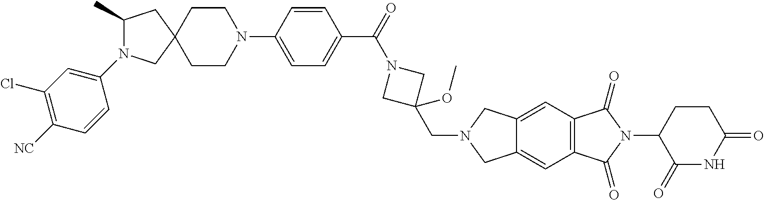 [SMILES: COC1(CN2Cc3cc4c(cc3C2)C(=O)N(C2CCC(=O)NC2=O)C4=O)CN(C(=O)c2ccc(N3CCC4(CC3)C[C@H](C)N(c3ccc(C#N)c(Cl)c3)C4)cc2)C1] |
| A24 | 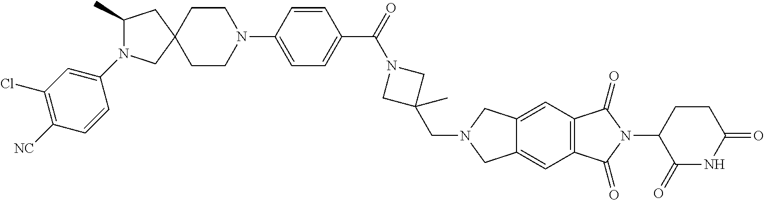 [SMILES: C[C@H]1CC2(CCN(c3ccc(C(=O)N4CC(C)(CN5Cc6cc7c(cc6C5)C(=O)N(C5CCC(=O)NC5=O)C7=O)C4)cc3)CC2)CN1c1ccc(C#N)c(Cl)c1] |
| A25 | 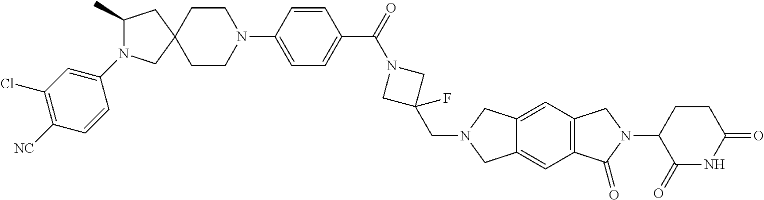 [SMILES: C[C@H]1CC2(CCN(c3ccc(C(=O)N4CC(F)(CN5Cc6cc7c(cc6C5)C(=O)N(C5CCC(=O)NC5=O)C7)C4)cc3)CC2)CN1c1ccc(C#N)c(Cl)c1] |
| A26 | 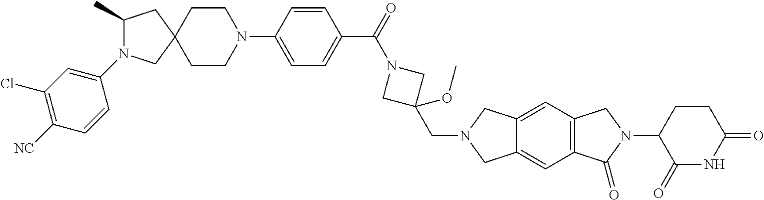 [SMILES: COC1(CN2Cc3cc4c(cc3C2)C(=O)N(C2CCC(=O)NC2=O)C4)CN(C(=O)c2ccc(N3CCC4(CC3)C[C@H](C)N(c3ccc(C#N)c(Cl)c3)C4)cc2)C1] |
| A27 | 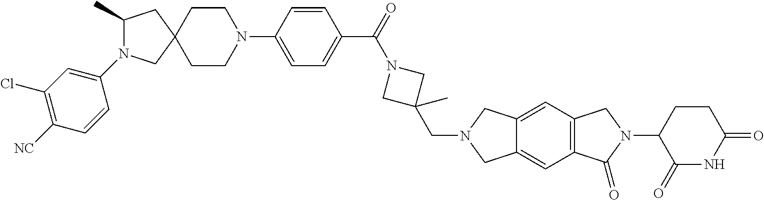 [SMILES: C[C@H]1CC2(CCN(c3ccc(C(=O)N4CC(C)(CN5Cc6cc7c(cc6C5)C(=O)N(C5CCC(=O)NC5=O)C7)C4)cc3)CC2)CN1c1ccc(C#N)c(Cl)c1] |
| A28 | 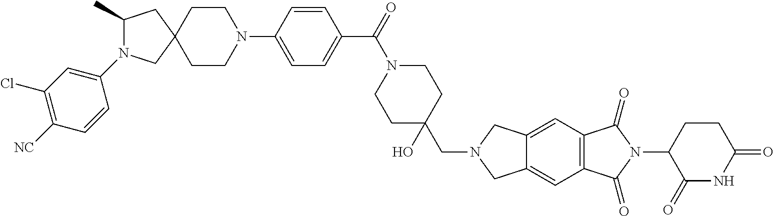 [SMILES: C[C@H]1CC2(CCN(c3ccc(C(=O)N4CCC(O)(CN5Cc6cc7c(cc6C5)C(=O)N(C5CCC(=O)NC5=O)C7=O)CC4)cc3)CC2)CN1c1ccc(C#N)c(Cl)c1] |
| A29 | 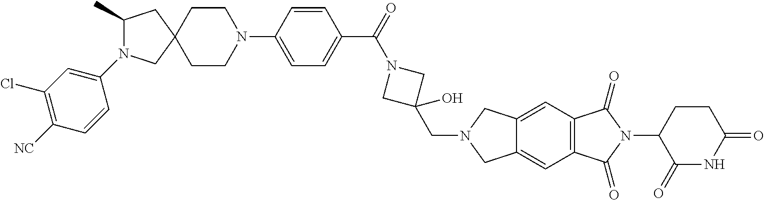 [SMILES: C[C@H]1CC2(CCN(c3ccc(C(=O)N4CC(O)(CN5Cc6cc7c(cc6C5)C(=O)N(C5CCC(=O)NC5=O)C7=O)C4)cc3)CC2)CN1c1ccc(C#N)c(Cl)c1] |
| A30 | 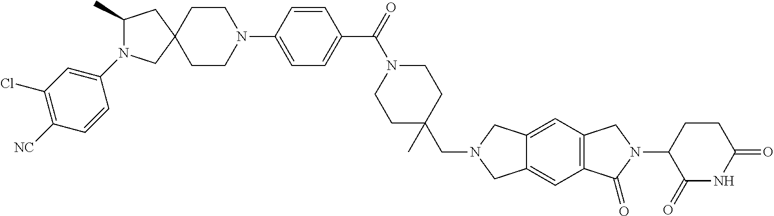 [SMILES: C[C@H]1CC2(CCN(c3ccc(C(=O)N4CCC(C)(CN5Cc6cc7c(cc6C5)C(=O)N(C5CCC(=O)NC5=O)C7)CC4)cc3)CC2)CN1c1ccc(C#N)c(Cl)c1] |
| A31 | 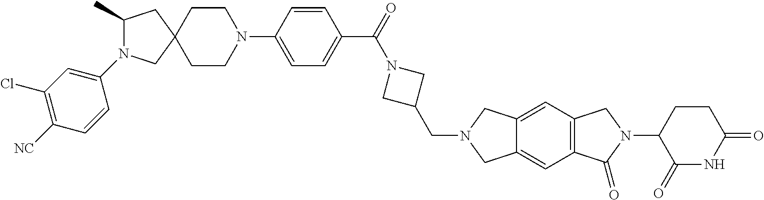 [SMILES: C[C@H]1CC2(CCN(c3ccc(C(=O)N4CC(CN5Cc6cc7c(cc6C5)C(=O)N(C5CCC(=O)NC5=O)C7)C4)cc3)CC2)CN1c1ccc(C#N)c(Cl)c1] |
| A32 | 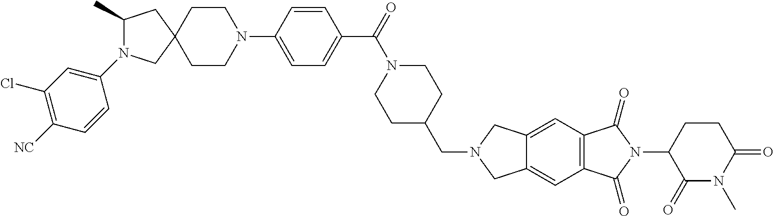 [SMILES: C[C@H]1CC2(CCN(c3ccc(C(=O)N4CCC(CN5Cc6cc7c(cc6C5)C(=O)N(C5CCC(=O)N(C)C5=O)C7=O)CC4)cc3)CC2)CN1c1ccc(C#N)c(Cl)c1] |
| A33 | 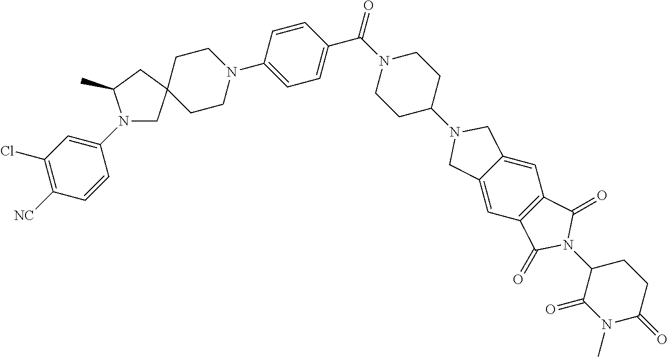 [SMILES: C[C@H]1CC2(CCN(c3ccc(C(=O)N4CCC(N5Cc6cc7c(cc6C5)C(=O)N(C5CCC(=O)N(C)C5=O)C7=O)CC4)cc3)CC2)CN1c1ccc(C#N)c(Cl)c1] |
| A34 | 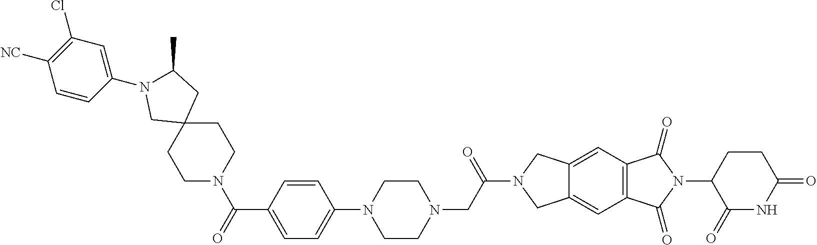 [SMILES: C[C@H]1CC2(CCN(C(=O)c3ccc(N4CCN(CC(=O)N5Cc6cc7c(cc6C5)C(=O)N(C5CCC(=O)NC5=O)C7=O)CC4)cc3)CC2)CN1c1ccc(C#N)c(Cl)c1] |
| A35 | 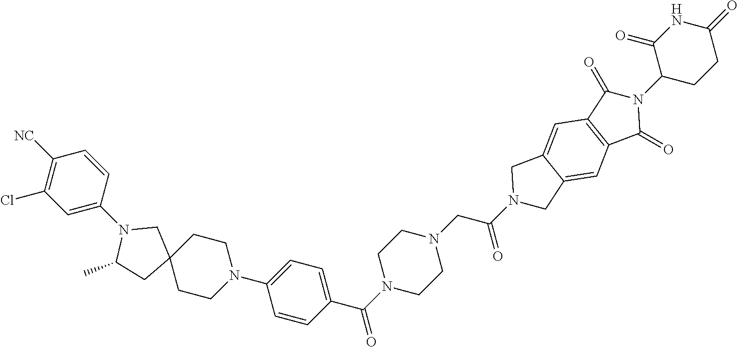 [SMILES: C[C@H]1CC2(CCN(c3ccc(C(=O)N4CCN(CC(=O)N5Cc6cc7c(cc6C5)C(=O)N(C5CCC(=O)NC5=O)C7=O)CC4)cc3)CC2)CN1c1ccc(C#N)c(Cl)c1] |
| A36 | 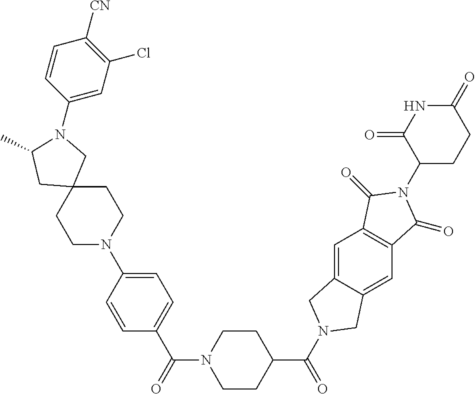 [SMILES: C[C@H]1CC2(CCN(c3ccc(C(=O)N4CCC(C(=O)N5Cc6cc7c(cc6C5)C(=O)N(C5CCC(=O)NC5=O)C7=O)CC4)cc3)CC2)CN1c1ccc(C#N)c(Cl)c1] |
| A37 | 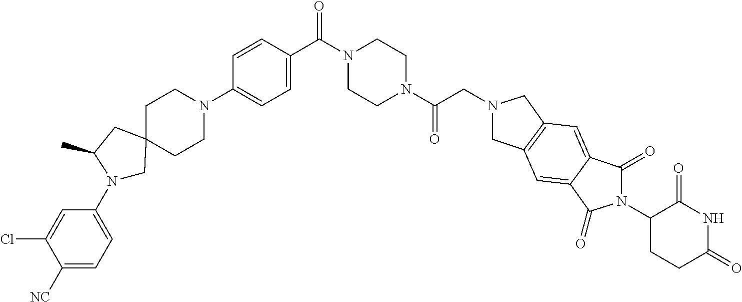 [SMILES: C[C@H]1CC2(CCN(c3ccc(C(=O)N4CCN(C(=O)CN5Cc6cc7c(cc6C5)C(=O)N(C5CCC(=O)NC5=O)C7=O)CC4)cc3)CC2)CN1c1ccc(C#N)c(Cl)c1] |
| A38 | 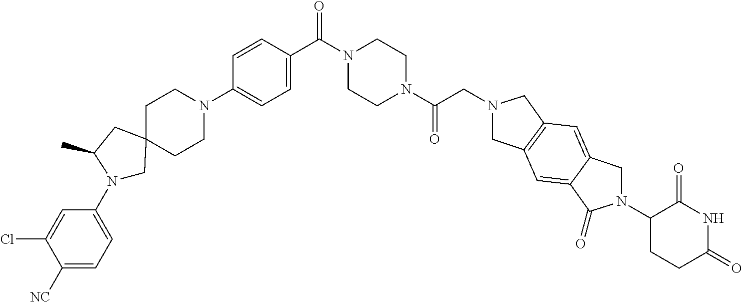 [SMILES: C[C@H]1CC2(CCN(c3ccc(C(=O)N4CCN(C(=O)CN5Cc6cc7c(cc6C5)C(=O)N(C5CCC(=O)NC5=O)C7)CC4)cc3)CC2)CN1c1ccc(C#N)c(Cl)c1] |
| A39 | 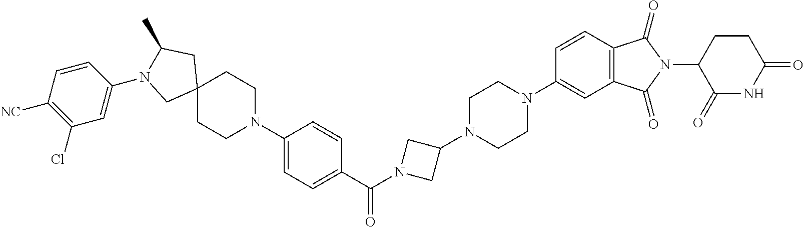 [SMILES: C[C@H]1CC2(CCN(c3ccc(C(=O)N4CC(N5CCN(c6ccc7c(c6)C(=O)N(C6CCC(=O)NC6=O)C7=O)CC5)C4)cc3)CC2)CN1c1ccc(C#N)c(Cl)c1] |
| A40 | 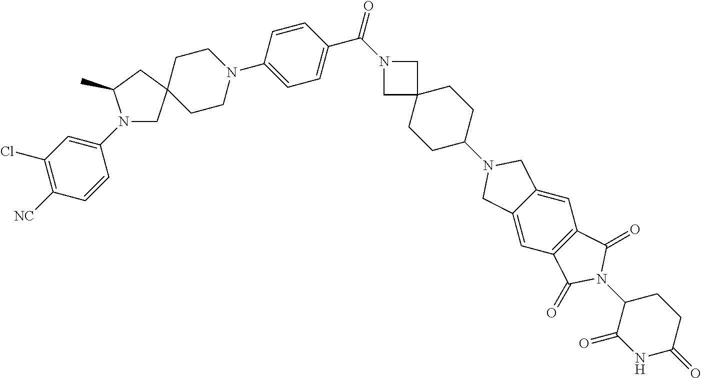 [SMILES: C[C@H]1CC2(CCN(c3ccc(C(=O)N4CC5(CCC(N6Cc7cc8c(cc7C6)C(=O)N(C6CCC(=O)NC6=O)C8=O)CC5)C4)cc3)CC2)CN1c1ccc(C#N)c(Cl)c1] |
| A41 | 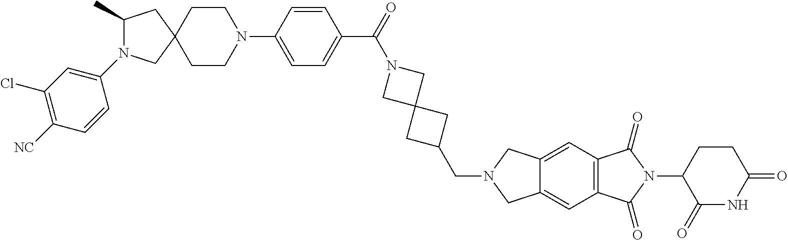 [SMILES: C[C@H]1CC2(CCN(c3ccc(C(=O)N4CC5(CC(CN6Cc7cc8c(cc7C6)C(=O)N(C6CCC(=O)NC6=O)C8=O)C5)C4)cc3)CC2)CN1c1ccc(C#N)c(Cl)c1] |
| A42 | 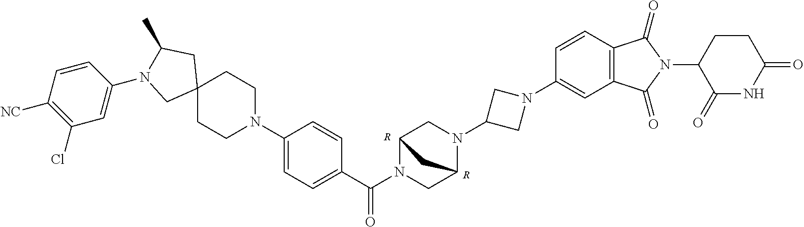 |
| A43 | 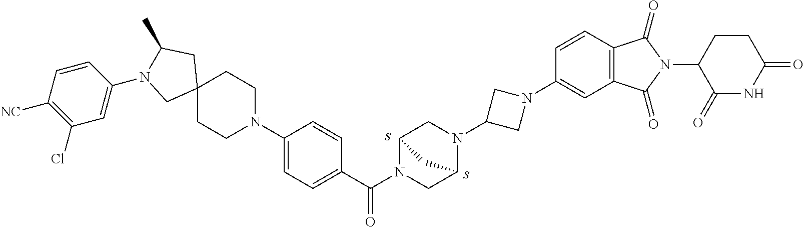 |
| A44 | 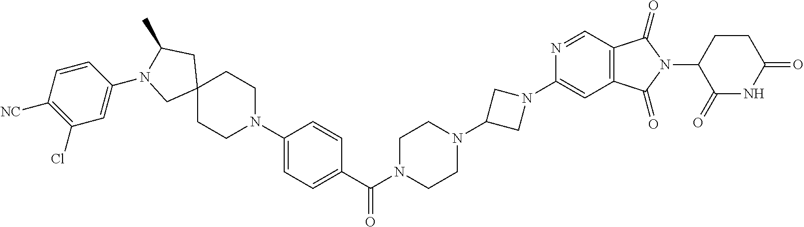 [SMILES: C[C@H]1CC2(CCN(c3ccc(C(=O)N4CCN(C5CN(c6cc7c(cn6)C(=O)N(C6CCC(=O)NC6=O)C7=O)C5)CC4)cc3)CC2)CN1c1ccc(C#N)c(Cl)c1] |
| A45 | 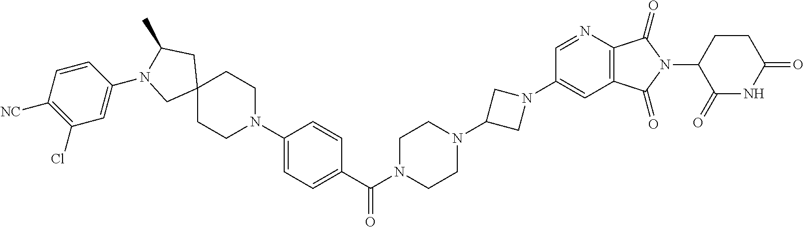 [SMILES: C[C@H]1CC2(CCN(c3ccc(C(=O)N4CCN(C5CN(c6cnc7c(c6)C(=O)N(C6CCC(=O)NC6=O)C7=O)C5)CC4)cc3)CC2)CN1c1ccc(C#N)c(Cl)c1] |
| A46 | 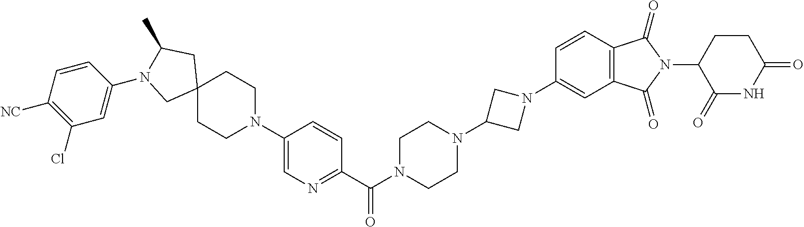 [SMILES: C[C@H]1CC2(CCN(c3ccc(C(=O)N4CCN(C5CN(c6ccc7c(c6)C(=O)N(C6CCC(=O)NC6=O)C7=O)C5)CC4)nc3)CC2)CN1c1ccc(C#N)c(Cl)c1] |
| A47 | 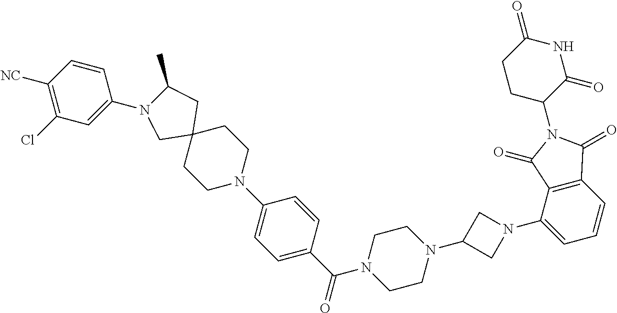 [SMILES: C[C@H]1CC2(CCN(c3ccc(C(=O)N4CCN(C5CN(c6cccc7c6C(=O)N(C6CCC(=O)NC6=O)C7=O)C5)CC4)cc3)CC2)CN1c1ccc(C#N)c(Cl)c1] |
| A48 | 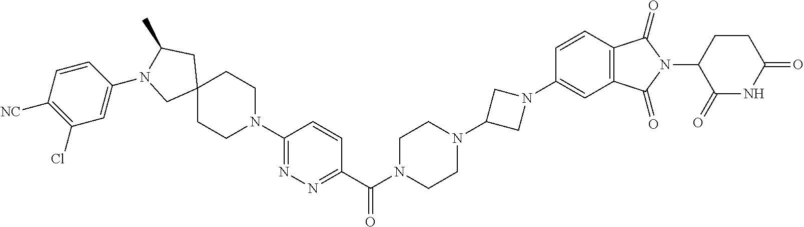 [SMILES: C[C@H]1CC2(CCN(c3ccc(C(=O)N4CCN(C5CN(c6ccc7c(c6)C(=O)N(C6CCC(=O)NC6=O)C7=O)C5)CC4)nn3)CC2)CN1c1ccc(C#N)c(Cl)c1] |
| A49 | 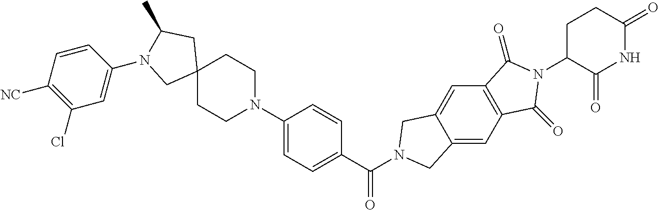 [SMILES: C[C@H]1CC2(CCN(c3ccc(C(=O)N4Cc5cc6c(cc5C4)C(=O)N(C4CCC(=O)NC4=O)C6=O)cc3)CC2)CN1c1ccc(C#N)c(Cl)c1] |
| A50 | 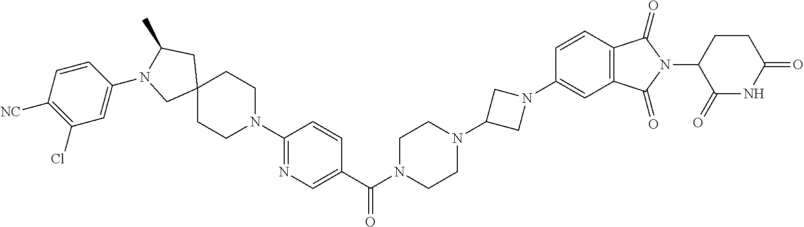 [SMILES: C[C@H]1CC2(CCN(c3ccc(C(=O)N4CCN(C5CN(c6ccc7c(c6)C(=O)N(C6CCC(=O)NC6=O)C7=O)C5)CC4)cn3)CC2)CN1c1ccc(C#N)c(Cl)c1] |
| A51 | 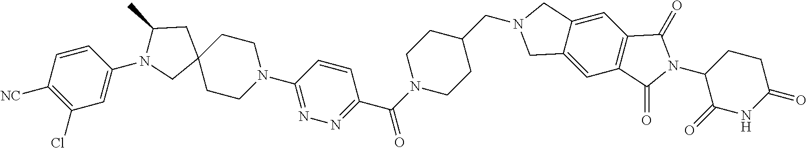 [SMILES: C[C@H]1CC2(CCN(c3ccc(C(=O)N4CCC(CN5Cc6cc7c(cc6C5)C(=O)N(C5CCC(=O)NC5=O)C7=O)CC4)nn3)CC2)CN1c1ccc(C#N)c(Cl)c1] |
| A52 | 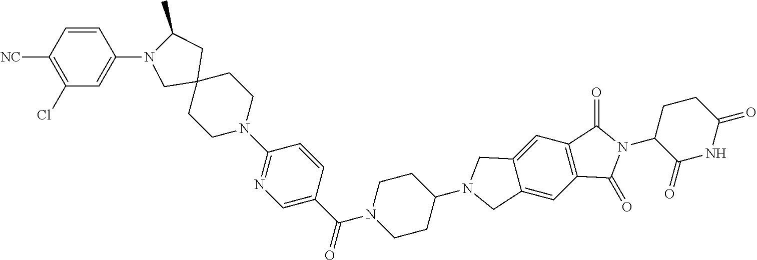 [SMILES: C[C@H]1CC2(CCN(c3ccc(C(=O)N4CCC(N5Cc6cc7c(cc6C5)C(=O)N(C5CCC(=O)NC5=O)C7=O)CC4)cn3)CC2)CN1c1ccc(C#N)c(Cl)c1] |
| A53 | 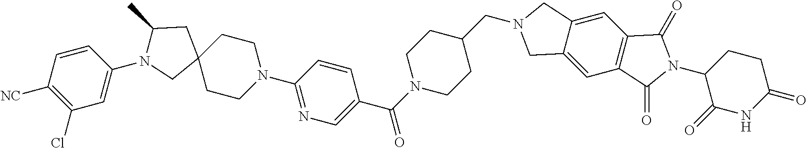 [SMILES: C[C@H]1CC2(CCN(c3ccc(C(=O)N4CCC(CN5Cc6cc7c(cc6C5)C(=O)N(C5CCC(=O)NC5=O)C7=O)CC4)cn3)CC2)CN1c1ccc(C#N)c(Cl)c1] |
| A54 | 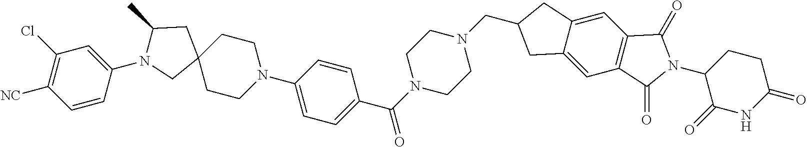 [SMILES: C[C@H]1CC2(CCN(c3ccc(C(=O)N4CCN(CC5Cc6cc7c(cc6C5)C(=O)N(C5CCC(=O)NC5=O)C7=O)CC4)cc3)CC2)CN1c1ccc(C#N)c(Cl)c1] |
| A55 | 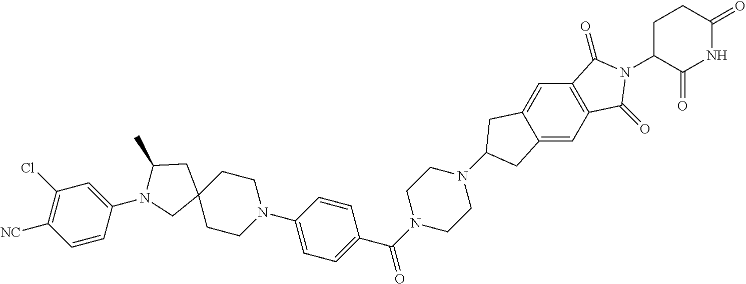 [SMILES: C[C@H]1CC2(CCN(c3ccc(C(=O)N4CCN(C5Cc6cc7c(cc6C5)C(=O)N(C5CCC(=O)NC5=O)C7=O)CC4)cc3)CC2)CN1c1ccc(C#N)c(Cl)c1] |
| A56 | 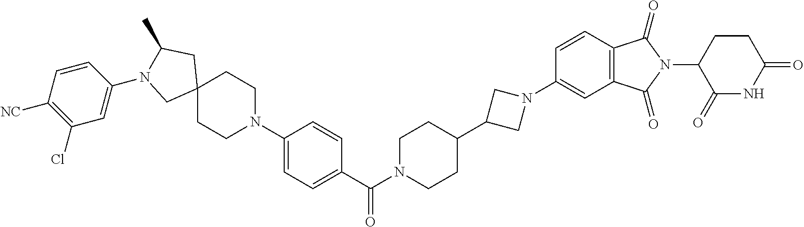 [SMILES: C[C@H]1CC2(CCN(c3ccc(C(=O)N4CCC(C5CN(c6ccc7c(c6)C(=O)N(C6CCC(=O)NC6=O)C7=O)C5)CC4)cc3)CC2)CN1c1ccc(C#N)c(Cl)c1] |
| A57 | 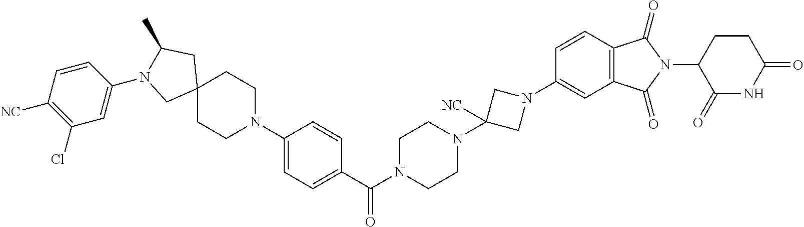 [SMILES: C[C@H]1CC2(CCN(c3ccc(C(=O)N4CCN(C5(C#N)CN(c6ccc7c(c6)C(=O)N(C6CCC(=O)NC6=O)C7=O)C5)CC4)cc3)CC2)CN1c1ccc(C#N)c(Cl)c1] |
| A58 | 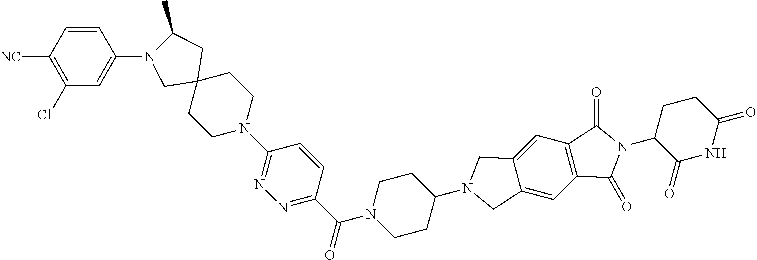 [SMILES: C[C@H]1CC2(CCN(c3ccc(C(=O)N4CCC(N5Cc6cc7c(cc6C5)C(=O)N(C5CCC(=O)NC5=O)C7=O)CC4)nn3)CC2)CN1c1ccc(C#N)c(Cl)c1] |
| A59 | 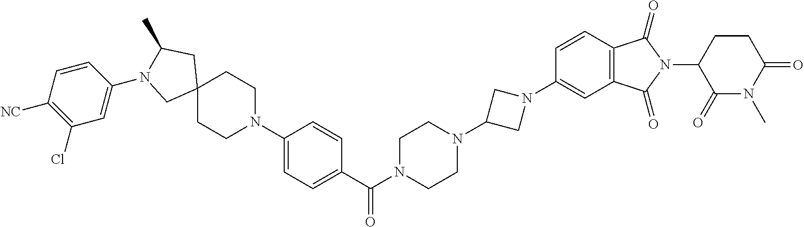 [SMILES: C[C@H]1CC2(CCN(c3ccc(C(=O)N4CCN(C5CN(c6ccc7c(c6)C(=O)N(C6CCC(=O)N(C)C6=O)C7=O)C5)CC4)cc3)CC2)CN1c1ccc(C#N)c(Cl)c1] |
| A60 | 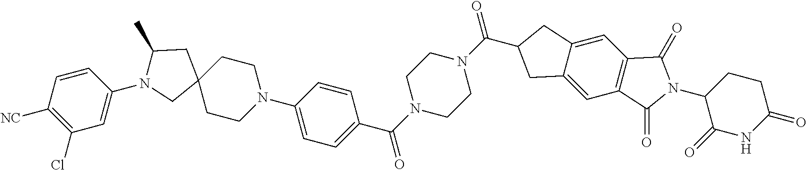 [SMILES: C[C@H]1CC2(CCN(c3ccc(C(=O)N4CCN(C(=O)C5Cc6cc7c(cc6C5)C(=O)N(C5CCC(=O)NC5=O)C7=O)CC4)cc3)CC2)CN1c1ccc(C#N)c(Cl)c1] |
| A61 | 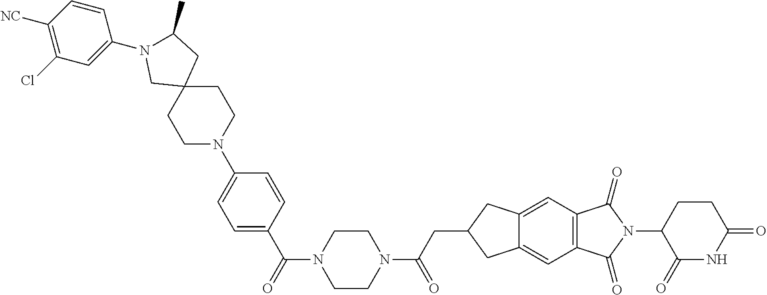 [SMILES: C[C@H]1CC2(CCN(c3ccc(C(=O)N4CCN(C(=O)CC5Cc6cc7c(cc6C5)C(=O)N(C5CCC(=O)NC5=O)C7=O)CC4)cc3)CC2)CN1c1ccc(C#N)c(Cl)c1] |
| A62 | 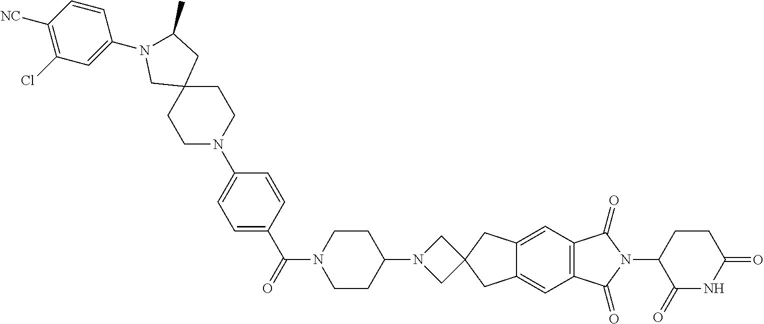 [SMILES: C[C@H]1CC2(CCN(c3ccc(C(=O)N4CCC(N5CC6(Cc7cc8c(cc7C6)C(=O)N(C6CCC(=O)NC6=O)C8=O)C5)CC4)cc3)CC2)CN1c1ccc(C#N)c(Cl)c1] |
| A63 | 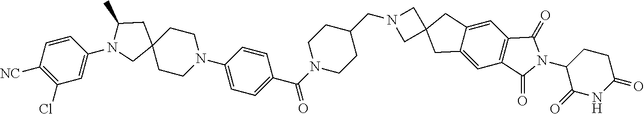 [SMILES: C[C@H]1CC2(CCN(c3ccc(C(=O)N4CCC(CN5CC6(Cc7cc8c(cc7C6)C(=O)N(C6CCC(=O)NC6=O)C8=O)C5)CC4)cc3)CC2)CN1c1ccc(C#N)c(Cl)c1] |
| A64 | 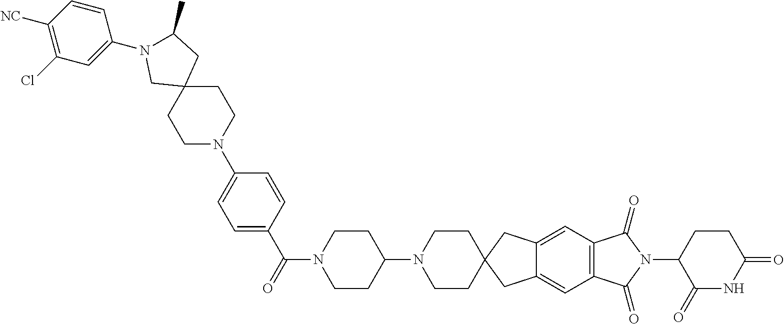 [SMILES: C[C@H]1CC2(CCN(c3ccc(C(=O)N4CCC(N5CCC6(CC5)Cc5cc7c(cc5C6)C(=O)N(C5CCC(=O)NC5=O)C7=O)CC4)cc3)CC2)CN1c1ccc(C#N)c(Cl)c1] |
| A65 | 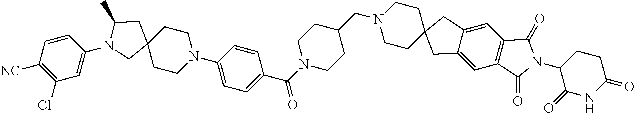 [SMILES: C[C@H]1CC2(CCN(c3ccc(C(=O)N4CCC(CN5CCC6(CC5)Cc5cc7c(cc5C6)C(=O)N(C5CCC(=O)NC5=O)C7=O)CC4)cc3)CC2)CN1c1ccc(C#N)c(Cl)c1] |
| A66 | 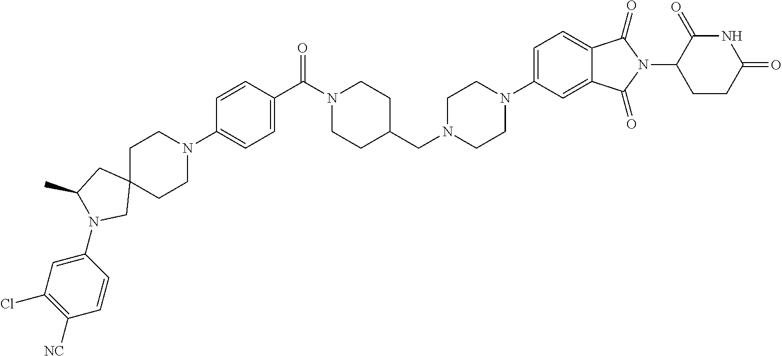 [SMILES: C[C@H]1CC2(CCN(c3ccc(C(=O)N4CCC(CN5CCN(c6ccc7c(c6)C(=O)N(C6CCC(=O)NC6=O)C7=O)CC5)CC4)cc3)CC2)CN1c1ccc(C#N)c(Cl)c1] |
| A67 | 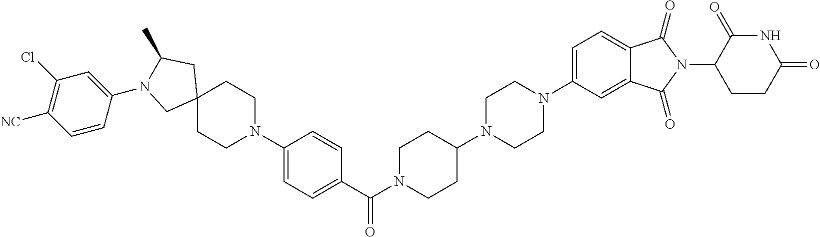 [SMILES: C[C@H]1CC2(CCN(c3ccc(C(=O)N4CCC(N5CCN(c6ccc7c(c6)C(=O)N(C6CCC(=O)NC6=O)C7=O)CC5)CC4)cc3)CC2)CN1c1ccc(C#N)c(Cl)c1] |
| A68 | 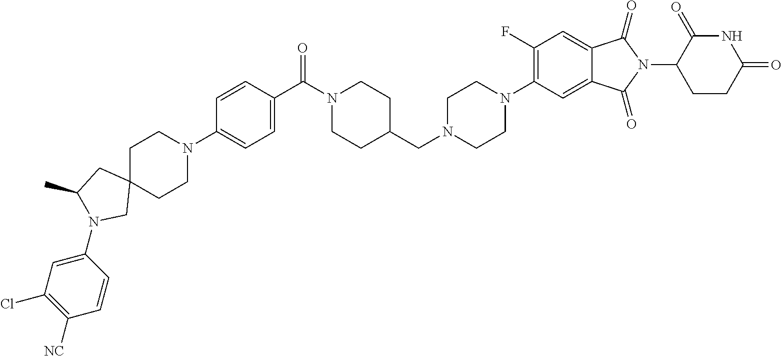 [SMILES: C[C@H]1CC2(CCN(c3ccc(C(=O)N4CCC(CN5CCN(c6cc7c(cc6F)C(=O)N(C6CCC(=O)NC6=O)C7=O)CC5)CC4)cc3)CC2)CN1c1ccc(C#N)c(Cl)c1] |
| A69 | 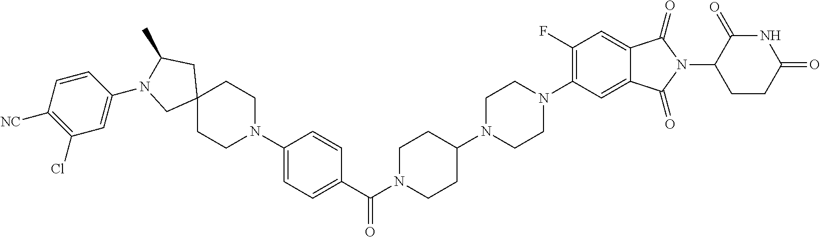 [SMILES: C[C@H]1CC2(CCN(c3ccc(C(=O)N4CCC(N5CCN(c6cc7c(cc6F)C(=O)N(C6CCC(=O)NC6=O)C7=O)CC5)CC4)cc3)CC2)CN1c1ccc(C#N)c(Cl)c1] |
| A70 | 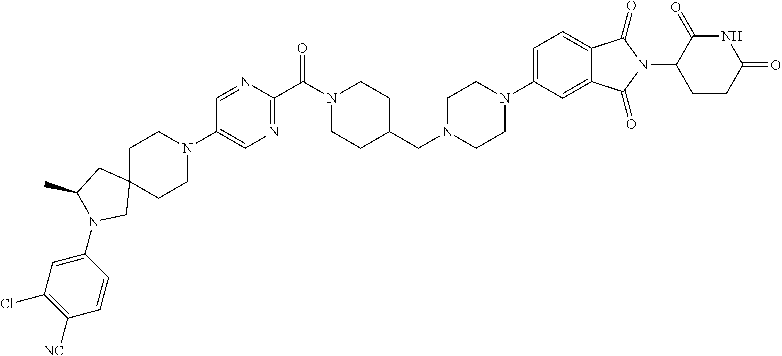 [SMILES: C[C@H]1CC2(CCN(c3cnc(C(=O)N4CCC(CN5CCN(c6ccc7c(c6)C(=O)N(C6CCC(=O)NC6=O)C7=O)CC5)CC4)nc3)CC2)CN1c1ccc(C#N)c(Cl)c1] |
| A71 | 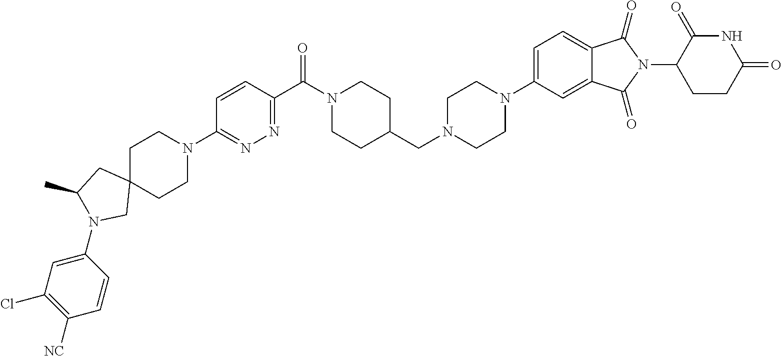 [SMILES: C[C@H]1CC2(CCN(c3ccc(C(=O)N4CCC(CN5CCN(c6ccc7c(c6)C(=O)N(C6CCC(=O)NC6=O)C7=O)CC5)CC4)nn3)CC2)CN1c1ccc(C#N)c(Cl)c1] |
| A72 | 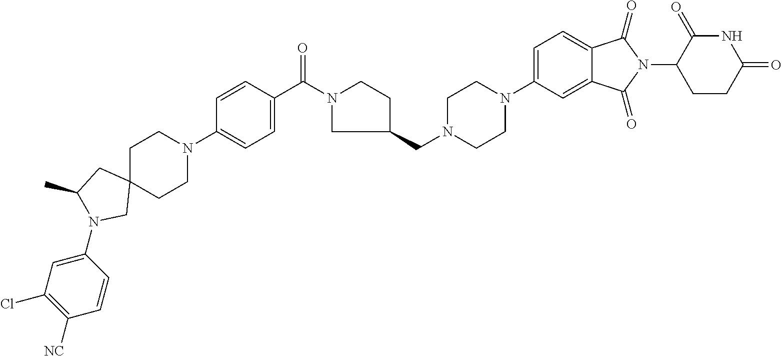 [SMILES: C[C@H]1CC2(CCN(c3ccc(C(=O)N4CC[C@@H](CN5CCN(c6ccc7c(c6)C(=O)N(C6CCC(=O)NC6=O)C7=O)CC5)C4)cc3)CC2)CN1c1ccc(C#N)c(Cl)c1] |
| A73 | 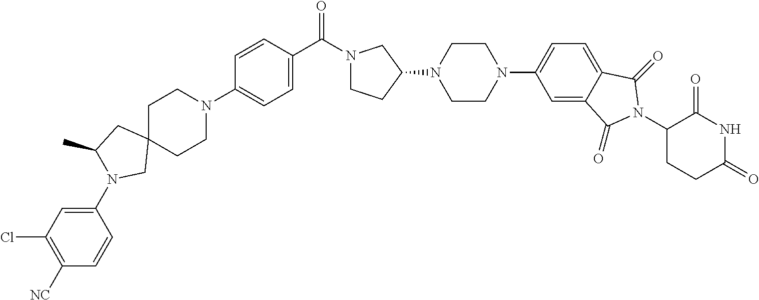 [SMILES: C[C@H]1CC2(CCN(c3ccc(C(=O)N4CC[C@@H](N5CCN(c6ccc7c(c6)C(=O)N(C6CCC(=O)NC6=O)C7=O)CC5)C4)cc3)CC2)CN1c1ccc(C#N)c(Cl)c1] |
| A74 | 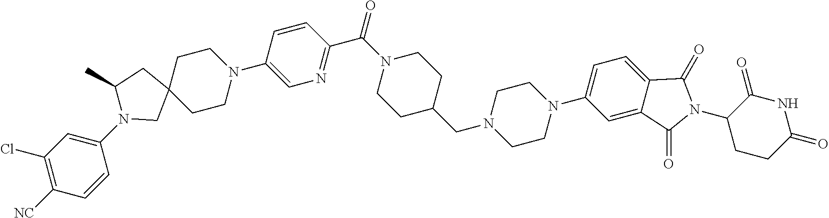 [SMILES: C[C@H]1CC2(CCN(c3ccc(C(=O)N4CCC(CN5CCN(c6ccc7c(c6)C(=O)N(C6CCC(=O)NC6=O)C7=O)CC5)CC4)nc3)CC2)CN1c1ccc(C#N)c(Cl)c1] |
| A75 | 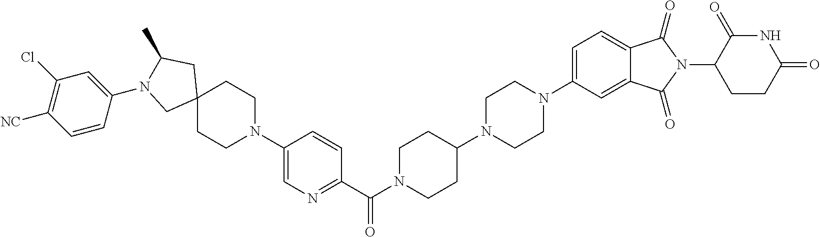 [SMILES: C[C@H]1CC2(CCN(c3ccc(C(=O)N4CCC(N5CCN(c6ccc7c(c6)C(=O)N(C6CCC(=O)NC6=O)C7=O)CC5)CC4)nc3)CC2)CN1c1ccc(C#N)c(Cl)c1] |
| A76 | 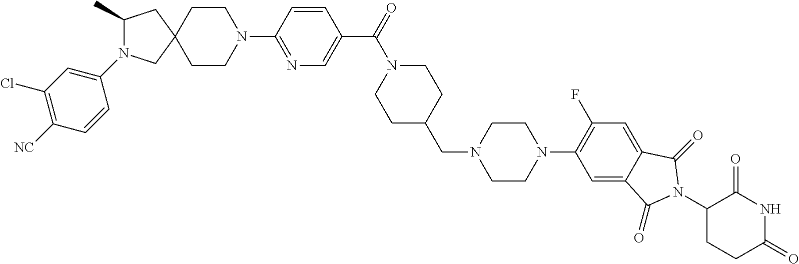 [SMILES: C[C@H]1CC2(CCN(c3ccc(C(=O)N4CCC(CN5CCN(c6cc7c(cc6F)C(=O)N(C6CCC(=O)NC6=O)C7=O)CC5)CC4)cn3)CC2)CN1c1ccc(C#N)c(Cl)c1] |
| A77 | 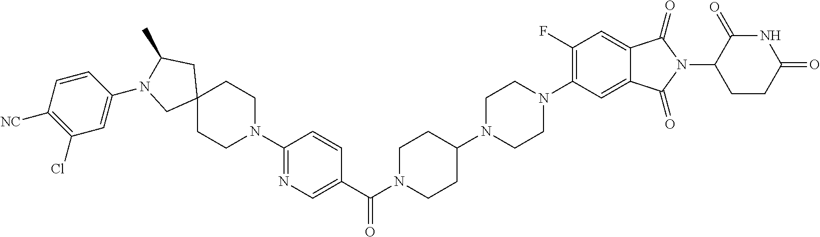 [SMILES: C[C@H]1CC2(CCN(c3ccc(C(=O)N4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| A78 | 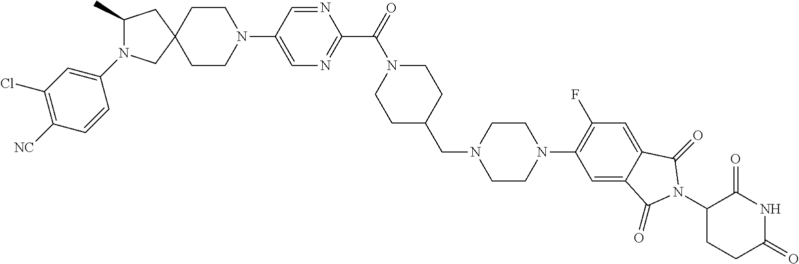 [SMILES: C[C@H]1CC2(CCN(c3cnc(C(=O)N4CCC(CN5CCN(c6cc7c(cc6F)C(=O)N(C6CCC(=O)NC6=O)C7=O)CC5)CC4)nc3)CC2)CN1c1ccc(C#N)c(Cl)c1] |
| A79 | 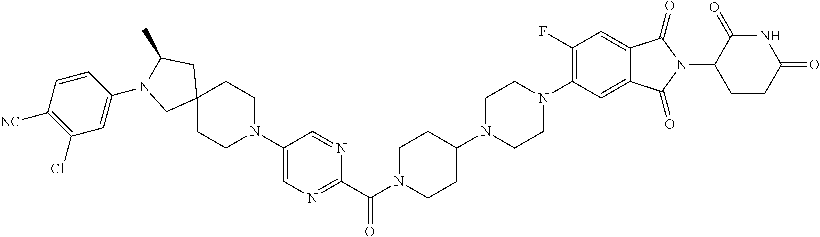 [SMILES: C[C@H]1CC2(CCN(c3cnc(C(=O)N4CCC(N5CCN(c6cc7c(cc6F)C(=O)N(C6CCC(=O)NC6=O)C7=O)CC5)CC4)nc3)CC2)CN1c1ccc(C#N)c(Cl)c1] |
| A80 | 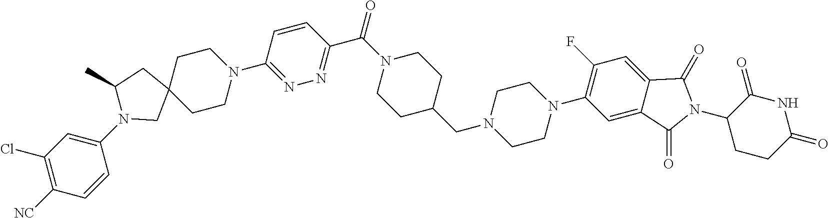 [SMILES: C[C@H]1CC2(CCN(c3ccc(C(=O)N4CCC(CN5CCN(c6cc7c(cc6F)C(=O)N(C6CCC(=O)NC6=O)C7=O)CC5)CC4)nn3)CC2)CN1c1ccc(C#N)c(Cl)c1] |
| A81 | 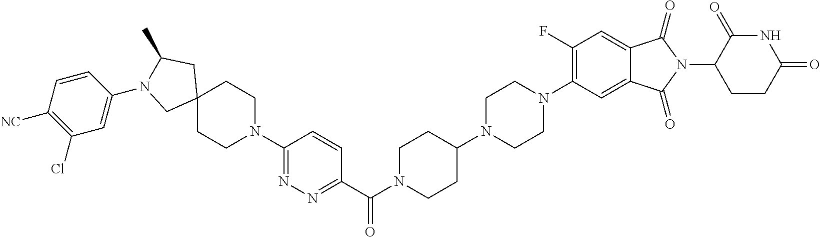 [SMILES: C[C@H]1CC2(CCN(c3ccc(C(=O)N4CCC(N5CCN(c6cc7c(cc6F)C(=O)N(C6CCC(=O)NC6=O)C7=O)CC5)CC4)nn3)CC2)CN1c1ccc(C#N)c(Cl)c1] |
| A82 | 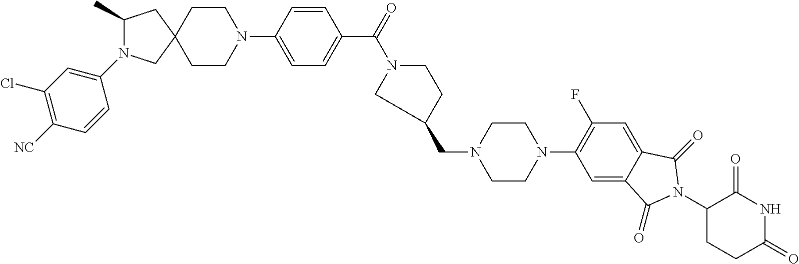 [SMILES: C[C@H]1CC2(CCN(c3ccc(C(=O)N4CC[C@@H](CN5CCN(c6cc7c(cc6F)C(=O)N(C6CCC(=O)NC6=O)C7=O)CC5)C4)cc3)CC2)CN1c1ccc(C#N)c(Cl)c1] |
| A83 | 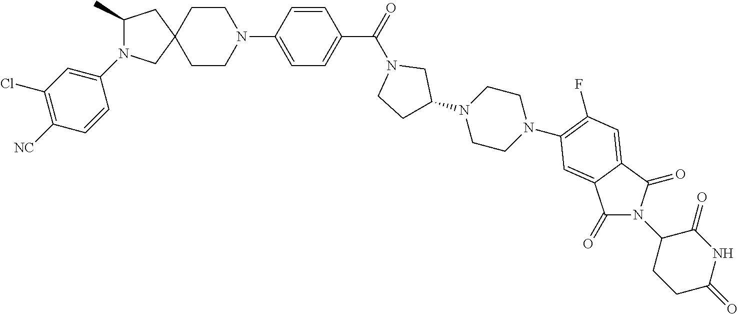 [SMILES: C[C@H]1CC2(CCN(c3ccc(C(=O)N4CC[C@@H](N5CCN(c6cc7c(cc6F)C(=O)N(C6CCC(=O)NC6=O)C7=O)CC5)C4)cc3)CC2)CN1c1ccc(C#N)c(Cl)c1] |
| A84 | 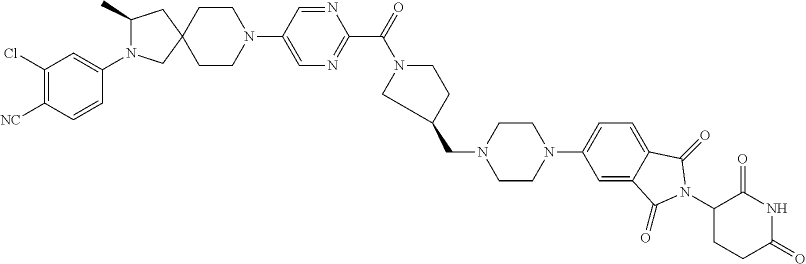 [SMILES: C[C@H]1CC2(CCN(c3cnc(C(=O)N4CC[C@@H](CN5CCN(c6ccc7c(c6)C(=O)N(C6CCC(=O)NC6=O)C7=O)CC5)C4)nc3)CC2)CN1c1ccc(C#N)c(Cl)c1] |
| A85 | 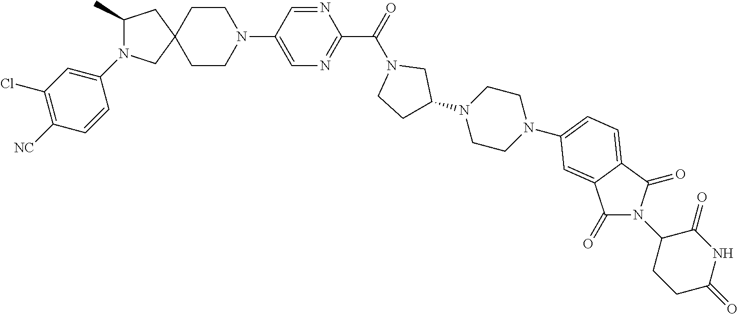 [SMILES: C[C@H]1CC2(CCN(c3cnc(C(=O)N4CC[C@@H](N5CCN(c6ccc7c(c6)C(=O)N(C6CCC(=O)NC6=O)C7=O)CC5)C4)nc3)CC2)CN1c1ccc(C#N)c(Cl)c1] |
| A86 | 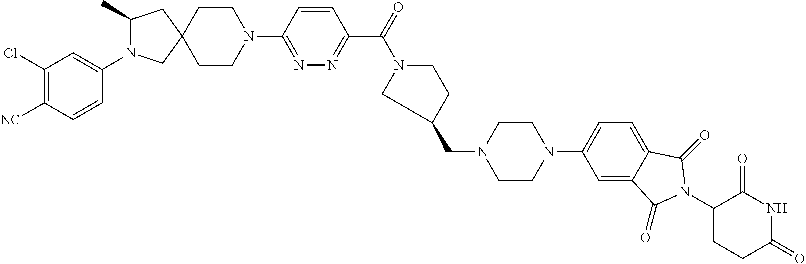 [SMILES: C[C@H]1CC2(CCN(c3ccc(C(=O)N4CC[C@@H](CN5CCN(c6ccc7c(c6)C(=O)N(C6CCC(=O)NC6=O)C7=O)CC5)C4)nn3)CC2)CN1c1ccc(C#N)c(Cl)c1] |
| A87 | 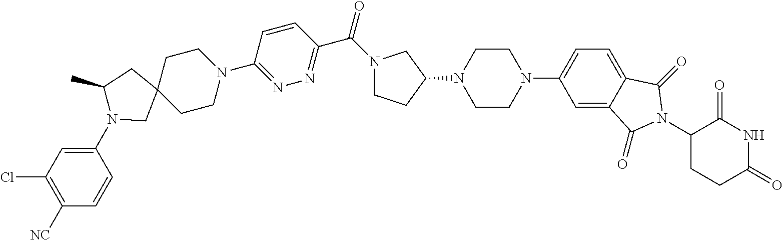 [SMILES: C[C@H]1CC2(CCN(c3ccc(C(=O)N4CC[C@@H](N5CCN(c6ccc7c(c6)C(=O)N(C6CCC(=O)NC6=O)C7=O)CC5)C4)nn3)CC2)CN1c1ccc(C#N)c(Cl)c1] |
| A88 | 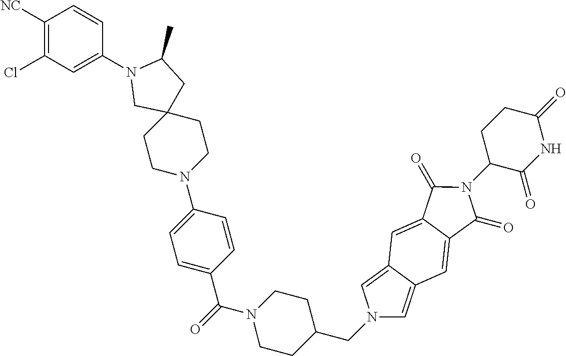 [SMILES: C[C@H]1CC2(CCN(c3ccc(C(=O)N4CCC(Cn5cc6cc7c(cc6c5)C(=O)N(C5CCC(=O)NC5=O)C7=O)CC4)cc3)CC2)CN1c1ccc(C#N)c(Cl)c1] |
| A89 | 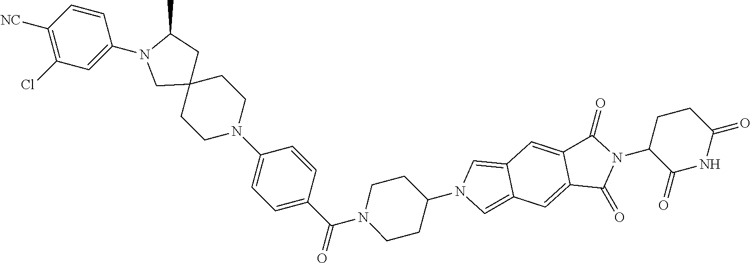 [SMILES: C[C@H]1CC2(CCN(c3ccc(C(=O)N4CCC(n5cc6cc7c(cc6c5)C(=O)N(C5CCC(=O)NC5=O)C7=O)CC4)cc3)CC2)CN1c1ccc(C#N)c(Cl)c1] |
| A90 | 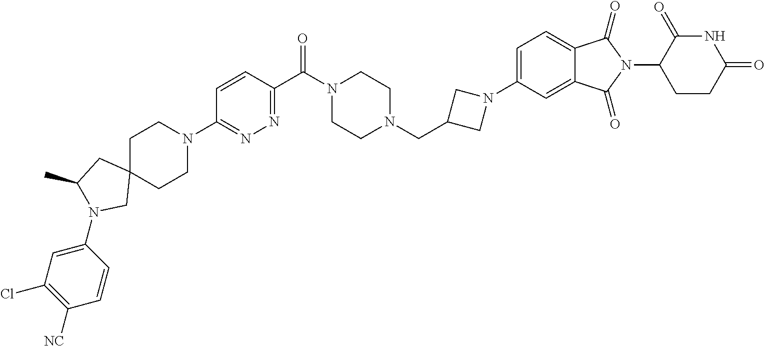 [SMILES: C[C@H]1CC2(CCN(c3ccc(C(=O)N4CCN(CC5CN(c6ccc7c(c6)C(=O)N(C6CCC(=O)NC6=O)C7=O)C5)CC4)nn3)CC2)CN1c1ccc(C#N)c(Cl)c1] |
| A91 | 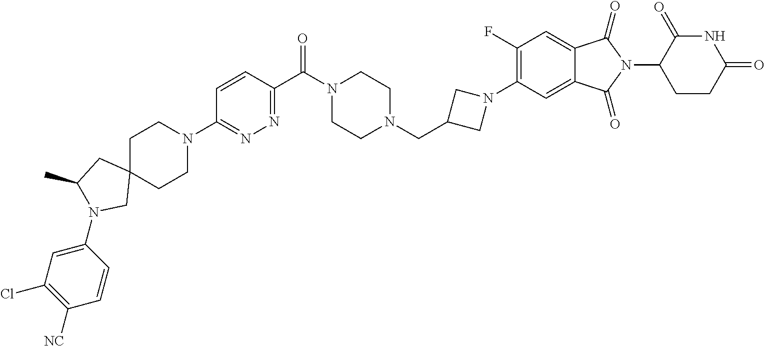 [SMILES: C[C@H]1CC2(CCN(c3ccc(C(=O)N4CCN(CC5CN(c6cc7c(cc6F)C(=O)N(C6CCC(=O)NC6=O)C7=O)C5)CC4)nn3)CC2)CN1c1ccc(C#N)c(Cl)c1] |
| A92 | 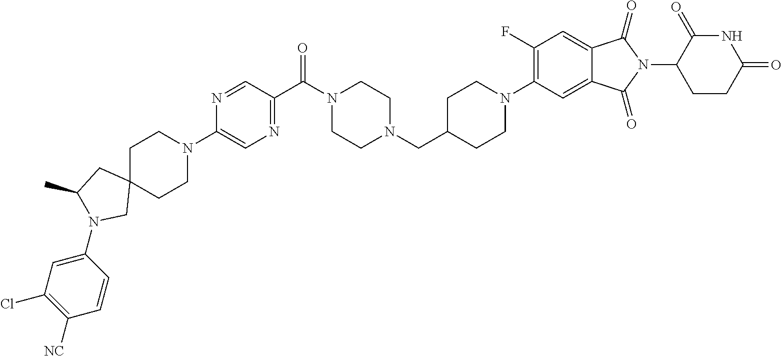 [SMILES: C[C@H]1CC2(CCN(c3cnc(C(=O)N4CCN(CC5CCN(c6cc7c(cc6F)C(=O)N(C6CCC(=O)NC6=O)C7=O)CC5)CC4)cn3)CC2)CN1c1ccc(C#N)c(Cl)c1] |
| A93 | 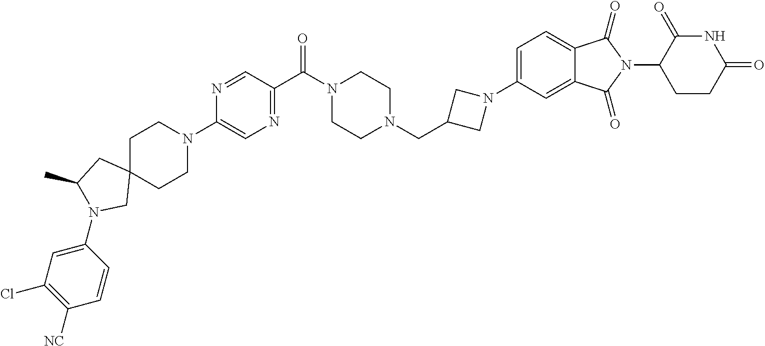 [SMILES: C[C@H]1CC2(CCN(c3cnc(C(=O)N4CCN(CC5CN(c6ccc7c(c6)C(=O)N(C6CCC(=O)NC6=O)C7=O)C5)CC4)cn3)CC2)CN1c1ccc(C#N)c(Cl)c1] |
| A94 | 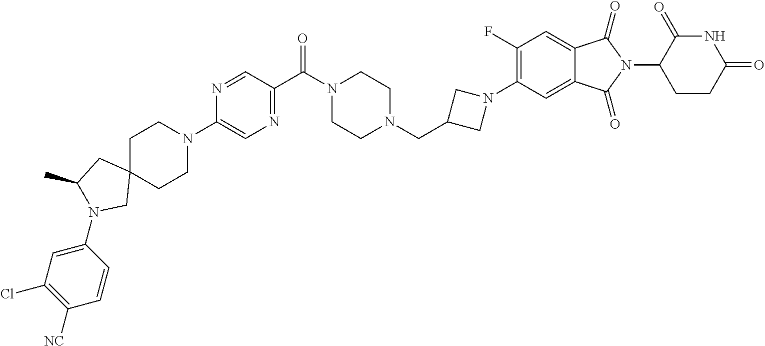 [SMILES: C[C@H]1CC2(CCN(c3cnc(C(=O)N4CCN(CC5CN(c6cc7c(cc6F)C(=O)N(C6CCC(=O)NC6=O)C7=O)C5)CC4)cn3)CC2)CN1c1ccc(C#N)c(Cl)c1] |
| A95 | 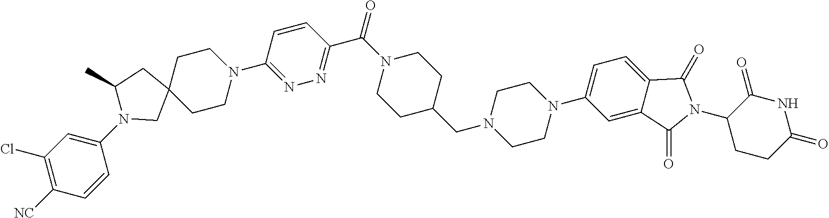 [SMILES: C[C@H]1CC2(CCN(c3ccc(C(=O)N4CCC(CN5CCN(c6ccc7c(c6)C(=O)N(C6CCC(=O)NC6=O)C7=O)CC5)CC4)nn3)CC2)CN1c1ccc(C#N)c(Cl)c1] |
| A96 | 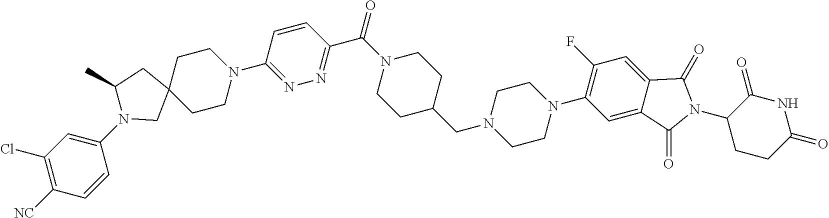 [SMILES: C[C@H]1CC2(CCN(c3ccc(C(=O)N4CCC(CN5CCN(c6cc7c(cc6F)C(=O)N(C6CCC(=O)NC6=O)C7=O)CC5)CC4)nn3)CC2)CN1c1ccc(C#N)c(Cl)c1] |
| A97 | 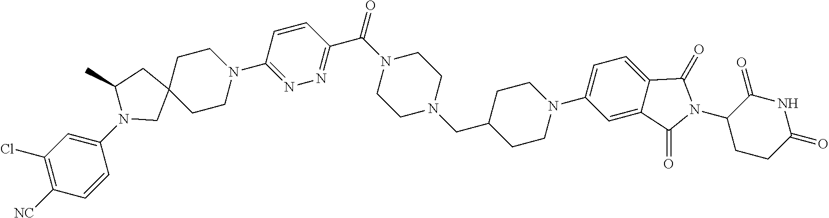 [SMILES: C[C@H]1CC2(CCN(c3ccc(C(=O)N4CCN(CC5CCN(c6ccc7c(c6)C(=O)N(C6CCC(=O)NC6=O)C7=O)CC5)CC4)nn3)CC2)CN1c1ccc(C#N)c(Cl)c1] |
| A98 | 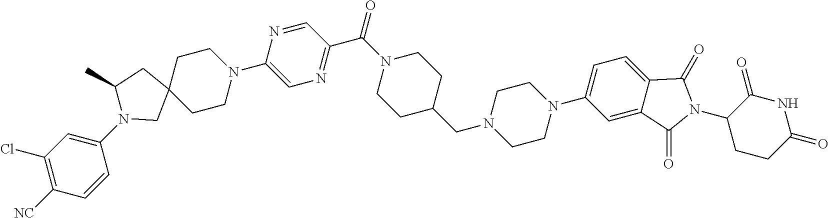 [SMILES: C[C@H]1CC2(CCN(c3cnc(C(=O)N4CCC(CN5CCN(c6ccc7c(c6)C(=O)N(C6CCC(=O)NC6=O)C7=O)CC5)CC4)cn3)CC2)CN1c1ccc(C#N)c(Cl)c1] |
| A99 | 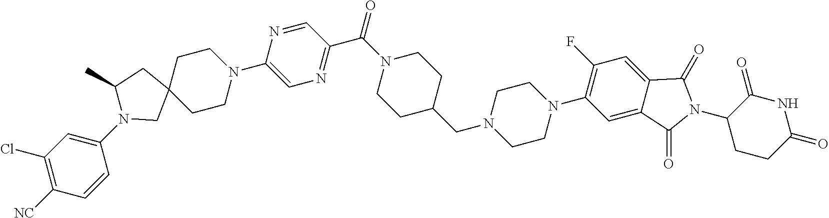 [SMILES: C[C@H]1CC2(CCN(c3cnc(C(=O)N4CCC(CN5CCN(c6cc7c(cc6F)C(=O)N(C6CCC(=O)NC6=O)C7=O)CC5)CC4)cn3)CC2)CN1c1ccc(C#N)c(Cl)c1] |
| A100 | 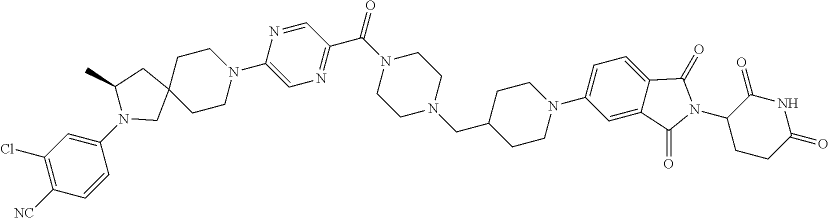 [SMILES: C[C@H]1CC2(CCN(c3cnc(C(=O)N4CCN(CC5CCN(c6ccc7c(c6)C(=O)N(C6CCC(=O)NC6=O)C7=O)CC5)CC4)cn3)CC2)CN1c1ccc(C#N)c(Cl)c1] |
| A101 | 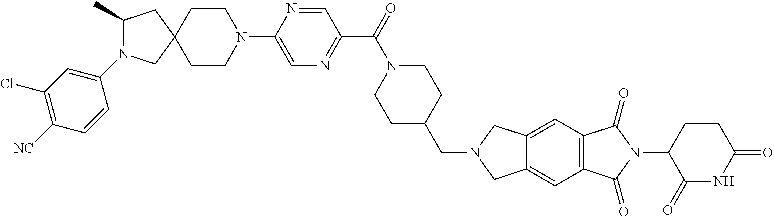 [SMILES: C[C@H]1CC2(CCN(c3cnc(C(=O)N4CCC(CN5Cc6cc7c(cc6C5)C(=O)N(C5CCC(=O)NC5=O)C7=O)CC4)cn3)CC2)CN1c1ccc(C#N)c(Cl)c1] |
| A102 | 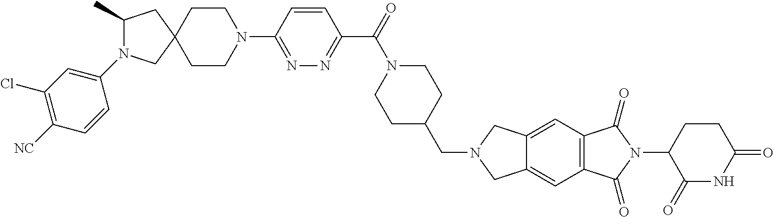 [SMILES: C[C@H]1CC2(CCN(c3ccc(C(=O)N4CCC(CN5Cc6cc7c(cc6C5)C(=O)N(C5CCC(=O)NC5=O)C7=O)CC4)nn3)CC2)CN1c1ccc(C#N)c(Cl)c1] |
| A103 | 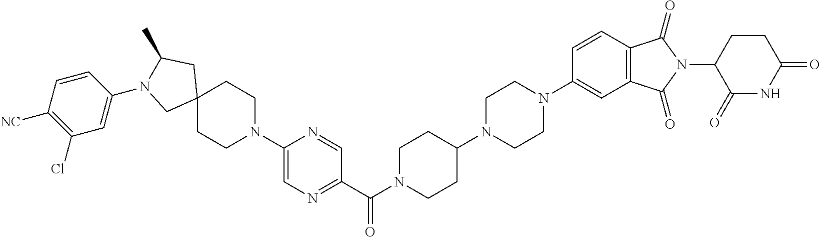 [SMILES: C[C@H]1CC2(CCN(c3cnc(C(=O)N4CCC(N5CCN(c6ccc7c(c6)C(=O)N(C6CCC(=O)NC6=O)C7=O)CC5)CC4)cn3)CC2)CN1c1ccc(C#N)c(Cl)c1] |
| A104 | 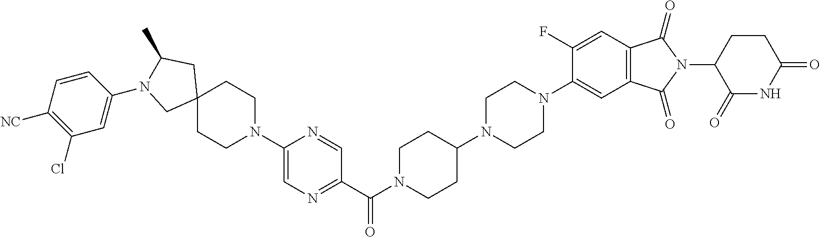 [SMILES: C[C@H]1CC2(CCN(c3cnc(C(=O)N4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| A105 | 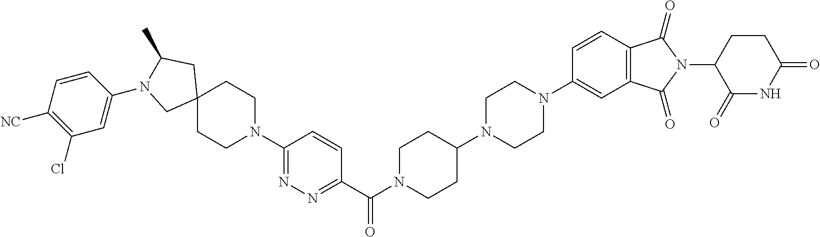 [SMILES: C[C@H]1CC2(CCN(c3ccc(C(=O)N4CCC(N5CCN(c6ccc7c(c6)C(=O)N(C6CCC(=O)NC6=O)C7=O)CC5)CC4)nn3)CC2)CN1c1ccc(C#N)c(Cl)c1] |
| A106 | 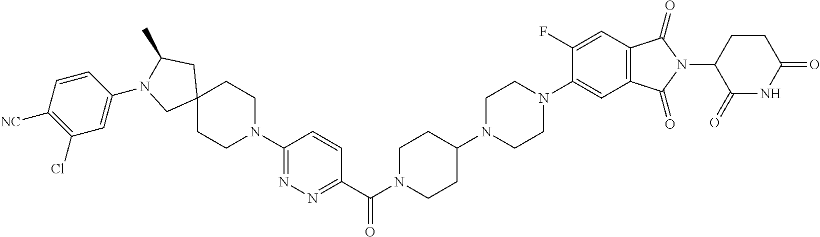 [SMILES: C[C@H]1CC2(CCN(c3ccc(C(=O)N4CCC(N5CCN(c6cc7c(cc6F)C(=O)N(C6CCC(=O)NC6=O)C7=O)CC5)CC4)nn3)CC2)CN1c1ccc(C#N)c(Cl)c1] |
| A107 | 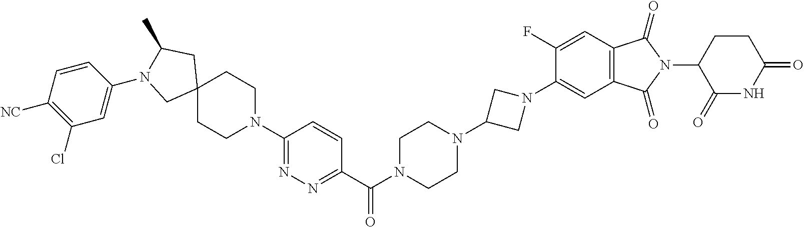 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)nn3)CC2)CN1c1ccc(C#N)c(Cl)c1] |
| A108 | 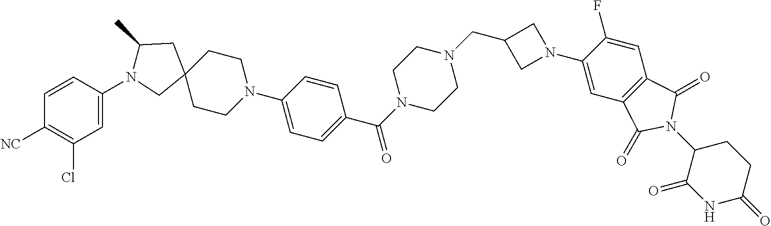 [SMILES: C[C@H]1CC2(CCN(c3ccc(C(=O)N4CCN(CC5CN(c6cc7c(cc6F)C(=O)N(C6CCC(=O)NC6=O)C7=O)C5)CC4)cc3)CC2)CN1c1ccc(C#N)c(Cl)c1] |
| A109 | 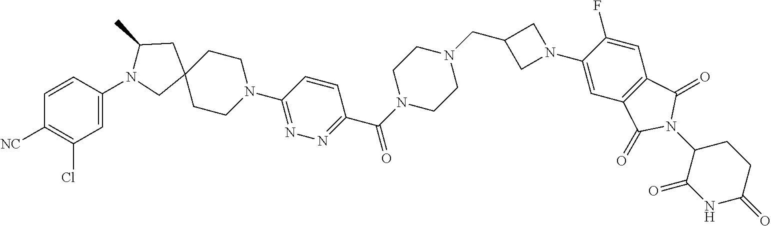 [SMILES: C[C@H]1CC2(CCN(c3ccc(C(=O)N4CCN(CC5CN(c6cc7c(cc6F)C(=O)N(C6CCC(=O)NC6=O)C7=O)C5)CC4)nn3)CC2)CN1c1ccc(C#N)c(Cl)c1] |
| A110 | 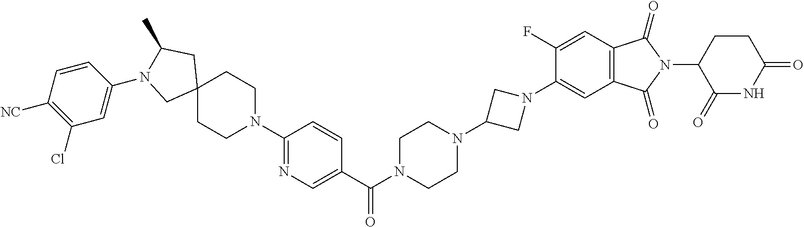 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cn3)CC2)CN1c1ccc(C#N)c(Cl)c1] |
| A111 | 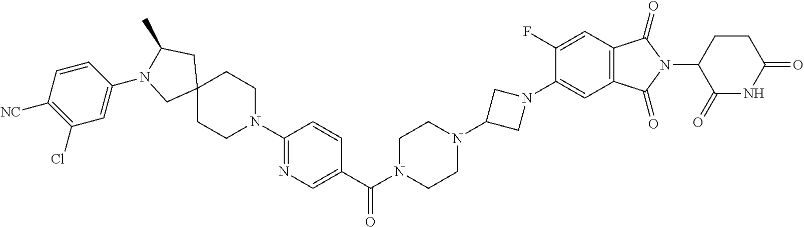 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cn3)CC2)CN1c1ccc(C#N)c(Cl)c1] |
| A112 | 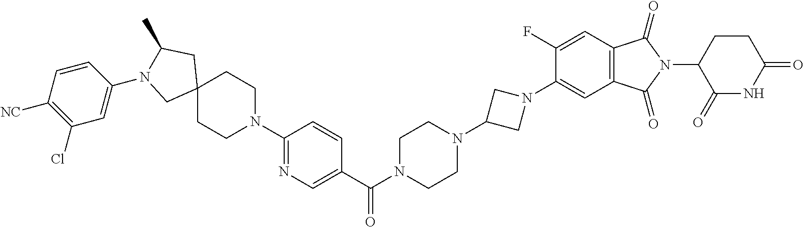 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cn3)CC2)CN1c1ccc(C#N)c(Cl)c1] |
| A113 | 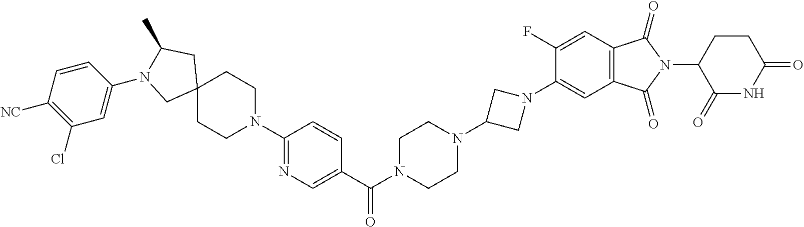 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cn3)CC2)CN1c1ccc(C#N)c(Cl)c1] |
| A114 | 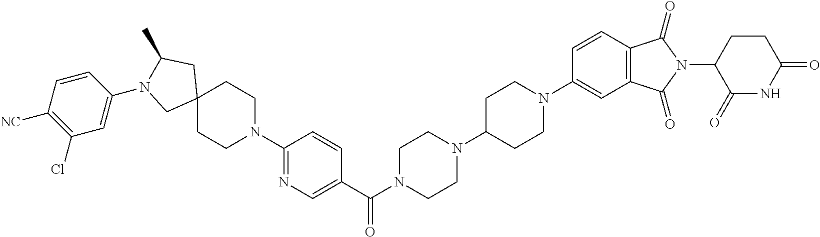 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| A115 | 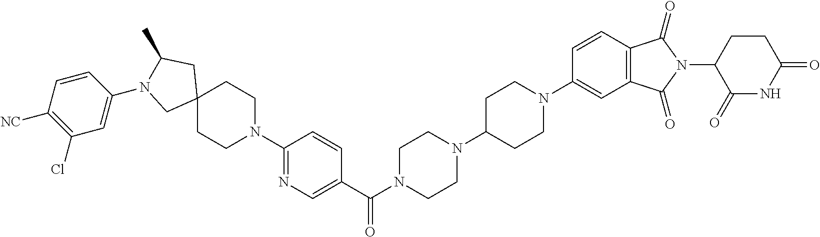 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| A116 | 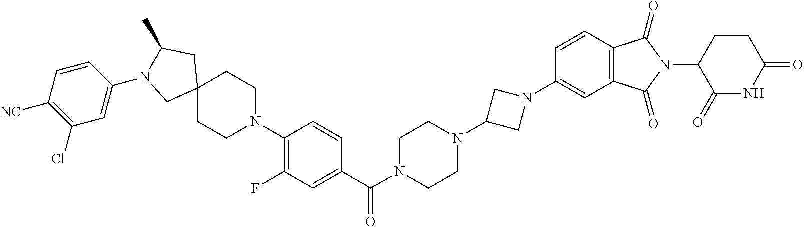 [SMILES: C[C@H]1CC2(CCN(c3ccc(C(=O)N4CCN(C5CN(c6ccc7c(c6)C(=O)N(C6CCC(=O)NC6=O)C7=O)C5)CC4)cc3F)CC2)CN1c1ccc(C#N)c(Cl)c1] |
| A117 | 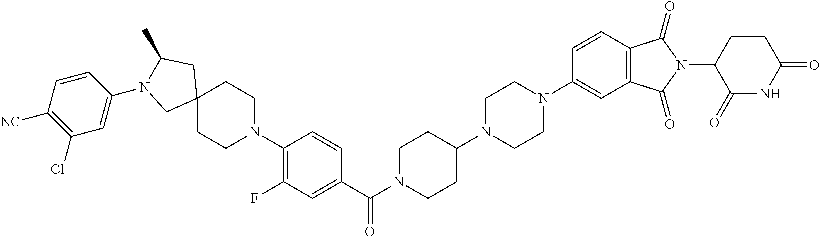 [SMILES: C[C@H]1CC2(CCN(c3ccc(C(=O)N4CCC(N5CCN(c6ccc7c(c6)C(=O)N(C6CCC(=O)NC6=O)C7=O)CC5)CC4)cc3F)CC2)CN1c1ccc(C#N)c(Cl)c1] |
| A118 | 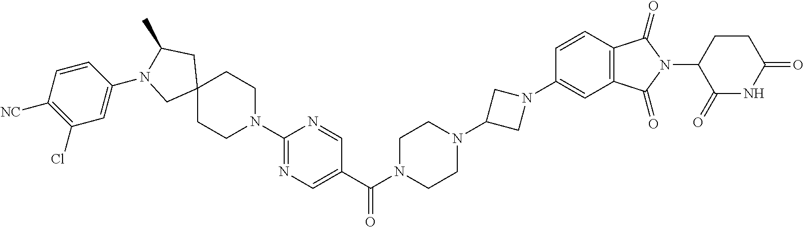 [SMILES: C[C@H]1CC2(CCN(c3ncc(C(=O)N4CCN(C5CN(c6ccc7c(c6)C(=O)N(C6CCC(=O)NC6=O)C7=O)C5)CC4)cn3)CC2)CN1c1ccc(C#N)c(Cl)c1] |
| A119 | 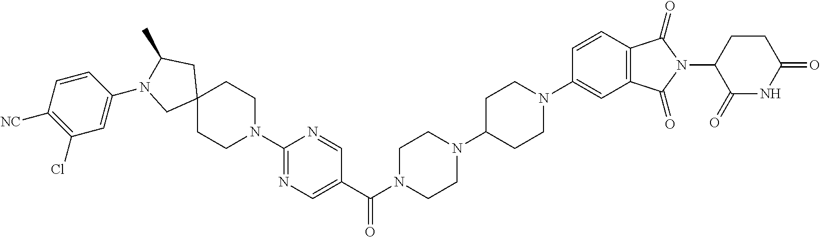 [SMILES: C[C@H]1CC2(CCN(c3ncc(C(=O)N4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| A120 | 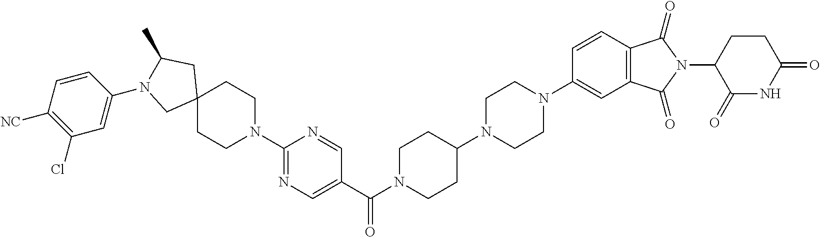 [SMILES: C[C@H]1CC2(CCN(c3ncc(C(=O)N4CCC(N5CCN(c6ccc7c(c6)C(=O)N(C6CCC(=O)NC6=O)C7=O)CC5)CC4)cn3)CC2)CN1c1ccc(C#N)c(Cl)c1] |
| A121 | 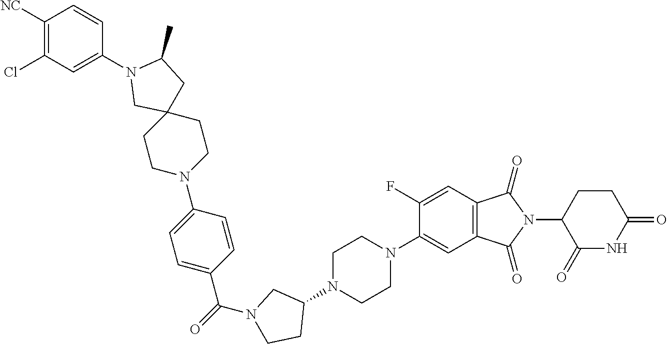 [SMILES: C[C@H]1CC2(CCN(c3ccc(C(=O)N4CC[C@@H](N5CCN(c6cc7c(cc6F)C(=O)N(C6CCC(=O)NC6=O)C7=O)CC5)C4)cc3)CC2)CN1c1ccc(C#N)c(Cl)c1] |
| A122 | 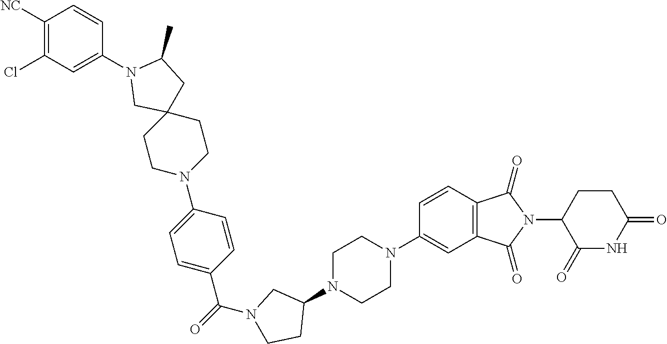 [SMILES: C[C@H]1CC2(CCN(c3ccc(C(=O)N4CC[C@H](N5CCN(c6ccc7c(c6)C(=O)N(C6CCC(=O)NC6=O)C7=O)CC5)C4)cc3)CC2)CN1c1ccc(C#N)c(Cl)c1] |
| A123 | 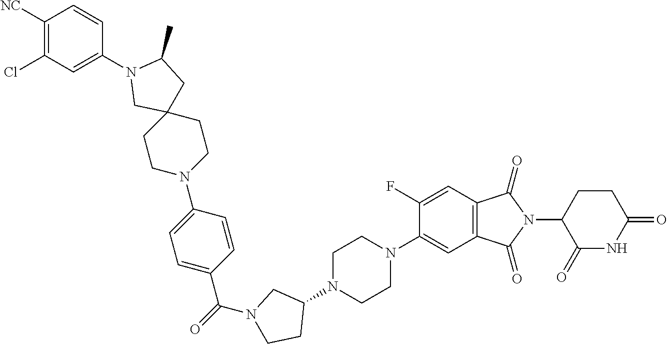 [SMILES: C[C@H]1CC2(CCN(c3ccc(C(=O)N4CC[C@@H](N5CCN(c6cc7c(cc6F)C(=O)N(C6CCC(=O)NC6=O)C7=O)CC5)C4)cc3)CC2)CN1c1ccc(C#N)c(Cl)c1] |
| A124 | 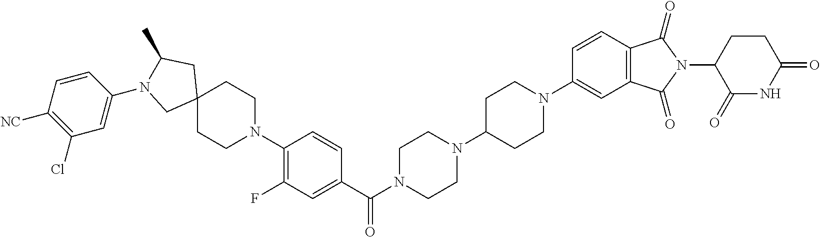 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A125 | 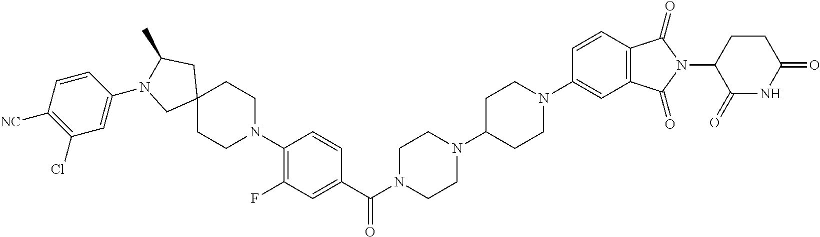 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A126 | 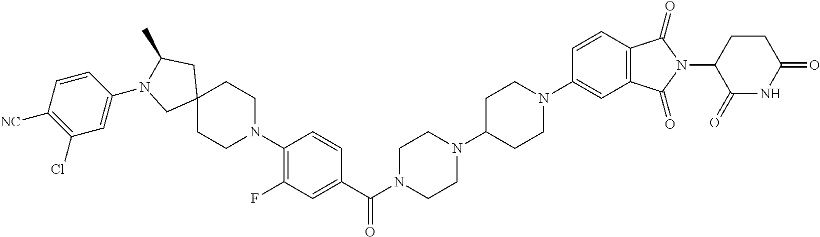 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A127 | 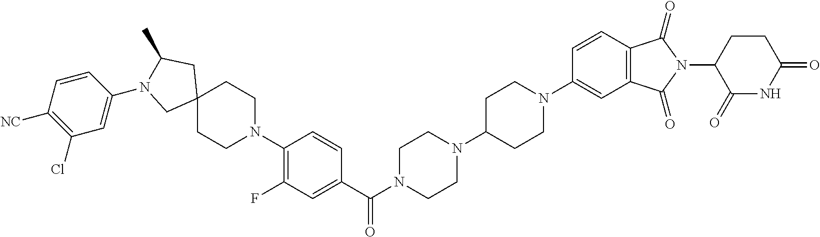 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A128 | 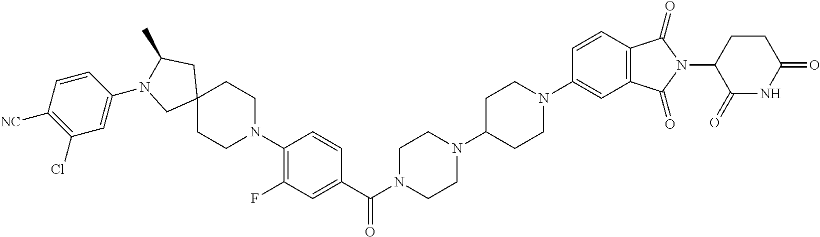 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A129 | 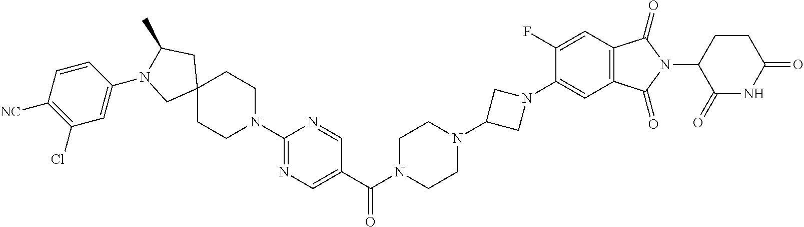 [SMILES: C[C@H]1CC2(CCN(c3ncc(C(=O)N4CCN(C5CN(c6cc7c(cc6F)C(=O)N(C6CCC(=O)NC6=O)C7=O)C5)CC4)cn3)CC2)CN1c1ccc(C#N)c(Cl)c1] |
| A130 | 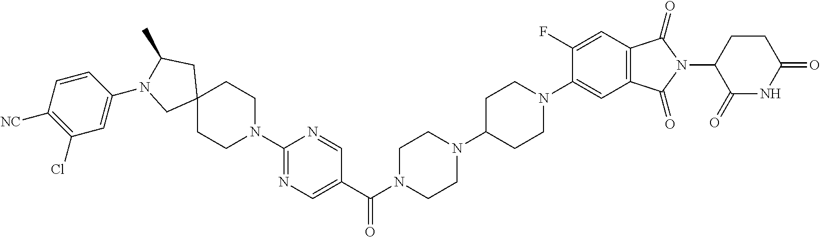 [SMILES: C[C@H]1CC2(CCN(c3ncc(C(=O)N4CCN(C5CCN(c6cc7c(cc6F)C(=O)N(C6CCC(=O)NC6=O)C7=O)CC5)CC4)cn3)CC2)CN1c1ccc(C#N)c(Cl)c1] |
| A131 | 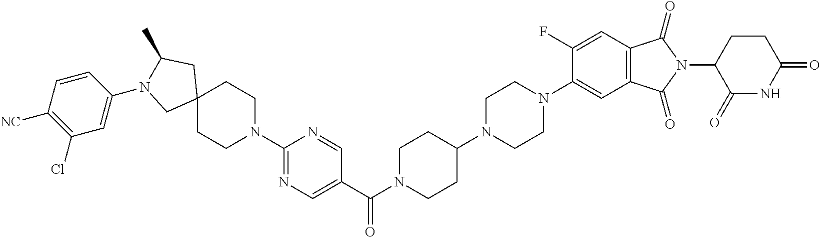 [SMILES: C[C@H]1CC2(CCN(c3ncc(C(=O)N4CCC(N5CCN(c6cc7c(cc6F)C(=O)N(C6CCC(=O)NC6=O)C7=O)CC5)CC4)cn3)CC2)CN1c1ccc(C#N)c(Cl)c1] |
| A132 | 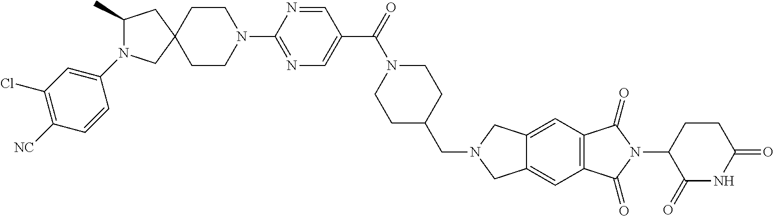 [SMILES: C[C@H]1CC2(CCN(c3ncc(C(=O)N4CCC(CN5Cc6cc7c(cc6C5)C(=O)N(C5CCC(=O)NC5=O)C7=O)CC4)cn3)CC2)CN1c1ccc(C#N)c(Cl)c1] |
| A133 | 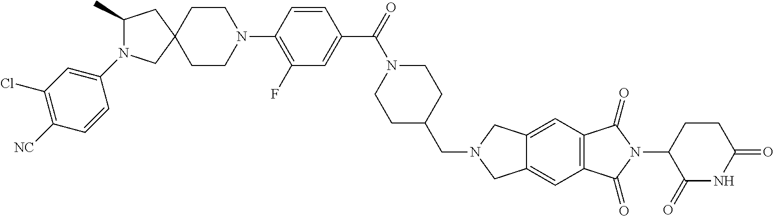 [SMILES: C[C@H]1CC2(CCN(c3ccc(C(=O)N4CCC(CN5Cc6cc7c(cc6C5)C(=O)N(C5CCC(=O)NC5=O)C7=O)CC4)cc3F)CC2)CN1c1ccc(C#N)c(Cl)c1] |
| A134 | 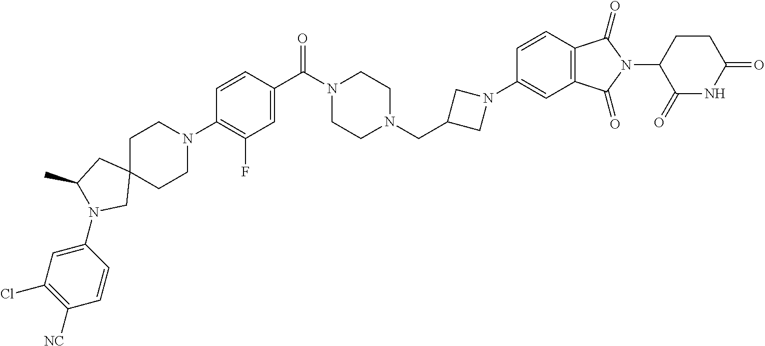 [SMILES: C[C@H]1CC2(CCN(c3ccc(C(=O)N4CCN(CC5CN(c6ccc7c(c6)C(=O)N(C6CCC(=O)NC6=O)C7=O)C5)CC4)cc3F)CC2)CN1c1ccc(C#N)c(Cl)c1] |
| A135 | 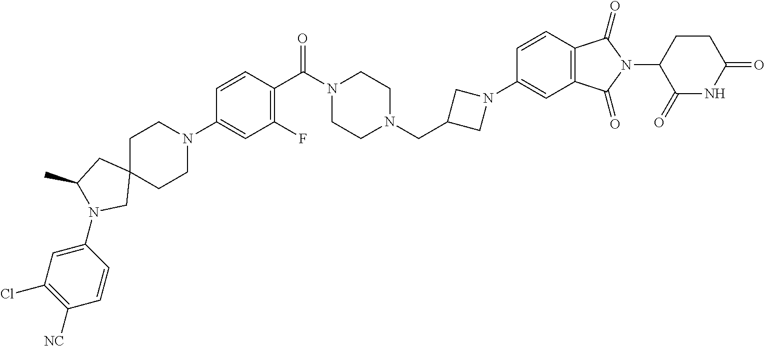 [SMILES: C[C@H]1CC2(CCN(c3ccc(C(=O)N4CCN(CC5CN(c6ccc7c(c6)C(=O)N(C6CCC(=O)NC6=O)C7=O)C5)CC4)c(F)c3)CC2)CN1c1ccc(C#N)c(Cl)c1] |
| A136 | 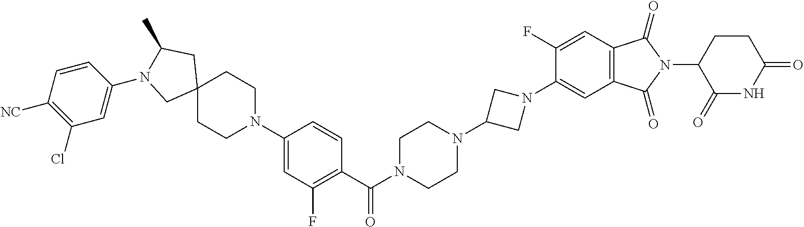 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)c(F)c3)CC2)CN1c1ccc(C#N)c(Cl)c1] |
| A137 | 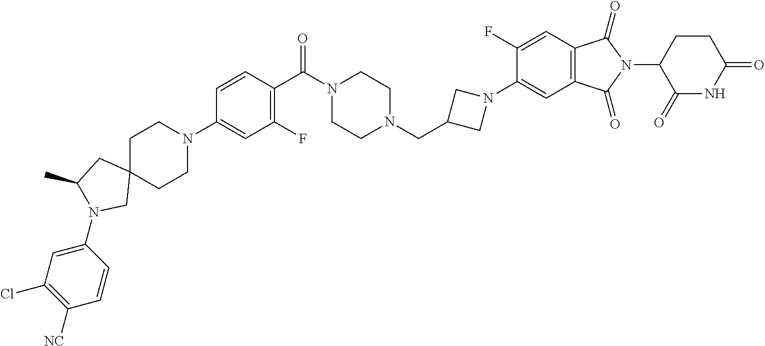 [SMILES: C[C@H]1CC2(CCN(c3ccc(C(=O)N4CCN(CC5CN(c6cc7c(cc6F)C(=O)N(C6CCC(=O)NC6=O)C7=O)C5)CC4)c(F)c3)CC2)CN1c1ccc(C#N)c(Cl)c1] |
| A138 | 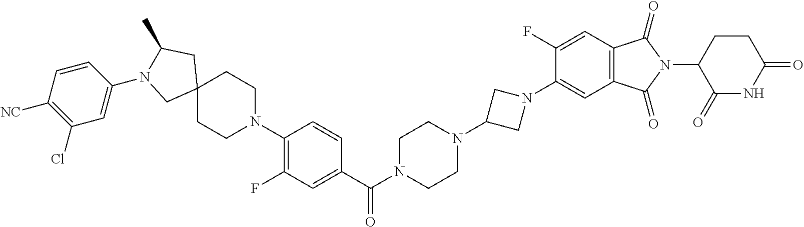 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)cc3F)CC2)CN1c1ccc(C#N)c(Cl)c1] |
| A139 | 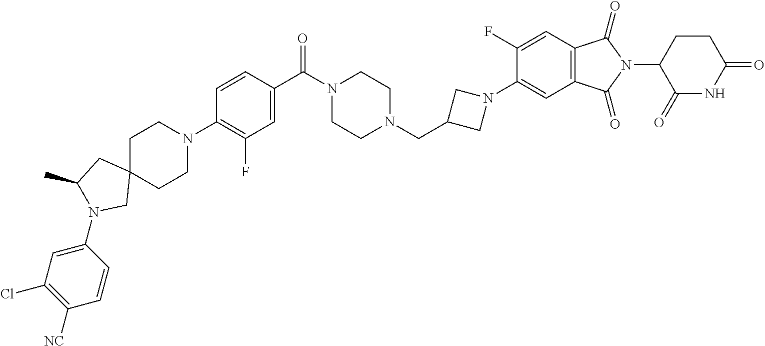 [SMILES: C[C@H]1CC2(CCN(c3ccc(C(=O)N4CCN(CC5CN(c6cc7c(cc6F)C(=O)N(C6CCC(=O)NC6=O)C7=O)C5)CC4)cc3F)CC2)CN1c1ccc(C#N)c(Cl)c1] |
| A140 | 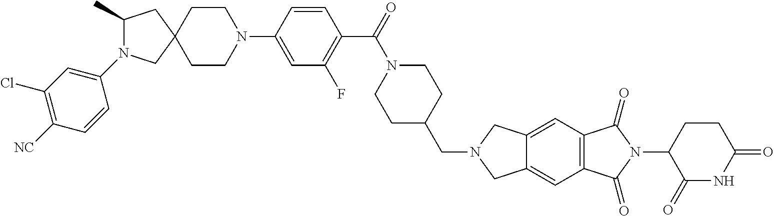 [SMILES: C[C@H]1CC2(CCN(c3ccc(C(=O)N4CCC(CN5Cc6cc7c(cc6C5)C(=O)N(C5CCC(=O)NC5=O)C7=O)CC4)c(F)c3)CC2)CN1c1ccc(C#N)c(Cl)c1] |
| A141 | 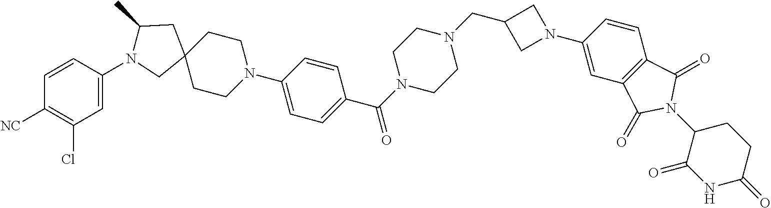 [SMILES: C[C@H]1CC2(CCN(c3ccc(C(=O)N4CCN(CC5CN(c6ccc7c(c6)C(=O)N(C6CCC(=O)NC6=O)C7=O)C5)CC4)cc3)CC2)CN1c1ccc(C#N)c(Cl)c1] |
| A142 | 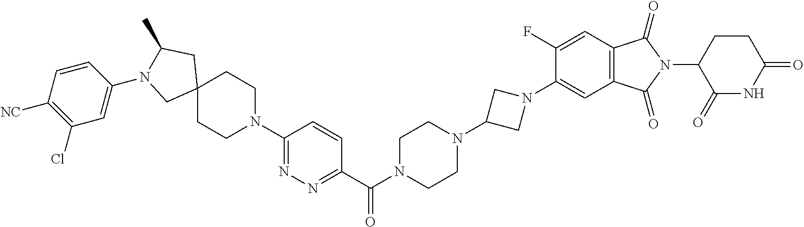 [SMILES: C[C@H]1CC2(CCN(c3ccc(C(=O)N4CCN(C5CN(c6cc7c(cc6F)C(=O)N(C6CCC(=O)NC6=O)C7=O)C5)CC4)nn3)CC2)CN1c1ccc(C#N)c(Cl)c1] |
| A143 | 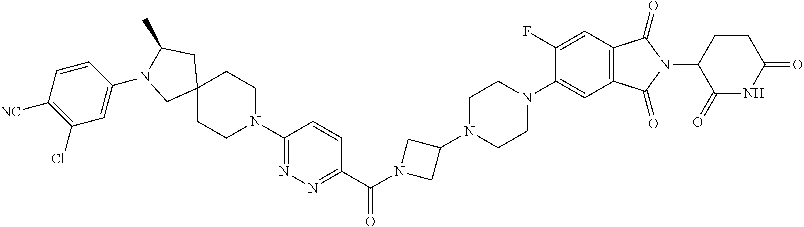 [SMILES: C[C@H]1CC2(CCN(c3ccc(C(=O)N4CC(N5CCN(c6cc7c(cc6F)C(=O)N(C6CCC(=O)NC6=O)C7=O)CC5)C4)nn3)CC2)CN1c1ccc(C#N)c(Cl)c1] |
| A144 | 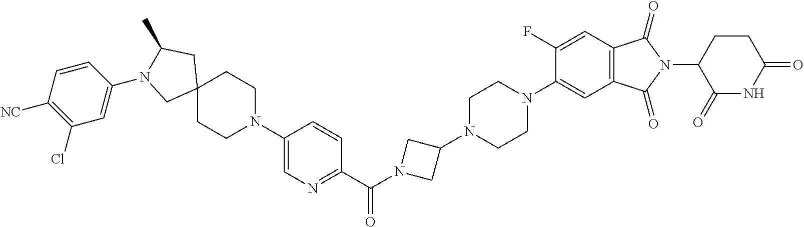 [SMILES: C[C@H]1CC2(CCN(c3ccc(C(=O)N4CC(N5CCN(c6cc7c(cc6F)C(=O)N(C6CCC(=O)NC6=O)C7=O)CC5)C4)nc3)CC2)CN1c1ccc(C#N)c(Cl)c1] |
| A145 | 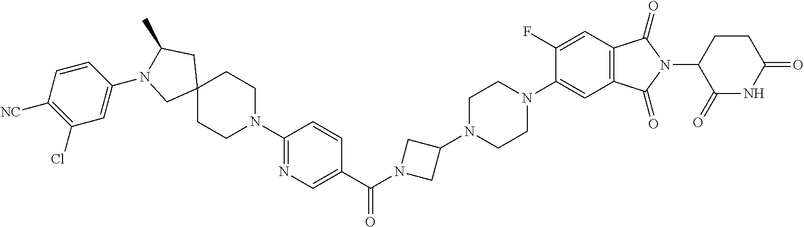 [SMILES: C[C@H]1CC2(CCN(c3ccc(C(=O)N4CC(N5CCN(c6cc7c(cc6F)C(=O)N(C6CCC(=O)NC6=O)C7=O)CC5)C4)cn3)CC2)CN1c1ccc(C#N)c(Cl)c1] |
| A146 | 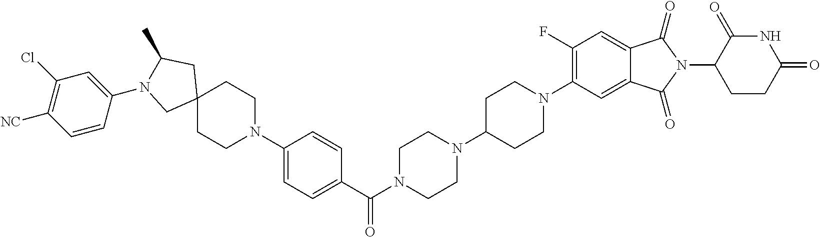 [SMILES: C[C@H]1CC2(CCN(c3ccc(C(=O)N4CCN(C5CCN(c6cc7c(cc6F)C(=O)N(C6CCC(=O)NC6=O)C7=O)CC5)CC4)cc3)CC2)CN1c1ccc(C#N)c(Cl)c1] |
| A147 | 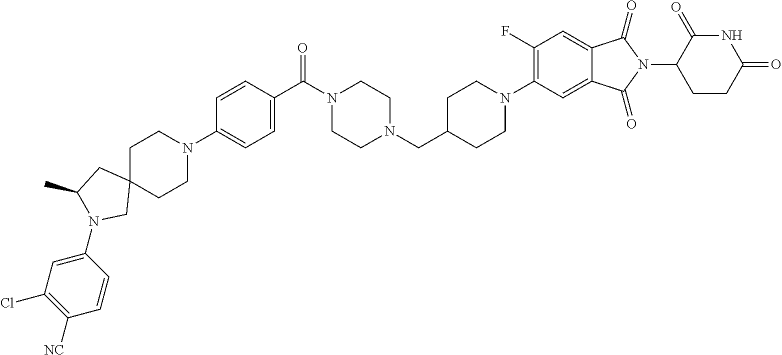 [SMILES: C[C@H]1CC2(CCN(c3ccc(C(=O)N4CCN(CC5CCN(c6cc7c(cc6F)C(=O)N(C6CCC(=O)NC6=O)C7=O)CC5)CC4)cc3)CC2)CN1c1ccc(C#N)c(Cl)c1] |
| A148 | 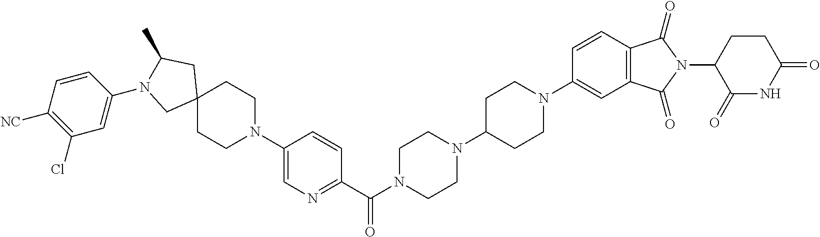 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)nc3)CC2)CN1c1ccc(C#N)c(Cl)c1] |
| A149 | 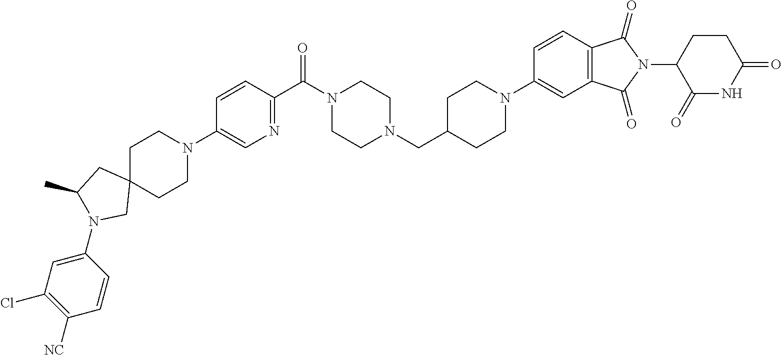 [SMILES: C[C@H]1CC2(CCN(c3ccc(C(=O)N4CCN(CC5CCN(c6ccc7c(c6)C(=O)N(C6CCC(=O)NC6=O)C7=O)CC5)CC4)nc3)CC2)CN1c1ccc(C#N)c(Cl)c1] |
| A150 | 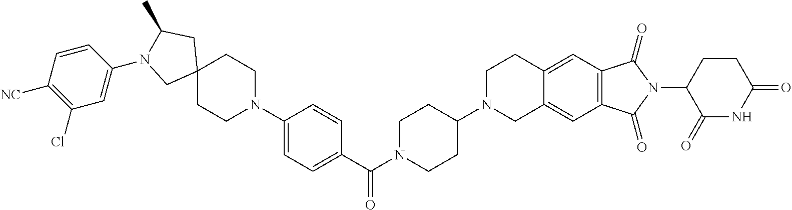 [SMILES: C[C@H]1CC2(CCN(c3ccc(C(=O)N4CCC(N5CCc6cc7c(cc6C5)C(=O)N(C5CCC(=O)NC5=O)C7=O)CC4)cc3)CC2)CN1c1ccc(C#N)c(Cl)c1] |
| A151 | 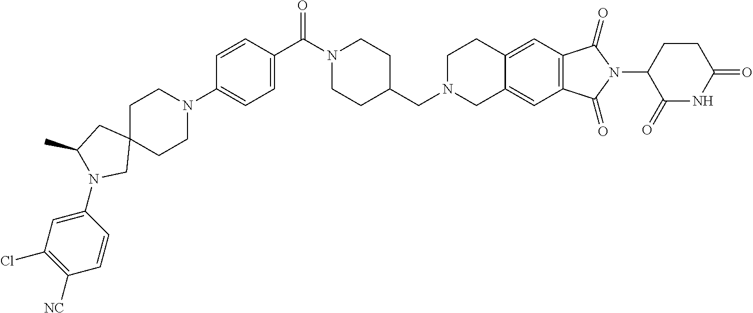 [SMILES: C[C@H]1CC2(CCN(c3ccc(C(=O)N4CCC(CN5CCc6cc7c(cc6C5)C(=O)N(C5CCC(=O)NC5=O)C7=O)CC4)cc3)CC2)CN1c1ccc(C#N)c(Cl)c1] |
| A152 | 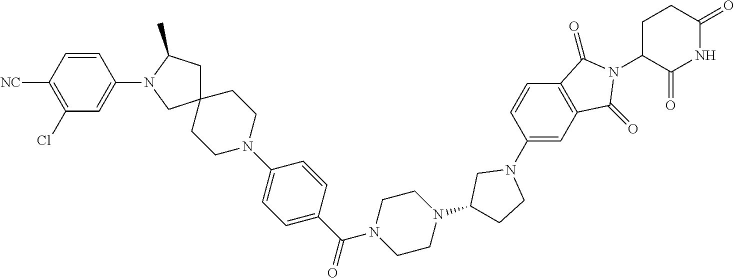 [SMILES: C[C@H]1CC2(CCN(c3ccc(C(=O)N4CCN([C@H]5CCN(c6ccc7c(c6)C(=O)N(C6CCC(=O)NC6=O)C7=O)C5)CC4)cc3)CC2)CN1c1ccc(C#N)c(Cl)c1] |
| A153 | 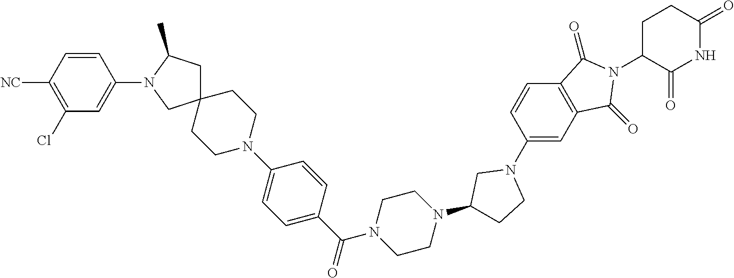 [SMILES: C[C@H]1CC2(CCN(c3ccc(C(=O)N4CCN([C@@H]5CCN(c6ccc7c(c6)C(=O)N(C6CCC(=O)NC6=O)C7=O)C5)CC4)cc3)CC2)CN1c1ccc(C#N)c(Cl)c1] |
| A154 | 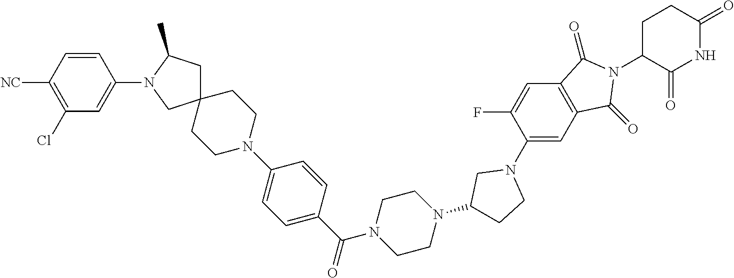 |
| A155 | 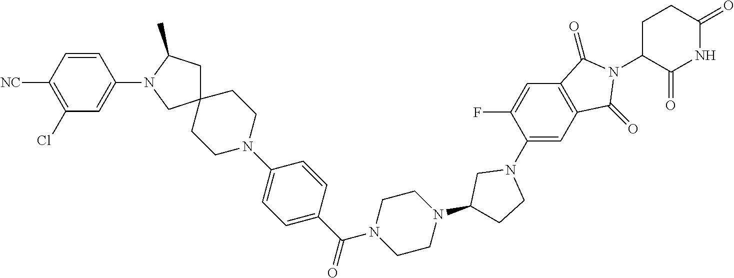 [SMILES: C[C@H]1CC2(CCN(c3ccc(C(=O)N4CCN([C@@H]5CCN(c6cc7c(cc6F)C(=O)N(C6CCC(=O)NC6=O)C7=O)C5)CC4)cc3)CC2)CN1c1ccc(C#N)c(Cl)c1] |
| A156 | 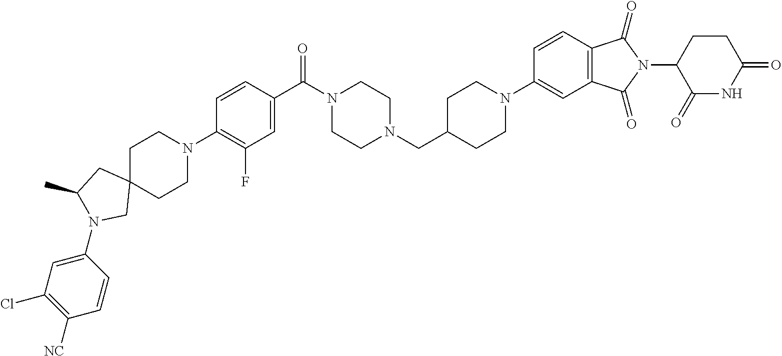 [SMILES: C[C@H]1CC2(CCN(c3ccc(C(=O)N4CCN(CC5CCN(c6ccc7c(c6)C(=O)N(C6CCC(=O)NC6=O)C7=O)CC5)CC4)cc3F)CC2)CN1c1ccc(C#N)c(Cl)c1] |
| A157 | 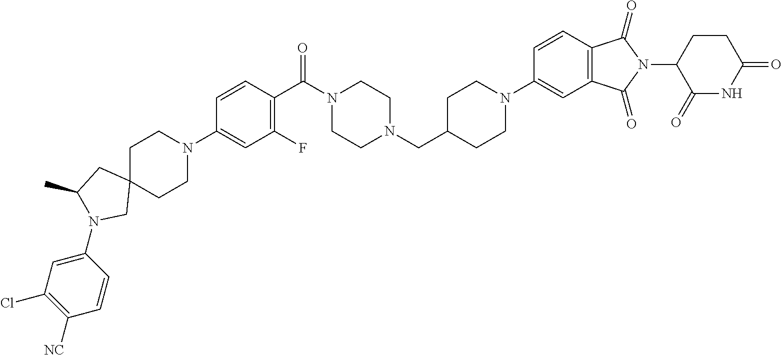 [SMILES: C[C@H]1CC2(CCN(c3ccc(C(=O)N4CCN(CC5CCN(c6ccc7c(c6)C(=O)N(C6CCC(=O)NC6=O)C7=O)CC5)CC4)c(F)c3)CC2)CN1c1ccc(C#N)c(Cl)c1] |
| A158 | 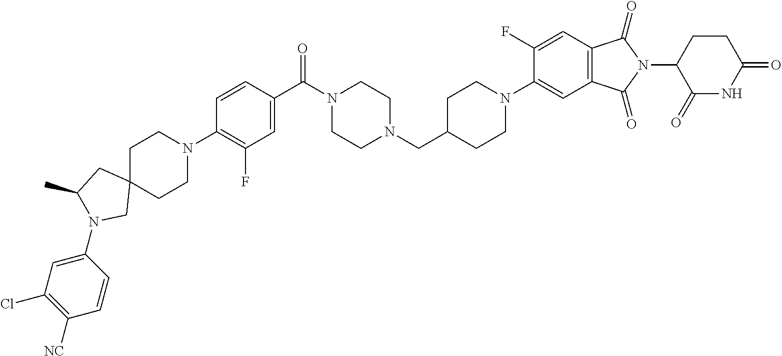 [SMILES: C[C@H]1CC2(CCN(c3ccc(C(=O)N4CCN(CC5CCN(c6cc7c(cc6F)C(=O)N(C6CCC(=O)NC6=O)C7=O)CC5)CC4)cc3F)CC2)CN1c1ccc(C#N)c(Cl)c1] |
| A159 | 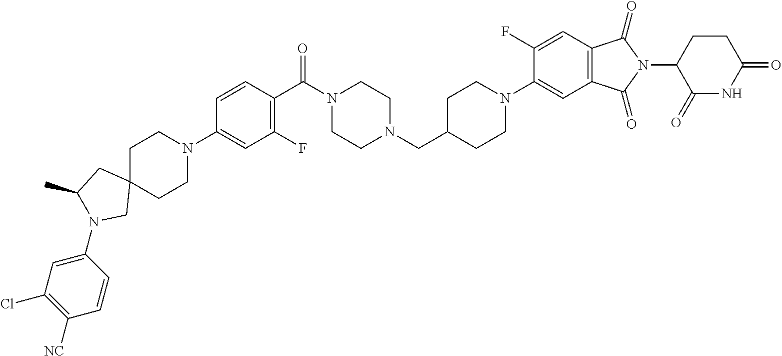 [SMILES: C[C@H]1CC2(CCN(c3ccc(C(=O)N4CCN(CC5CCN(c6cc7c(cc6F)C(=O)N(C6CCC(=O)NC6=O)C7=O)CC5)CC4)c(F)c3)CC2)CN1c1ccc(C#N)c(Cl)c1] |
| A160 | 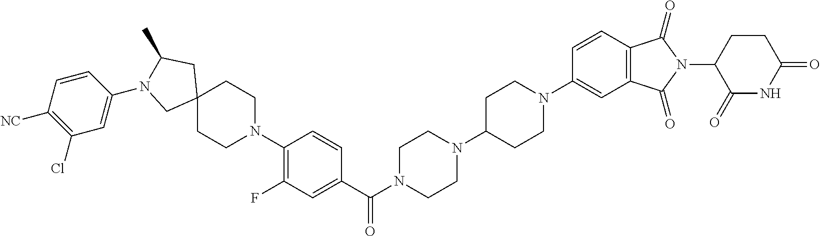 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)cc3F)CC2)CN1c1ccc(C#N)c(Cl)c1] |
| A161 | 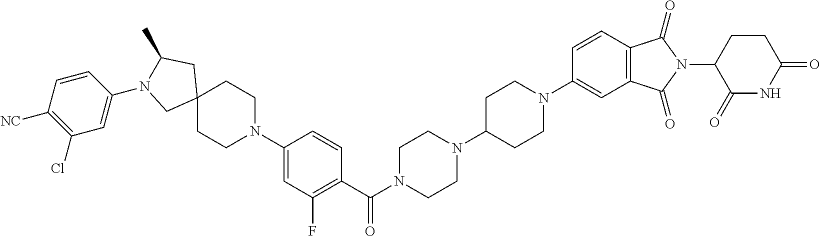 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)c(F)c3)CC2)CN1c1ccc(C#N)c(Cl)c1] |
| A162 | 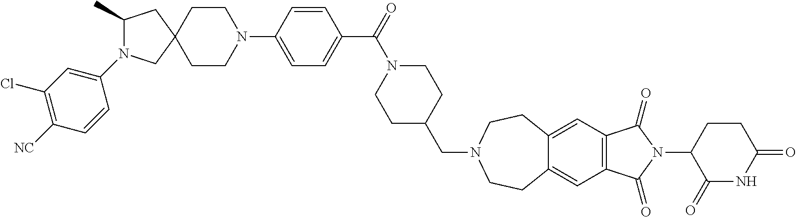 [SMILES: C[C@H]1CC2(CCN(c3ccc(C(=O)N4CCC(CN5CCc6cc7c(cc6CC5)C(=O)N(C5CCC(=O)NC5=O)C7=O)CC4)cc3)CC2)CN1c1ccc(C#N)c(Cl)c1] |
| A163 | 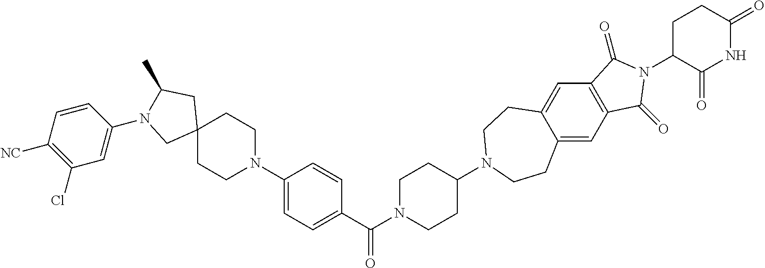 [SMILES: C[C@H]1CC2(CCN(c3ccc(C(=O)N4CCC(N5CCc6cc7c(cc6CC5)C(=O)N(C5CCC(=O)NC5=O)C7=O)CC4)cc3)CC2)CN1c1ccc(C#N)c(Cl)c1] |
| A164 | 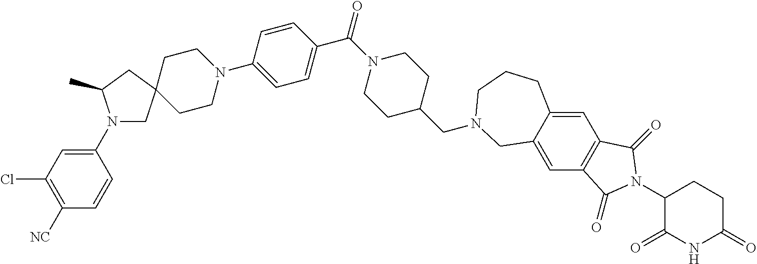 [SMILES: C[C@H]1CC2(CCN(c3ccc(C(=O)N4CCC(CN5CCCc6cc7c(cc6C5)C(=O)N(C5CCC(=O)NC5=O)C7=O)CC4)cc3)CC2)CN1c1ccc(C#N)c(Cl)c1] |
| A165 | 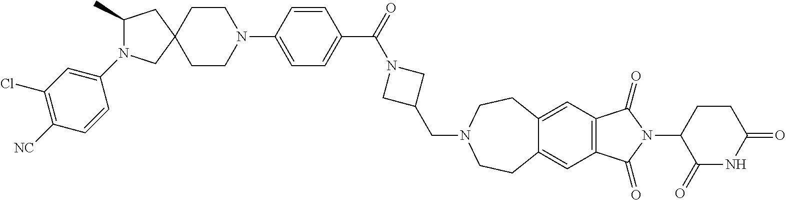 [SMILES: C[C@H]1CC2(CCN(c3ccc(C(=O)N4CC(CN5CCc6cc7c(cc6CC5)C(=O)N(C5CCC(=O)NC5=O)C7=O)C4)cc3)CC2)CN1c1ccc(C#N)c(Cl)c1] |
| A166 | 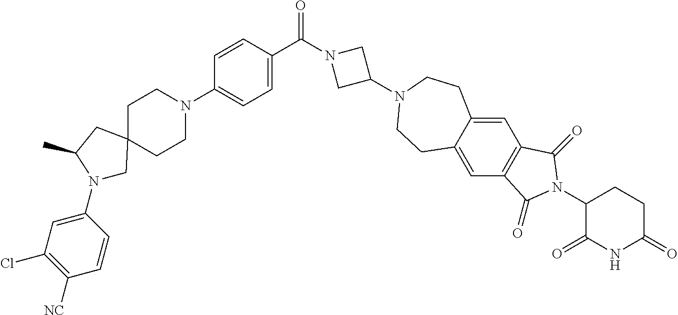 [SMILES: C[C@H]1CC2(CCN(c3ccc(C(=O)N4CC(N5CCc6cc7c(cc6CC5)C(=O)N(C5CCC(=O)NC5=O)C7=O)C4)cc3)CC2)CN1c1ccc(C#N)c(Cl)c1] |
| A167 | 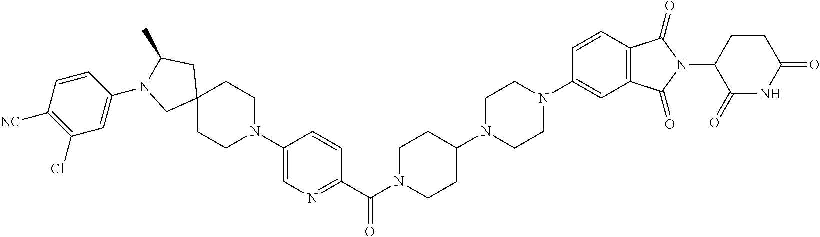 [SMILES: C[C@H]1CC2(CCN(c3ccc(C(=O)N4CCC(N5CCN(c6ccc7c(c6)C(=O)N(C6CCC(=O)NC6=O)C7=O)CC5)CC4)nc3)CC2)CN1c1ccc(C#N)c(Cl)c1] |
| A168 | 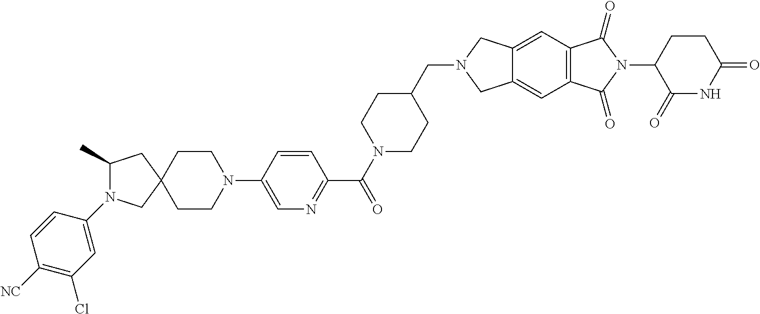 [SMILES: C[C@H]1CC2(CCN(c3ccc(C(=O)N4CCC(CN5Cc6cc7c(cc6C5)C(=O)N(C5CCC(=O)NC5=O)C7=O)CC4)nc3)CC2)CN1c1ccc(C#N)c(Cl)c1] |
| A169 | 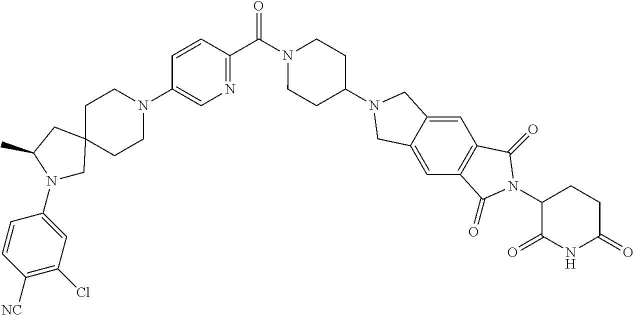 [SMILES: C[C@H]1CC2(CCN(c3ccc(C(=O)N4CCC(N5Cc6cc7c(cc6C5)C(=O)N(C5CCC(=O)NC5=O)C7=O)CC4)nc3)CC2)CN1c1ccc(C#N)c(Cl)c1] |
| A170 | 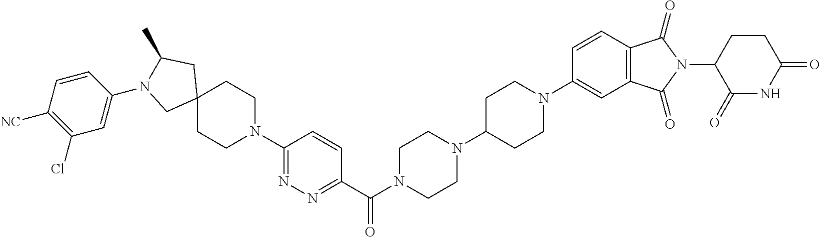 [SMILES: C[C@H]1CC2(CCN(c3ccc(C(=O)N4CCN(C5CCN(c6ccc7c(c6)C(=O)N(C6CCC(=O)NC6=O)C7=O)CC5)CC4)nn3)CC2)CN1c1ccc(C#N)c(Cl)c1] |
| A171 | 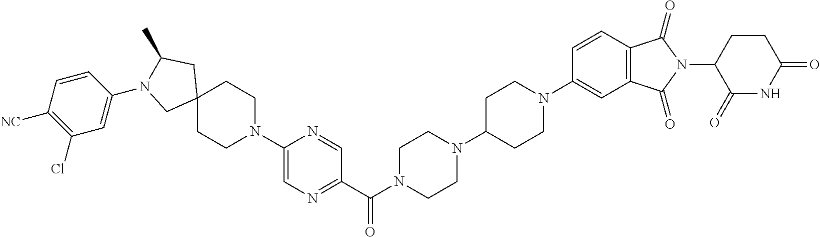 [SMILES: C[C@H]1CC2(CCN(c3cnc(C(=O)N4CCN(C5CCN(c6ccc7c(c6)C(=O)N(C6CCC(=O)NC6=O)C7=O)CC5)CC4)cn3)CC2)CN1c1ccc(C#N)c(Cl)c1] |
| A172 | 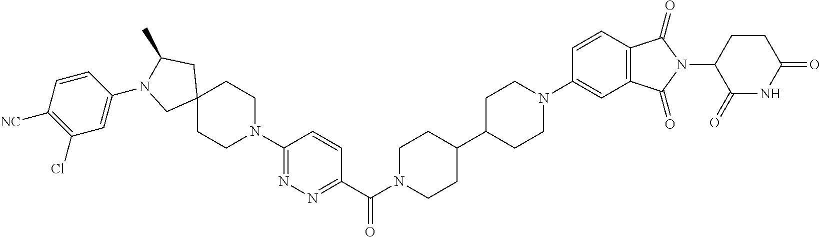 [SMILES: C[C@H]1CC2(CCN(c3ccc(C(=O)N4CCC(C5CCN(c6ccc7c(c6)C(=O)N(C6CCC(=O)NC6=O)C7=O)CC5)CC4)nn3)CC2)CN1c1ccc(C#N)c(Cl)c1] |
| A173 | 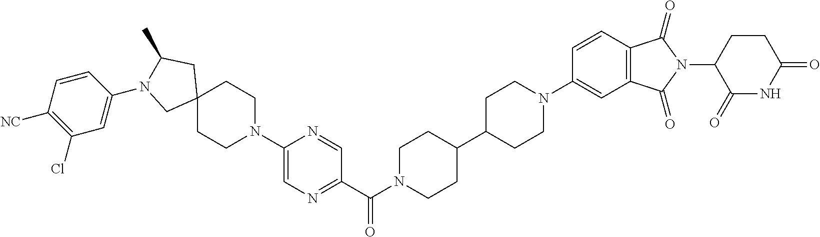 [SMILES: C[C@H]1CC2(CCN(c3cnc(C(=O)N4CCC(C5CCN(c6ccc7c(c6)C(=O)N(C6CCC(=O)NC6=O)C7=O)CC5)CC4)cn3)CC2)CN1c1ccc(C#N)c(Cl)c1] |
| A174 | 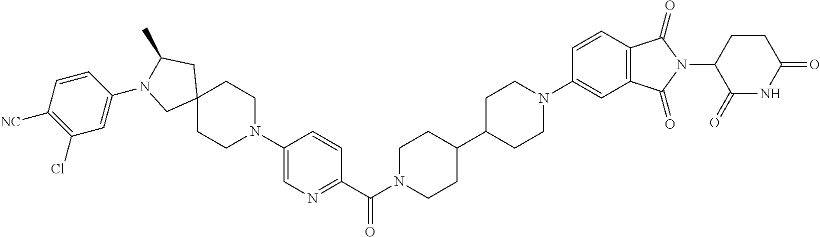 [SMILES: C[C@H]1CC2(CCN(c3ccc(C(=O)N4CCC(C5CCN(c6ccc7c(c6)C(=O)N(C6CCC(=O)NC6=O)C7=O)CC5)CC4)nc3)CC2)CN1c1ccc(C#N)c(Cl)c1] |
| A175 | 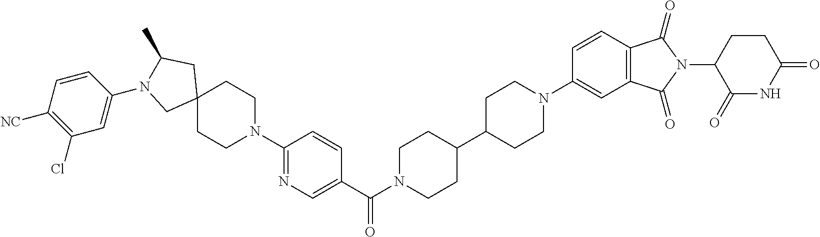 [SMILES: C[C@H]1CC2(CCN(c3ccc(C(=O)N4CCC(C5CCN(c6ccc7c(c6)C(=O)N(C6CCC(=O)NC6=O)C7=O)CC5)CC4)cn3)CC2)CN1c1ccc(C#N)c(Cl)c1] |
| A176 | 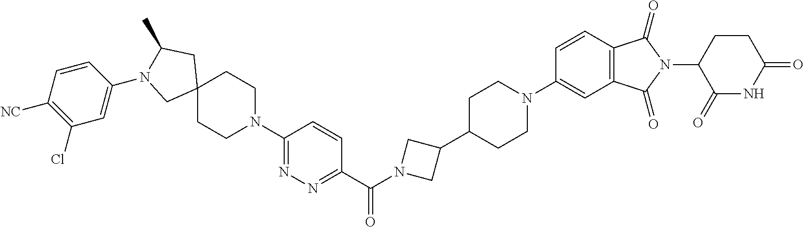 [SMILES: C[C@H]1CC2(CCN(c3ccc(C(=O)N4CC(C5CCN(c6ccc7c(c6)C(=O)N(C6CCC(=O)NC6=O)C7=O)CC5)C4)nn3)CC2)CN1c1ccc(C#N)c(Cl)c1] |
| A177 | 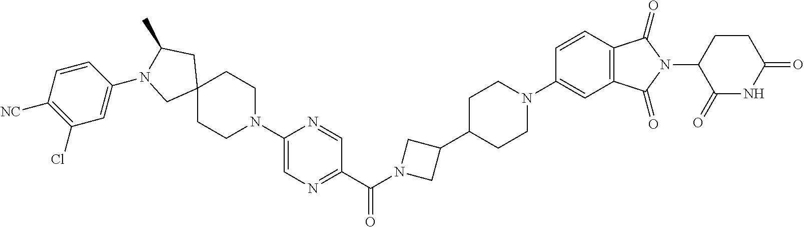 [SMILES: C[C@H]1CC2(CCN(c3cnc(C(=O)N4CC(C5CCN(c6ccc7c(c6)C(=O)N(C6CCC(=O)NC6=O)C7=O)CC5)C4)cn3)CC2)CN1c1ccc(C#N)c(Cl)c1] |
| A178 | 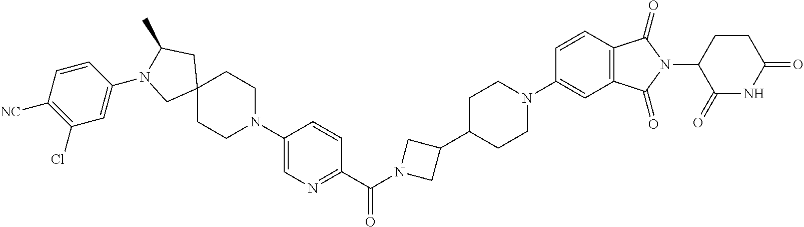 [SMILES: C[C@H]1CC2(CCN(c3ccc(C(=O)N4CC(C5CCN(c6ccc7c(c6)C(=O)N(C6CCC(=O)NC6=O)C7=O)CC5)C4)nc3)CC2)CN1c1ccc(C#N)c(Cl)c1] |
| A179 | 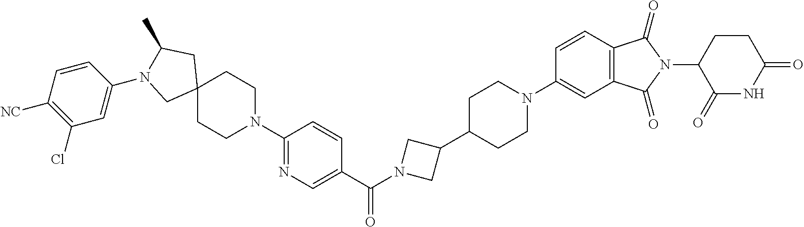 [SMILES: C[C@H]1CC2(CCN(c3ccc(C(=O)N4CC(C5CCN(c6ccc7c(c6)C(=O)N(C6CCC(=O)NC6=O)C7=O)CC5)C4)cn3)CC2)CN1c1ccc(C#N)c(Cl)c1] |
| A180 | 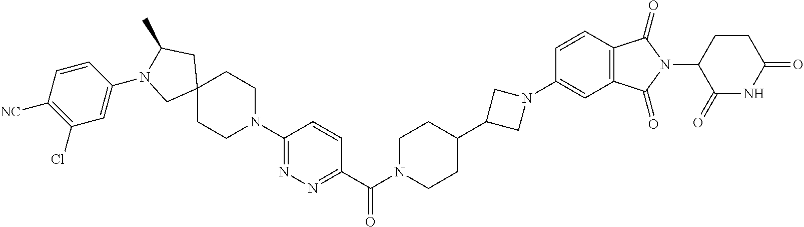 [SMILES: C[C@H]1CC2(CCN(c3ccc(C(=O)N4CCC(C5CN(c6ccc7c(c6)C(=O)N(C6CCC(=O)NC6=O)C7=O)C5)CC4)nn3)CC2)CN1c1ccc(C#N)c(Cl)c1] |
| A181 | 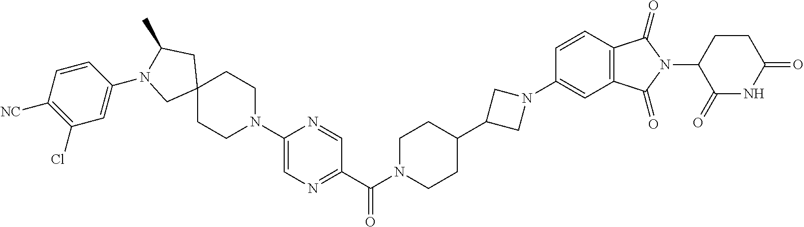 [SMILES: C[C@H]1CC2(CCN(c3cnc(C(=O)N4CCC(C5CN(c6ccc7c(c6)C(=O)N(C6CCC(=O)NC6=O)C7=O)C5)CC4)cn3)CC2)CN1c1ccc(C#N)c(Cl)c1] |
| A182 | 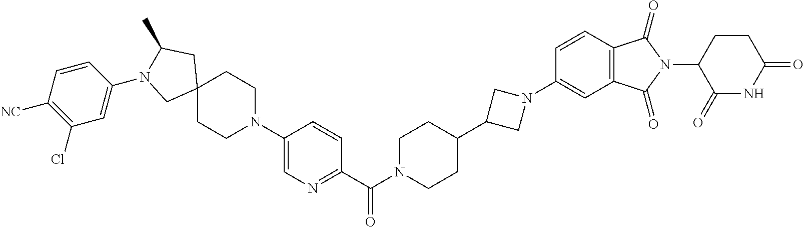 [SMILES: C[C@H]1CC2(CCN(c3ccc(C(=O)N4CCC(C5CN(c6ccc7c(c6)C(=O)N(C6CCC(=O)NC6=O)C7=O)C5)CC4)nc3)CC2)CN1c1ccc(C#N)c(Cl)c1] |
| A183 | 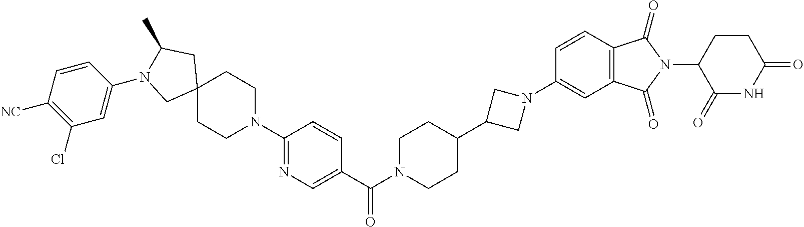 [SMILES: C[C@H]1CC2(CCN(c3ccc(C(=O)N4CCC(C5CN(c6ccc7c(c6)C(=O)N(C6CCC(=O)NC6=O)C7=O)C5)CC4)cn3)CC2)CN1c1ccc(C#N)c(Cl)c1] |
| A184 | 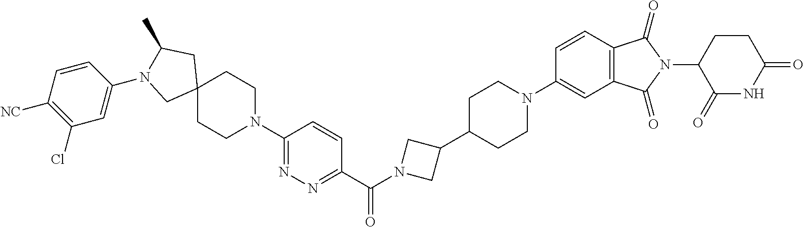 [SMILES: C[C@H]1CC2(CCN(c3ccc(C(=O)N4CC(C5CCN(c6ccc7c(c6)C(=O)N(C6CCC(=O)NC6=O)C7=O)CC5)C4)nn3)CC2)CN1c1ccc(C#N)c(Cl)c1] |
| A185 | 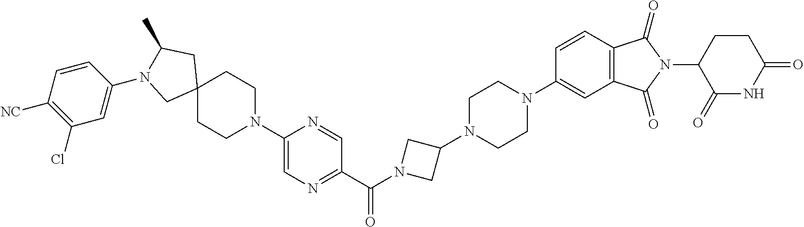 [SMILES: C[C@H]1CC2(CCN(c3cnc(C(=O)N4CC(N5CCN(c6ccc7c(c6)C(=O)N(C6CCC(=O)NC6=O)C7=O)CC5)C4)cn3)CC2)CN1c1ccc(C#N)c(Cl)c1] |
| A186 | 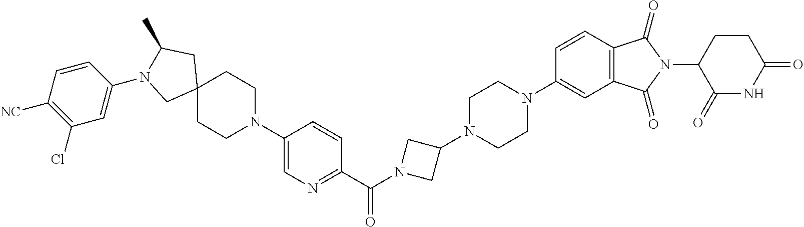 [SMILES: C[C@H]1CC2(CCN(c3ccc(C(=O)N4CC(N5CCN(c6ccc7c(c6)C(=O)N(C6CCC(=O)NC6=O)C7=O)CC5)C4)nc3)CC2)CN1c1ccc(C#N)c(Cl)c1] |
| A187 | 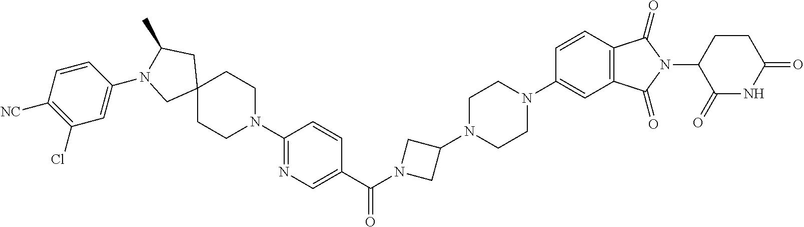 [SMILES: C[C@H]1CC2(CCN(c3ccc(C(=O)N4CC(N5CCN(c6ccc7c(c6)C(=O)N(C6CCC(=O)NC6=O)C7=O)CC5)C4)cn3)CC2)CN1c1ccc(C#N)c(Cl)c1] |
| A188 | 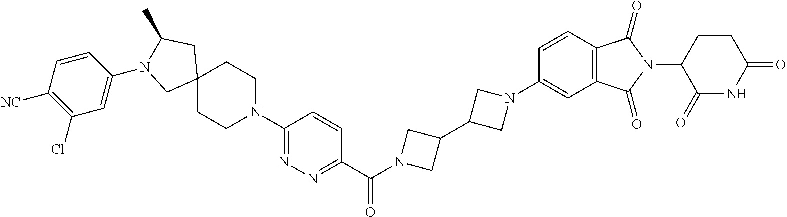 [SMILES: C[C@H]1CC2(CCN(c3ccc(C(=O)N4CC(C5CN(c6ccc7c(c6)C(=O)N(C6CCC(=O)NC6=O)C7=O)C5)C4)nn3)CC2)CN1c1ccc(C#N)c(Cl)c1] |
| A189 | 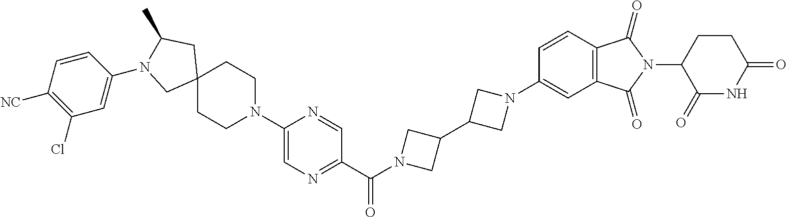 [SMILES: C[C@H]1CC2(CCN(c3cnc(C(=O)N4CC(C5CN(c6ccc7c(c6)C(=O)N(C6CCC(=O)NC6=O)C7=O)C5)C4)cn3)CC2)CN1c1ccc(C#N)c(Cl)c1] |
| A190 | 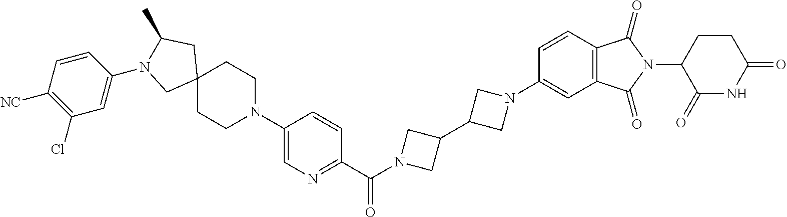 [SMILES: C[C@H]1CC2(CCN(c3ccc(C(=O)N4CC(C5CN(c6ccc7c(c6)C(=O)N(C6CCC(=O)NC6=O)C7=O)C5)C4)nc3)CC2)CN1c1ccc(C#N)c(Cl)c1] |
| A191 | 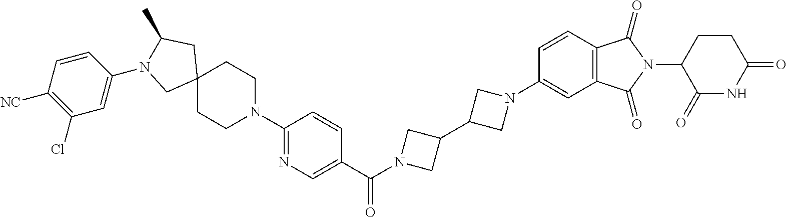 [SMILES: C[C@H]1CC2(CCN(c3ccc(C(=O)N4CC(C5CN(c6ccc7c(c6)C(=O)N(C6CCC(=O)NC6=O)C7=O)C5)C4)cn3)CC2)CN1c1ccc(C#N)c(Cl)c1] |
| A192 | 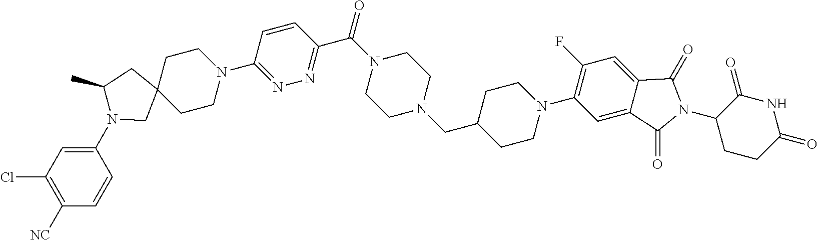 [SMILES: C[C@H]1CC2(CCN(c3ccc(C(=O)N4CCN(CC5CCN(c6cc7c(cc6F)C(=O)N(C6CCC(=O)NC6=O)C7=O)CC5)CC4)nn3)CC2)CN1c1ccc(C#N)c(Cl)c1] |

In some embodiments, the disclosure provides a pharmaceutical composition comprising a Compound of the Disclosure and a pharmaceutically acceptable carrier or excipient.

Compounds of the Disclosure contain an asymmetric carbon atom. In some embodiments, Compounds of the Disclosure are racemic compounds. In other embodiments, Compounds of the Disclosure are enantiomerically enriched, e.g., the enantiomeric excess or “ee” of the compound is about 5% or more as measured by chiral HPLC. In some embodiments, the ee is about 10%. In some embodiments, the ee is about 20%. In some embodiments, the ee is about 30%. In some embodiments, the ee is about 40%. In some embodiments, the ee is about 50%. In some embodiments, the ee is about 60%. In some embodiments, the ee is about 70%. In some embodiments, the ee is about 80%. In some embodiments, the ee is about 85%. In some embodiments, the ee is about 90%. In some embodiments, the ee is about 91%. In some embodiments, the ee is about 92%. In some embodiments, the ee is about 93%. In some embodiments, the ee is about 94%. In some embodiments, the ee is about 95%. In some embodiments, the ee is about 96%. In some embodiments, the ee is about 97%. In some embodiments, the ee is about 98%. In some embodiments, the ee is about 99%.

In some embodiments, the cereblon binding portion of a Compound of the Disclosure, e.g., B¹ is B¹-1, B¹-2, B¹-3, B¹-4, or B¹-5, is enantiomerically enriched. In some embodiments, the cereblon binding portion of the molecule is racemic. The present disclosure encompasses all possible stereoisomeric, e.g., diastereomeric, forms of Compounds of the Disclosure. For example, all possible stereoisomers of Compounds of the Disclosure are encompassed when E portion of Formula I is entantiomerically enriched and the cereblon binding portion of the molecule is racemic. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure, including pharmaceutically acceptable salts. As used herein, the “pharmaceutically acceptable salt” refers to non-toxic salt forms of Compounds of the Disclosure. See e.g., Gupta et al., Molecules 23:1719 (2018). Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include the actual compound as well as pharmaceutically acceptable salts, hydrates, or solvates thereof.

The present disclosure also encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### METHODS OF USE

In some aspects, the present disclosure provides methods of degrading an AR protein in a subject, comprising administering to the subject a Compound of the Disclosure.

In some aspects, the present disclosure provides uses of a Compound of the Disclosure in the manufacture of a medicament for degrading an AR protein in a subject.

In some aspects, the present disclosure provides Compounds of the Disclosure for use in degrading an AR protein in a subject.

In some aspects, the present disclosure provides methods of treating or preventing a disease (e.g., a disease associated with degradation of an AR protein) in a subject in need thereof, comprising administering to the subject a Compound of the Disclosure (e.g., in a therapeutically effective amount).

In some aspects, the present disclosure provides methods of treating a disease (e.g., a disease associated with degradation of an AR protein) in a subject in need thereof, comprising administering to the subject a Compound of the Disclosure (e.g., in a therapeutically effective amount).

In some aspects, the present disclosure provides uses of a Compound of the Disclosure in the manufacture of a medicament for treating or preventing a disease (e.g., a disease associated with degradation of an AR protein) in a subject in need thereof.

In some aspects, the present disclosure provides uses of a Compound of the Disclosure in the manufacture of a medicament for treating a disease (e.g., a disease associated with degradation of an AR protein) in a subject in need thereof.

In some aspects, the present disclosure provides Compounds of the Disclosure for use in treating or preventing a disease (e.g., a disease associated with degradation of an AR protein) in a subject in need thereof.

In some aspects, the present disclosure provides Compounds of the Disclosure for use in treating a disease (e.g., a disease associated with degradation of an AR protein) in a subject in need thereof.

In some embodiments, the subject is a mammal.

In some embodiments, the subject is a human.

In some embodiments, the subject is a biological sample (e.g., a cell population).

In some embodiments, the disease is a cancer.

Compounds of the Disclosure degrade AR protein and are thus useful in the treatment of a variety of diseases and conditions. In particular, Compounds of the Disclosure are useful in methods of treating a disease or condition wherein degradation AR proteins provides a benefit, for example, cancers and proliferative diseases. The therapeutic methods of the disclosure comprise administering a therapeutically effective amount of a Compound of the Disclosure to a subject, e.g., a cancer patient, in need thereof. The present methods also encompass administering a second therapeutic agent to the subject in combination with the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as AR protein degraders for the treatment of a variety of diseases and conditions wherein degradation of AR proteins has a beneficial effect. Compounds of the Disclosure typically have DC₅₀ (the drug concentration that results in 50% AR protein degradation) values of less than 100 μM, e.g., less than 50 μM, less than 25 μM, and less than 5 μM, less than about 1 μM, less than about 0.5 μM, or less than about 0.1 μM. In some embodiments, Compounds of the Disclosure typically have DC₅₀ values of less than about 0.01 μM. In some embodiments, Compounds of the Disclosure typically have DC₅₀ values of less than about 0.001 μM. In some embodiments, the present disclosure relates to a method of treating an individual suffering from a disease or condition wherein degradation of AR proteins provides a benefit comprising administering a therapeutically effective amount of a Compound of the Disclosure to an individual in need thereof.

Since Compounds of the Disclosure are degraders of AR protein, a number of diseases and conditions mediated by AR can be treated by employing these compounds. The present disclosure is thus directed generally to a method for treating a condition or disorder responsive to degradation of AR in an animal, e.g., a human, suffering from, or at risk of suffering from, the condition or disorder, the method comprising administering to the animal an effective amount of one or more Compounds of the Disclosure.

The present disclosure is further directed to a method of degrading AR protein in a subject in need thereof, said method comprising administering to the subject an effective amount of at least one Compound of the Disclosure.

In some aspects, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading AR. Examples of treatable cancers include, but are not limited to, any one or more of the cancers of Table I.

TABLE I

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In some embodiments, the cancer is a solid tumor. In some embodiments, the cancer a hematological cancer. Exemplary hematological cancers include, but are not limited to, the cancers listed in Table II. In some embodiments, the hematological cancer is acute lymphocytic leukemia, chronic lymphocytic leukemia (including B-cell chronic lymphocytic leukemia), or acute myeloid leukemia.

TABLE II

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In some embodiments, the cancer is a leukemia, for example a leukemia selected from acute monocytic leukemia, acute myelogenous leukemia, chronic myelogenous leukemia, chronic lymphocytic leukemia and mixed lineage leukemia (MLL). In some embodiments the cancer is NUT-midline carcinoma. In some embodiments the cancer is multiple myeloma. In some embodiments the cancer is a lung cancer such as small cell lung cancer (SCLC). In some embodiments the cancer is a neuroblastoma. In some embodiments the cancer is Burkitt's lymphoma. In some embodiments the cancer is cervical cancer. In some embodiments the cancer is esophageal cancer. In some embodiments the cancer is ovarian cancer. In some embodiments the cancer is colorectal cancer. In some embodiments, the cancer is prostate cancer. In some embodiments, the cancer is breast cancer.

In some embodiments, the cancer is selected from acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In some embodiments, Compounds of the Disclosure are administered to a subject in need thereof to treat breast cancer, ovarian cancer, or prostate cancer. In some embodiments, the cancer is breast cancer. In some embodiments, the cancer is ovarian cancer. In some embodiments, the cancer is prostate cancer. In some embodiments, the cancer is metastatic castration-resistant prostate cancer.

In some embodiments, Compounds of the Disclosure are administered to a subject in need thereof to treat a sebum-related diseases, e.g., seborrhea, acne, hyperplasia, and sebaceous adenoma.

In some embodiments, Compounds of the Disclosure are administered to a subject in need thereof as transgender therapy, e.g., to lower serum testosterone levels.

In some embodiments, Compounds of the Disclosure are administered to a subject in need thereof to treat hirsutism.

In some embodiments, Compounds of the Disclosure are administered to a subject in need thereof to treat hair loss (alopecia).

In some embodiments, Compounds of the Disclosure are administered to a subject in need thereof to treat hidradenitis suppurativa.

The methods of the present disclosure can be accomplished by administering a Compound of the Disclosure as the neat compound or as a pharmaceutical composition. Administration of a pharmaceutical composition, or neat Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered.

In some embodiments, a Compound of the Disclosure is administered as a single agent to treat a disease or condition wherein degradation of AR protein provides a benefit. In some embodiments, a Compound of the Disclosure is administered in conjunction with a second therapeutic agent useful in the treatment of a disease or condition wherein degradation of AR protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered as a single pharmaceutical composition or two separate pharmaceutical compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more doses of the second therapeutic agent can be administered. The Compound of the Disclosure therefore can be used in conjunction with one or more second therapeutic agents, for example, but not limited to, anticancer agents.

In methods of the present disclosure, a therapeutically effective amount of a Compound of the Disclosure, typically formulated in accordance with pharmaceutical practice, is administered to a subject, e.g., a human cancer patient, in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the AR protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In some embodiments, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DM1, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, Cl-1033, EKB-569, GW-2016, antibodies EL1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

In some embodiments, the second therapeutically active agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in some embodiments, a Compound of the Disclosure is administered in combination with an immune checkpoint inhibitor is selected from a PD-1 inhibitor, a PD-L1 inhibitor, a CTLA-4 inhibitor, a LAG3 inhibitor, a TIM3 inhibitor, and a cd47 inhibitor.

In some embodiments, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In some embodiments, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In some embodiments, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In some embodiments, the immune checkpoint inhibitor is a LAG3 inhibitor. LAG3, Lymphocyte Activation Gene 3, is a negative co-stimulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In some embodiments, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In some embodiments, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In some embodiments, “antibody” is meant to include soluble receptors that do not possess the Fc portion of the antibody. In some embodiments, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In some embodiments, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject.

Embodiment I. A method of treating a subject, the method comprising administering to the subject a therapeutically effective amount of a Compound of the Disclosure, wherein the subject has cancer, a chronic autoimmune disorder, an inflammatory condition, a proliferative disorder, sepsis, or a viral infection.

Embodiment II. The method Embodiment I, wherein the subject has cancer, e.g., any one of more of the cancers of Table I or Table II.

Embodiment III. The method of Embodiment II, wherein the cancer is prostate cancer or breast cancer.

Embodiment IV. The method of Embodiment II, wherein the cancer is breast cancer.

Embodiment V. The method of Embodiment II, wherein the cancer is prostate cancer, e.g., metastatic castration-resistant prostate cancer.

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. A pharmaceutical composition comprising a Compound of the Disclosure and a pharmaceutically acceptable excipient for use in treating cancer, a chronic autoimmune disorder, an inflammatory condition, a proliferative disorder, sepsis, or a viral infection.

Embodiment VIII. The pharmaceutical composition of Embodiment VII for use in treating cancer.

Embodiment IX. The pharmaceutical composition of Embodiment VIII, wherein the cancer is prostate cancer or breast cancer.

Embodiment X. The pharmaceutical composition of Embodiment VIII, wherein the cancer is breast cancer.

Embodiment XI. The pharmaceutical composition of Embodiment VIII, wherein the cancer is prostate cancer, e.g., metastatic castration-resistant prostate cancer.

Embodiment XII. A Compound of the Disclosure for use in treatment of cancer, a chronic autoimmune disorder, an inflammatory condition, a proliferative disorder, sepsis, or a viral infection.

Embodiment XIII. The compound of Embodiment XIII for use in treating cancer.

Embodiment XIV. The compound of Embodiment XIII, wherein the cancer is breast cancer.

Embodiment XV. The compound of Embodiment XIII, wherein the cancer is prostate cancer, e.g., metastatic castration-resistant prostate cancer.

Embodiment XVI. Use of a Compound of the Disclosure for the manufacture of a medicament for treatment of cancer, a chronic autoimmune disorder, an inflammatory condition, a proliferative disorder, sepsis, or a viral infection.

Embodiment XVII. The use of Embodiment XVI for the treatment of cancer.

Embodiment XVIII. The use of Embodiment XVII, wherein the cancer is prostate cancer or breast cancer.

Embodiment XIV. The use of Embodiment XVII, wherein the cancer is breast cancer.

Embodiment XX. The use of Embodiment XVII, wherein the cancer is prostate cancer, e.g., metastatic castration-resistant prostate cancer.

Embodiment XXI. A method of reducing AR protein within a cell of a subject in need thereof, the method comprising administering to the patient a Compound of the Disclosure. In some embodiments, the AR protein is reduced by about 50% or less, e.g., 1%, about 2%, about 3%, about 4%, about 5%, about 10%, about 15%, about 20%, about 25%, about 30%, about 35%, about 40%, or about 45%. In some embodiments, the AR protein is reduced by about 51% or more, e.g., about 55%, about 60%, about 65%, about 70%, about 75%, about 80%, about 85%, about 90%, or about 95%.

Embodiment XXII. A method of treating a subject, the method comprising administering to the subject a therapeutically effective amount of a Compound of the Disclosure, wherein the subject has seborrhea, acne, hyperplasia, sebaceous adenoma, hirsutism, alopecia, or hidradenitis suppurativa, or the subject is in need of transgender therapy, e.g., to lower serum testosterone levels.

Embodiment XXIII. A pharmaceutical composition comprising a Compound of the Disclosure and a pharmaceutically acceptable excipient for use in treating seborrhea, acne, hyperplasia, sebaceous adenoma, hirsutism, alopecia, or hidradenitis suppurativa, or for use in transgender therapy, e.g., to lower serum testosterone levels.

Embodiment XXIV. A Compound of the Disclosure for use in treatment of seborrhea, acne, hyperplasia, sebaceous adenoma, hirsutism, alopecia, or hidradenitis suppurativa, or for transgender therapy, e.g., to lower serum testosterone levels.

Embodiment XXV. Use of a Compound of the Disclosure for the manufacture of a medicament for treatment of seborrhea, acne, hyperplasia, sebaceous adenoma, hirsutism, alopecia, or hidradenitis suppurativa, or for transgender therapy, e.g., to lower serum testosterone levels.

### Methods of Synthesis

In some aspects, the present disclosure provides methods of preparing a Compound of Disclosure.

In some aspects, the present disclosure provides compounds obtainable by, or obtained by, or directly obtained by a method for preparing a Compound of Disclosure.

Exemplary non-limiting methods of making Compounds of the Disclosure are provided in Synthetic Schemes as described in Examples 1-14.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assay involves evaluation of AR degradation activity and cell growth inhibition, e.g., in LNCaP and VCaP cells.

In some embodiments, the LNCaP cells are grown in RPMI 1640 (Invitrogen).

In some embodiments, the VCaP cells are grown in DMEM with Glutamax (Invitrogen).

### Pharmaceutical Compositions

In some aspects, the present disclosure provides pharmaceutical compositions comprising a Compound of Disclosure, or a pharmaceutically acceptable salt, solvate, or stereoisomer thereof, and a pharmaceutically acceptable excipient.

In some embodiments, the pharmaceutically suitable or acceptable carrier is selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. By way of example only, the dose of the compound described herein for methods of treating a disease as described herein is about 0.001 to about 1 mg/kg body weight of the subject per day.

### Kits

In some embodiments, the present disclosure provides kits which comprise a Compound of the Disclosure (or a composition comprising a Compound of the Disclosure) packaged in a manner that facilitates its use to practice methods of the present disclosure. In some embodiments, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In some embodiments, the compound or composition is packaged in a unit dosage form. The kit further can include a device suitable for administering the composition according to the intended route of administration.

### EXEMPLARY EMBODIMENTS

Embodiment 1. A compound of Formula I or a pharmaceutically acceptable salt or solvate thereof, wherein:
A¹ is selected from phenylenyl, 5-membered heteroarylenyl, and 6-membered heteroarylenyl;
X² is selected from —C(═O)—, —S(═O)₂—, —O—, and —CR^{4c}R^{4d}—; or X² is absent;
   R^{4c} and R^{4d} are independently selected from hydrogen and C₁-C₄ alkyl;
   L is -J¹-J²-J³-J⁴-J⁵-; wherein J¹ is attached to X²;
   J¹ is selected from cycloalkylenyl and heterocyclenyl; or J¹ is absent;
   J² is selected from —(CH₂)_{b}—, —C(═O)—, —CH═CH—, and —C≡C—;
   b is 0, 1, 2, or 3;
   J³ is selected from alkylenyl, heteroalkylenyl, cycloalkylenyl, heterocyclenyl, phenylenyl, and heteroarylenyl; or
   J³ is absent;
   J⁴ is selected from alkylenyl, cycloalkylenyl, and heterocyclenyl; or J⁴ is absent;
   J⁵ is selected from —(CH₂)_{c}—, —O—, —N(R⁵)—, and —C(═O)—;
   c is 0, 1, 2, or 3;
   R⁵ is selected from hydrogen and C₁-C₄ alkyl;
B¹ is selected from B¹-1, B¹-2, B¹-3, B¹-4, B¹-5, and B¹-6;
   Q¹ is selected from —CR^{2a}═ and —N═;
   Q² is selected from —CR^{2b}═ and —N═;
   Q is selected from —CR^{2c}═ and —N═;
   R^{2a}, R^{2b}, R^{2c}, R^{2d}, and R^{2e} are independently selected from hydrogen, halo, amino, C₁-C₃ alkyl, and C₁-C₃ alkoxy;
   R³ is selected from hydrogen, deuterium, fluoro, and C₁-C₄ alkyl;
   m is 1, 2, or 3;
   n is 1, 2, or 3;
   Z and Z¹ are —C(═O)—; or
   Z is —C(═O)— and Z¹ is —CR^{6a}R^{6b}—; or
   Z is —CR^{6a}R^{6b}— and Z¹ is —C(═O)—;
   Z³ is selected from —CR^{6c}R^{6d}— and —C(═O)—;
   R^{6a} and R^{6b} are independently selected from hydrogen and C₁-C₃ alkyl; or
   R^{6c} and R^{6d} taken together with the carbon to which they are attached from a C₃-C₆ cycloalkyl;
   R⁸ is selected from hydrogen and C₁-C₃ alkyl; and
   R¹⁰ is selected from hydrogen and C₁-C₄ alkyl, with the proviso the compound of Formula I is not a compound described in Table 1A.

Embodiment 2. The compound of Embodiment 1 of Formula II or a pharmaceutically acceptable salt or solvate thereof, wherein:
G¹ is selected from —CR^{5a}═ and —N═;
G² is selected from —CR^{5b}═ and —N═;
G³ is selected from —CR^{5c}═ and —N═;
G⁴ is selected from —CR^{5d}═ and —N═; and
R^{5a}, R^{5b}, R^{5c}, and R^{5d} are independently selected from hydrogen, halo, C₁-C₃ alkyl, and C₁-C₃ alkoxy.

Embodiment 3. The compound of Embodiment 2, or a pharmaceutically acceptable salt or solvate thereof, wherein G¹ is —CR^{5a}═.

Embodiment 4. The compound of Embodiment 3, or a pharmaceutically acceptable salt or solvate thereof, wherein R^{5a} is hydrogen.

Embodiment 5. The compound of Embodiment 2, or a pharmaceutically acceptable salt or solvate thereof, wherein G¹ is —N═.

Embodiment 6. The compound of any one of Embodiments 2-5, or a pharmaceutically acceptable salt or solvate thereof, wherein G² is —CR^{5b}═.

Embodiment 7. The compound of Embodiment 6, or a pharmaceutically acceptable salt or solvate thereof, wherein R^{5b} is hydrogen.

Embodiment 8. The compound of any one of Embodiments 2-5, or a pharmaceutically acceptable salt or solvate thereof, wherein G² is —N═.

Embodiment 9. The compound of any one of Embodiments 2-8, or a pharmaceutically acceptable salt or solvate thereof, wherein G³ is —CR^{5c}═.

Embodiment 10. The compound of Embodiment 9, or a pharmaceutically acceptable salt or solvate thereof, wherein R^{5c} is hydrogen.

Embodiment 11. The compound of any one of Embodiments 2-8, or a pharmaceutically acceptable salt or solvate thereof, wherein G³ is —N═.

Embodiment 12. The compound of any one of Embodiments 2-11, or a pharmaceutically acceptable salt or solvate thereof, wherein G⁴ is —CR^{5d}═.

Embodiment 13. The compound of Embodiment 12, or a pharmaceutically acceptable salt or solvate thereof, wherein R^{5d} is hydrogen.

Embodiment 14. The compound of any one of Embodiments 2-11, or a pharmaceutically acceptable salt or solvate thereof, wherein G⁴ is —N═.

Embodiment 15. The compound of Embodiment 1 of Formula III or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

Embodiment 16. The compound of Embodiment 1 of Formula IV or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

Embodiment 17. The compound of Embodiment 1 of Formula V or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

Embodiment 18. The compound of any one of Embodiments 15-17, or a pharmaceutically acceptable salt or solvate thereof, wherein G⁵ is —S—.

Embodiment 19. The compound of any one of Embodiments 15-17, or a pharmaceutically acceptable salt or solvate thereof, wherein G⁵ is —O—.

Embodiment 20. The compound of any one of Embodiments 15-17, or a pharmaceutically acceptable salt or solvate thereof, wherein G⁵ is —NR⁹—.

Embodiment 21. The compound of any one of Embodiments 15-20, or a pharmaceutically acceptable salt or solvate thereof, wherein G is —CH═.

Embodiment 22. The compound of any one of Embodiments 15-20, or a pharmaceutically acceptable salt or solvate thereof, wherein G is —N═.

Embodiment 23. The compound of any one of Embodiments 15-22, or a pharmaceutically acceptable salt or solvate thereof, wherein G⁶ is —CH═.

Embodiment 24. The compound of any one of Embodiments 15-22, or a pharmaceutically acceptable salt or solvate thereof, wherein G⁶ is —N═.

Embodiment 25. The compound of Embodiment 1 of Formula VI or a pharmaceutically acceptable salt or solvate thereof.

Embodiment 26. The compound of any one of Embodiments 1-25, or a pharmaceutically acceptable salt or solvate thereof, wherein X² is —C(═O)—.

Embodiment 27. The compound of any one of Embodiments 1-25, or a pharmaceutically acceptable salt or solvate thereof, wherein X² is —S(═O)₂—.

Embodiment 28. The compound of any one of Embodiments 1-25, or a pharmaceutically acceptable salt or solvate thereof, wherein X² is —O—.

Embodiment 29. The compound of any one of Embodiments 1-25, or a pharmaceutically acceptable salt or solvate thereof, wherein X² is —CR^{4c}R^{4d}—.

Embodiment 30. The compound of Embodiment 29, or a pharmaceutically acceptable salt or solvate thereof, wherein R^{4c} and R^{4d} are hydrogen.

Embodiment 31. The compound of any one of Embodiments 1-30, or a pharmaceutically acceptable salt or solvate thereof, wherein X² is absent.

Embodiment 32. The compound of any one of Embodiments 1-31, or a pharmaceutically acceptable salt or solvate thereof, wherein J¹ is cycloalkylenyl.

Embodiment 33. The compound of any one of Embodiments 1-31, or a pharmaceutically acceptable salt or solvate thereof, wherein J¹ is heterocyclenyl.

Embodiment 34. The compound of Embodiment 33, or a pharmaceutically acceptable salt or solvate thereof, wherein J¹ is selected from J¹-1, J¹-2, J¹-3, J¹-4, J¹-5, J¹-6, J¹-7, J¹-8, J¹-9, J¹-10, J¹-11, J¹-12, and J¹-13.

Embodiment 35. The compound of any one of Embodiments 1-31, or a pharmaceutically acceptable salt or solvate thereof, wherein J¹ is absent.

Embodiment 36. The compound of any one of Embodiments 1-35, or a pharmaceutically acceptable salt or solvate thereof, wherein J² is selected from —(CH₂)_{b}— and —C≡C—; and b is 0, 1, or 2.

Embodiment 37. The compound of Embodiment 36, or a pharmaceutically acceptable salt or solvate thereof, wherein J² is —(CH₂)_{b}—; and b is 0.

Embodiment 38. The compound of Embodiment 36, or a pharmaceutically acceptable salt or solvate thereof, wherein J² is —(CH₂)_{b}—; and b is 1.

Embodiment 39. The compound of Embodiment 36, or a pharmaceutically acceptable salt or solvate thereof, wherein J² is —C≡C—.

Embodiment 40. The compound of any one of Embodiments 1-39, or a pharmaceutically acceptable salt or solvate thereof, wherein J³ is selected from cycloalkylenyl and heterocyclenyl.

Embodiment 41. The compound of any one of Embodiments 1-39, or a pharmaceutically acceptable salt or solvate thereof, wherein J³ is absent.

Embodiment 42. The compound of any one of Embodiments 1-41, or a pharmaceutically acceptable salt or solvate thereof, wherein J⁴ is selected from alkylenyl, cycloalkylenyl, and heterocyclenyl.

Embodiment 43. The compound of any one of Embodiments 1-41, or a pharmaceutically acceptable salt or solvate thereof, wherein J⁴ is absent.

Embodiment 44. The compound of any one of Embodiments 1-43, or a pharmaceutically acceptable salt or solvate thereof, wherein:
J⁵ is selected from —O— and —N(H)—; and
B¹ is selected from B¹-1, B¹-2, and B¹-3.

Embodiment 45. The compound of any one of Embodiments 1-43, or a pharmaceutically acceptable salt or solvate thereof, wherein:
J⁵ is selected from —(CH₂)_{c}— and —O—;
c is 0;
J⁴ is selected from J⁴-1, J⁴-2, J⁴-3, J⁴-4, J⁴-5, and J⁴-6;
R⁷ is selected from hydrogen, halo, cyano, hydroxy, C₁-C₃ alkyl, and C₁-C₃ alkoxy; and
B¹ is selected from B¹-1, B¹-2, B¹-3, and B¹-4.

Embodiment 46. The compound of any one of Embodiments 1-45, or a pharmaceutically acceptable salt or solvate thereof, wherein B¹ is B¹-1.

Embodiment 47. The compound of Embodiment 46, or a pharmaceutically acceptable salt or solvate thereof, wherein B¹-1 is B¹-1-B.

Embodiment 48. The compound of Embodiment 46, or a pharmaceutically acceptable salt or solvate thereof, wherein B¹-1 is B¹-1-C.

Embodiment 49. The compound of any one of Embodiments 46-48, or a pharmaceutically acceptable salt or solvate thereof, wherein Z and Z¹ are —C(═O)—.

Embodiment 50. The compound of any one of Embodiments 46-48, or a pharmaceutically acceptable salt or solvate thereof, wherein Z is —C(═O)— and Z¹ is —CR^{6a}R^{6b}—.

Embodiment 51. The compound of any one of Embodiments 46-48, or a pharmaceutically acceptable salt or solvate thereof, wherein Z is —CR^{6a}R^{6b}— and Z¹ is —C(═O)—.

Embodiment 52. The compound of any one of Embodiments 1-45, or a pharmaceutically acceptable salt or solvate thereof, wherein B¹ is B¹-2.

Embodiment 53. The compound of Embodiment 52, or a pharmaceutically acceptable salt or solvate thereof, wherein B¹-2 is B¹-2-B.

Embodiment 54. The compound of Embodiment 52, or a pharmaceutically acceptable salt or solvate thereof, wherein B¹-2 is B¹-2-C.

Embodiment 55. The compound of any one of Embodiments 1-45, or a pharmaceutically acceptable salt or solvate thereof, wherein B¹ is B¹-3.

Embodiment 56. The compound of Embodiment 55, or a pharmaceutically acceptable salt or solvate thereof, wherein B¹-3 is B¹-3-B.

Embodiment 57. The compound of Embodiment 55, or a pharmaceutically acceptable salt or solvate thereof, wherein B¹-3 is B¹-3-C.

Embodiment 58. The compound of any one of Embodiments 1-45, or a pharmaceutically acceptable salt or solvate thereof, wherein B¹ is B¹-4.

Embodiment 59. The compound of Embodiment 58, or a pharmaceutically acceptable salt or solvate thereof, wherein B¹-4 is B¹-4-B.

Embodiment 60. The compound of Embodiment 58, or a pharmaceutically acceptable salt or solvate thereof, wherein B¹-4 is B¹-4-C.

Embodiment 61. The compound of any one of Embodiments 52-60, or a pharmaceutically acceptable salt or solvate thereof, wherein R¹⁰ is C₁-C₃ alkyl.

Embodiment 62. The compound of any one of Embodiments 46-61, or a pharmaceutically acceptable salt or solvate thereof, wherein Q¹ is —N═.

Embodiment 63. The compound of any one of Embodiments 46-61, or a pharmaceutically acceptable salt or solvate thereof, wherein Q¹ is —CR^{2a}.

Embodiment 64. The compound of Embodiment 63, or a pharmaceutically acceptable salt or solvate thereof, wherein R^{2a} is selected from hydrogen and halo.

Embodiment 65. The compound of any one of Embodiments 46-64, or a pharmaceutically acceptable salt or solvate thereof, wherein Q² is —N═.

Embodiment 66. The compound of any one of Embodiments 46-64, or a pharmaceutically acceptable salt or solvate thereof, wherein Q² is —CR^{2b}.

Embodiment 67. The compound of Embodiment 66, or a pharmaceutically acceptable salt or solvate thereof, wherein R^{2b} is selected from hydrogen and halo.

Embodiment 68. The compound of any one of Embodiments 46-67, or a pharmaceutically acceptable salt or solvate thereof, wherein Q is —N═.

Embodiment 69. The compound of any one of Embodiments 46-67, or a pharmaceutically acceptable salt or solvate thereof, wherein Q is —CR^{2c}.

Embodiment 70. The compound of Embodiment 69, or a pharmaceutically acceptable salt or solvate thereof, wherein R^{2c} is selected from hydrogen and halo.

Embodiment 71. The compound of any one of Embodiments 1-43, wherein:
J¹ is selected from —(CH₂)_{c}— and —C(═O)—;
c is 0, 1, 2, or 3; and
B¹ is selected from B¹-5 and B¹-6, or a pharmaceutically acceptable salt or solvate thereof.

Embodiment 72. The compound of any one of Embodiments 1-43 or 71, or a pharmaceutically acceptable salt or solvate thereof, wherein B¹ is B¹-5.

Embodiment 73. The compound of Embodiment 72, or a pharmaceutically acceptable salt or solvate thereof, wherein B¹-5 is B¹-5-B.

Embodiment 74. The compound of Embodiment 72, or a pharmaceutically acceptable salt or solvate thereof, wherein B¹-5 is B¹-5-C.

Embodiment 75. The compound of any one of Embodiments 72-74, or a pharmaceutically acceptable salt or solvate thereof, wherein m is 1.

Embodiment 76. The compound of any one of Embodiments 72-74, or a pharmaceutically acceptable salt or solvate thereof, wherein m is 2.

Embodiment 77. The compound of any one of Embodiments 72-76, or a pharmaceutically acceptable salt or solvate thereof, wherein n is 1.

Embodiment 78. The compound of any one of Embodiments 72-76, or a pharmaceutically acceptable salt or solvate thereof, wherein n is 2.

Embodiment 79. The compound of any one of Embodiments 72-78, or a pharmaceutically acceptable salt or solvate thereof, wherein R^{2d} and R^{2d} are independently selected from hydrogen and halo.

Embodiment 80. The compound of any one of Embodiments 72-79, or a pharmaceutically acceptable salt or solvate thereof, wherein Z³ is —C(═O)—.

Embodiment 81. The compound of any one of Embodiments 72-79, or a pharmaceutically acceptable salt or solvate thereof, wherein Z³ is —CR^{6a}R^{6b}—.

Embodiment 82. The compound of any one of Embodiments 1-43 or 71, or a pharmaceutically acceptable salt or solvate thereof, wherein B¹ is B¹-6.

Embodiment 83. The compound of Embodiment 82, or a pharmaceutically acceptable salt or solvate thereof, wherein B¹-6 is B¹-6-B.

Embodiment 84. The compound of Embodiment 82, or a pharmaceutically acceptable salt or solvate thereof, wherein B¹-6 is B¹-6-C.

Embodiment 85. The compound of any one of Embodiments 82-84, or a pharmaceutically acceptable salt or solvate thereof, wherein Z and Z¹ are —C(═O)—.

Embodiment 86. The compound of any one of Embodiments 82-84, or a pharmaceutically acceptable salt or solvate thereof, wherein Z is —C(═O)— and Z¹ is —CR^{6a}R^{6b}—.

Embodiment 87. The compound of any one of Embodiments 82-84, or a pharmaceutically acceptable salt or solvate thereof, wherein Z is —CR^{6a}R^{6b}— and Z¹ is —C(═O)—.

Embodiment 88. The compound of any one of Embodiments 82-87, or a pharmaceutically acceptable salt or solvate thereof, wherein R^{2d} and R^{2d} are independently selected from hydrogen and halo.

Embodiment 89. The compound of any one of Embodiments 1-88, or a pharmaceutically acceptable salt or solvate thereof, wherein R³ is hydrogen.

Embodiment 90. The compound of any one of Embodiments 1-89, or a pharmaceutically acceptable salt or solvate thereof, wherein R⁸ is hydrogen.

Embodiment 91. The compound of any one of Embodiments 1-43, or a pharmaceutically acceptable salt or solvate thereof, wherein B¹ is selected from:

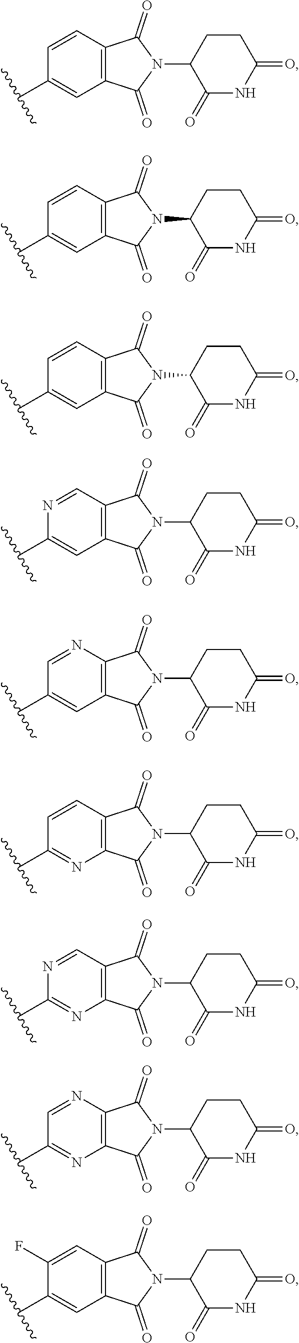

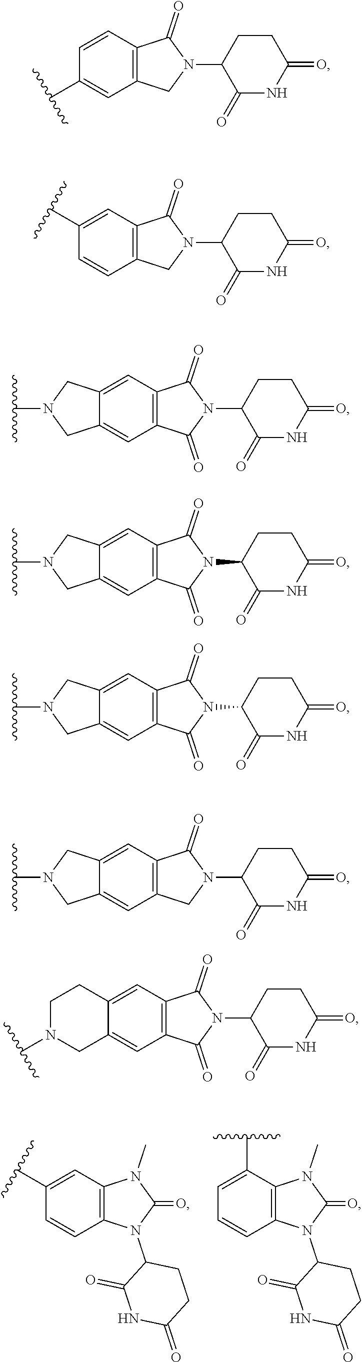

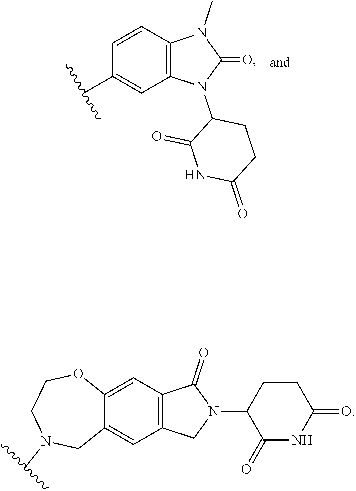 [SMILES: *c1ccc2c(c1)n(C1CCC(=O)NC1=O)c(=O)n2C ; *N1CCOc2cc3c(cc2C1)CN(C1CCC(=O)NC1=O)C3=O]

Embodiment 92. The compound of Embodiment 91, or a pharmaceutically acceptable salt or solvate thereof, wherein B¹ is:

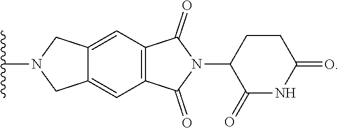 [SMILES: *N1Cc2cc3c(cc2C1)C(=O)N(C1CCC(=O)NC1=O)C3=O]

Embodiment 93. The compound of Embodiment 91, or a pharmaceutically acceptable salt or solvate thereof wherein B¹ is:

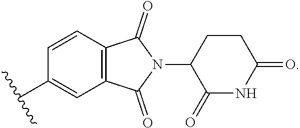 [SMILES: *c1ccc2c(c1)C(=O)N(C1CCC(=O)NC1=O)C2=O]

Embodiment 94. The compound of Embodiment 1 selected from any one or more of the compounds of Table 1, or a pharmaceutically acceptable salt or solvate thereof.

Embodiment 95. A pharmaceutical composition comprising the compound of any one of Embodiments 1-94, or a pharmaceutically acceptable salt or solvate thereof, and a pharmaceutically acceptable excipient.

Embodiment 96. A method of treating cancer, seborrhea, acne, hyperplasia, sebaceous adenoma, hirsutism, alopecia, or hidradenitis suppurativa, in a subject in need thereof, the method comprising administering to the subject a therapeutically effective amount of the compound of any one of Embodiments 1-94, or a pharmaceutically acceptable salt or solvate thereof.

Embodiment 97. The method of Embodiment 96, wherein the cancer is breast cancer, ovarian cancer, or prostate cancer.

Embodiment 98. The pharmaceutical composition of Embodiment 95 for use in treating cancer seborrhea, acne, hyperplasia, sebaceous adenoma, hirsutism, alopecia, or hidradenitis suppurativa.

Embodiment 99. The pharmaceutical composition of Embodiment 98, wherein the cancer is breast cancer, ovarian cancer, or prostate cancer.

Embodiment 100. A compound of any one of Embodiments 1-94, or a pharmaceutically acceptable salt or solvate thereof, for use in treating of cancer, seborrhea, acne, hyperplasia, sebaceous adenoma, hirsutism, alopecia, or hidradenitis suppurativa.

Embodiment 101. The compound for use of Embodiment 100, wherein the cancer is breast cancer, ovarian cancer, or prostate cancer.

Embodiment 102. Use of a compound of any one of Embodiments 1-94, or a pharmaceutically acceptable salt or solvate thereof, for the manufacture of a medicament for treatment of cancer, seborrhea, acne, hyperplasia, sebaceous adenoma, hirsutism, alopecia, or hidradenitis suppurativa.

Embodiment 103. The use of Embodiment 102, wherein the cancer is breast cancer, ovarian cancer, or prostate cancer.

Embodiment 104. A method of treating a subject, the method comprising administering to the subject a therapeutically effective amount of a compound of any one of Embodiments 1-94, wherein the subject is in need of transgender therapy.

Embodiment 105. A method of reducing androgen receptor protein within a cell of a patient in need thereof, the method comprising administering to the subject a compound of any one of Embodiments 1-94, or a pharmaceutically acceptable salt or solvate thereof.

Embodiment 106. A kit comprising the compound of any one of Embodiments 1-94, or a pharmaceutically acceptable salt or solvate thereof, and instructions for administering the compound, or a pharmaceutically acceptable salt or solvate thereof, to a subject having cancer, seborrhea, acne, hyperplasia, sebaceous adenoma, hirsutism, alopecia, or hidradenitis suppurativa.

### Definitions

The term “a disease or condition wherein degradation of androgen receptor (AR) provides a benefit” and the like pertains to a disease or condition in which the androgen receptor is important or necessary, e.g., for the onset, progress, expression of that disease or condition, or a disease or a condition which is known to be treated by an AR degrader. Examples of such conditions include, but are not limited to, a cancer. One of ordinary skill in the art is readily able to determine whether a compound treats a disease or condition mediated by an AR degrader for any particular cell type, for example, by assays which conveniently can be used to assess the activity of particular compounds.

The term “androgen receptor degrader,” “AR degrader,” and the like refer to a heterobifunctional small molecule that degrades AR protein. AR degraders contain a first ligand which binds to AR protein, a second ligand for an E3 ligase system, and a chemical linker that tethers the first and second ligands. Representative Compounds of the Disclosure that degrade AR protein are disclosed in Table 1.

The term “second therapeutic agent” refers to a therapeutic agent different from a Compound of the Disclosure and that is known to treat the disease or condition of interest. For example, when a cancer is the disease or condition of interest, the second therapeutic agent can be a known chemotherapeutic drug, like taxol, or radiation, for example. In some embodiments, a Compound of the Disclosure and a second therapeutic agent are administered concurrently (e.g., simultaneously or sequentially). In some embodiments, a Compound of the Disclosure and a second therapeutic agent are administered in temporal proximity.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are degraders of AR and can be used in treating or preventing diseases and conditions wherein degradation of AR provides a benefit.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In some embodiments, the components of the combination therapies are administered at about 1 minute to about 24 hours apart.

As used herein, the term “temporal proximity” refers to that administration of one therapeutic agent (e.g., a Compound of the Disclosure) occurs within a time period before or after the administration of another therapeutic agent (e.g., a second therapeutic agent), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In some embodiments, the alkyl is a C₁-C₁₀ alkyl. In some embodiments, the alkyl is a C₁-C₆ alkyl. In some embodiments, the alkyl is a C₁-C₄ alkyl. In some embodiments, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56a}, —C(═O)R⁵⁷, —S(═O)R^{56e}, or —S(═O)₂R⁵⁸; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and
R⁵⁹ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In some embodiments, the alkenyl group is a C₂-C₆ alkenyl group. In some embodiments, the alkenyl group is a C₂-C₄ alkenyl group. In some embodiments, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In some embodiments, the alkynyl is a C₂-C₆ alkynyl. In some embodiments, the alkynyl is a C₂-C₄ alkynyl. In some embodiments, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In some embodiments, the alkyl is substituted by one, two, or three fluorine and/or chlorine atoms. In some embodiments, the alkyl is substituted by one, two, or three fluorine atoms. In some embodiments, the alkyl is a C₁-C₆ alkyl. In some embodiments, the alkyl is a C₁-C₄ alkyl. In some embodiments, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In some embodiments, the alkyl is a C₁-C₆ alkyl. In some embodiments, the alkyl is a C₁-C₄ alkyl. In some embodiments, the alkyl is a C₁ or C₂ alkyl. In some embodiments, the hydroxyalkyl is a monohydroxyalkyl group, i.e., substituted with one hydroxy group. In some embodiments, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In some embodiments, the alkyl is a C₁-C₆ alkyl and resulting alkoxy is thus referred to as a “C₁-C₆ alkoxy.” In some embodiments, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In some embodiments, the haloalkyl group is a C₁-C₆ haloalkyl. In some embodiments, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In some embodiments, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In some embodiments, the alkoxy is a C₁-C₆ alkoxy. In some embodiments, the alkoxy is a C₁-C₄ alkoxy. In some embodiments, the alkyl is a C₁-C₆ alkyl. In some embodiments, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In some embodiments, one —CH₂— group is replaced with one —O— group. In some embodiments, two —CH₂— groups are replaced with two —O— groups. In some embodiments, three —CH₂— groups are replaced with three —O— groups. In some embodiments, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In some embodiments, the cycloalkyl is bicyclic, i.e., it has two rings. In some embodiments, the cycloalkyl is monocyclic, i.e., it has one ring. In some embodiments, the cycloalkyl is a C₃₋₈ cycloalkyl. In some embodiments, the cycloalkyl is a C₃₋₆ cycloalkyl, i.e., cyclopropyl, cyclobutyl, cyclopentyl, or cyclohexyl. In some embodiments, the cycloalkyl is a C₅ cycloalkyl, i.e., cyclopentyl. In some embodiments, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56a}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

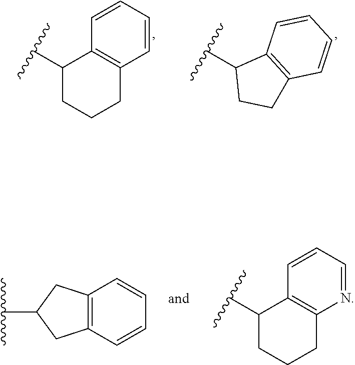 [SMILES: *C1CCCc2ccccc21 ; *C1CCc2ccccc21 ; *C1Cc2ccccc2C1 ; *C1CCCc2ncccc21]

Non-limiting exemplary optionally substituted cycloalkyl groups include:

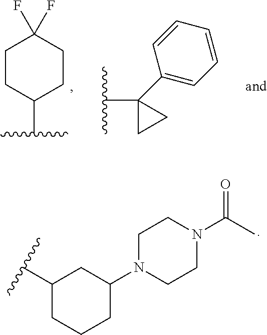

The term “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂. The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In some embodiments, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In some embodiments, the heterocyclo group is a 5- to 8-membered cyclic group containing one ring and one or two nitrogen atoms and, optionally, one —CH₂— group is replaced with one —C(═O)— group. In some embodiments, the heterocyclo group is a 5- or 6-membered cyclic group containing one ring and one or two nitrogen atoms and, optionally, one —CH₂— group is replaced with one —C(═O)— group. In some embodiments, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

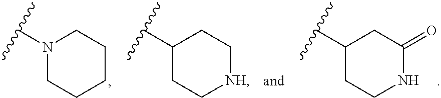

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56a}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

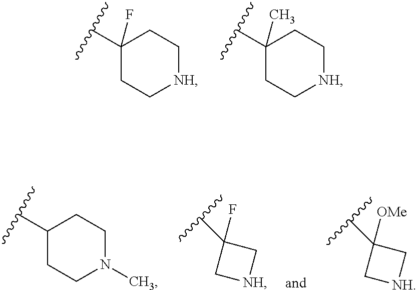

In some embodiments, the heterocyclo group is a spiroheterocyclo. The term “spiroheterocyclo” as used herein by itself or part of another group refers to an optionally substituted heterocyclo group containing seven to eighteen ring members, wherein:
(i) a first and second ring are connected through a quaternary carbon atom, i.e., a spirocarbon;
(ii) the first ring is an optionally substituted mono- or bicyclic heterocyclo containing a nitrogen atom; and
(iii) the second ring is either:
(a) an optionally substituted mono- or bicyclic cycloalkyl; or
(b) an optionally substituted mono- or bicyclic heterocyclo containing a nitrogen atom.

In some embodiments, the first ring is an optionally substituted monocyclic 4- to 9-membered heterocyclo containing a nitrogen atom. In some embodiments, the second ring is an optionally substituted monocyclic C₃₋₈ cycloalkyl. In some embodiments, the second ring is a monocyclic C₃₋₈ cycloalkyl substituted with a hydroxy group. In some embodiments, the second ring is an optionally substituted monocyclic 4- to 9-membered heterocyclo containing a nitrogen atom. Non-limiting exemplary spiroheterocyclo groups include:

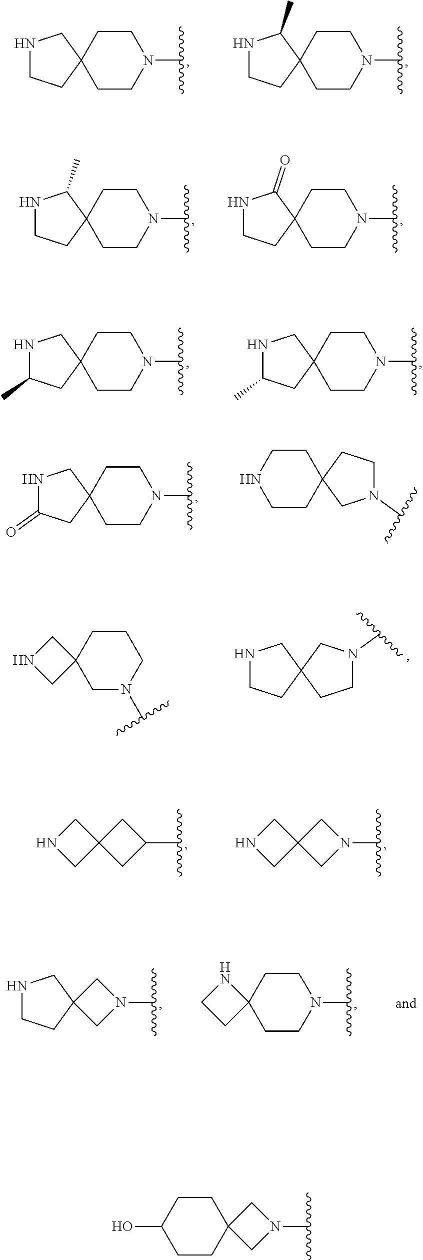

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In some embodiments, the aryl group is phenyl or naphthyl. In some embodiments, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56a}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In some embodiments, the optionally substituted aryl is an optionally substituted phenyl. In some embodiments, the optionally substituted phenyl has four substituents. In some embodiments, the optionally substituted phenyl has three substituents. In some embodiments, the optionally substituted phenyl has two substituents. In some embodiments, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups, e.g., a phenyl group, having fused optionally substituted cycloalkyl groups and fused optionally substituted heterocyclo groups. Non-limiting examples include:

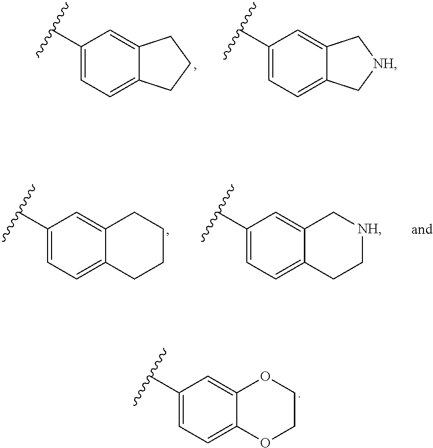 [SMILES: *c1ccc2c(c1)CCC2 ; *c1ccc2c(c1)CNC2 ; *c1ccc2c(c1)CCCC2 ; *c1ccc2c(c1)CNCC2 ; "and" ; *c1ccc2c(c1)OCCO2]

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In some embodiments, the heteroaryl has three heteroatoms. In some embodiments, the heteroaryl has two heteroatoms. In some embodiments, the heteroaryl has one heteroatom. In some embodiments, the heteroaryl is a 5- to 10-membered heteroaryl. In some embodiments, the heteroaryl has 5 ring atoms, e.g., thienyl, a 5-membered heteroaryl having four carbon atoms and one sulfur atom. In some embodiments, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In some embodiments, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In some embodiments, the optionally substituted heteroaryl has two substituents. In some embodiments, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In some embodiments, the alkyl is substituted with one cyano group. In some embodiments, the alkyl is a C₁-C₆ alkyl In some embodiments, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In some embodiments, the cycloalkyl group(s) is an optionally substituted C₃-C₆ cycloalkyl. In some embodiments, the alkyl is a C₁-C₆ alkyl. In some embodiments, the alkyl is a C₁-C₄ alkyl. In some embodiments, the alkyl is a C₁ or C₂ alkyl. In some embodiments, the alkyl is substituted with one optionally substituted cycloalkyl group. In some embodiments, the alkyl is substituted with two optionally substituted cycloalkyl groups. Non-limiting exemplary (cycloalkyl)alkyl groups include:

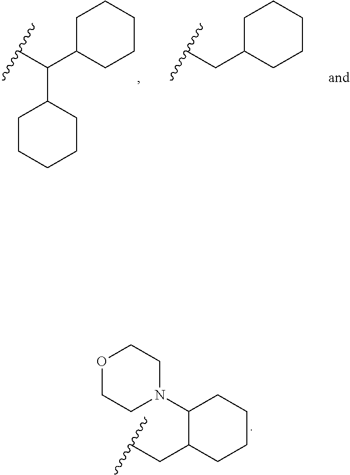 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In some embodiments, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In some embodiments, the alkyl is substituted with one optionally substituted 5- to 8-membered heterocyclo group. In some embodiments, alkyl is a C₁-C₆ alkyl. In some embodiments, alkyl is a C₁-C₄ alkyl. The heterocyclo group can be linked to the alkyl group through a carbon or nitrogen atom. Non-limiting exemplary (heterocyclo)alkyl groups include:

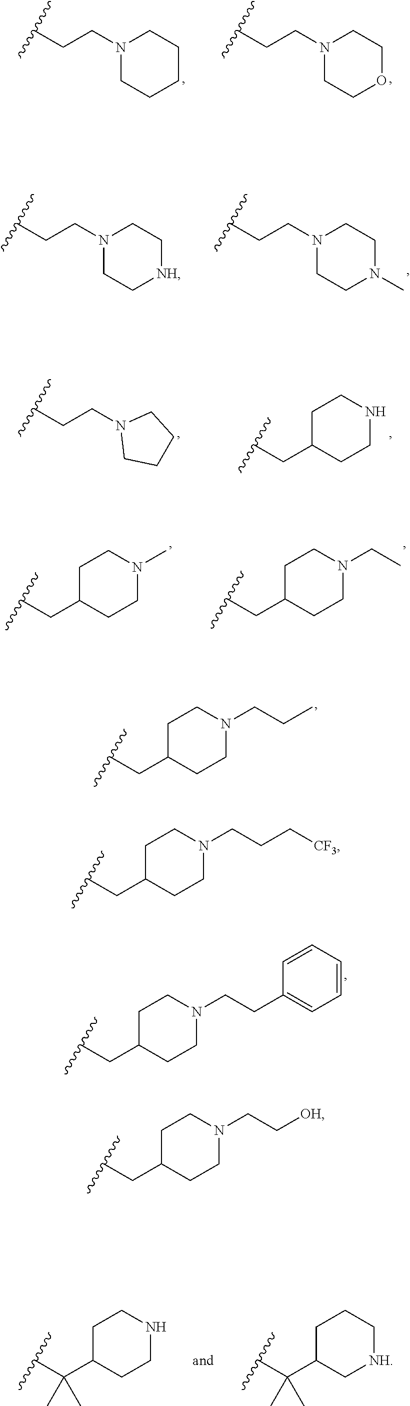

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In some embodiments, the alkyl group is substituted with one optionally substituted 5- to 14-membered heteroaryl group. In some embodiments, the alkyl group is substituted with two optionally substituted 5- to 14-membered heteroaryl groups. In some embodiments, the alkyl group is substituted with one optionally substituted 5- to 9-membered heteroaryl group. In some embodiments, the alkyl group is substituted with two optionally substituted 5- to 9-membered heteroaryl groups. In some embodiments, the alkyl group is substituted with one optionally substituted 5- or 6-membered heteroaryl group. In some embodiments, the alkyl group is substituted with two optionally substituted 5- or 6-membered heteroaryl groups. In some embodiments, the alkyl group is a C₁-C₆ alkyl. In some embodiments, the alkyl group is a C₁-C₄ alkyl. In some embodiments, the alkyl group is a C₁ or C₂ alkyl. Non-limiting exemplary (heteroaryl)alkyl groups include:

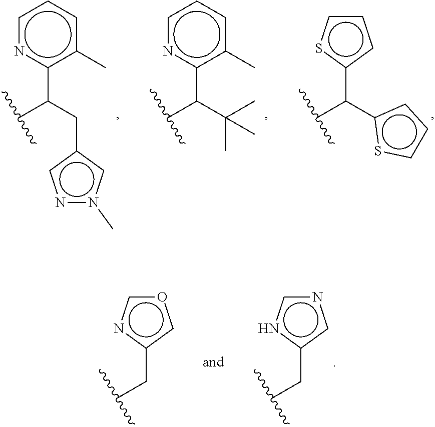

The term “(amino)(heteroaryl)alkyl” as used herein by itself or as part of another group refers to an alkyl group substituted with one optionally substituted heteroaryl group and one amino group. In some embodiments, the heteroaryl is an optionally substituted 5- to 9-membered heteroaryl group. In some embodiments, the heteroaryl is an optionally substituted 5- or 6-membered heteroaryl group. In some embodiments, the alkyl is a C₁-C₆ alkyl. In some embodiments, the alkyl is a C₁-C₄ alkyl. In some embodiments, the alkyl is a C₁ or C₂ alkyl. A non-limiting exemplary (amino)(heteroaryl)alkyl group is:

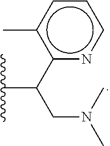

The terms “aralkyl” or “(aryl)alkyl” as used herein by themselves or as part of another group refers to an alkyl substituted with one, two, or three optionally substituted aryl groups. In some embodiments, the alkyl is substituted with one optionally substituted aryl group. In some embodiments, the alkyl is substituted with two optionally substituted aryl groups. In some embodiments, the aryl is an optionally substituted phenyl or optionally substituted naphthyl. In some embodiments, the aryl is an optionally substituted phenyl. In some embodiments, the alkyl is a C₁-C₆ alkyl. In some embodiments, the alkyl is a C₁-C₄ alkyl. In some embodiments, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In some embodiments, R^{60a} and R^{60b} are each independently hydrogen or C₁-C₆ alkyl.

The term “(amido)(aryl)alkyl” as used herein by itself or as part of another group refers to an alkyl group substituted with one amido group and one optionally substituted aryl group. In some embodiments, the aryl group is an optionally substituted phenyl. In some embodiments, the alkyl is a C₁-C₆ alkyl. In some embodiments, the alkyl is a C₁-C₄ alkyl. Non-limiting exemplary (amido)(aryl)alkyl groups include:

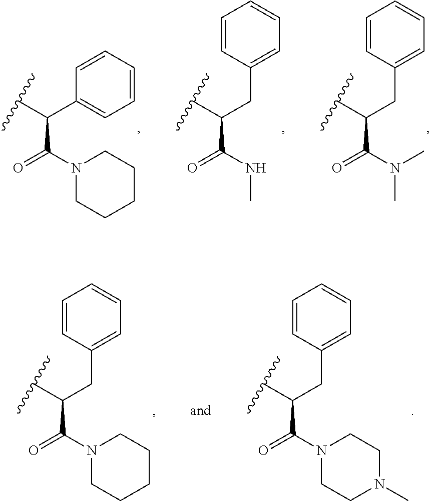 [SMILES: *[C@H](C(=O)N1CCCCC1)c1ccccc1 ; *[C@@H](Cc1ccccc1)C(=O)NC ; *[C@@H](Cc1ccccc1)C(=O)N(C)C ; *[C@@H](Cc1ccccc1)C(=O)N1CCCCC1 ; *[C@@H](Cc1ccccc1)C(=O)N1CCN(C)CC1]

The term “(amino)(aryl)alkyl” as used herein by itself or as part of another group refers to an alkyl group substituted with one amino group and one optionally substituted aryl group. In some embodiments, the amino group is —NH₂, alkylamino, or dialkylamino. In some embodiments, the aryl group is an optionally substituted phenyl. In some embodiments, the alkyl is a C₁-C₆ alkyl. In some embodiments, the alkyl is a C₁-C₄ alkyl. Non-limiting exemplary (amino)(aryl)alkyl groups include:

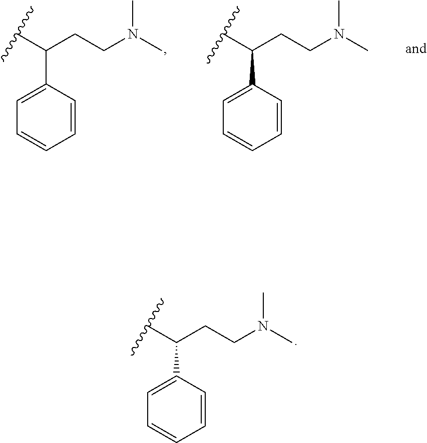 [SMILES: *C(CCN(C)C)c1ccccc1 ; *[C@@H](CCN(C)C)c1ccccc1 ; *[C@H](CCN(C)C)c1ccccc1]

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In some embodiments, the amino is —NH₂.

In some embodiments, the amino is an “alkylamino,” i.e., an amino group wherein R^{55a} is C₁₋₆ alkyl and R^{55b} is hydrogen. In some embodiments, R^{55a} is C₁-C₄ alkyl. Non-limiting exemplary alkylamino groups include —N(H)CH₃ and —N(H)CH₂CH₃.

In some embodiments, the amino is a “dialkylamino,” i.e., an amino group wherein R^{55a} and R^{55b} are each independently C₁₋₆ alkyl. In some embodiments, R^{55a} and R^{55b} are each independently C₁-C₄ alkyl. Non-limiting exemplary dialkylamino groups include —N(CH₃)₂ and —N(CH₃)CH₂CH(CH₃)₂.

In some embodiments, the amino is a “hydroxyalkylamino,” i.e., an amino group wherein R^{55a} is (hydroxyl)alkyl and R^{55b} is hydrogen or C₁-C₄ alkyl.

In some embodiments, the amino is a “cycloalkylamino,” i.e., an amino group wherein R^{55a} is optionally substituted cycloalkyl and R^{55b} is hydrogen or C₁-C₄ alkyl.

In some embodiments, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In some embodiments, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

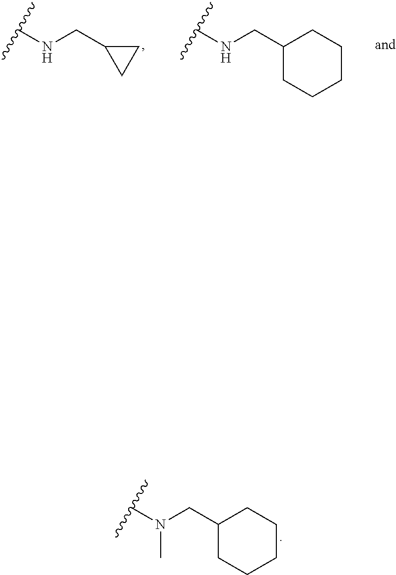

In some embodiments, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

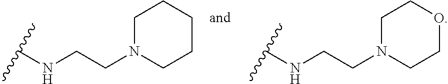 [SMILES: *NCCN1CCCCC1 ; *NCCN1CCOCC1]

The term “(amino)alkyl” as used herein by itself or as part of another group refers to an alkyl substituted with one amino group. In some embodiments, the amino group is —NH₂. In some embodiments, the amino group is an alkylamino. In some embodiments, the amino group is a dialkylamino. In some embodiments, the alkyl is a C₁-C₆ alkyl. In some embodiments, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The term “heteroarylenyl” as used herein by itself or part of another group refers to a divalent form of an optionally substituted heteroaryl group, e.g., a 5- to 9-membered heteroarylenyl. In some embodiments, the heteroarylenyl is a 6-membered heteroarylenyl, e.g., a heteroarylenyl derived from pyridine, pyrimidine, pyrazine, or pyridazine. In some embodiments, the heteroarylenyl is a 5-membered heteroarylenyl, e.g., a heteroarylenyl derived from oxazole, thiazole, isothiazole, thiophene, pyrazole, other 5-membered heteroaryl group. In some embodiments, the heteroarylenyl is a bicyclic 9-membered heteroarylenyl. Non-limiting exemplary heteroarylenyl groups include:

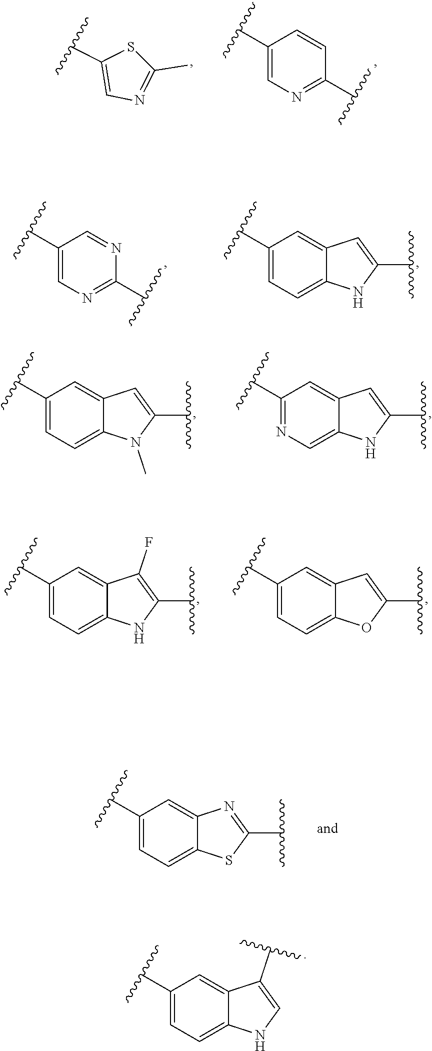

In the present disclosure, the term “alkylenyl” as used herein by itself or part of another group refers to a divalent form of an alkyl group, wherein the alkyl group is either unsubstituted or substituted with one or two groups independently selected from optionally substituted phenyl and optionally substituted 5- or 6-membered heteroaryl. In some embodiments, the alkylenyl is a divalent form of a C₁₋₁₂ alkyl, i.e., a C₁-C₁₂ alkylenyl. In some embodiments, the alkylenyl is a divalent form of a C₁₋₁₀ alkyl, i.e., a C₁-C₁₀ alkylenyl. In some embodiments, the alkylenyl is a divalent form of a C₁₋₈ alkyl, i.e., a C₁-C₈ alkylenyl. In some embodiments, the alkylenyl is a divalent form of an unsubstituted C₁₋₆ alkyl, i.e., a C₁-C₆ alkylenyl. In some embodiments, the alkylenyl is a divalent form of an unsubstituted C₁₋₄ alkyl, i.e., a C₁-C₈ alkylenyl. In some embodiments, the alkylenyl is a divalent form of a C₁₋₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In some embodiments, the heteroalkylenyl is a divalent form of a 3- to 20-membered heteroalkyl, i.e., a 3- to 20-membered heteroalkylenyl. In some embodiments, the heteroalkylenyl is a divalent form of a 3- to 10-membered heteroalkyl, i.e., a 3- to 10-membered heteroalkylenyl. In some embodiments, the heteroalkylenyl is a divalent form of a 3- to 8-membered heteroalkyl, i.e., a 3- to 8-membered heteroalkylenyl. In some embodiments, the heteroalkylenyl is a divalent form of a 3- to 6-membered heteroalkyl, i.e., a 3- to 6-membered heteroalkylenyl. In some embodiments, the heteroalkylenyl is a divalent form of a 3- or 4-membered heteroalkyl, i.e., a 3- or 4-membered heteroalkylenyl. In some embodiments, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted heterocyclo group. In some embodiments, the heterocyclenyl is a divalent form of a 4- to 14-membered heterocyclo group, i.e., a 4- to 14-membered heterocyclenyl. In some embodiments, the heterocyclenyl is a divalent form of a 4- to 10-membered heterocyclo group, i.e., a 4- to 10-membered heterocyclenyl. In some embodiments, the heterocyclenyl is a divalent form of a 4- to 8-membered heterocyclo group, i.e., a 4- to 8-membered heterocyclenyl. In some embodiments, the heterocyclenyl is a divalent form of an optionally substituted azetidine. In some embodiments, the heterocyclenyl is a divalent form of an optionally substituted piperidinyl. In some embodiments, the heterocyclenyl is a divalent form of an optionally substituted piperazinyl. Non-limiting exemplary heterocyclenyl groups include:

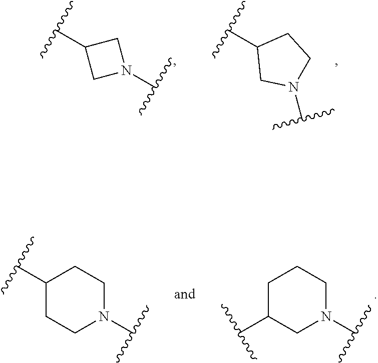 [SMILES: *C1CN(*)C1 ; *C1CCN(*)C1 ; *C1CCN(*)CC1 ; *C1CCCN(*)C1]

In some embodiments, the heterocyclenyl is a spiroheterocyclenyl.

The term “spiroheterocyclenyl” as used herein by itself or part of another group refers to a divalent form of a spiroheterocyclo. Non-limiting exemplary spiroheterocyclenyl groups include:

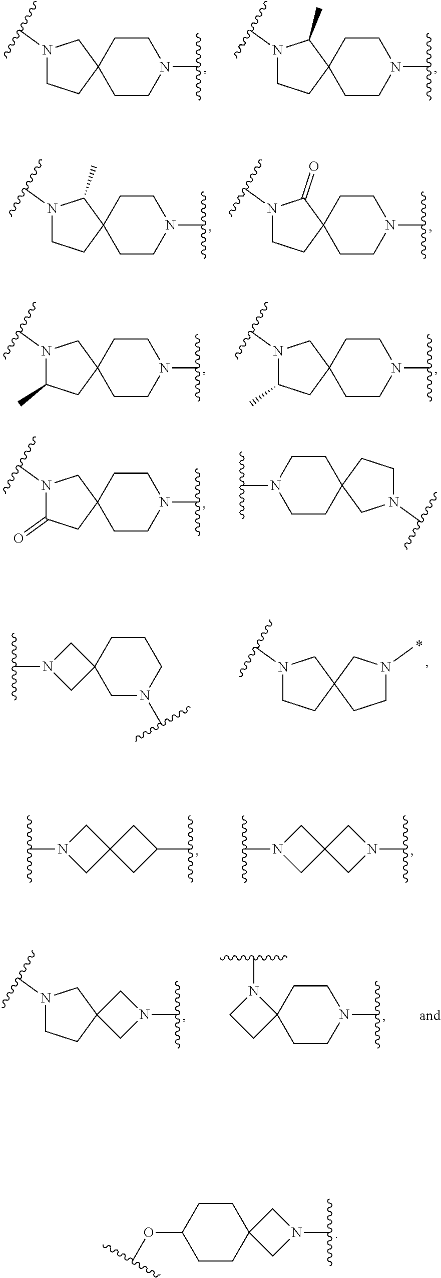

The term “cycloalkylenyl” as used herein by itself or part of another group refers to a divalent form of an optionally substituted C₄-C₆ cycloalkyl group. In some embodiments, the cycloalkylenyl is a 4-membered cycloalkylenyl. In some embodiments, the cycloalkylenyl is a 5-membered cycloalkylenyl. In some embodiments, the cycloalkylenyl is a 6-membered cycloalkylenyl. Non-limiting exemplary cycloalkylenyl groups include:

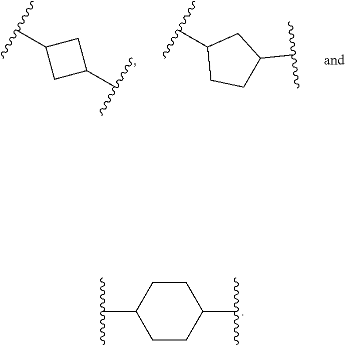 [SMILES: *C1CC(*)C1 ; *C1CCC(*)C1 ; *C1CCC(*)CC1]

The term “phenylenyl” as used herein by itself or part of another group refers to a divalent form of an optionally substituted phenyl group. Non-limiting exemplary phenylenyl groups include:

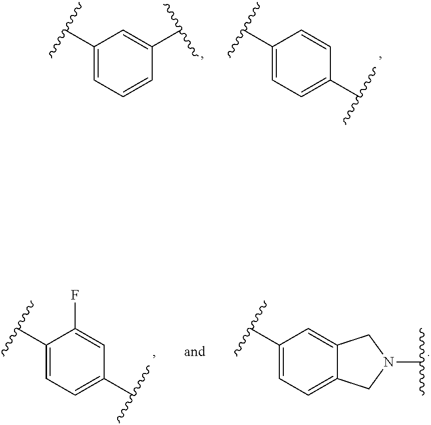 [SMILES: *c1cccc(*)c1 ; *c1ccc(*)cc1 ; *c1ccc(*)c(F)c1 ; *c1ccc2c(c1)CN(*)C2]

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O ³¹P, ³²P, ³⁵S, ¹⁸F, and ³⁶Cl, respectively, e.g., ³H, ¹¹C, and ¹⁴C. In some embodiments, provided is a composition wherein substantially all of the atoms at a position within the Compound of the Disclosure are replaced by an atom having a different atomic mass or mass number. In some embodiments, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

As noted above, Compounds of the Disclosure contain one or more asymmetric carbon atoms and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In some embodiments, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Examples

### Example 1. Synthesis of 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile (Cpd. No. 143)

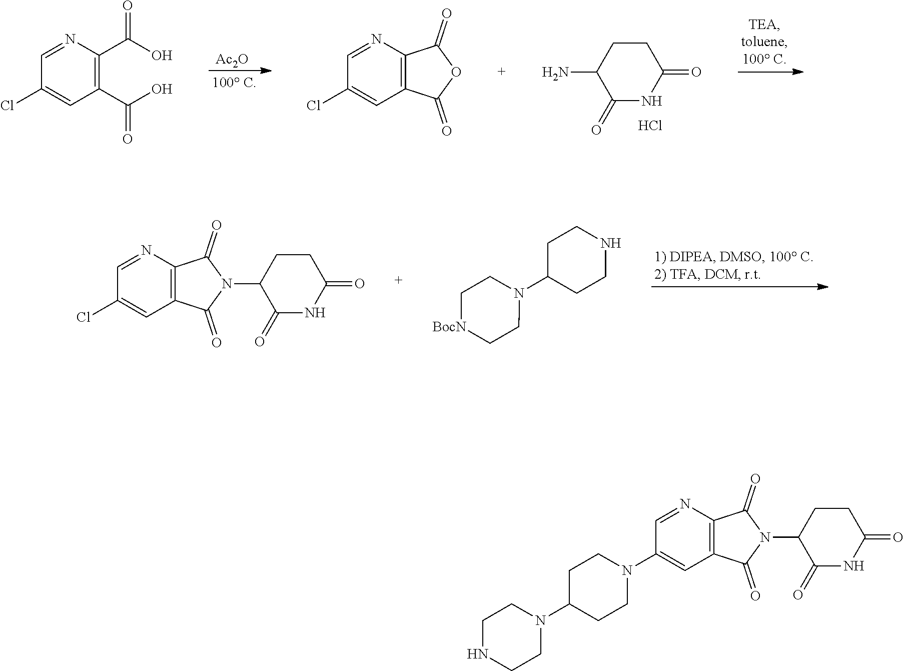

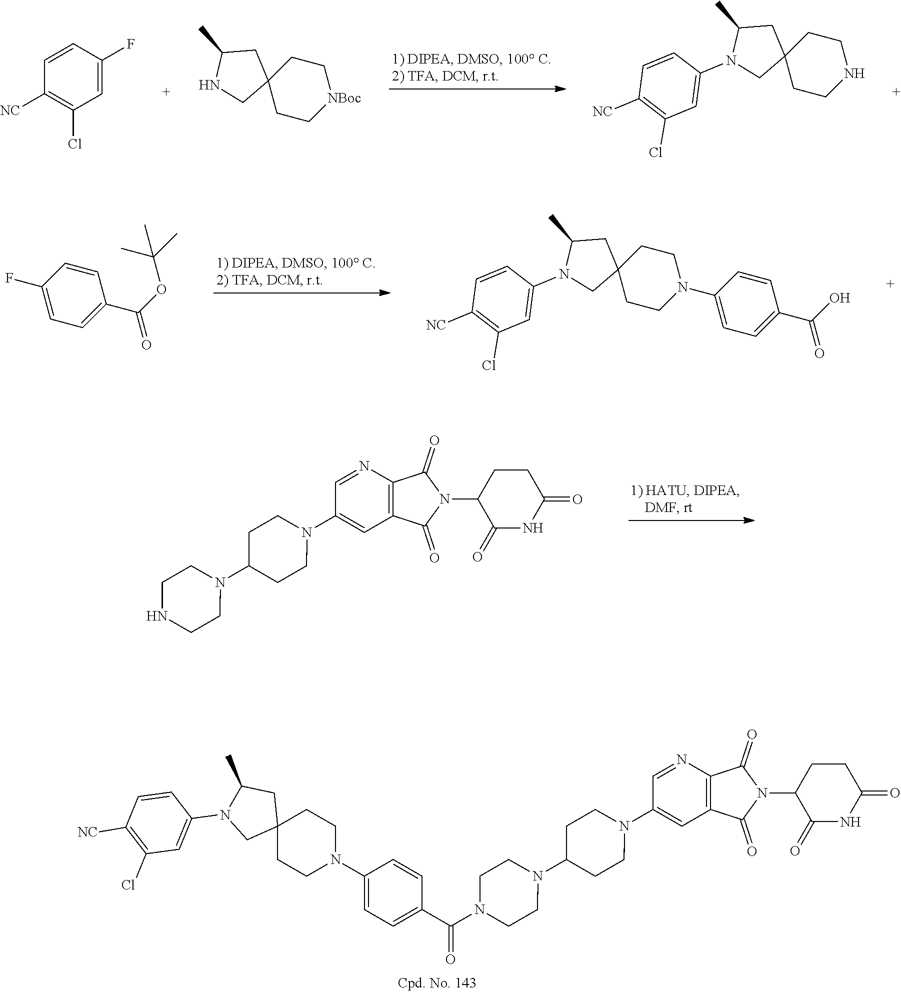

### Step 1: Synthesis of 3-chlorofuro[3,4-b]pyridine-5,7-dione

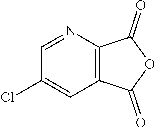 [SMILES: O=C1OC(=O)c2ncc(Cl)cc21]

5-chloropyridine-2,3-dicarboxylic acid was dissolved in Ac₂O and the reaction mixture was stirred at 100° C. for 4 h. The solvent was evaporated and the residue was used for next step without further purification. ESI-MS: 182.97.

### Step 2: Synthesis of 3-chloro-6-(2,6-dioxopiperidin-3-yl)-5H-pyrrolo[3,4-b]pyridine-5,7(6H)-dione

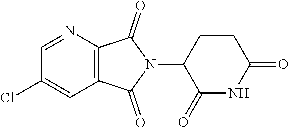 [SMILES: O=C1CCC(N2C(=O)c3cc(Cl)cnc3C2=O)C(=O)N1]

3-chlorofuro[3,4-b]pyridine-5,7-dione and 3-aminopiperidine-2,6-dione hydrogen chloride were dissolved in toluene and TEA was added into the mixture. Then, the reaction mixture was stirred at 100° C. for 6 h. The solvent was evaporated and compound 3-chloro-6-(2,6-dioxopiperidin-3-yl)-5H-pyrrolo[3,4-b]pyridine-5,7(6H)-dione was purified by flash column chromatography on silica gel. ESI-MS: 293.02.

### Step 3: Synthesis of 6-(2,6-dioxopiperidin-3-yl)-3-(4-(piperazin-1-yl)piperidin-1-yl)-5H-pyrrolo[3,4-b]pyridine-5,7(6H)-dione

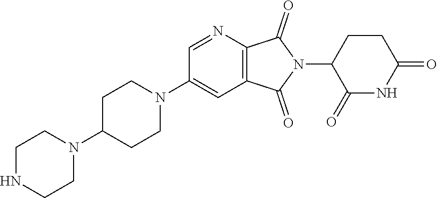 [SMILES: O=C1CCC(N2C(=O)c3cc(N4CCC(N5CCNCC5)CC4)cnc3C2=O)C(=O)N1]

3-chloro-6-(2,6-dioxopiperidin-3-yl)-5H-pyrrolo[3,4-b]pyridine-5,7(6H)-dione and tert-butyl 4-(piperidin-4-yl)piperazine-1-carboxylate were dissolved in DMSO. To this solution was added DIPEA, and the reaction mixture was stirred at 100° C. for 6 h. The Boc-compound was obtained by removing the solvent under vacuum and purified by flash column. 6-(2,6-dioxopiperidin-3-yl)-3-(4-(piperazin-1-yl)piperidin-1-yl)-5H-pyrrolo[3,4-b]pyridine-5,7(6H)-dione was obtained by removing the Boc group using TFA in DCM. ESI-MS: 426.20.

### Step 4: Synthesis of (S)-2-chloro-4-(3-methyl-2,8-diazaspiro[4.5]decan-2-yl)benzonitrile

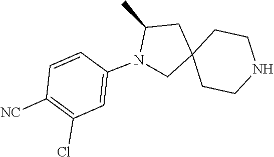 [SMILES: C[C@H]1CC2(CCNCC2)CN1c1ccc(C#N)c(Cl)c1]

2-Chloro-4-fluorobenzonitrile and tert-butyl (S)-3-methyl-2,8-diazaspiro[4.5]decane-8-carboxylate were dissolved in DMSO. To this solution was added DIPEA (5 eq.), and the reaction mixture was stirred at 100° C. for 4 h. The reaction mixture was partitioned between water and ethyl acetate. The organic phase was separated, washed with water, and dried over Na₂SO₄. The Boc protected compound was obtained by removing the solvent under vacuum and purifying by flash column chromatography on silica gel. (S)-2-Chloro-4-(3-methyl-2,8-diazaspiro[4.5]decan-2-yl)benzonitrile was obtained by removing the Boc group using TFA in DCM. ESI-MS: 289.13.

### Step 5: Synthesis of (S)-4-(2-(3-chloro-4-cyanophenyl)-3-methyl-2,8-diazaspiro[4.5]decan-8-yl)benzoic acid

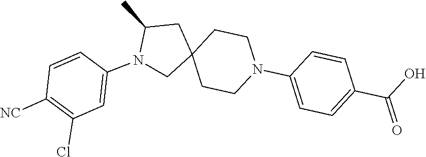 [SMILES: C[C@H]1CC2(CCN(c3ccc(C(=O)O)cc3)CC2)CN1c1ccc(C#N)c(Cl)c1]

(S)-2-Chloro-4-(3-methyl-2,8-diazaspiro[4.5]decan-2-yl)benzonitrile and tert-butyl 4-fluorobenzoate were dissolved in DMSO. To this solution was added DIPEA, and the reaction mixture was stirred at 100° C. for 6 h. The t-Bu ester compound was obtained by removing the solvent under vacuum and purified by flash column. (S)-4-(2-(3-Chloro-4-cyanophenyl)-3-methyl-2,8-diazaspiro[4.5]decan-8-yl)benzoic acid was obtained by removing the t-Bu group using TFA in DCM. ESI-MS: 409.16.

### Step 6: Synthesis of 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile (Cpd. No. 143)

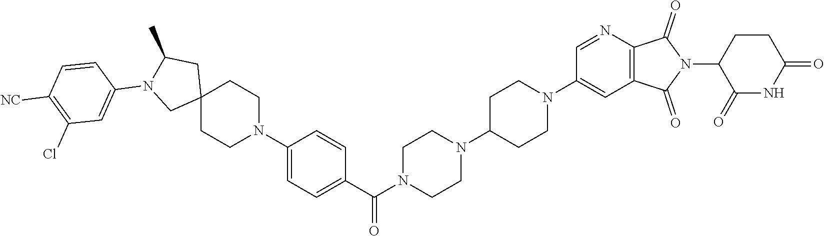 [SMILES: C[C@H]1CC2(CCN(c3ccc(C(=O)N4CCN(C5CCN(c6cnc7c(c6)C(=O)N(C6CCC(=O)NC6=O)C7=O)CC5)CC4)cc3)CC2)CN1c1ccc(C#N)c(Cl)c1]

(S)-4-(2-(3-Chloro-4-cyanophenyl)-3-methyl-2,8-diazaspiro[4.5]decan-8-yl)benzoic acid and 6-(2,6-dioxopiperidin-3-yl)-3-(4-(piperazin-1-yl)piperidin-1-yl)-5H-pyrrolo[3,4-b]pyridine-5,7(6H)-dione were dissolved in DMF. To this solution was added DIPEA (5 eq.) and HATU (1.2 eq.), and the reaction mixture was stirred at r.t. for 1 h. The reaction mixture was partitioned between water and ethyl acetate. The organic phase was separated, washed with water, dried over Na₂SO₄, and purified by flash column chromatography on silica gel to give 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile. LC-MS(ESI) m/z (M+H)⁺: 818.38; calcd: 818.36; >95% purity.

### Example 2. Synthesis of 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)-2-oxoethyl)piperidine-4-carbonyl)phenyl)-3-methyl-2,8-diazaspiro[4.5]decan-2-yl)benzonitrile (Cpd. No. 147)

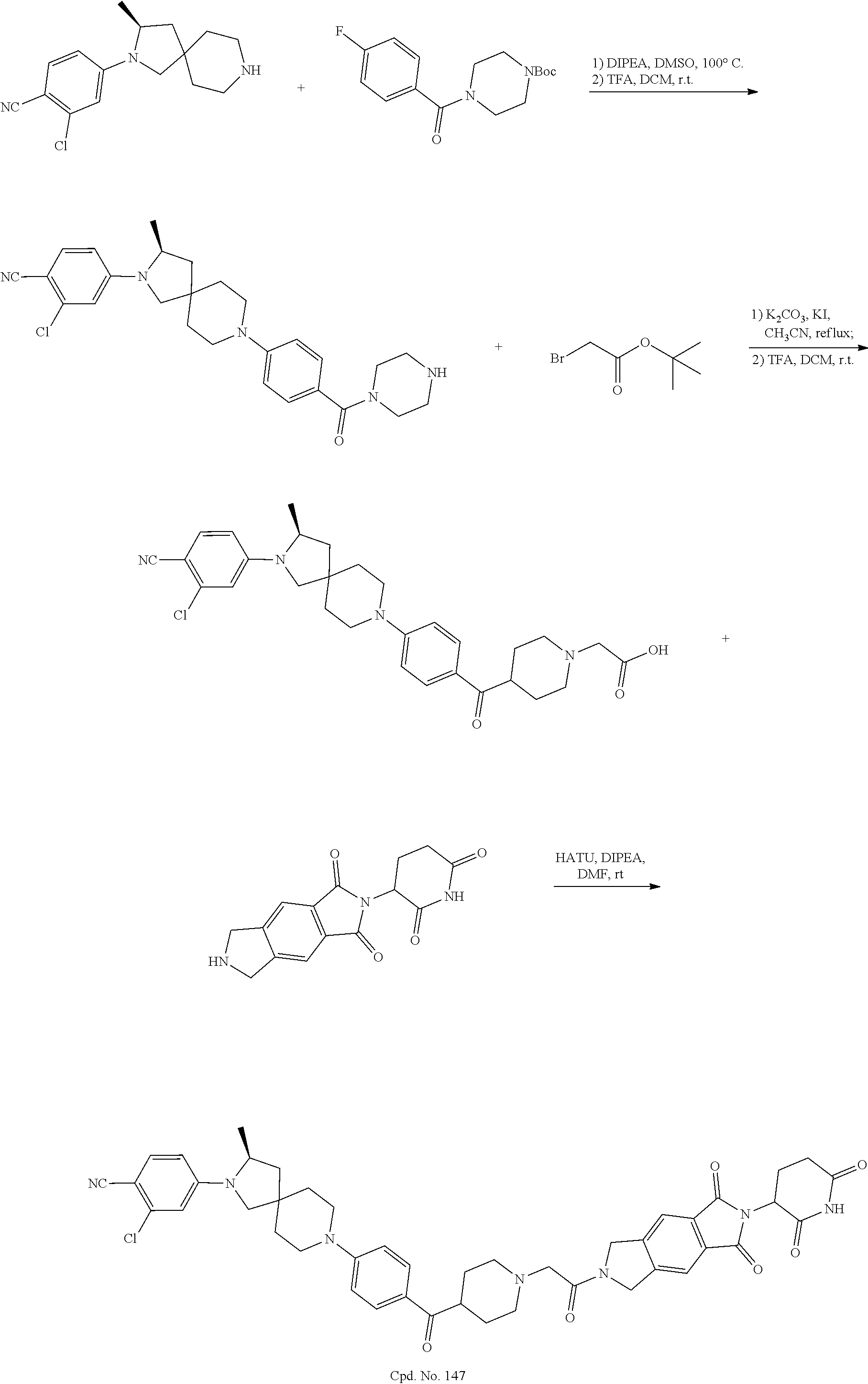

### Step 1: Synthesis of (S)-2-chloro-4-(3-methyl-8-(4-(piperidine-4-carbonyl)phenyl)-2,8-diazaspiro[4.5]decan-2-yl)benzonitrile

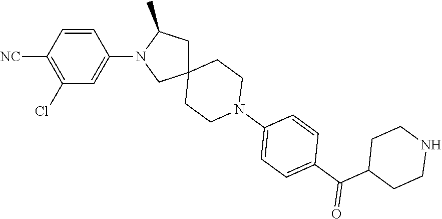 [SMILES: C[C@H]1CC2(CCN(c3ccc(C(=O)C4CCNCC4)cc3)CC2)CN1c1ccc(C#N)c(Cl)c1]

(S)-2-Chloro-4-(3-methyl-2,8-diazaspiro[4.5]decan-2-yl)benzonitrile and tert-butyl 4-(4-fluorobenzoyl)piperidine-1-carboxylate were dissolved in DMSO. To this solution was added DIPEA, and the reaction mixture was stirred at 100° C. for 6 h. The Boc-compound was obtained by removing the solvent under vacuum and purified by flash column. (S)-2-chloro-4-(3-methyl-8-(4-(piperidine-4-carbonyl)phenyl)-2,8-diazaspiro[4.5]decan-2-yl)benzonitrile was obtained by removing the Boc group using TFA in DCM. ESI-MS: 476.23.

### Step 2: Synthesis of (S)-2-(4-(4-(2-(3-chloro-4-cyanophenyl)-3-methyl-2,8-diazaspiro[4.5]decan-8-yl)benzoyl)piperidin-1-yl)acetic acid

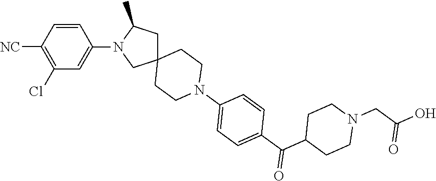 [SMILES: C[C@H]1CC2(CCN(c3ccc(C(=O)C4CCN(CC(=O)O)CC4)cc3)CC2)CN1c1ccc(C#N)c(Cl)c1]

(S)-2-chloro-4-(3-methyl-8-(4-(piperidine-4-carbonyl)phenyl)-2,8-diazaspiro[4.5]decan-2-yl)benzonitrile and tert-butyl 2-bromoacetate were dissolved in CH₃CN. To this solution was added K₂CO₃, KI, and the reaction mixture was stirred at 100° C. for 4 h. The t-Bu-compound was obtained by removing the solvent under vacuum and purified by flash column. (S)-2-(4-(4-(2-(3-chloro-4-cyanophenyl)-3-methyl-2,8-diazaspiro[4.5]decan-8-yl)benzoyl)piperidin-1-yl)acetic acid was obtained by removing the t-Bu group using TFA in DCM. ESI-MS: 534.24.

### Step 3: Synthesis of 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)-2-oxoethyl)piperidine-4-carbonyl)phenyl)-3-methyl-2,8-diazaspiro[4.5]decan-2-yl)benzonitrile (Cpd. No. 147)

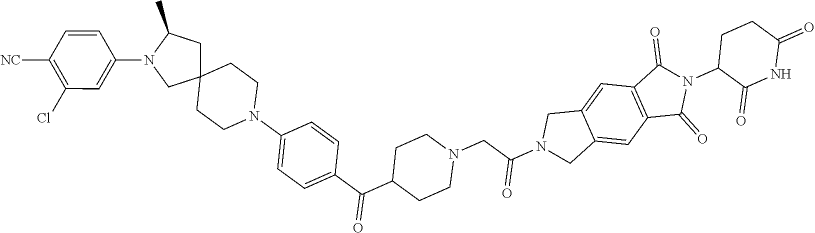 [SMILES: C[C@H]1CC2(CCN(c3ccc(C(=O)C4CCN(CC(=O)N5Cc6cc7c(cc6C5)C(=O)N(C5CCC(=O)NC5=O)C7=O)CC4)cc3)CC2)CN1c1ccc(C#N)c(Cl)c1]

(S)-2-(4-(4-(2-(3-chloro-4-cyanophenyl)-3-methyl-2,8-diazaspiro[4.5]decan-8-yl)benzoyl)piperidin-1-yl)acetic acid and 2-(2,6-dioxopiperidin-3-yl)-6,7-dihydropyrrolo[3,4-f]isoindole-1,3(2H,5H)-dione were dissolved in DMF. To this solution was added DIPEA (5 eq.) and HATU (1.2 eq.), and the reaction mixture was stirred at r.t. for 1 h. The reaction mixture was partitioned between water and ethyl acetate. The organic phase was separated, washed with water, dried over Na₂SO₄, and purified by flash column chromatography on silica gel to give 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)-2-oxoethyl)piperidine-4-carbonyl)phenyl)-3-methyl-2,8-diazaspiro[4.5]decan-2-yl)benzonitrile. ¹HNMR (400 MHz, DMSO-d₆) δ 11.14 (s, 1H), 9.83 (s, 1H), 8.06-7.83 (m, 4H), 7.59 (d, J=8.8 Hz, 1H), 7.01 (d, J=8.1 Hz, 2H), 6.79 (s, 1H), 6.65 (d, J=8.9 Hz, 1H), 5.18 (dd, J=12.6, 5.1 Hz, 1H), 5.06-4.74 (m, 4H), 4.37 (d, J=20.8 Hz, 2H), 4.03 (dd, J=12.7, 6.3 Hz, 1H), 3.82-3.10 (m, 12H), 2.91 (dd, J=16.7, 9.4 Hz, 1H), 2.60 (dd, J=22.9, 15.2 Hz, 2H), 2.25 (dd, J=12.4, 7.8 Hz, 1H), 1.79-1.43 (m, 5H), 1.21 (d, J=5.8 Hz, 3H). LC-MS(ESI) m/z (M+H)⁺: 816.37; calcd: 816.33; >95% purity.

### Example 3. Synthesis of 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)acetyl)piperidine-4-carbonyl)phenyl)-3-methyl-2,8-diazaspiro[4.5]decan-2-yl)benzonitrile (Cpd. No. 148)

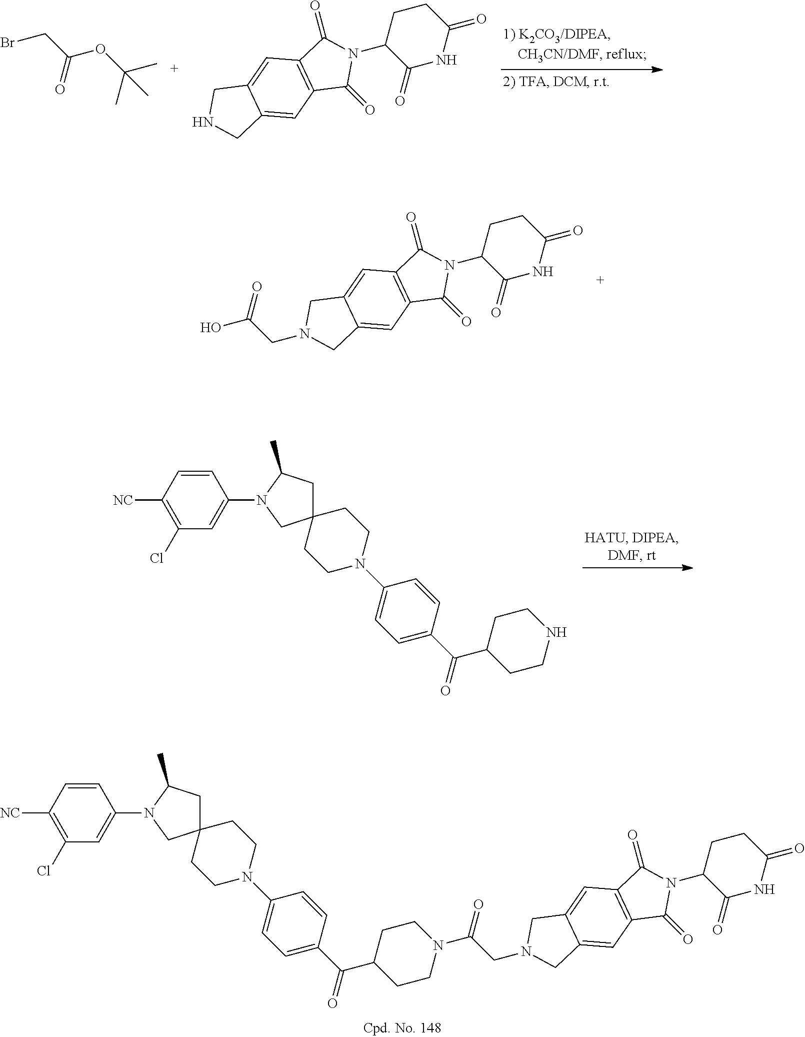

### Step 1: Synthesis of 2-(6-(2,6-dioxopiperidin-3-yl)-5,7-dioxo-3,5,6,7-tetrahydropyrrolo[3,4-f]isoindol-2(1H)-yl)acetic acid

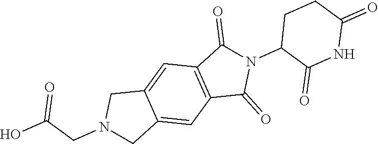 [SMILES: O=C(O)CN1Cc2cc3c(cc2C1)C(=O)N(C1CCC(=O)NC1=O)C3=O]

(2-(2,6-dioxopiperidin-3-yl)-6,7-dihydropyrrolo[3,4-f]isoindole-1,3(2H,5H)-dione, DIPEA and K₂CO₃ were dissolved in CH₃CN/DMF (10:1). To this solution was added tert-butyl 2-bromoacetate and the reaction mixture was stirred at 100° C. for 2 h. The t-Bu-compound was obtained by removing the solvent under vacuum and purified by flash column. 2-(6-(2,6-dioxopiperidin-3-yl)-5,7-dioxo-3,5,6,7-tetrahydropyrrolo[3,4-f]isoindol-2(1H)-yl)acetic acid was obtained by removing the t-Bu group using TFA in DCM. ESI-MS: 358.11.

### Step 2: Synthesis of 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)acetyl)piperidine-4-carbonyl)phenyl)-3-methyl-2,8-diazaspiro[4.5]decan-2-yl)benzonitrile (Cpd. No. 148)

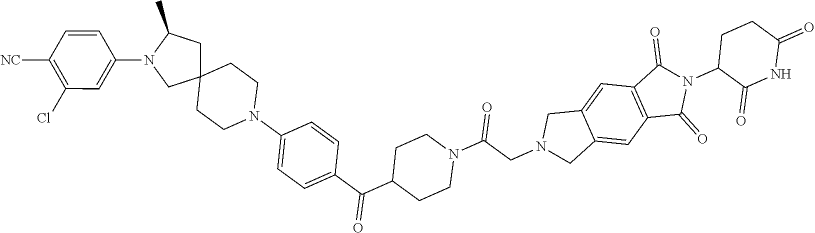 [SMILES: C[C@H]1CC2(CCN(c3ccc(C(=O)C4CCN(C(=O)CN5Cc6cc7c(cc6C5)C(=O)N(C5CCC(=O)NC5=O)C7=O)CC4)cc3)CC2)CN1c1ccc(C#N)c(Cl)c1]

2-(6-(2,6-dioxopiperidin-3-yl)-5,7-dioxo-3,5,6,7-tetrahydropyrrolo[3,4-f]isoindol-2(1H)-yl)acetic acid and (S)-2-chloro-4-(3-methyl-8-(4-(piperidine-4-carbonyl)phenyl)-2,8-diazaspiro[4.5]decan-2-yl)benzonitrile were dissolved in DMF. To this solution was added DIPEA (5 eq.) and HATU (1.2 eq.), and the reaction mixture was stirred at r.t. for 1 h. The reaction mixture was partitioned between water and ethyl acetate. The organic phase was separated, washed with water, dried over Na₂SO₄, and purified by flash column chromatography on silica gel to give 2-chloro-4-((3S)-8-(4-(1-(2-(6-(2,6-dioxopiperidin-3-yl)-5,7-dioxo-3,5,6,7-tetrahydropyrrolo[3,4-f]isoindol-2(1H)-yl)acetyl)piperidine-4-carbonyl)phenyl)-3-methyl-2,8-diazaspiro[4.5]decan-2-yl)benzonitrile. ¹H NMR (400 MHz, DMSO) δ 11.15 (s, 1H), 8.02 (s, 2H), 7.88 (d, J=8.4 Hz, 2H), 7.60 (d, J=8.8 Hz, 1H), 7.00 (d, J=8.3 Hz, 2H), 6.80 (s, 1H), 6.66 (d, J=8.3 Hz, 1H), 5.21-5.16 (m, 1H), 4.69 (dd, J=37.0, 16.0 Hz, 4H), 4.44 (d, J=12.3 Hz, 1H), 4.04 (d, J=6.2 Hz, 1H), 3.66 (d, J=11.5 Hz, 2H), 3.50-3.22 (m, 7H), 2.98-2.86 (m, 2H), 2.67-2.45 (m, 3H), 2.25 (dd, J=12.3, 7.8 Hz, 1H), 2.09 (d, J=5.6 Hz, 1H), 1.79-1.59 (m, 5H), 1.48 (s, 3H), 1.21 (d, J=5.7 Hz, 3H). LC-MS(ESI) m/z (M+H)⁺: 816.35; calcd: 816.33; >95% purity.

### Example 4. Synthesis of 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile (Cpd. No. 167)

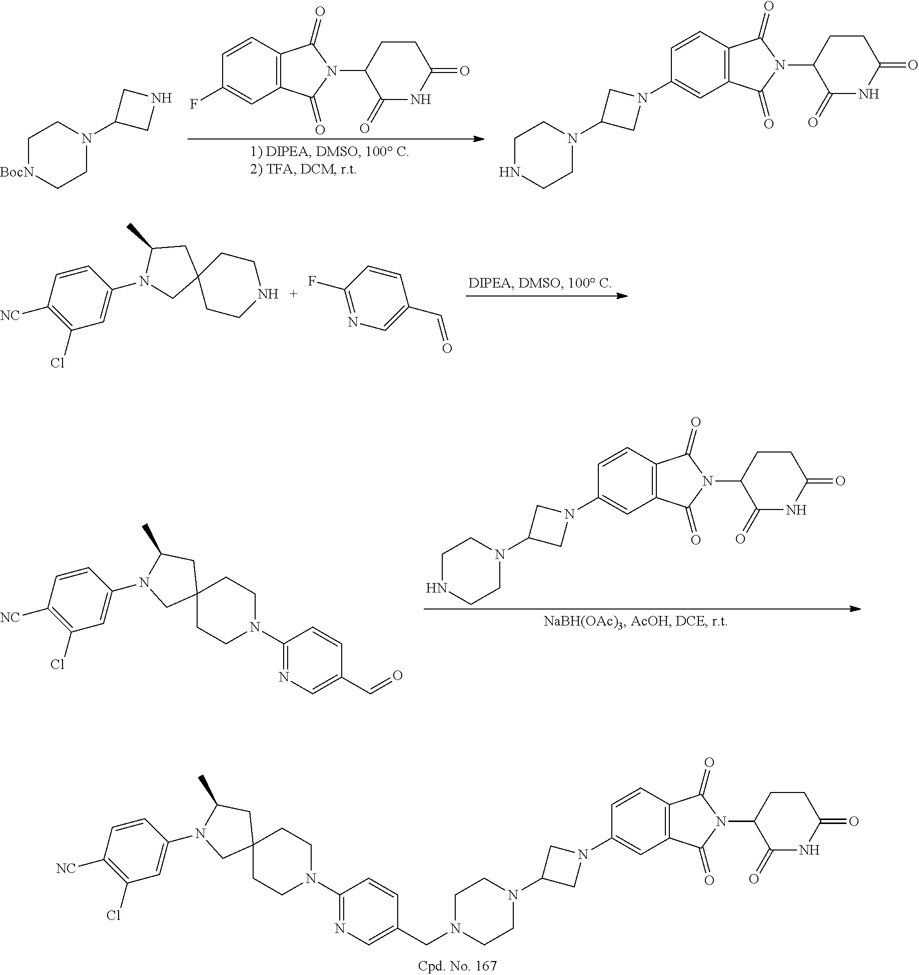

### Step 1: Synthesis of 2-(2,6-dioxopiperidin-3-yl)-5-(3-(piperazin-1-yl)azetidin-1-yl)isoindoline-1,3-dione

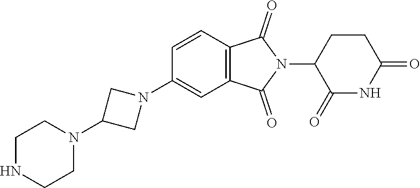 [SMILES: O=C1CCC(N2C(=O)c3ccc(N4CC(N5CCNCC5)C4)cc3C2=O)C(=O)N1]

tert-butyl 4-(azetidin-3-yl)piperazine-1-carboxylate and 2-(2,6-dioxopiperidin-3-yl)-5-fluoroisoindoline-1,3-dione were dissolved in DMSO. To this solution was added DIPEA, and the reaction mixture was stirred at 100° C. for 6 h. The Boc-compound was obtained by removing the solvent under vacuum and purified by flash column. 2-(2,6-dioxopiperidin-3-yl)-5-(3-(piperazin-1-yl)azetidin-1-yl)isoindoline-1,3-dione was obtained by removing the Boc group using TFA in DCM. ESI-MS: 397.18.

### Step 2: Synthesis of (S)-2-chloro-4-(8-(5-formylpyridin-2-yl)-3-methyl-2,8-diazaspiro[4.5]decan-2-yl)benzonitrile

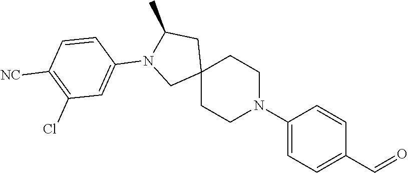 [SMILES: C[C@H]1CC2(CCN(c3ccc(C=O)cc3)CC2)CN1c1ccc(C#N)c(Cl)c1]

(S)-2-chloro-4-(3-methyl-2,8-diazaspiro[4.5]decan-2-yl)benzonitrile and 6-fluoronicotinaldehyde were dissolved in DMSO. To this solution was added DIPEA (5 eq.), and the reaction mixture was stirred at 100° C. for 4 h. The reaction mixture was partitioned between water and ethyl acetate. The organic phase was separated, washed with water, and dried over Na₂SO₄. The (S)-2-chloro-4-(8-(5-formylpyridin-2-yl)-3-methyl-2,8-diazaspiro[4.5]decan-2-yl)benzonitrile was obtained by removing the solvent under vacuum and purifying by flash column chromatography on silica gel. ESI-MS: 394.16.

### Step 3: Synthesis of 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile (Cpd. No. 167)

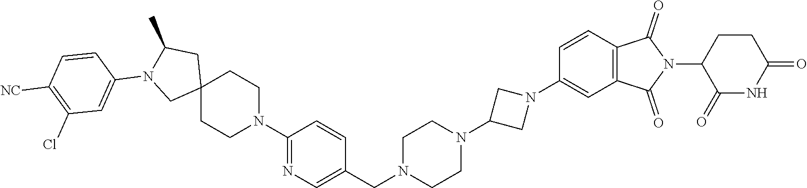 [SMILES: C[C@H]1CC2(CCN(c3ccc(CN4CCN(C5CN(c6ccc7c(c6)C(=O)N(C6CCC(=O)NC6=O)C7=O)C5)CC4)cn3)CC2)CN1c1ccc(C#N)c(Cl)c1]

2-(2,6-dioxopiperidin-3-yl)-5-(3-(piperazin-1-yl)azetidin-1-yl)isoindoline-1,3-dione and (S)-2-chloro-4-(8-(5-formylpyridin-2-yl)-3-methyl-2,8-diazaspiro[4.5]decan-2-yl)benzonitrile were dissolved in DCE. To this solution was added NaBH(OAc)₃ and AcOH, and the reaction mixture was stirred at r.t. for 4 h. The reaction mixture was evaporated and purified by flash column chromatography on silica gel to give 2-chloro-4-((3S)-8-(4-(4-(1-(6-(2,6-dioxopiperidin-3-yl)-5,7-dioxo-6,7-dihydro-5H-pyrrolo[3,4-b]pyridin-3-yl)piperidin-4-yl)piperazine-1-carbonyl)phenyl)-3-methyl-2,8-diazaspiro[4.5]decan-2-yl)benzonitrile. LC-MS(ESI) m/z (M+H)⁺: 776.38; calcd: 776.35; >95% purity.

### Example 5. Synthesis of 2-chloro-4-((3S)-8-(5-((4-(1-(2-(2,6-dioxopiperidin-3-yl)-6-fluoro-1,3-dioxoisoindolin-5-yl)azetidin-3-yl)piperazin-1-yl)methyl)pyridin-2-yl)-3-methyl-2,8-diazaspiro[4.5]decan-2-yl)benzonitrile (Cpd. No. 153)

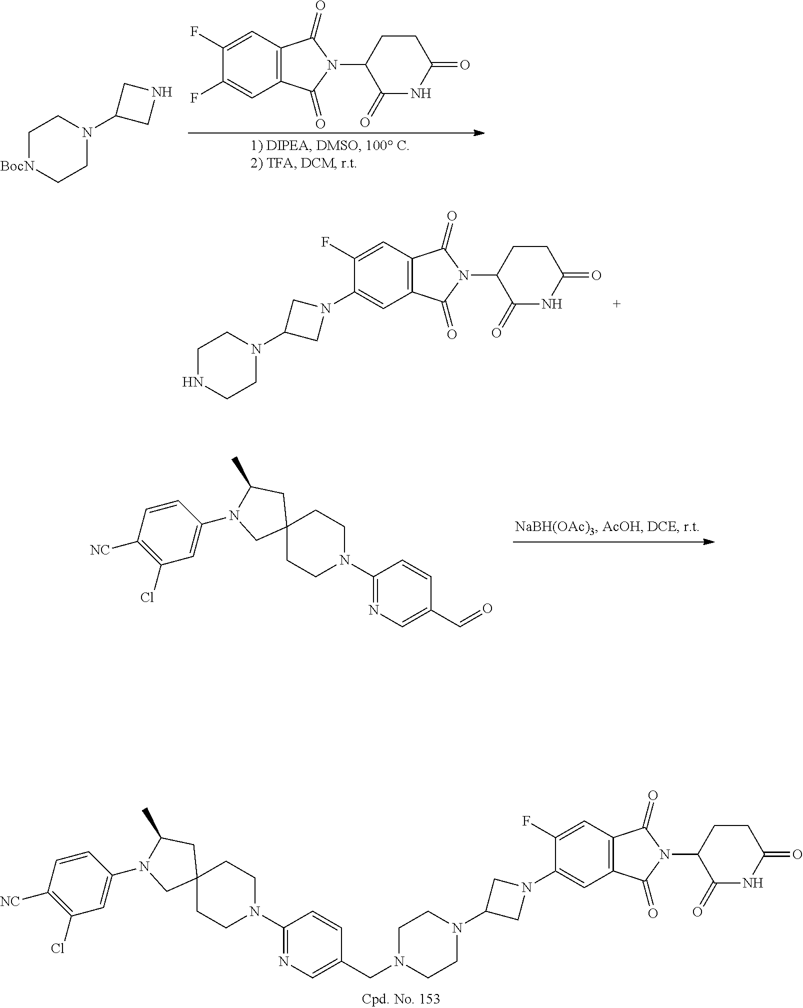

### Step 1: Synthesis of 2-(2,6-dioxopiperidin-3-yl)-5-fluoro-6-(3-(piperazin-1-yl)azetidin-1-yl)isoindoline-1,3-dione

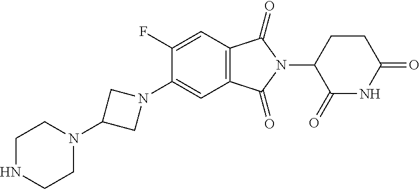 [SMILES: O=C1CCC(N2C(=O)c3cc(F)c(N4CC(N5CCNCC5)C4)cc3C2=O)C(=O)N1]

tert-butyl 4-(azetidin-3-yl)piperazine-1-carboxylate and 2-(2,6-dioxopiperidin-3-yl)-5,6-difluoroisoindoline-1,3-dione were dissolved in DMSO. To this solution was added DIPEA, and the reaction mixture was stirred at 100° C. for 6 h. The Boc-compound was obtained by removing the solvent under vacuum and purified by flash column. 2-(2,6-dioxopiperidin-3-yl)-5-fluoro-6-(3-(piperazin-1-yl)azetidin-1-yl)isoindoline-1,3-dione was obtained by removing the Boc group using TFA in DCM. ESI-MS: 415.17.

### Step 2: Synthesis of 2-chloro-4-((3S)-8-(5-((4-(1-(2-(2,6-dioxopiperidin-3-yl)-6-fluoro-1,3-dioxoisoindolin-5-yl)azetidin-3-yl)piperazin-1-yl)methyl)pyridin-2-yl)-3-methyl-2,8-diazaspiro[4.5]decan-2-yl)benzonitrile (Cpd. No. 153)

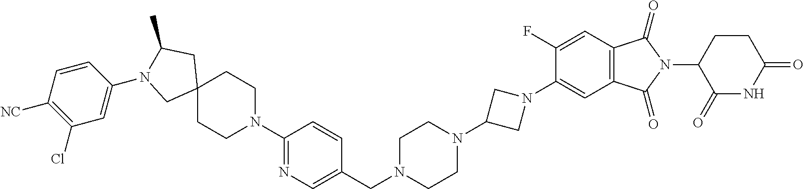 [SMILES: C[C@H]1CC2(CCN(c3ccc(CN4CCN(C5CN(c6cc7c(cc6F)C(=O)N(C6CCC(=O)NC6=O)C7=O)C5)CC4)cn3)CC2)CN1c1ccc(C#N)c(Cl)c1]

2-(2,6-dioxopiperidin-3-yl)-5-fluoro-6-(3-(piperazin-1-yl)azetidin-1-yl)isoindoline-1,3-dione and (S)-2-chloro-4-(8-(5-formylpyridin-2-yl)-3-methyl-2,8-diazaspiro[4.5]decan-2-yl)benzonitrile were dissolved in DCE. To this solution was added NaBH(OAc)₃ and AcOH, and the reaction mixture was stirred at r.t. for 4 h. The reaction mixture was evaporated and purified by flash column chromatography on silica gel to give 2-chloro-4-((3S)-8-(5-((4-(1-(2-(2,6-dioxopiperidin-3-yl)-6-fluoro-1,3-dioxoisoindolin-5-yl)azetidin-3-yl)piperazin-1-yl)methyl)pyridin-2-yl)-3-methyl-2,8-diazaspiro[4.5]decan-2-yl)benzonitrile. LC-MS(ESI) m/z (M+H)⁺: 794.38; calcd: 794.34; >95% purity.

### Example 6. Synthesis of 2-chloro-4-((3S)-8-(6-((4-(1-(2-(2,6-dioxopiperidin-3-yl)-1,3-dioxoisoindolin-5-yl)azetidin-3-yl)piperazin-1-yl)methyl)pyridin-3-yl)-3-methyl-2,8-diazaspiro[4.5]decan-2-yl)benzonitrile (Cpd. No. 169)

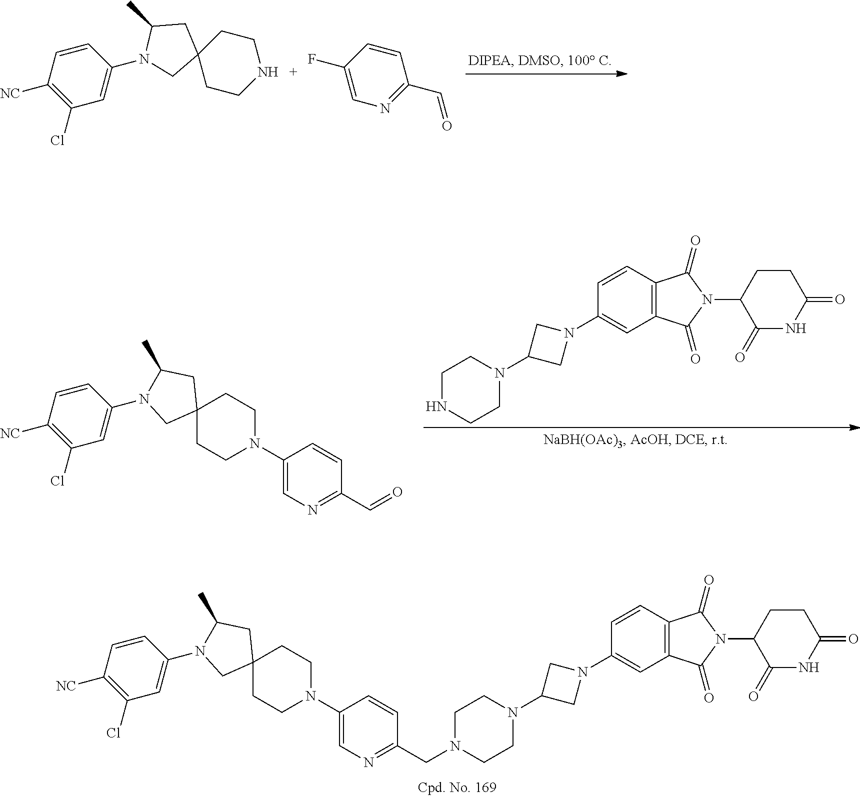 [SMILES: C[C@H]1CC2(CCNCC2)CN1c1ccc(C#N)c(Cl)c1 ; O=Cc1ccc(F)cn1 ; C[C@H]1CC2(CCN(c3ccc(C=O)nc3)CC2)CN1c1ccc(C#N)c(Cl)c1 ; O=C1CCC(N2C(=O)c3ccc(N4CC(N5CCNCC5)C4)cc3C2=O)C(=O)N1 ; C[C@H]1CC2(CCN(c3ccc(CN4CCN(C5CN(c6ccc7c(c6)C(=O)N(C6CCC(=O)NC6=O)C7=O)C5)CC4)nc3)CC2)CN1c1ccc(C#N)c(Cl)c1]

### Step 1: Synthesis of (S)-2-chloro-4-(8-(6-formylpyridin-3-yl)-3-methyl-2,8-diazaspiro[4.5]decan-2-yl)benzonitrile

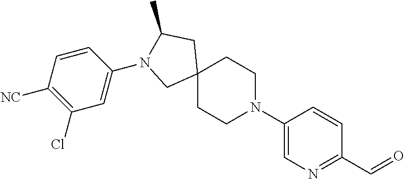 [SMILES: C[C@H]1CC2(CCN(c3ccc(C=O)nc3)CC2)CN1c1ccc(C#N)c(Cl)c1]

(S)-2-chloro-4-(3-methyl-2,8-diazaspiro[4.5]decan-2-yl)benzonitrile and 5-fluoropicolinaldehyde were dissolved in DMSO. To this solution was added DIPEA (5 eq.), and the reaction mixture was stirred at 100° C. for 4 h. The reaction mixture was partitioned between water and ethyl acetate. The organic phase was separated, washed with water, and dried over Na₂SO₄. The (S)-2-chloro-4-(8-(6-formylpyridin-3-yl)-3-methyl-2,8-diazaspiro[4.5]decan-2-yl)benzonitrile was obtained by removing the solvent under vacuum and purifying by flash column chromatography on silica gel. ESI-MS: 394.16.

### Step 2: Synthesis of 2-chloro-4-((3S)-8-(6-((4-(1-(2-(2,6-dioxopiperidin-3-yl)-1,3-dioxoisoindolin-5-yl)azetidin-3-yl)piperazin-1-yl)methyl)pyridin-3-yl)-3-methyl-2,8-diazaspiro[4.5]decan-2-yl)benzonitrile (Cpd. No. 169)

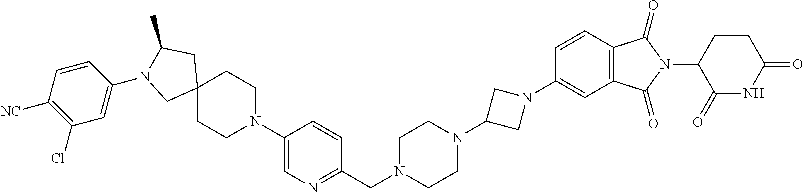 [SMILES: C[C@H]1CC2(CCN(c3ccc(CN4CCN(C5CN(c6ccc7c(c6)C(=O)N(C6CCC(=O)NC6=O)C7=O)C5)CC4)nc3)CC2)CN1c1ccc(C#N)c(Cl)c1]

2-(2,6-dioxopiperidin-3-yl)-5-(3-(piperazin-1-yl)azetidin-1-yl)isoindoline-1,3-dione and (S)-2-chloro-4-(8-(6-formylpyridin-3-yl)-3-methyl-2,8-diazaspiro[4.5]decan-2-yl)benzonitrile were dissolved in DCE. To this solution was added NaBH(OAc)₃ and AcOH, and the reaction mixture was stirred at r.t. for 4 h. The reaction mixture was evaporated and purified by flash column chromatography on silica gel to give 2-chloro-4-((3S)-8-(6-((4-(1-(2-(2,6-dioxopiperidin-3-yl)-1,3-dioxoisoindolin-5-yl)azetidin-3-yl)piperazin-1-yl)methyl)pyridin-3-yl)-3-methyl-2,8-diazaspiro[4.5]decan-2-yl)benzonitrile. LC-MS(ESI) m/z (M+H)⁺: 776.37; calcd: 776.35; >95% purity.

### Example 7. Synthesis of 2-chloro-4-((3S)-8-(2-((4-(1-(2-(2,6-dioxopiperidin-3-yl)-1,3-dioxoisoindolin-5-yl)azetidin-3-yl)piperazin-1-yl)methyl)pyrimidin-5-yl)-3-methyl-2,8-diazaspiro[4.5]decan-2-yl)benzonitrile (Cpd. No. 170)

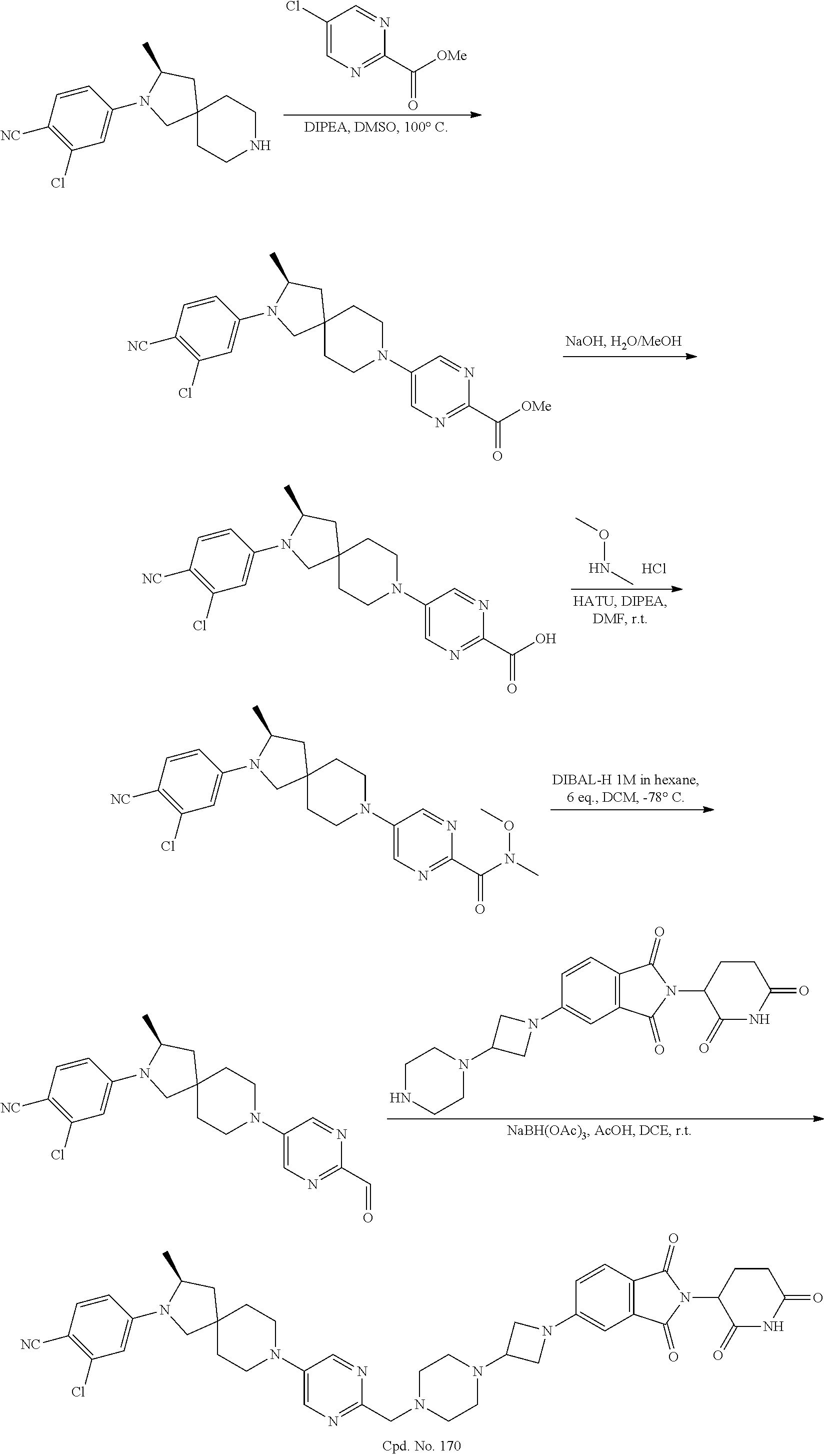

### Step 1: Synthesis of methyl (S)-5-(2-(3-chloro-4-cyanophenyl)-3-methyl-2,8-diazaspiro[4.5]decan-8-yl)pyrimidine-2-carboxylate

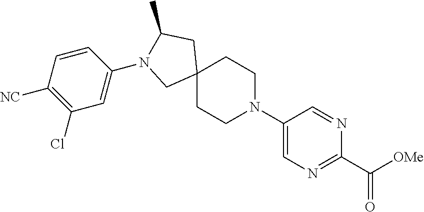 [SMILES: COC(=O)c1ncc(N2CCC3(CC2)C[C@H](C)N(c2ccc(C#N)c(Cl)c2)C3)cn1]

(S)-2-chloro-4-(3-methyl-2,8-diazaspiro[4.5]decan-2-yl)benzonitrile and methyl 5-chloropyrimidine-2-carboxylate were dissolved in DMSO. To this solution was added DIPEA (5 eq.), and the reaction mixture was stirred at 100° C. for 4 h. The reaction mixture was partitioned between water and ethyl acetate. The organic phase was separated, washed with water, and dried over Na₂SO₄. The methyl (S)-5-(2-(3-chloro-4-cyanophenyl)-3-methyl-2,8-diazaspiro[4.5]decan-8-yl)pyrimidine-2-carboxylate was obtained by removing the solvent under vacuum and purifying by flash column chromatography on silica gel. ESI-MS: 425.16.

### Step 2: Synthesis of (S)-5-(2-(3-chloro-4-cyanophenyl)-3-methyl-2,8-diazaspiro[4.5]decan-8-yl)pyrimidine-2-carboxylic acid

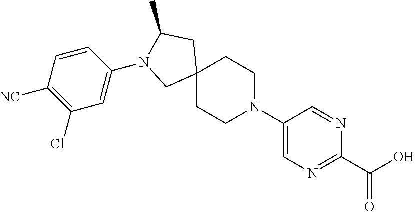 [SMILES: C[C@H]1CC2(CCN(c3cnc(C(=O)O)nc3)CC2)CN1c1ccc(C#N)c(Cl)c1]

NaOH (2 eq.) was added to a solution of methyl (S)-5-(2-(3-chloro-4-cyanophenyl)-3-methyl-2,8-diazaspiro[4.5]decan-8-yl)pyrimidine-2-carboxylate in MeOH/H₂O and stirred at rt for 2 h. Then the MeOH was removed under reduced pressure, the pH was adjusted to acidity with 2M HCl and the mixture was extracted with EtOAc. The solvent was removed to afford the product (S)-5-(2-(3-chloro-4-cyanophenyl)-3-methyl-2,8-diazaspiro[4.5]decan-8-yl)pyrimidine-2-carboxylic acid which was used without further purification. ESI-MS: 411.15.

### Step 3: Synthesis of (S)-5-(2-(3-chloro-4-cyanophenyl)-3-methyl-2,8-diazaspiro[4.5]decan-8-yl)-N-methoxy-N-methylpyrimidine-2-carboxamide

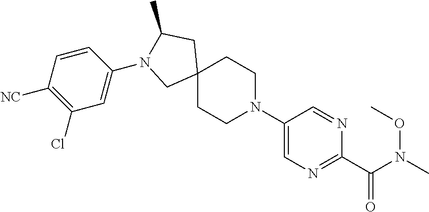 [SMILES: CON(C)C(=O)c1ncc(N2CCC3(CC2)C[C@H](C)N(c2ccc(C#N)c(Cl)c2)C3)cn1]

(S)-5-(2-(3-chloro-4-cyanophenyl)-3-methyl-2,8-diazaspiro[4.5]decan-8-yl)pyrimidine-2-carboxylic acid and N,O-dimethylhydroxylamine, hydrogen chloride were dissolved in DMF. To the solution was added DIPEA (5 eq.) and HATU (1.2 eq.), and the reaction mixture was stirred at r.t. for 1 hour. The reaction mixture was extracted by EA, washed by water, and the organic phase was dried by Na₂SO₄. (S)-5-(2-(3-chloro-4-cyanophenyl)-3-methyl-2,8-diazaspiro[4.5]decan-8-yl)-N-methoxy-N-methylpyrimidine-2-carboxamide was obtained by removing the solvent under vacuum and purified by flash column chromatography on silica gel. ESI-MS: 454.19.

### Step 4: Synthesis of (S)-2-chloro-4-(8-(2-formylpyrimidin-5-yl)-3-methyl-2,8-diazaspiro[4.5]decan-2-yl)benzonitrile

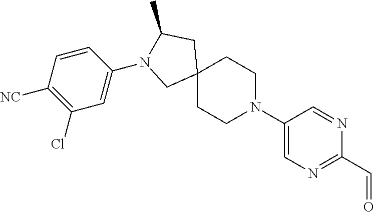 [SMILES: C[C@H]1CC2(CCN(c3cnc(C=O)nc3)CC2)CN1c1ccc(C#N)c(Cl)c1]

(S)-5-(2-(3-chloro-4-cyanophenyl)-3-methyl-2,8-diazaspiro[4.5]decan-8-yl)-N-methoxy-N-methylpyrimidine-2-carboxamide was dissolved in DCM. To the solution was added DIBAL-H (1.0 M in hexane, 6 eq.) and the reaction mixture was stirred at −78° C. for 0.5 hour. (S)-2-chloro-4-(8-(2-formylpyrimidin-5-yl)-3-methyl-2,8-diazaspiro[4.5]decan-2-yl)benzonitrile was obtained by removing the solvent under vacuum and purified by flash column chromatography on silica gel. ESI-MS: 395.15.

### Step 5: Synthesis of 2-chloro-4-((3S)-8-(2-((4-(1-(2-(2,6-dioxopiperidin-3-yl)-1,3-dioxoisoindolin-5-yl)azetidin-3-yl)piperazin-1-yl)methyl)pyrimidin-5-yl)-3-methyl-2,8-diazaspiro[4.5]decan-2-yl)benzonitrile (Cpd. No. 170)

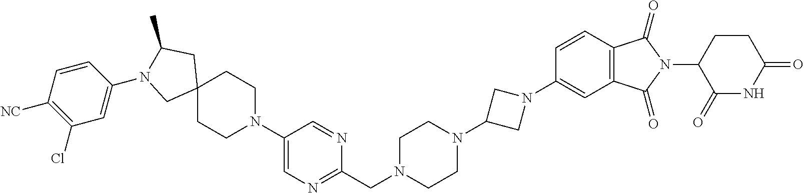 [SMILES: C[C@H]1CC2(CCN(c3cnc(CN4CCN(C5CN(c6ccc7c(c6)C(=O)N(C6CCC(=O)NC6=O)C7=O)C5)CC4)nc3)CC2)CN1c1ccc(C#N)c(Cl)c1]

2-(2,6-dioxopiperidin-3-yl)-5-(3-(piperazin-1-yl)azetidin-1-yl)isoindoline-1,3-dione and (S)-2-chloro-4-(8-(2-formylpyrimidin-5-yl)-3-methyl-2,8-diazaspiro[4.5]decan-2-yl)benzonitrile were dissolved in DCE. To this solution was added NaBH(OAc)₃ and AcOH, and the reaction mixture was stirred at r.t. for 4 h. The reaction mixture was evaporated and purified by flash column chromatography on silica gel to give 2-chloro-4-((3S)-8-(2-((4-(1-(2-(2,6-dioxopiperidin-3-yl)-1,3-dioxoisoindolin-5-yl)azetidin-3-yl)piperazin-1-yl)methyl)pyrimidin-5-yl)-3-methyl-2,8-diazaspiro[4.5]decan-2-yl)benzonitrile. LC-MS(ESI) m/z (M+H)⁺: 777.37; calcd: 777.34; >95% purity.

### Example 8. Synthesis of 2-chloro-4-((3S)-8-(5-((1′-(2-(2,6-dioxopiperidin-3-yl)-1,3-dioxoisoindolin-5-yl)-[4,4′-bipiperidin]-1-yl)methyl)pyridin-2-yl)-3-methyl-2,8-diazaspiro[4.5]decan-2-yl)benzonitrile (Cpd. No. 180)

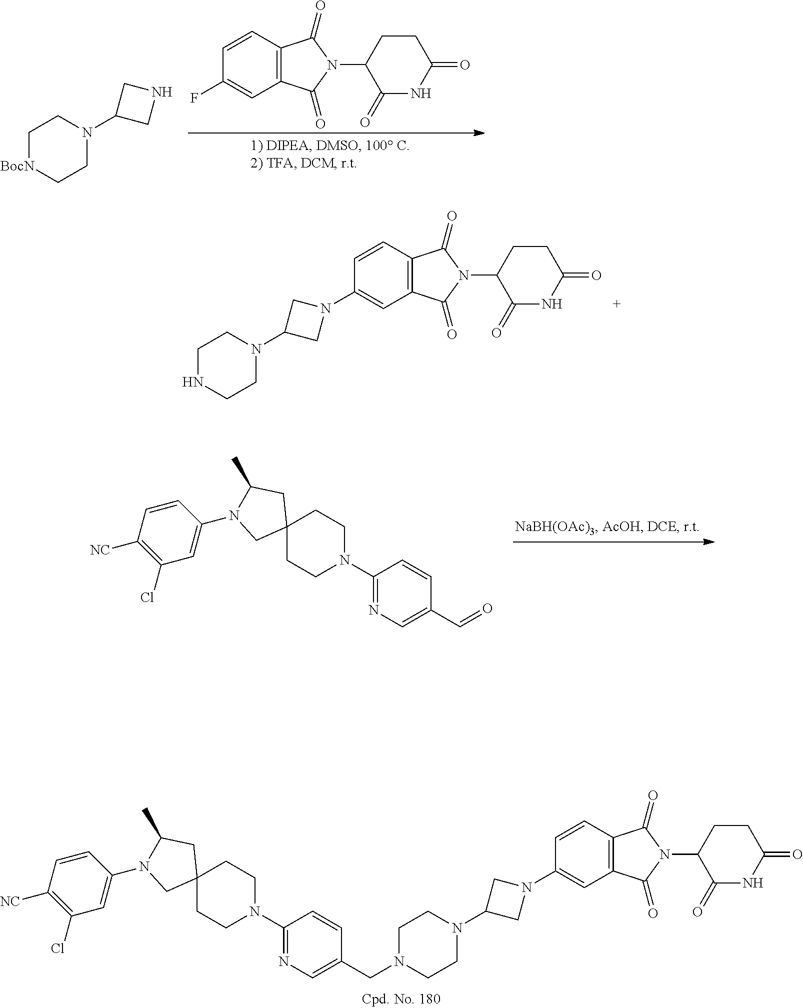

### Step 1: Synthesis of 5-([4,4′-bipiperidin]-1-yl)-2-(2,6-dioxopiperidin-3-yl)isoindoline-1,3-dione

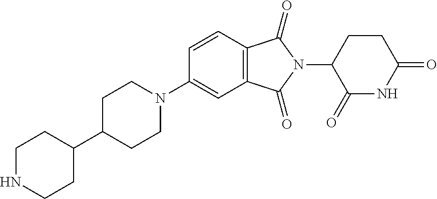 [SMILES: O=C1CCC(N2C(=O)c3ccc(N4CCC(C5CCNCC5)CC4)cc3C2=O)C(=O)N1]

tert-butyl [4,4′-bipiperidine]-1-carboxylate and 2-(2,6-dioxopiperidin-3-yl)-5,6-difluoroisoindoline-1,3-dione were dissolved in DMSO. To this solution was added DIPEA, and the reaction mixture was stirred at 100° C. for 6 h. The Boc-compound was obtained by removing the solvent under vacuum and purified by flash column. 5-([4,4′-bipiperidin]-1-yl)-2-(2,6-dioxopiperidin-3-yl)isoindoline-1,3-dione was obtained by removing the Boc group using TFA in DCM. ESI-MS: 424.21.

### Step 2: Synthesis of 2-chloro-4-((3S)-8-(5-((1′-(2-(2,6-dioxopiperidin-3-yl)-1,3-dioxoisoindolin-5-yl)-[4,4′-bipiperidin]-1-yl)methyl)pyridin-2-yl)-3-methyl-2,8-diazaspiro[4.5]decan-2-yl)benzonitrile (Cpd. No. 180)

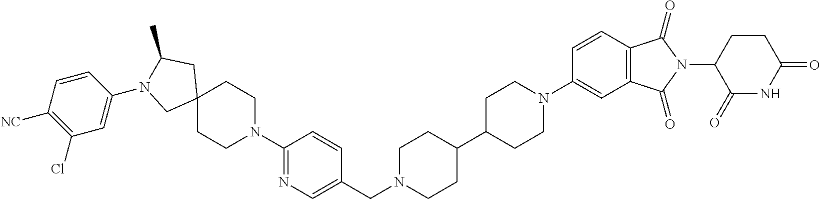 [SMILES: C[C@H]1CC2(CCN(c3ccc(CN4CCC(C5CCN(c6ccc7c(c6)C(=O)N(C6CCC(=O)NC6=O)C7=O)CC5)CC4)cn3)CC2)CN1c1ccc(C#N)c(Cl)c1]

5-([4,4′-bipiperidin]-1-yl)-2-(2,6-dioxopiperidin-3-yl)isoindoline-1,3-dione and (S)-2-chloro-4-(8-(5-formylpyridin-2-yl)-3-methyl-2,8-diazaspiro[4.5]decan-2-yl)benzonitrile were dissolved in DCE. To this solution was added NaBH(OAc)₃ and AcOH, and the reaction mixture was stirred at r.t. for 4 h. The reaction mixture was evaporated and purified by flash column chromatography on silica gel to give 2-chloro-4-((3 S)-8-(5-((1′-(2-(2,6-dioxopiperidin-3-yl)-1,3-dioxoisoindolin-5-yl)-[4,4′-bipiperidin]-1-yl)methyl)pyridin-2-yl)-3-methyl-2,8-diazaspiro[4.5]decan-2-yl)benzonitrile. ¹H NMR (400 MHz, DMSO-d₆) δ 11.08 (s, 1H), 10.06 (s, 1H), 8.14 (d, J=2.0 Hz, 1H), 7.68-7.55 (m, 3H), 7.30 (d, J=1.5 Hz, 1H), 7.27-7.16 (m, 1H), 6.88 (d, J=8.9 Hz, 1H), 6.79 (s, 1H), 6.66 (dd, J=9.0, 2.2 Hz, 1H), 5.07 (dd, J=12.8, 5.4 Hz, 1H), 4.23-3.98 (m, 5H), 3.47-3.31 (m, 14H), 2.85 (dt, J=37.2, 17.4 Hz, 5H), 2.60-2.48 (m, 2H), 2.25 (dd, J=12.7, 7.7 Hz, 1H), 2.05-1.97 (m, 1H), 1.93-1.85 (m, 2H), 1.79-1.56 (m, 5H), 1.20 (d, J=6.0 Hz, 3H). LC-MS(ESI) m/z (M+H)⁺: 803.36; calcd: 803.38; >95% purity.

### Example 9. Synthesis of 2-chloro-4-((3S)-8-(4-(4-(3-((2-(2,6-dioxopiperidin-3-yl)-1,3-dioxoisoindolin-5-yl)amino)azetidin-1-yl)piperidine-1-carbonyl)phenyl)-3-methyl-2,8-diazaspiro[4.5]decan-2-yl)benzonitrile (Cpd. No. 206)

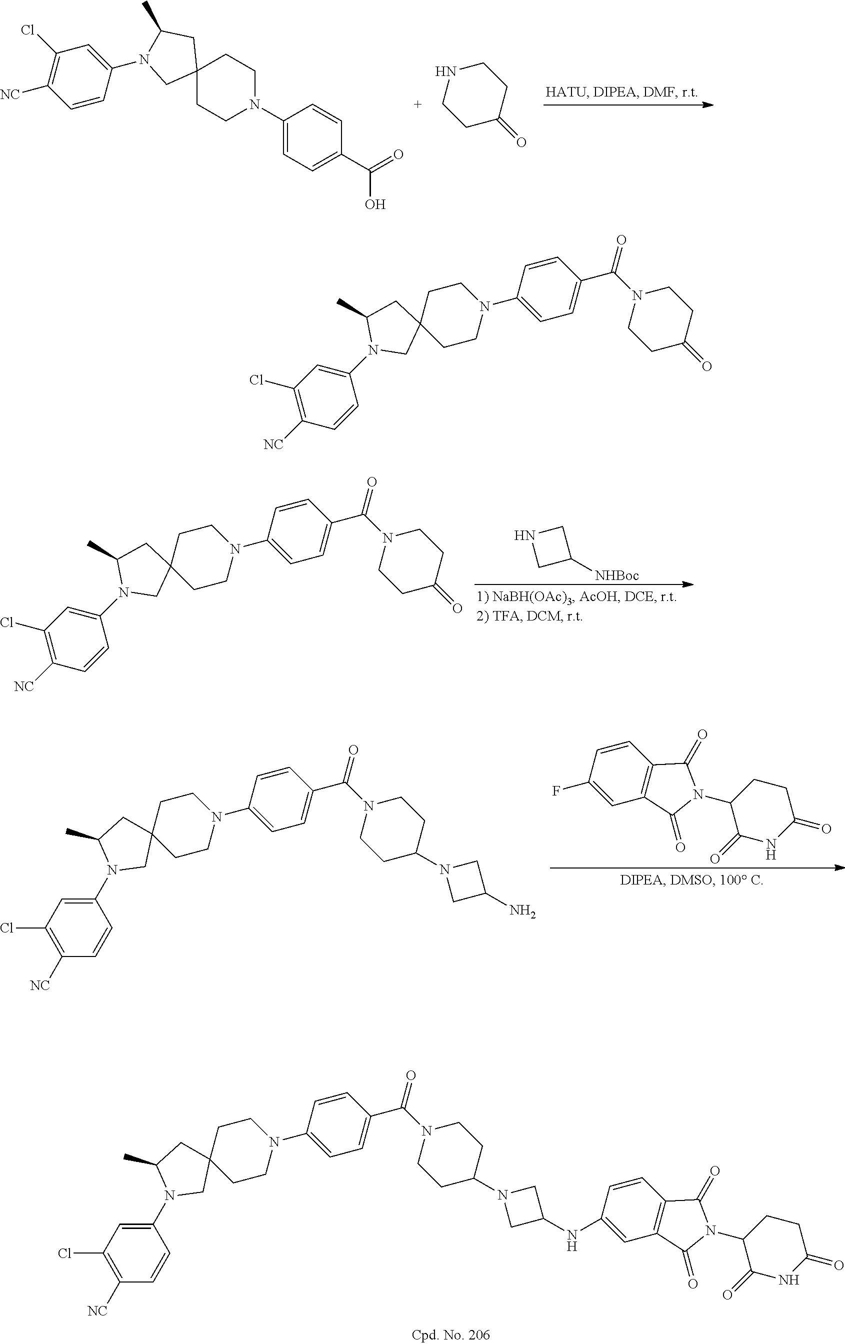

### Step 1: Synthesis of (S)-2-chloro-4-(3-methyl-8-(4-(4-oxopiperidine-1-carbonyl)phenyl)-2,8-diazaspiro[4.5]decan-2-yl)benzonitrile

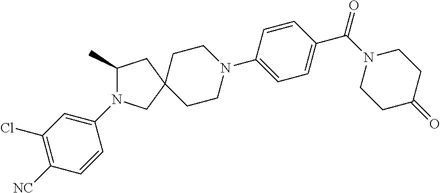 [SMILES: C[C@H]1CC2(CCN(c3ccc(C(=O)N4CCC(=O)CC4)cc3)CC2)CN1c1ccc(C#N)c(Cl)c1]

(S)-4-(2-(3-chloro-4-cyanophenyl)-3-methyl-2,8-diazaspiro[4.5]decan-8-yl)benzoic acid and piperidin-4-one were dissolved in DMF. To this solution was added DIPEA (5 eq.) and HATU (1.2 eq.), and the reaction mixture was stirred at r.t. for 1 h. The reaction mixture was partitioned between water and ethyl acetate. The organic phase was separated, washed with water, dried over Na₂SO₄, and purified by flash column chromatography on silica gel to give (S)-2-chloro-4-(3-methyl-8-(4-(4-oxopiperidine-1-carbonyl)phenyl)-2,8-diazaspiro[4.5]decan-2-yl)benzonitrile. ESI-MS: 490.21.

### Step 2: Synthesis of (S)-4-(8-(4-(4-(3-aminoazetidin-1-yl)piperidine-1-carbonyl)phenyl)-3-methyl-2,8-diazaspiro[4.5]decan-2-yl)-2-chlorobenzonitrile

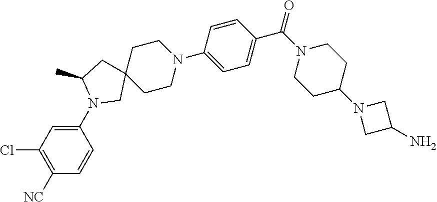 [SMILES: C[C@H]1CC2(CCN(c3ccc(C(=O)N4CCC(N5CC(N)C5)CC4)cc3)CC2)CN1c1ccc(C#N)c(Cl)c1]

(S)-2-chloro-4-(3-methyl-8-(4-(4-oxopiperidine-1-carbonyl)phenyl)-2,8-diazaspiro[4.5]decan-2-yl)benzonitrile and tert-butyl azetidin-3-ylcarbamate were dissolved in DCE. To this solution was added NaBH(OAc)₃ and AcOH, and the reaction mixture was stirred at r.t. for 4 h. The reaction mixture was evaporated and purified by flash column chromatography on silica gel to give the Boc-compound. (S)-4-(8-(4-(4-(3-aminoazetidin-1-yl)piperidine-1-carbonyl)phenyl)-3-methyl-2,8-diazaspiro[4.5]decan-2-yl)-2-chlorobenzonitrile was obtained by removing the Boc group using TFA in DCM. ESI-MS: 546.29.

### Step 3: Synthesis of 2-chloro-4-((3S)-8-(4-(4-(3-((2-(2,6-dioxopiperidin-3-yl)-1,3-dioxoisoindolin-5-yl)amino)azetidin-1-yl)piperidine-1-carbonyl)phenyl)-3-methyl-2,8-diazaspiro[4.5]decan-2-yl)benzonitrile

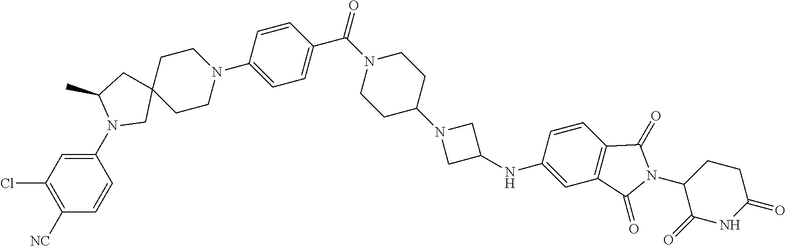 [SMILES: C[C@H]1CC2(CCN(c3ccc(C(=O)N4CCC(N5CC(Nc6ccc7c(c6)C(=O)N(C6CCC(=O)NC6=O)C7=O)C5)CC4)cc3)CC2)CN1c1ccc(C#N)c(Cl)c1]

(S)-4-(8-(4-(4-(3-aminoazetidin-1-yl)piperidine-1-carbonyl)phenyl)-3-methyl-2,8-diazaspiro[4.5]decan-2-yl)-2-chlorobenzonitrile and 2-(2,6-dioxopiperidin-3-yl)-5-fluoroisoindoline-1,3-dione were dissolved in DMSO. To this solution was added DIPEA, and the reaction mixture was stirred at 100° C. for 6 h. The 2-chloro-4-((3S)-8-(4-(4-(3-((2-(2,6-dioxopiperidin-3-yl)-1,3-dioxoisoindolin-5-yl)amino)azetidin-1-yl)piperidine-1-carbonyl)phenyl)-3-methyl-2,8-diazaspiro[4.5]decan-2-yl)benzonitrile was obtained by removing the solvent under vacuum and purified by flash column. LC-MS(ESI) m/z (M+H)⁺: 803.38; calcd: 803.35; >95% purity.

### Example 10. Synthesis of 2-chloro-4-((3S)-8-(4-(4-(3-((2-(2,6-dioxopiperidin-3-yl)-1,3-dioxoisoindolin-5-yl)(methyl)amino)azetidin-1-yl)piperidine-1-carbonyl)phenyl)-3-methyl-2,8-diazaspiro[4.5]decan-2-yl)benzonitrile (Cpd. No. 207)

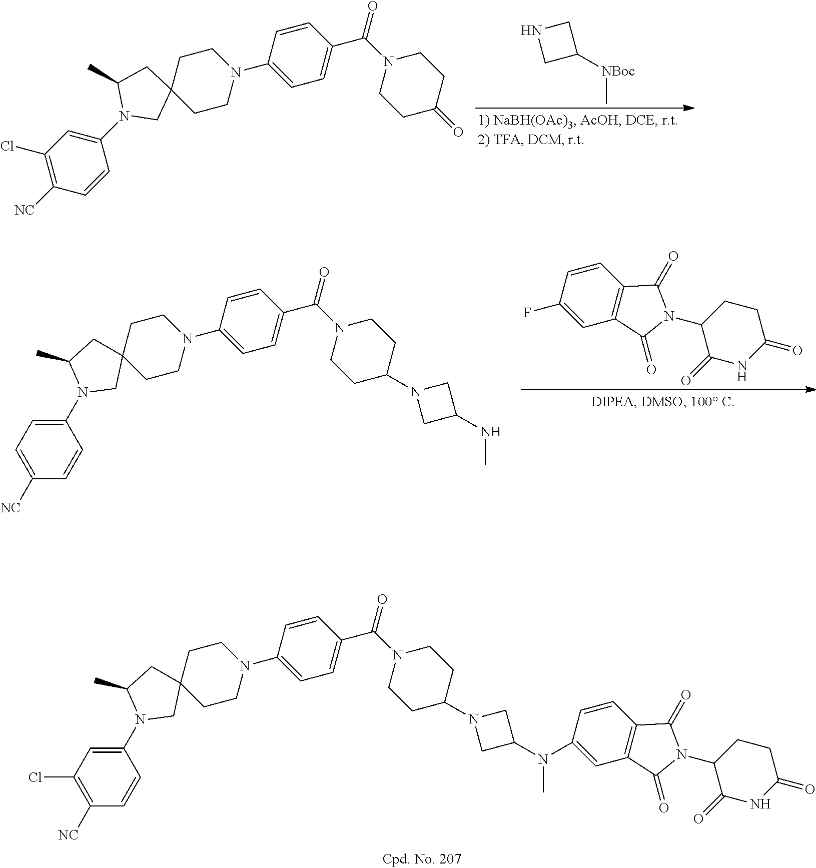 [SMILES: C[C@H]1CC2(CCN(c3ccc(C(=O)N4CCC(=O)CC4)cc3)CC2)CN1c1ccc(C#N)c(Cl)c1 ; CN(C(=O)OC(C)(C)C)C1CNC1 ; CNC1CN(C2CCN(C(=O)c3ccc(N4CCC5(CC4)C[C@H](C)N(c4ccc(C#N)cc4)C5)cc3)CC2)C1 ; O=C1CCC(N2C(=O)c3ccc(F)cc3C2=O)C(=O)N1 ; C[C@H]1CC2(CCN(c3ccc(C(=O)N4CCC(N5CC(N(C)c6ccc7c(c6)C(=O)N(C6CCC(=O)NC6=O)C7=O)C5)CC4)cc3)CC2)CN1c1ccc(C#N)c(Cl)c1]

### Step 1: Synthesis of (S)-2-chloro-4-(3-methyl-8-(4-(4-(3-(methylamino)azetidin-1--yl)piperidine-1-carbonyl)phenyl)-2,8-diazaspiro[4.5]decan-2-yl)benzonitrile

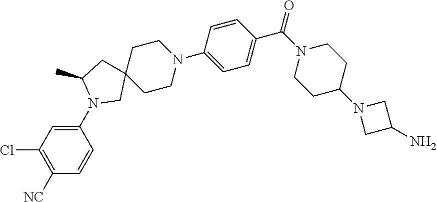 [SMILES: C[C@H]1CC2(CCN(c3ccc(C(=O)N4CCC(N5CC(N)C5)CC4)cc3)CC2)CN1c1ccc(C#N)c(Cl)c1]

(S)-2-chloro-4-(3-methyl-8-(4-(4-oxopiperidine-1l-carbonyl)phenyl)-2,8-diazaspiro[4.5]decan-2-yl)benzonitrile and tert-butyl azetidin-3-yl(methyl)carbamate were dissolved in DCE. To this solution was added NaBH(OAc)₃ and AcOH, and the reaction mixture was stirred at r.t. for 4 h. The reaction mixture was evaporated and purified by flash column chromatography on silica gel to give the Boc-compound. (S)-2-chloro-4-(3-methyl-8-(4-(4-(3-(methylamino)azetidin-1l-yl)piperidine-1-carbonyl)phenyl)-2,8-diazaspiro[4.5]decan-2-yl)benzonitrile was obtained by removing the Boc group using TFA in DCM. ESI-MS: 560.30.

### Step 2: Synthesis of 2-chloro-4-((3S)-8-(4-(4-(3-((2-(2,6-dioxopiperidin-3-yl)-1,3-dioxoisoindolin-5-yl)(methyl)amino)azetidin-1-yl)piperidine-1-carbonyl)phenyl)-3-methyl-2,8-diazaspiro[4.5]decan-2-yl)benzonitrile

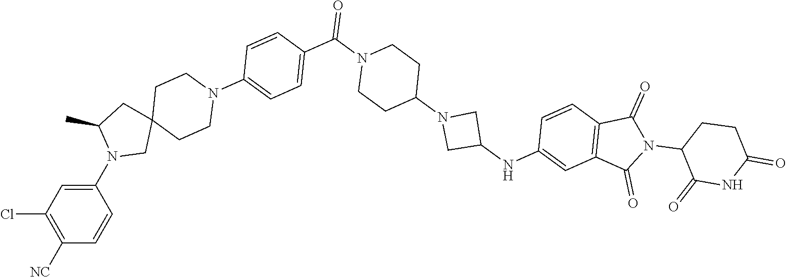 [SMILES: C[C@H]1CC2(CCN(c3ccc(C(=O)N4CCC(N5CC(Nc6ccc7c(c6)C(=O)N(C6CCC(=O)NC6=O)C7=O)C5)CC4)cc3)CC2)CN1c1ccc(C#N)c(Cl)c1]

(S)-2-chloro-4-(3-methyl-8-(4-(4-(3-(methylamino)azetidin-1-yl)piperidine-1-carbonyl)phenyl)-2,8-diazaspiro[4.5]decan-2-yl)benzonitrile and 2-(2,6-dioxopiperidin-3-yl)-5-fluoroisoindoline-1,3-dione were dissolved in DMSO. To this solution was added DIPEA, and the reaction mixture was stirred at 100° C. for 6 h. The 2-chloro-4-((3 S)-8-(4-(4-(3-((2-(2,6-dioxopiperidin-3-yl)-1,3-dioxoisoindolin-5-yl)(methyl)amino)azetidin-1-yl)piperidine-1-carbonyl)phenyl)-3-methyl-2,8-diazaspiro[4.5]decan-2-yl)benzonitrile was obtained by removing the solvent under vacuum and purified by flash column. LC-MS(ESI) m/z (M+H)⁺: 817.38; calcd: 817.36; >95% purity.

### Example 11. Synthesis of 2-chloro-4-((3S)-8-(4-(4-((1-(2-(2,6-dioxopiperidin-3-yl)-1,3-dioxoisoindolin-5-yl)azetidin-3-yl)methyl)piperazin-1-yl)phenyl)-3-methyl-2,8-diazaspiro[4.5]decan-2-yl)benzonitrile (Cpd. No. 28)

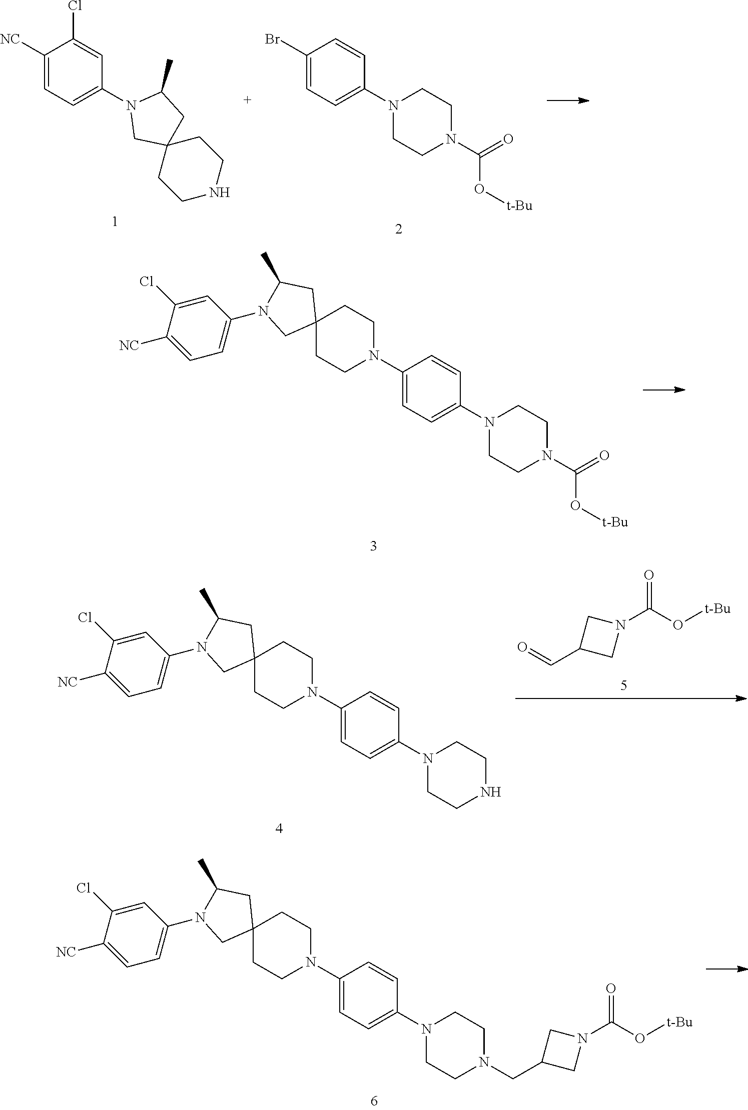 [SMILES: C[C@H]1CC2(CCNCC2)CN1c1ccc(C#N)c(Cl)c1 ; CC(C)(C)OC(=O)N1CCN(c2ccc(Br)cc2)CC1 ; C[C@H]1CC2(CCN(c3ccc(N4CCN(C(=O)OC(C)(C)C)CC4)cc3)CC2)CN1c1ccc(C#N)c(Cl)c1 ; C[C@H]1CC2(CCN(c3ccc(N4CCNCC4)cc3)CC2)CN1c1ccc(C#N)c(Cl)c1 ; CC(C)(C)OC(=O)N1CC(C=O)C1 ; C[C@H]1CC2(CCN(c3ccc(N4CCN(CC5CN(C(=O)OC(C)(C)C)C5)CC4)cc3)CC2)CN1c1ccc(C#N)c(Cl)c1]

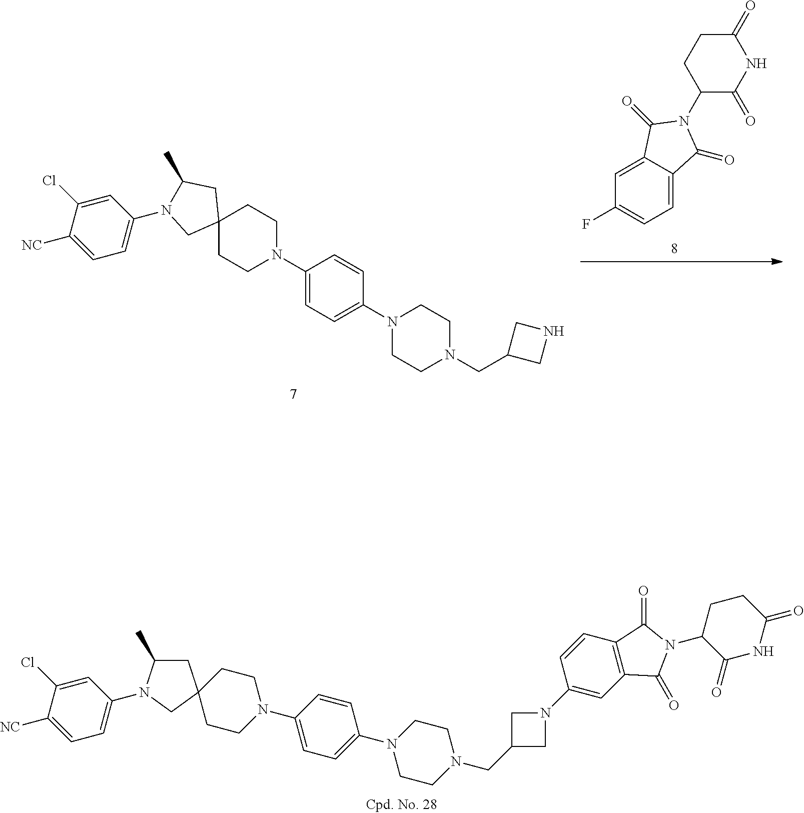 [SMILES: C[C@H]1CC2(CCN(c3ccc(N4CCN(CC5CNC5)CC4)cc3)CC2)CN1c1ccc(C#N)c(Cl)c1 ; O=C1CCC(N2C(=O)c3ccc(F)cc3C2=O)C(=O)N1 ; C[C@H]1CC2(CCN(c3ccc(N4CCN(CC5CN(c6ccc7c(c6)C(=O)N(C6CCC(=O)NC6=O)C7=O)C5)CC4)cc3)CC2)CN1c1ccc(C#N)c(Cl)c1]

Step 1: Compound 1 (1.0 eq), compound 2 (1.0 eq), Pd₂(dba)₃ (0.1 eq), Xphos (0.1 eq) and Cs₂CO₃ (4.0 eq) were dissolved in dioxane. The mixture was degassed and stirred at 110° C. overnight. The reaction mixture was concentrated with silica gel, and purified by Combiflash using DCM and MeOH to afford compound 3.

Step 2: Compound 3 was dissolved in DCM and TFA (10×) was added. All the volatiles were removed after 0.5 h to afford compound 4.

Step 3: Compound 4 (1.0 eq) and compound 5 (1.5 eq), AcOH (3.0 eq) were dissolved in DCE and stirred at rt for 4 h. NaBH(AcO)₃ (4.5 eq) was added. The reaction was complete in ˜3 h. The reaction mixture was directly purified by Combiflash with DCM and MeOH to afford compound 6.

Step 4: Compound 6 was dissolved in DCM and TFA (10×) was added. All the volatiles were removed after 0.5 h to afford compound 7.

Step 5: Compound 7 (1.0 eq), compound 8 (1.3 eq), and DIPEA (5.0 eq) were dissolved in DMF and stirred at 100° C. overnight. The reaction mixture was acidified with TFA, diluted with water, and purified by prep HPLC to give Cpd. No. 28 in 38% yield. UPLC-MS: 3.6 min, 775.21

The following compounds were prepared using a similar protocol.

Cpd. No. 30: UPLC-MS: 3.9 min, 803.15; prep HPLC: 39%.

Cpd. No. 31: UPLC-MS: 4.2 min, 776.29; prep HPLC: 41.5%.

Cpd. No. 34: UPLC-MS: 4.3 min, 776.24; prep HPLC: 41%.

Cpd. No. 43: UPLC-MS: 4.3 min, 775.34, prep HPLC 43%.

Cpd. No. 45: UPLC-MS: 4.2 min, 775.36, prep HPLC 43%.

Cpd. No. 47: UPLC-MS: 3.5 min, 776.99; prep HPLC: 37%.

Cpd. No. 82: UPLC-MS: 4.6 min, 777.26; prep HPLC: 39%.

### Example 12. Synthesis of 2-chloro-4-((3S)-8-(4-(4-((1-(2-(2,6-dioxopiperidin-3-yl)-1,3-dioxoisoindolin-5-yl)azetidin-3-yl)(methyl)amino)piperidin-1-yl)phenyl)-3-methyl-2,8-diazaspiro[4.5]decan-2-yl)benzonitrile (Cpd. No. 119)

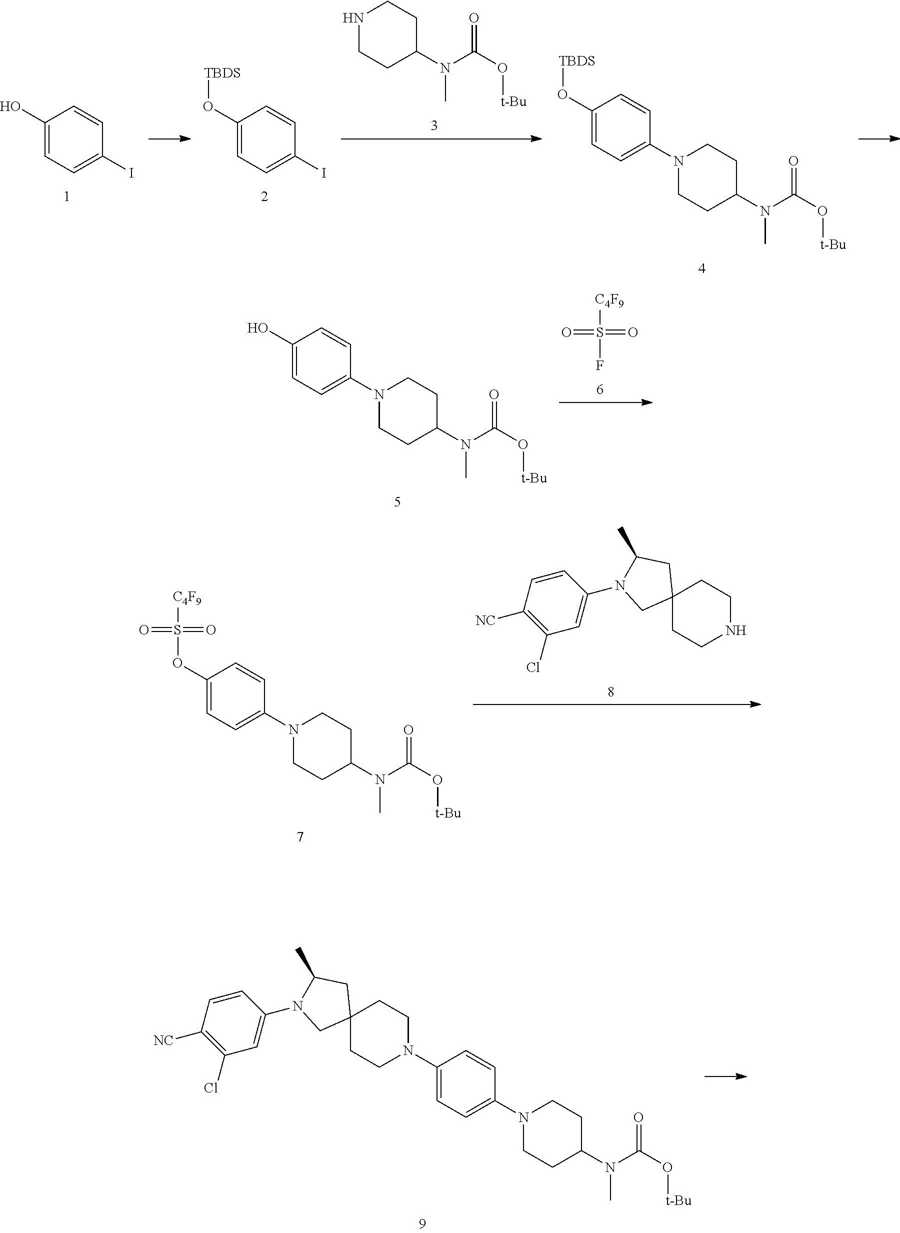

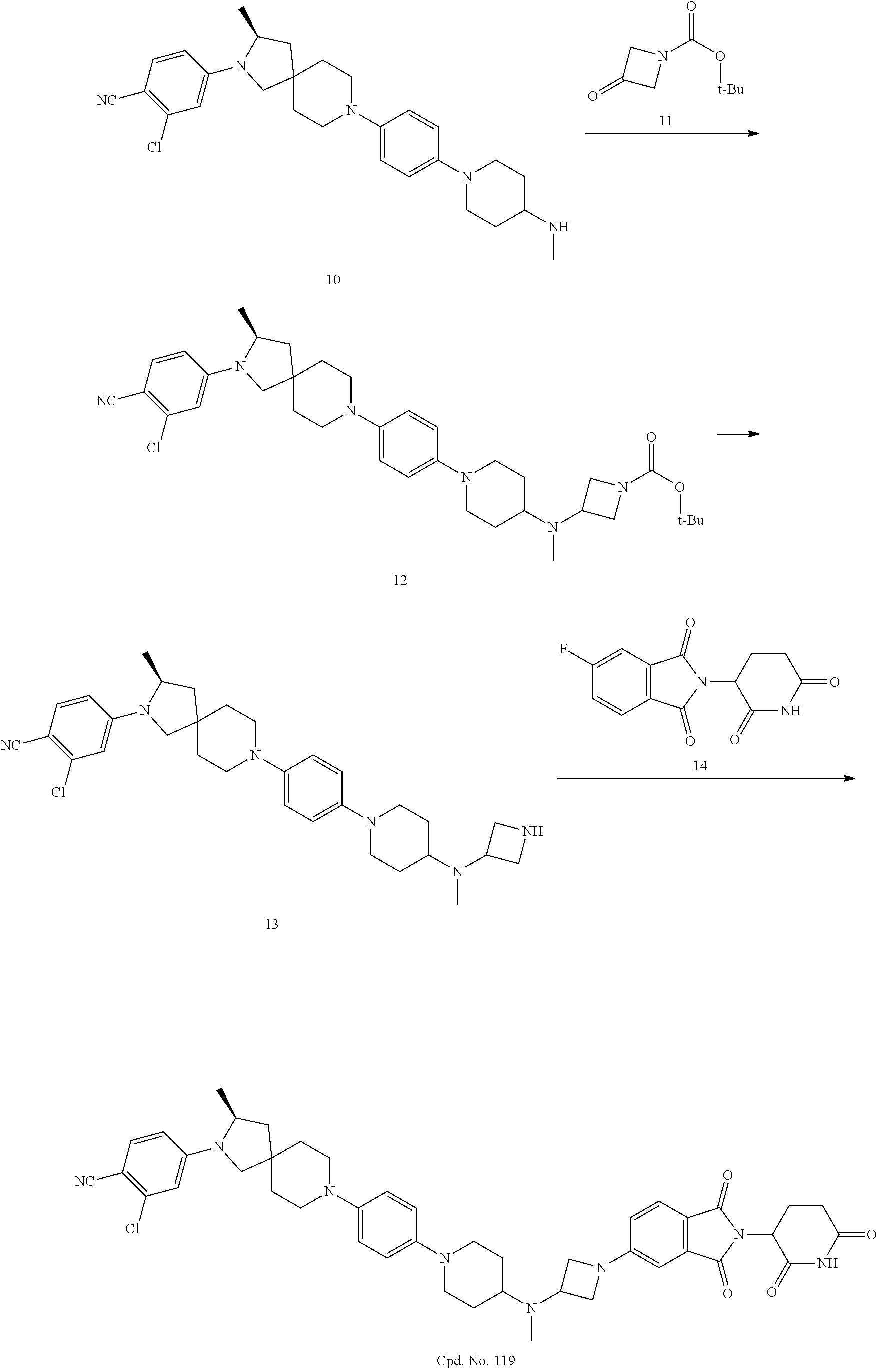 [SMILES: CNC1CCN(c2ccc(N3CCC4(CC3)C[C@H](C)N(c3ccc(C#N)c(Cl)c3)C4)cc2)CC1 ; CC(C)(C)OC(=O)N1CC(=O)C1 ; C[C@H]1CC2(CCN(c3ccc(N4CCC(N(C)C5CN(C(=O)OC(C)(C)C)C5)CC4)cc3)CC2)CN1c1ccc(C#N)c(Cl)c1 ; C[C@H]1CC2(CCN(c3ccc(N4CCC(N(C)C5CNC5)CC4)cc3)CC2)CN1c1ccc(C#N)c(Cl)c1 ; O=C1CCC(N2C(=O)c3ccc(F)cc3C2=O)C(=O)N1 ; C[C@H]1CC2(CCN(c3ccc(N4CCC(N(C)C5CN(c6ccc7c(c6)C(=O)N(C6CCC(=O)NC6=O)C7=O)C5)CC4)cc3)CC2)CN1c1ccc(C#N)c(Cl)c1]

Step 1: Compound 1 (1.0 eq) (10×), imidazole (3×), and TBSCl (1.5 eq) in DMF were stirred at 0° C. overnight. The reaction mixture was partitioned with H₂O and EtOAc. Organic layer was dried, concentrated and purified with Combiflash using Hexane and EtOA to give compound 2.

Step 2: Compound 2 (1.0 eq), compound 3 (1.0 eq), Pd₂(dba)₃ (0.1 eq), Xphos (0.1 eq), and Cs₂CO₃ (4.0 eq) were dissolved in dioxane. The mixture was degassed and stirred at 110° C. overnight. The reaction mixture was concentrated with silica gel and purified by Combiflash using hexane and EtOAc to afford compound 4 approximated 35% yield.

Step 3: Compound 4 was dissolved in THF at 0° C. and TBFA (1 N THF, 3 eq) was added. The deprotection was complete in approximately 2 h. Compound 5 was obtained from Combiflash with Hexane and EtOAc.

Step 4: Compound 5 (1.0 eq), compound 6 (2 eq), and K₂CO₃ (3.0 eq) were dissolved in THF/acetonitrile at rt. The reaction complete in approximately 2 days. Compound 7 was obtained from Combiflash with Hexane and EtOAc.

Step 5: Compound 7 (1.0 eq), compound 8 (1.0 eq), Pd(AcO)₂ (0.1 eq), Binap (0.1 eq), and Cs₂CO₃ (4.0 eq) were dissolved in dioxane. The mixture was degassed and stirred at 110° C. overnight. The reaction mixture was concentrated with silica gel and purified by Combiflash using hexane and EtOAc to afford compound 9 in about 30% yield.

Step 6: Compound 9 was dissolved in DCM and TFA (10×) was added. All the volatiles were removed after 0.5 h to afford compound 10.

Step 7: Compound 10 (1.0 eq), compound 11 (1.5 eq), AcOH (3.0 eq) were dissolved in DCE and stirred at rt for 0.5 h. NaBH(AcO)₃ (4.5 eq) was added. The reaction was completed in ˜3 h. The reaction mixture was purified by Combiflash with DCM and MeOH to afford compound 12.

Step 8: Compound 12 was dissolved in DCM and TFA (10×) was added. All the volatiles were removed after 0.5 h to afford compound 13.

Step 9: Compound 13 (1.0 eq), compound 14 (1.3 eq), and DIPEA (5.0 eq) were dissolved in DMF and stirred at 100° C. overnight. The reaction mixture was acidified with TFA, diluted with water, and purified by prep HPLC to give Cpd. No. 119.

### Example 13. Synthesis of 2-chloro-4-((3S)-8-(5-((1-(1-(2-(2,6-dioxopiperidin-3-yl)-1,3-dioxoisoindolin-5-yl)azetidin-3-yl)piperidin-4-yl)oxy)pyridin-2-yl)-3-methyl-2,8-diazaspiro-[4.5]decan-2-yl)benzonitrile (Cpd. No. 61)

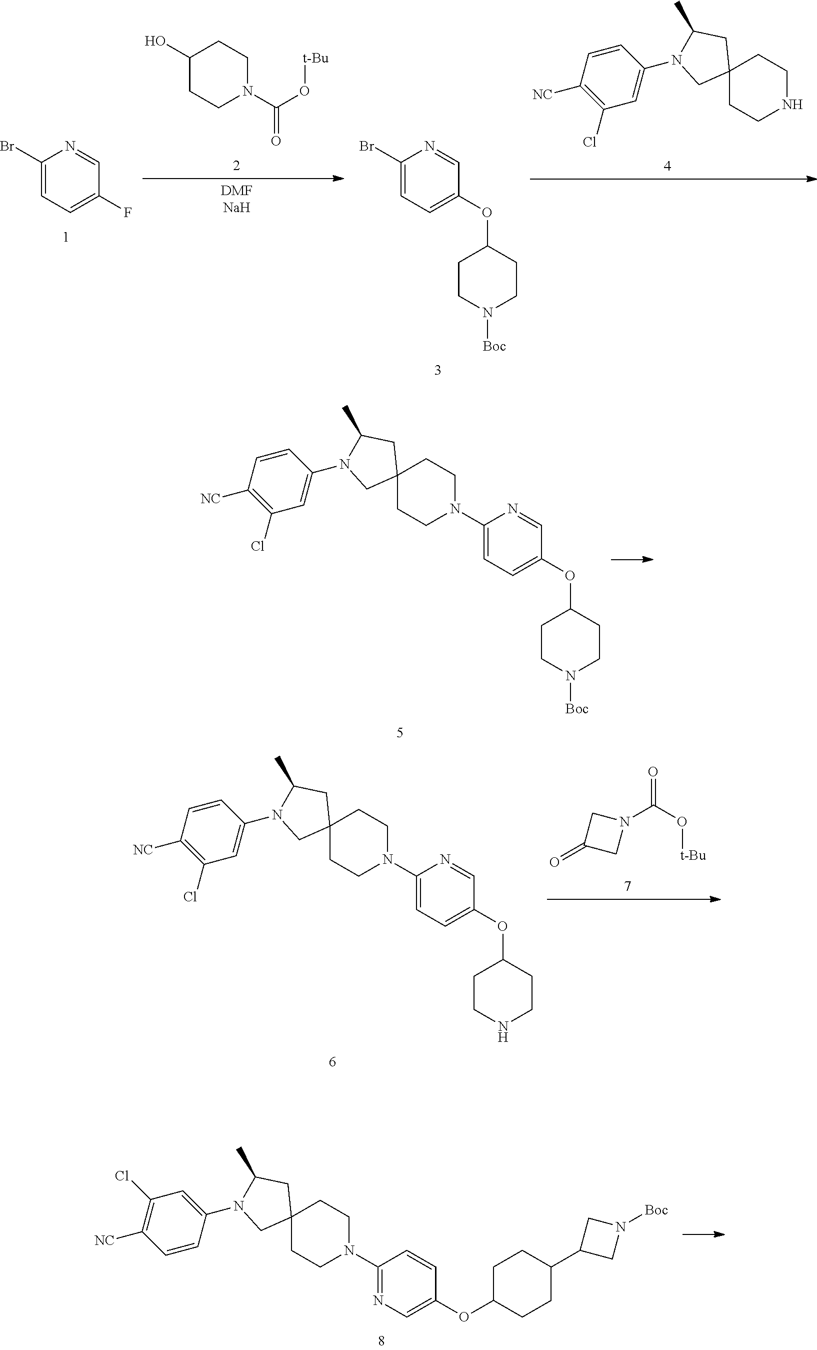

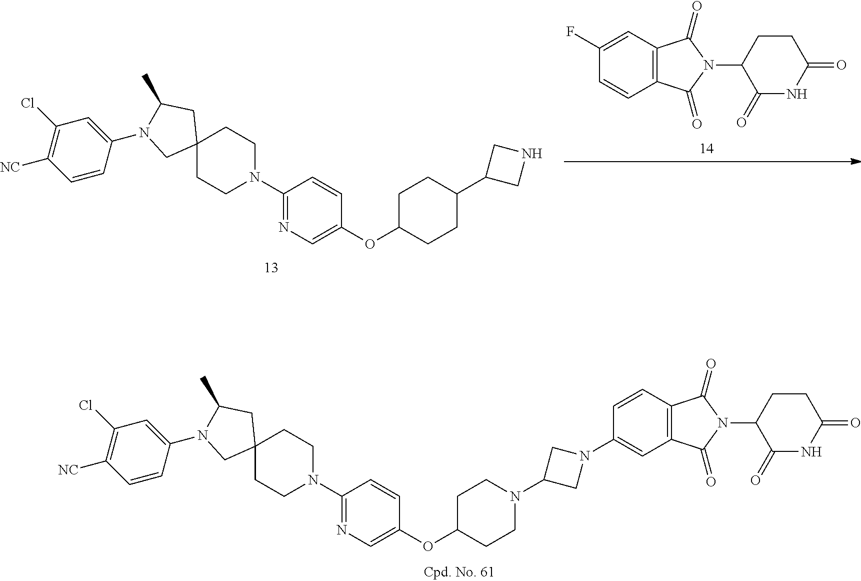 [SMILES: C[C@H]1CC2(CCN(c3ccc(OC4CCC(C5CNC5)CC4)cn3)CC2)CN1c1ccc(C#N)c(Cl)c1 ; O=C1CCC(N2C(=O)c3ccc(F)cc3C2=O)C(=O)N1 ; C[C@H]1CC2(CCN(c3ccc(OC4CCN(C5CN(c6ccc7c(c6)C(=O)N(C6CCC(=O)NC6=O)C7=O)C5)CC4)cn3)CC2)CN1c1ccc(C#N)c(Cl)c1]

Step 1: Compound 2 (1.0 eq) was dissolved in DMF at 0° C. and NaH (60% mineral oil, 1.6 eq) was added slowly. After 0.5 h, the mixture was warmed to rt and sustained for 0.5 h. The reaction was cooled to 0° C., and compound 1 (1.1 eq) was added. The reaction was allowed to warm back to rt and stirred overnight The reaction mixture was partitioned between H₂O and EtOAc, separated, dried, and purified with Combiflash using Hexane and EtOAc to give compound 3.

Step 2: Compound 3 (1.0 eq), compound 4 (1.0 eq), Pd₂(dba)₃ (0.1 eq), Xphos (0.1 eq), and Cs₂CO₃ (4.0 eq) were dissolved in dioxane. The mixture was degassed and stirred at 110° C. overnight. The reaction mixture was concentrated with silica gel and purified by Combiflash using hexane and EtOAc to afford compound 5.

Cpd. No. 61 was prepared in 36% yield from compound 5 using the procedures described above. (UPLC-MS: 4.2 min, 777.14).

The following compounds were prepared using a similar protocol.

Cpd. No. 62: UPLC-MS: 4.0 min, 778.26; prep HPLC: 38%.

Cpd. No. 63: UPLC-MS: 4.6 min, 777.13; prep HPLC: 37%.

### Example 14. Synthesis of 2-chloro-4-((S)-8-(4-(4-((6-((R)-2,6-dioxopiperidin-3-yl)-5,7-dioxo-3,5,6,7-tetrahydropyrrolo[3,4-f]isoindol-2(1H)-yl)methyl)piperidine-1-carbonyl)-phenyl)-3-methyl-2,8-diazaspiro[4.5]decan-2-yl)benzonitrile (Cpd. No. 78) and 2-chloro-4-((S)-8-(4-(4-((6-((R)-2,6-dioxopiperidin-3-yl)-5,7-dioxo-3,5,6,7-tetrahydropyrrolo[3,4-f]isoindol-2(1H)-yl)methyl)piperidine-1-carbonyl)phenyl)-3-methyl-2,8-diazaspiro-[4.5]decan-2-yl)benzonitrile (Cpd. No. 79)

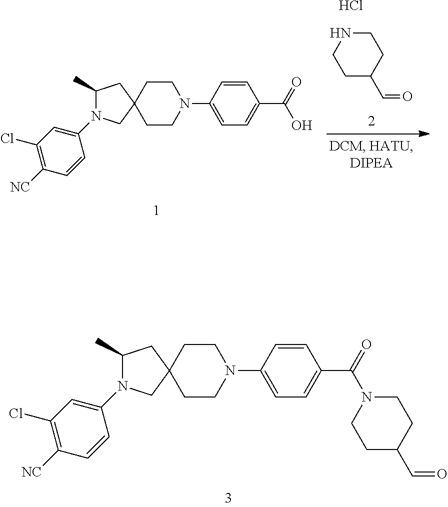 [SMILES: Cl ; C[C@H]1CC2(CCN(c3ccc(C(=O)O)cc3)CC2)CN1c1ccc(C#N)c(Cl)c1 ; O=CC1CCNCC1 ; C[C@H]1CC2(CCN(c3ccc(C(=O)N4CCC(C=O)CC4)cc3)CC2)CN1c1ccc(C#N)c(Cl)c1]

Compound 1 (1.0 eq) was dissolved in DCM and basified with DIPEA (3.0 eq). HATU (1.2 eq) was added to the above solution and stirred for 15 min. Compound 2 (1.05 eq) was dissolved in DCM and basified with DIPEA (2.0 eq). The compound 2 solution was added to the compound 1 solution, and the reaction mixture was stirred for 0.5 h. All volatiles were removed and the residue was purified by Combiflash with DCM and MeOH to afford compound 3 in 85% yield.

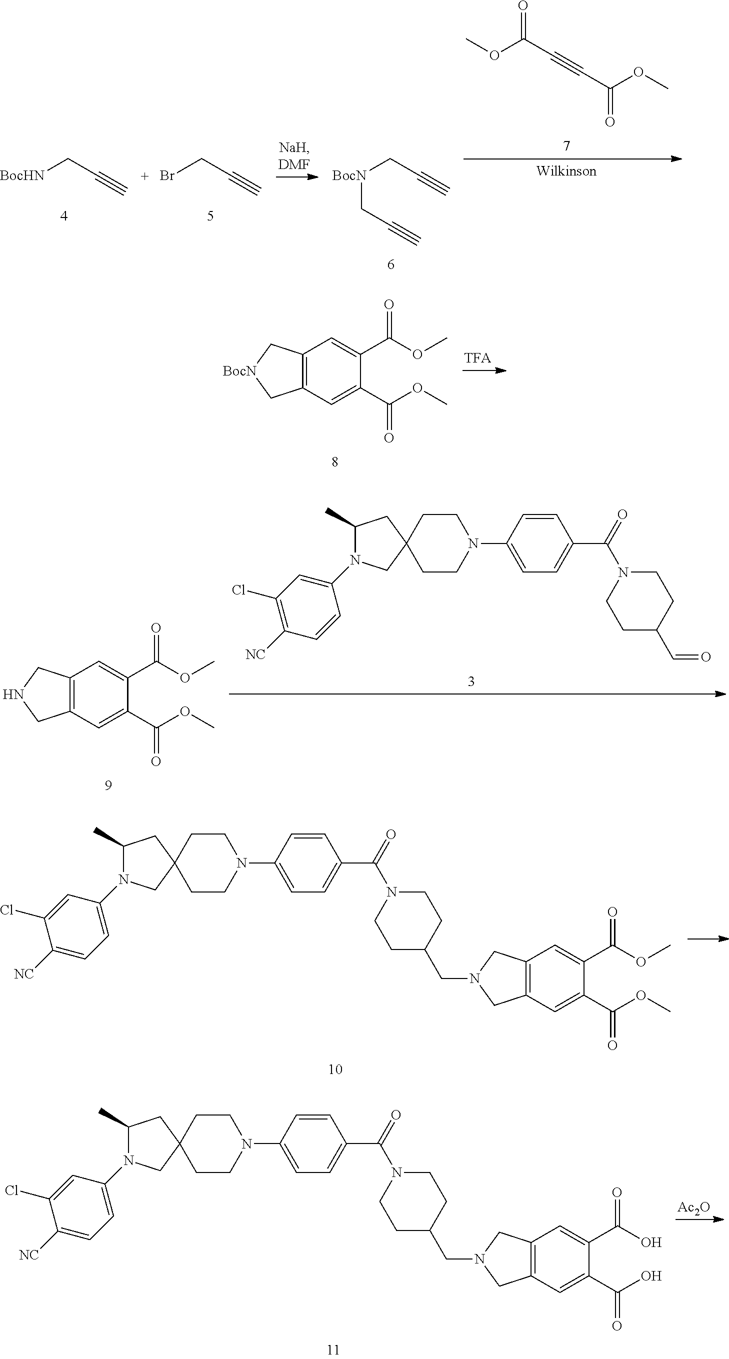

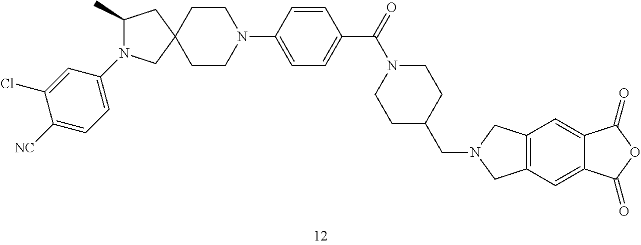 [SMILES: C[C@H]1CC2(CCN(c3ccc(C(=O)N4CCC(CN5Cc6cc7c(cc6C5)C(=O)OC7=O)CC4)cc3)CC2)CN1c1ccc(C#N)c(Cl)c1]

Compound 4 (1.0 eq) was dissolved in DMF (5×) and NaH (60% in mineral oil, 3.0 eq) was added at 0° C. After 0.5 h, compound 5 (1.2 eq) was added and the reaction mixture was stirred at 0° C. for 4 h. The reaction was quenched with H₂O and partitioned between EtOAc and H₂O. The organic layer was separated, dried, and purified by Combiflash with Hexane and EtOAc to afford compound 6 in 700% yield.

Compound 6 (1.0 eq), compound 7 (4.0 eq), and Wilkinson catalyst (0.2 eq) were dissolved in anhydrous toluene. The mixture was degassed and refluxed under Ar atmosphere for 18 h. The reaction mixture was concentrated with silica gel, and purified by Combiflash with Hexane and EtOAc to provide compound 8 in 3500 yield.

Compound 8 was dissolved in DCM and TFA (5×) was added at rt. The deprotection was completed in 0.5 h. The volatiles were removed to give compound 9 as off white powder.

Compound 9 (1.0 eq), compound 3 (1.3 eq) and AcOH (3.0 eq) were dissolved in DCE (10×). After 2 h, NaB(OAc)₃H (3.0 eq) was added. The reaction was complete in 3 h. The reaction mixture was directly placed on top of a silica gel column and eluted with DCM and MeOH to give compound 10 as white solid.

Compound 10 (1.0 eq) was dissolved in THF and NaOH (6 N) was added. The reaction was stirred at 60° C. for 2 h. The THF was removed and the residue was acidified with HCl to pH 1. The solution was lyophilized to provide white powder, which was purified by Combiflash with DCM and MeOH to give compound 11 as white solid.

Compound 11 (1.0 eq) was dissolved in THF (5×) and Ac₂O (2.0 eq) was added. The reaction was refluxed for 4 h. The reaction mixture was cooled to rt, and purified by Combiflash with DCM and MeOH to give compound 12 as white solid.

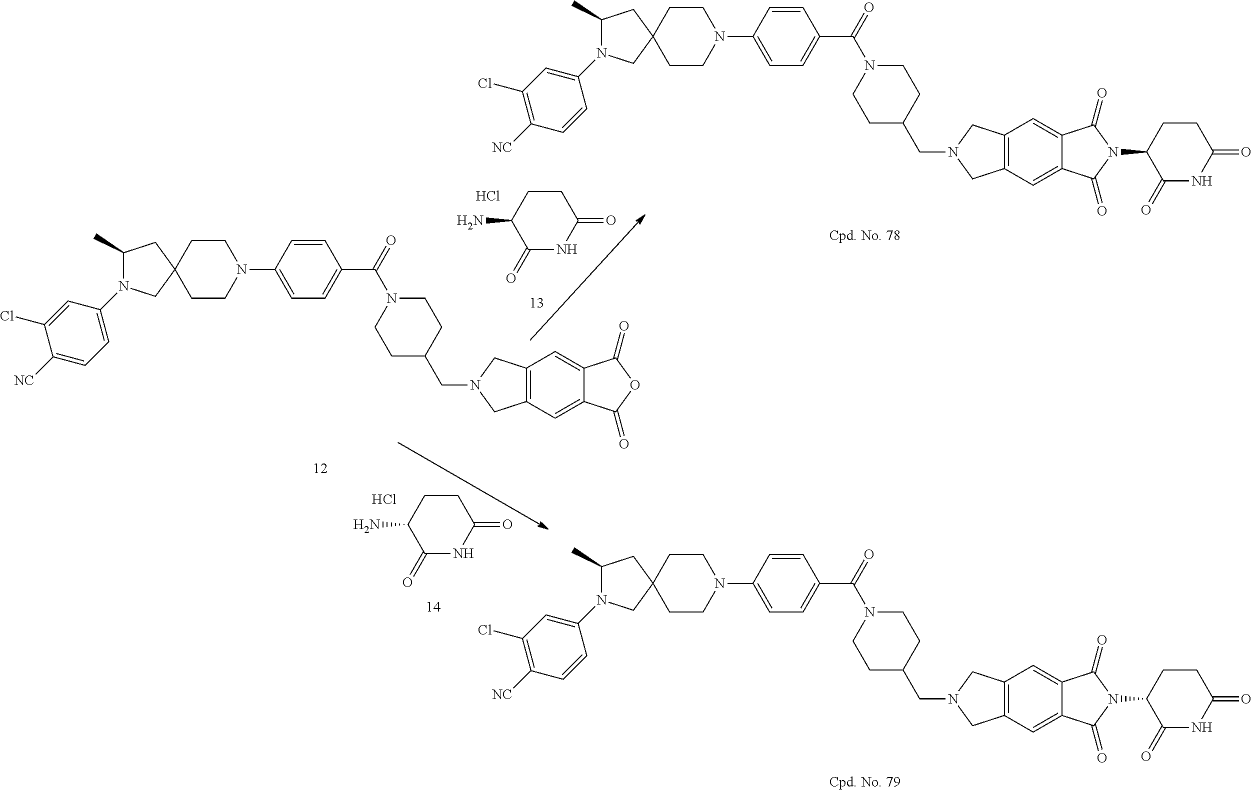

Compound 12 (1.0 eq) was dissolved in acetonitrile (10×), and DIPEA (2.0 eq) and compound 13 or 14 (1.0 eq) were added. The reaction mixture was refluxed for 18 h. The acetonitrile was removed, and the residue was diluted with H₂O, acidified with TFA, and purified by prep HPLC to give Cpd. No. 78 and Cpd. No. 79 in 37% yield. UPLC-MS. 3.6 min, 788.34.

### Example 15. Biological Activity of Exemplary Compounds

Biological Assays. LNCaP and VCaP cells used were purchased from American Type Culture Collection (ATCC). LNCaP were grown in RPMI 1640 (Invitrogen) and VCaP cells were grown in DMEM with Glutamax (Invitrogen). All of the cells were supplemented with 10% fetal bovine serum (Invitrogen) at 37° C. in a humidified 5% CO₂ incubator. The AR degradation activity an cellular potency of representative Compounds of the Disclosure are provided in Table A.

TABLE A

| | |
|---|---|
| Degradation of AR | Cellular potency |
| in VCaP cells | (IC₅₀) |

| Cpd. No. | DC₅₀ | Dmax | LNCaP | VCaP |
|---|---|---|---|---|
| 1 | ++++ | A | | |
| 2 | ++++ | A | +++ | +++ |
| 3 | ++++ | A | +++ | +++ |
| 4 | ++++ | B | +++ | +++ |
| 5 | ++++ | A | +++ | +++ |
| 6 | ++++ | A | +++ | +++ |
| 8 | +++ | B | ++ | ++ |
| 9 | ++++ | A | +++ | +++ |
| 10 | +++ | A | +++ | +++ |
| 13 | ++ | A | | |
| 14 | ++ | A | | |
| 15 | ++ | A | | |
| 16 | ++++ | A | +++ | +++ |
| 17 | ++ | B | ++ | ++ |
| 18 | +++ | A | +++ | +++ |
| 19 | ++ | B | ++ | ++ |
| 20 | ++ | B | ++ | ++ |
| 21 | ++++ | A | +++ | +++ |
| 22 | ++ | B | ++ | ++ |
| 23 | ++++ | A | +++ | +++ |
| 24 | ++++ | A | +++ | +++ |
| 25 | ++++ | A | +++ | +++ |
| 26 | ++++ | A | +++ | +++ |
| 27 | ++++ | A | +++ | +++ |
| 28 | ++++ | A | +++ | +++ |
| 29 | ++ | A | ++ | ++ |
| 30 | ++++ | A | +++ | +++ |
| 31 | ++++ | A | +++ | +++ |
| 32 | ++++ | A | +++ | +++ |
| 33 | +++ | B | +++ | +++ |
| 34 | ++++ | A | +++ | +++ |
| 35 | ++++ | A | +++ | +++ |
| 36 | ++++ | A | +++ | +++ |
| 37 | ++++ | A | +++ | +++ |
| 38 | ++++ | A | +++ | +++ |
| 39 | ++++ | A | +++ | +++ |
| 40 | +++ | A | ++ | ++ |
| 41 | +++ | A | ++ | ++ |
| 42 | +++ | A | ++ | ++ |
| 43 | ++++ | A | +++ | +++ |
| 44 | ++++ | A | +++ | +++ |
| 45 | ++++ | A | +++ | +++ |
| 47 | ++++ | A | +++ | +++ |
| 48 | ++++ | A | +++ | +++ |
| 49 | ++++ | A | +++ | +++ |
| 50 | +++ | A | ++ | ++ |
| 51 | +++ | A | ++ | ++ |
| 52 | +++ | A | ++ | ++ |
| 53 | +++ | A | ++ | ++ |
| 54 | +++ | A | ++ | ++ |
| 55 | +++ | A | ++ | ++ |
| 56 | +++ | A | ++ | ++ |
| 57 | ++++ | A | +++ | +++ |
| 58 | +++ | A | ++ | ++ |
| 59 | ++++ | A | +++ | +++ |
| 60 | ++++ | A | +++ | +++ |
| 61 | ++++ | A | +++ | +++ |
| 62 | ++++ | A | +++ | +++ |
| 63 | ++++ | A | +++ | +++ |
| 69 | ++++ | A | +++ | +++ |
| 70 | ++ | C | + | + |
| 71 | ++ | C | + | + |
| 72 | ++ | C | + | + |
| 73 | ++ | C | + | + |
| 74 | ++ | C | + | + |
| 82 | ++++ | A | +++ | +++ |
| 83 | ++++ | A | +++ | +++ |
| 84 | ++++ | A | | |
| 85 | ++++ | A | | |
| 86 | ++++ | A | | |
| 87 | ++++ | A | | |
| 88 | ++++ | A | | |
| 89 | ++++ | A | | |
| 90 | ++++ | A | +++ | +++ |
| 91 | ++++ | A | +++ | +++ |
| 92 | ++++ | A | +++ | +++ |
| 119 | ++++ | A | +++ | +++ |
| 120 | ++++ | A | +++ | +++ |
| 121 | ++ | B | ++ | ++ |
| 123 | ++++ | A | +++ | +++ |
| 124 | +++ | B | ++ | ++ |
| 125 | +++ | A | +++ | +++ |
| 131 | ++++ | A | +++ | +++ |
| 132 | ++++ | A | +++ | +++ |
| 133 | ++++ | A | +++ | +++ |
| 134 | ++++ | A | +++ | +++ |
| 135 | +++ | B | ++ | ++ |
| 136 | ++++ | A | +++ | +++ |
| 137 | +++ | B | ++ | ++ |
| 138 | +++ | B | ++ | ++ |
| 139 | +++ | B | ++ | ++ |
| 147 | ++++ | | +++ | ++ |
| 148 | ++++ | | +++ | ++ |
| 149 | ++++ | | +++ | ++ |
| 152 | ++++ | | ++ | ++ |
| 153 | ++++ | | +++ | ++ |
| 154 | ++++ | | +++ | ++ |
| 160 | ++++ | | +++ | + |
| 161 | ++++ | | +++ | +++ |
| 162 | ++++ | | +++ | +++ |
| 163 | ++++ | | +++ | +++ |
| 164 | ++++ | | ++ | +++ |
| 165 | +++ | | +++ | +++ |
| 166 | +++ | | ++ | +++ |
| 167 | ++++ | | +++ | +++ |
| 168 | +++ | | +++ | +++ |
| 169 | ++++ | | +++ | +++ |
| 170 | +++ | | +++ | ++ |
| 171 | +++ | | +++ | ++ |
| 172 | ++++ | | +++ | +++ |
| 173 | ++++ | | +++ | ++ |
| 174 | ++++ | | +++ | +++ |
| 175 | ++++ | | +++ | +++ |
| 176 | +++ | | +++ | +++ |
| 177 | ++++ | | +++ | +++ |
| 178 | +++ | | ++ | ++ |
| 179 | +++ | | +++ | ++ |
| 180 | ++++ | | +++ | ++ |
| 181 | ++++ | | +++ | ++ |
| 182 | ++++ | | +++ | ++ |
| 183 | ++++ | | ++ | ++ |
| 185 | ++++ | | ++ | ++ |
| 194 | +++ | | +++ | ++ |
| 195 | +++ | | +++ | ++ |
| 196 | ++++ | | +++ | ++ |
| 197 | ++++ | | +++ | +++ |
| 198 | +++ | | +++ | +++ |
| 202 | ++++ | | +++ | +++ |
| 203 | ++++ | | +++ | +++ |
| 204 | ++++ | | +++ | +++ |
| 205 | ++++ | | +++ | +++ |

DC₅₀: ++++ = <1 nM;
+++ = <10 nM;
++ = <100 nM;
+ = <1000 nM
Dmax: A = >90%;
B = 90%-70%;
C = 70%-50%;
D = <50%;
IC₅₀: +++ = <10 nM;
++ = <100 nM;
+ = <1000 nM

### REFERENCES

(1) Hamdy et al., “Outcomes after Monitoring, Surgery, or Radiotherapy for Localized Prostate Cancer,” N Engl J Med, 2016, 375, 1415-1424.
(2) Litwin, M. S.; Tan, H. J. The Diagnosis and Treatment of Prostate Cancer. JAMA, 2017, 317, 2532-2542.
(3) Karantanos et al., “Prostate cancer progression after androgen deprivation therapy: mechanisms of castrate resistance and novel therapeutic approaches,” Oncogene. 2013, 32, 5501-511.
(4) Harris et al., “Androgen deprivation therapy: progress in understanding mechanisms of resistance and optimizing androgen depletion,” Nat Clin Pract Urol, 2009, 6, 76-85.
(5) Narayanan et al., “Destroying the androgen receptor (AR)-potential strategy to treat advanced prostate cancer,” Oncoscience. 2017, 4, 175-177.
(6) Crowder et al., “Nuclear Androgen Receptor Regulates Testes Organization and Oocyte Maturation in Zebrafish,” Endocrinology. 2018, 159, 980-993.
(7) Sundén et al., “Synthesis and Biological Evaluation of Second-Generation Tropanol-Based Androgen Receptor Modulators,” J. Med. Chem. 2015, 58, 1569-1574.
(8) Oksala et al., “A Novel Nonsteroidal Compound for the Treatment of Castration-Resistant Prostate Cancer by blocking the Androgen Receptor and Inhibiting CYP17A1,” J Steroid Biochem Mol Biol. 2018, doi: 10.1016/j.jsbmb.2018.02.004.
(9) Watson et al., “Emerging mechanisms of resistance to androgen receptor inhibitors in prostate cancer,” Nat Rev Cancer. 2015, 15, 701-711.
(10) Guo et al., “Discovery of Aryloxy Tetramethylcyclobutanes as Novel Androgen Receptor Antagonists,” J. Med. Chem. 2011, 54, 7693-7704.
(11) Moilanen et al., “Discovery of ODM-201, a new generation androgen receptor inhibitor targeting resistance mechanisms to androgen signaling-directed prostate cancer therapies,” Sci Rep. 2015, 5, 12007.
(12) Guerrini et al., “A New Avenue toward Androgen Receptor Pan-antagonists: C2 Sterically Hindered Substitution of Hydroxy-propanamides,” J. Med. Chem. 2014, 57, 7263-7279.
(13) Jung et al., “Structure-activity relationship for thiohydantoin androgen receptor antagonists for castration-resistant prostate cancer (CRPC),” J Med. Chem. 2010, 53, 2779-2796.
(14) Yamamoto et al., “Design, synthesis, and biological evaluation of 4-arylmethyl-1-phenylpyrazole and 4-aryloxy-1-phenylpyrazole derivatives as novel androgen receptor antagonists,” Bioorg Med Chem. 2012, 20, 2338-2352.
(15) Balbas et al., “Overcoming mutation-based resistance to antiandrogens with rational drug design,” Elife. 2013, 2, e00499.
(16) Lottrup et al., “Identification of a novel androgen receptor mutation in a family with multiple components compatible with the testicular dysgenesis syndrome,” J Clin Endocrinol Metab. 2013, 98, 2223-2229.
(17) Zhu et al., “BMI1 regulates androgen receptor in prostate cancer independently of the polycomb repressive complex 1,” Nat Commun. 2018, 9, 500.
(18) Munuganti et al., “Identification of a potent antiandrogen that targets the BF3 site of the androgen receptor and inhibits enzalutamide-resistant prostate cancer,” Chem Biol. 2014, 21, 1476-485.
(19) Raina et al., “PROTAC-induced BET protein degradation as a therapy for castration-resistant prostate cancer,” Proc Natl Acad Sci USA. 2016, 113, 7124-7129.
(20) Zhou et al., “Discovery of a Small-Molecule Degrader of Bromodomain and Extra-Terminal (BET) Proteins with Picomolar Cellular Potencies and Capable of Achieving Tumor Regression,” J. Med. Chem. 2018, 61, 462-481.
(21) Gadd et al., “Structural basis of PROTAC cooperative recognition for selective protein degradation,” Nat Chem. Biol. 2017, 13, 514-521.
(22) Toure et al., “Small-molecule PROTACS: new approaches to protein degradation,” Angew. Chem. Int. Edn. 2016, 55, 1966-1973.
(23) Qin et al., “Discovery of QCA570 as an Exceptionally Potent and Efficacious Proteolysis Targeting Chimera (PROTAC) Degrader of the Bromodomain and Extra-Terminal (BET) Proteins Capable of Inducing Complete and Durable Tumor Regression,” J. Med. Chem. 2018, 61, 6685-6704.
(24) Hatcher et al., “Development of Highly Potent and Selective Steroidal Inhibitors and Degraders of CDK8, “ACS Med. Chem. Lett. 2018, 9, 540-545.
(25) Gollavilli et al., “EWS/ETS-Driven Ewing Sarcoma Requires BET Bromodomain Proteins,” Cancer Res. 2018, 78, 4760-4773.
(26) Bondeson et al., “Targeted Protein Degradation by Small Molecules. Annu Rev Pharmacol Toxicol,” 2017, 57, 107-123.
(27) Salami et al., “Androgen receptor degradation by the proteolysis-targeting chimera ARCC-4 outperforms enzalutamide in cellular models of prostate cancer drug resistance,” Commun Biol. 2018, 1, 100.
(28) Pal et al., “Identification of mechanisms of resistance to treatment with abiraterone acetate or enzalutamide in patients with castration-resistant prostate cancer (CRPC),” Cancer. 2018, 124, 1216-1224.
(29) Wang et al., “Blocking the Feedback Loop between Neuroendocrine Differentiation and Macrophages Improves the Therapeutic Effects of Enzalutamide (MDV3100) on Prostate Cancer,” Clin Cancer Res. 2018, 24, 708-723.
(30) Gustafson et al., “Small-Molecule-Mediated Degradation of the Androgen Receptor through Hydrophobic Tagging,” Angew. Chem. Int. Ed. 2015, 54, 9659-9662.
(31) Shibata et al., “Development of Protein Degradation Inducers of Androgen Receptor by Conjugation of Androgen Receptor Ligands and Inhibitor of Apoptosis Protein Ligands,” J. Med. Chem. 2018, 61, 543-575.
(32) Crew et al., US 20170327469 A1
(33) Pereira de Jésus-Tran et al., “Comparison of crystal structures of human androgen receptor ligand-binding domain complexed with various agonists reveals molecular determinants responsible for binding affinity,” Protein Sci. 2006, 15, 987-999.
(34) Galdeano et al., “Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities, “J. Med. Chem. 2014, 57, 8657-8663.
(35) Soares et al., “Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298),” J. Med. Chem. 2018, 61, 599-618.
(36) Buckley et al., “Targeting the von Hippel-Lindau E3 ubiquitin ligase using small molecules to disrupt the VHL/HIF-1α interaction, “J. Am. Chem. Soc. 2012, 134, 4465-4468.
(37) Frost et al., “Potent and selective chemical probe of hypoxic signalling downstream of HIF-alpha hydroxylation via VHL inhibition,” Nat Commun, 2016, 7, 13312-13312.
(38) Berlin et al., WO2016149668A1
(39) Ishoey et al., “Translation Termination Factor GSPT1 Is a Phenotypically Relevant Off-Target of Heterobifunctional Phthalimide Degraders,” ACS Chem. Biol. 2018, 13, 553-560.
(40) Powell et al., “Chemically Induced Degradation of Anaplastic Lymphoma Kinase (ALK),” J. Med. Chem. 2018, 61, 4249-4255.
(41) Liu et al., “Melatonin Inhibits Androgen Receptor Splice Variant-7 (AR-V7)-Induced Nuclear Factor-Kappa B (NF-κB) Activation and NF-κB Activator-Induced AR-V7 Expression in Prostate Cancer Cells: Potential Implications for the Use of Melatonin in Castration-Resistant Prostate Cancer (CRPC) Therapy,” Int J Mol Sci. 2017, 18, E1130.
(42) Sun et al., “Design, synthesis, and characterization of a potent, nonpeptide, cell-permeable, bivalent Smac mimetic that concurrently targets both the BIR2 and BIR3 domains in XIAP,” J. Am. Chem. Soc. 2007, 129, 15279-15294.
(43) Lu et al., “SM-164: a novel, bivalent Smac mimetic that induces apoptosis and tumor regression by concurrent removal of the blockade of cIAP-1/2 and XIAP,” Cancer Res. 2008, 68, 9384-9393.
(44) Bai et al., “Targeted Degradation of BET Proteins in Triple-Negative Breast Cancer,” Cancer Res. 2017, 77, 2476-2487.
(45) Stols et al., “A new vector for high-throughput, ligation-independent cloning encoding a tobacco etch virus protease cleavage site, “Protein Expr Purif 2002, 25, 8-15.
(46) Benoit, et al., “Seamless Insert-Plasmid Assembly at High Efficiency and Low Cost,” PLoS One. 2016, 11, e0153158.

### EQUIVALENTS

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I: 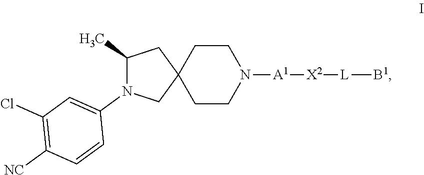
or a pharmaceutically acceptable salt or solvate thereof, wherein:
A¹ is selected from phenylenyl, 5-membered heteroarylenyl, and 6-membered heteroarylenyl;
X² is selected from —C(═O)—, —S(═O)₂—, —O—, and —CR^{4c}R^{4d}—; or X² is absent;
R^{4c} and R^{4d} are independently selected from hydrogen and C₁-C₄ alkyl;
L is -J¹-J²-J³-J⁴-J⁵-; wherein J¹ is attached to X²;
J¹ is selected from cycloalkylenyl and heterocyclenyl; or J¹ is absent;
J² is selected from —(CH₂)_{b}—, —C(═O)—, —CH═CH—, and —C≡C—;
b is 0, 1, 2, or 3;
J³ is selected from alkylenyl, heteroalkylenyl, cycloalkylenyl, heterocyclenyl, phenylenyl, and heteroarylenyl; or
J³ is absent;
J⁴ is selected from alkylenyl, cycloalkylenyl, and heterocyclenyl; or J⁴ is absent;
J⁵ is selected from —(CH₂)_{c}—, —O—, —N(R⁵)—, and —C(═O)—;
c is 0, 1, 2, or 3;
R⁵ is selected from hydrogen and C₁-C₄ alkyl;
B¹ is selected from: 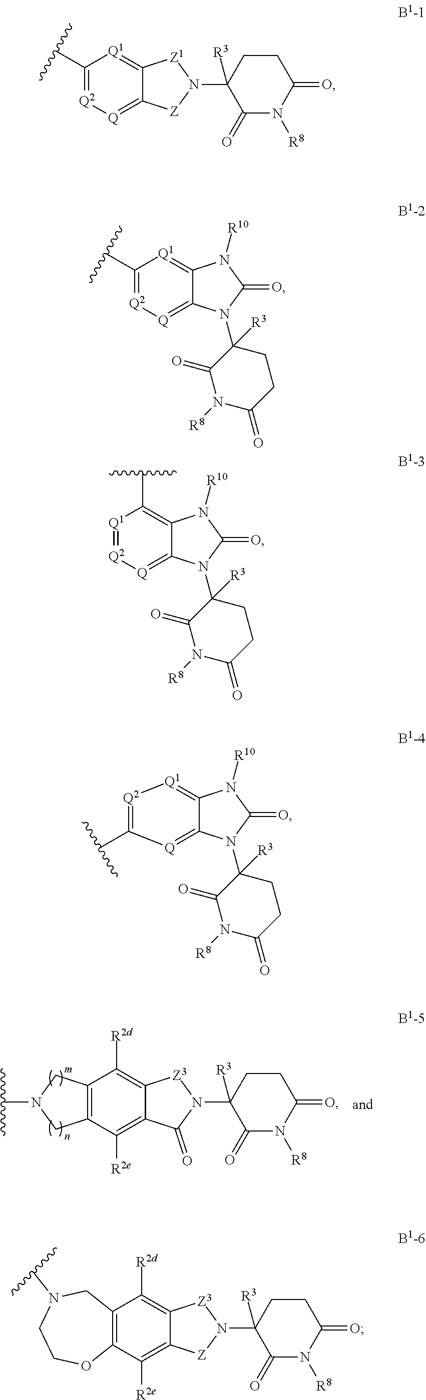
Q¹ is selected from —CR^{2a}═ and —N═;
Q² is selected from —CR^{2b}═ and —N═;
Q is selected from —CR^{2c}═ and —N═;
R^{2a}, R^{2b}, R^{2c}, R^{2d}, and R^{2e} are independently selected from hydrogen, halo, amino, C₁-C₃ alkyl, and C₁-C₃ alkoxy;
R³ is selected from hydrogen, deuterium, fluoro, and C₁-C₄ alkyl;
m is 1, 2, or 3;
n is 1, 2, or 3;
Z and Z¹ are —C(═O)—; or
Z is —C(═O)— and Z¹ is —CR^{6a}R^{6b}—; or
Z is —CR^{6a}R^{6b}— and Z¹ is —C(═O)—;
Z³ is selected from —CR^{6c}R^{6d}— and —C(═O)—;
R^{6a} and R^{6b} are independently selected from hydrogen and C₁-C₃ alkyl; or
R^{6c} and R^{6d} taken together with the carbon to which they are attached from a C₃-C₆ cycloalkyl;
R⁸ is selected from hydrogen and C₁-C₃ alkyl; and
R¹⁰ is selected from hydrogen and C₁-C₄ alkyl,
with the proviso the compound of Formula I is not a compound described in Table 1A.

2. The compound of claim 1, being of Formula II: 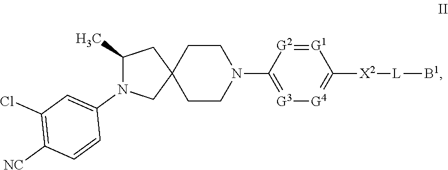
or a pharmaceutically acceptable salt or solvate thereof, wherein:
G¹ is selected from —CR^{5a}═ and —N═;
G² is selected from —CR^{5b}═ and —N═;
G³ is selected from —CR^{5c}═ and —N═;
G⁴ is selected from —CR^{5d}═ and —N═; and
R^{5a}, R^{5b}, R^{5c}, and R^{5d} are independently selected from hydrogen, halo, C₁-C₃ alkyl, and C₁-C₃ alkoxy.

3. The compound of claim 1, being of Formula III: 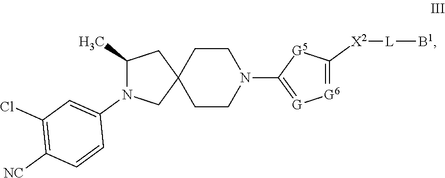
or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

4. The compound of claim 1, being of Formula IV: 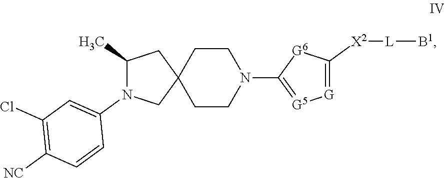
or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

5. The compound of claim 1, being of Formula V: 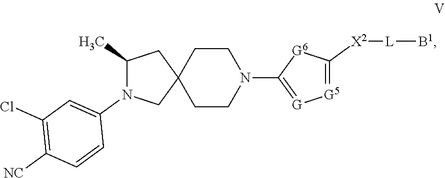
or a pharmaceutically acceptable salt or solvate thereof, wherein:
G is selected from —CH═ and —N═;
G⁵ is selected from —O—, —S—, and —NR⁹—;
G⁶ is selected from —CH═ and —N═; and
R⁹ is selected from hydrogen and C₁-C₃ alkyl.

6. The compound of claim 1, being of Formula VI: 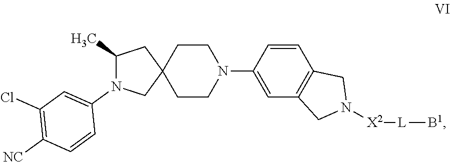
or a pharmaceutically acceptable salt or solvate thereof.

7. The compound of any one of the preceding claims, or a pharmaceutically acceptable salt or solvate thereof, wherein:
(i) X² is —C(═O)—, —S(═O)₂—, —O—, or —CR^{4c}R^{4d}—;
optionally, R^{4c} and R^{4d} are hydrogen; or
(ii) X² is absent.

8. The compound of any one of the preceding claims, or a pharmaceutically acceptable salt or solvate thereof, wherein:
(i) J¹ is selected from: 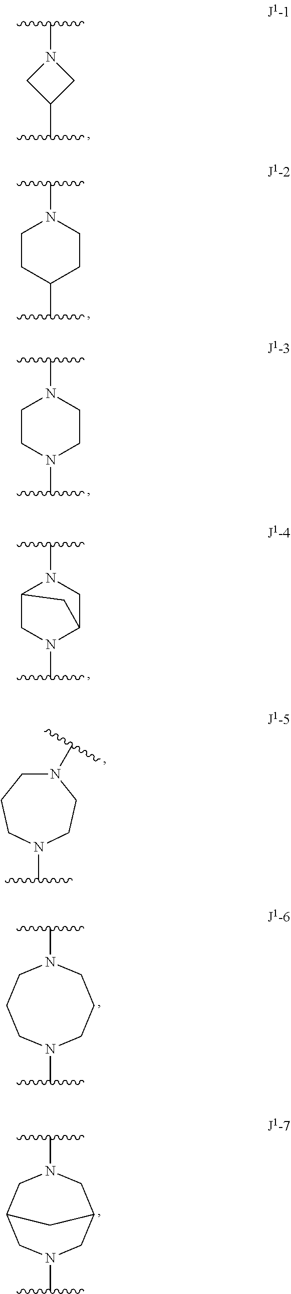 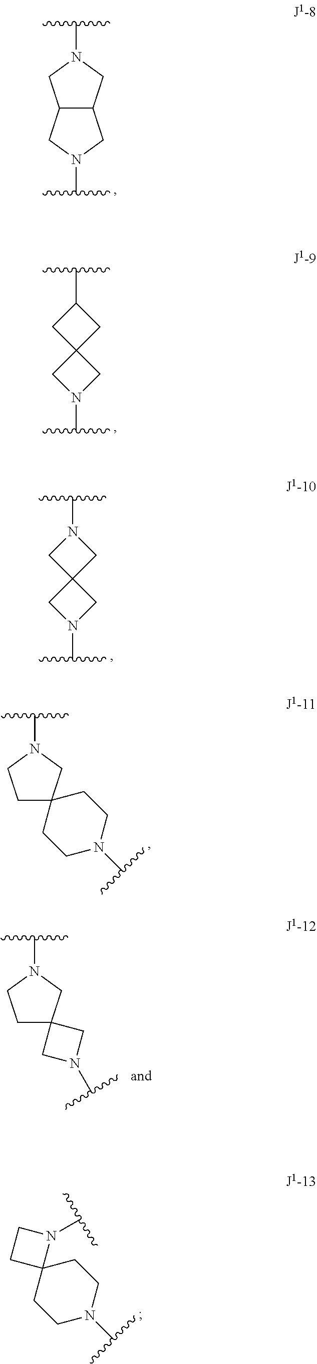
or
(ii) J¹ is absent; and
optionally, wherein:
(i) J² is selected from —(CH₂)_{b}— and —C≡C—; and b is 0, 1, or 2;
(ii) J² is —(CH₂)_{b}—; and b is 0;
(iii) J² is —(CH₂)_{b}—; and b is 1; or
(iv) J² is —C≡C—; and
optionally, wherein:
(i) J³ is selected from cycloalkylenyl and heterocyclenyl; or
(ii) J³ is absent; and
optionally, wherein:
(i) J⁴ is selected from alkylenyl, cycloalkylenyl, and heterocyclenyl; or
(ii) J⁴ is absent.

9. The compound of any one of the preceding claims, or a pharmaceutically acceptable salt or solvate thereof, wherein:
(i) J¹ is selected from —O— and —N(H)—; and B¹ is selected from B¹-1, B¹-2, and B¹-3; or
(ii) J¹ is selected from —(CH₂)_{c}— and —O—; c is 0; J⁴ is selected from J⁴-1, J⁴-2, J⁴-3, J⁴-4, J⁴-5, and J⁴-6; R⁷ is selected from hydrogen, halo, cyano, hydroxy, C₁-C₃ alkyl, and C₁-C₃ alkoxy; and B¹ is selected from B¹-1, B¹-2, B¹-3, and B¹-4.

10. The compound of any one of the preceding claims, or a pharmaceutically acceptable salt or solvate thereof, wherein:
(i) B¹ is B¹-1;
optionally, B¹-1 is B¹-1-B or B¹-1-C: 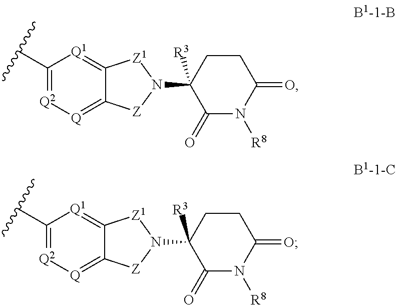
optionally, Z and Z¹ are —C(═O)—; Z is —C(═O)— and Z¹ is —CR^{6a}R^{6b}—; or Z is —CR^{6a}R^{6b}— and Z¹ is —C(═O)—;
(ii) B1 is B¹-2;
optionally, B¹-2 is B¹-2-B or B¹-2-C: 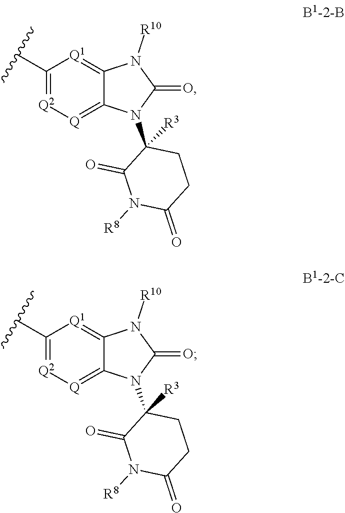
(iii) B1 is B¹-3;
optionally, B¹-3 is B¹-3-B or B¹-3-C: 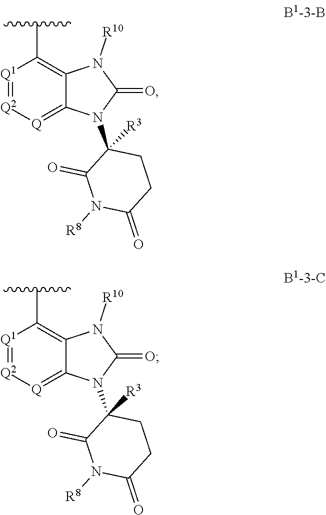
or
(iv) B¹ is B¹-4;
optionally, B¹-4 is B¹-4-B or B¹-4-C: 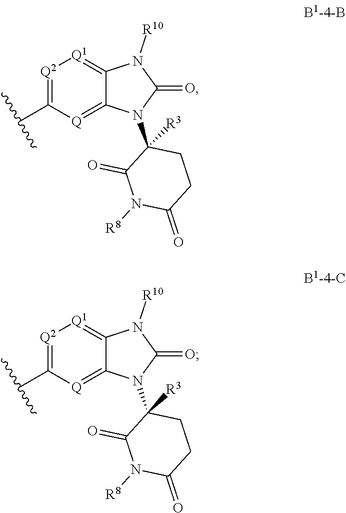
and
optionally, R¹⁰ is C₁-C₃ alkyl; and
optionally, Q¹ is —N═; or Q¹ is —CR^{2a}, optionally, R^{2a} is selected from hydrogen and halo; and
optionally, Q² is —N═; or Q² is —CR^{2b}, optionally, R^{2b} is selected from hydrogen and halo; and
optionally, Q is —N═; or Q is —CR^{2c}, optionally, R², is selected from hydrogen and halo.

11. The compound of any one of the preceding claims, wherein:
J¹ is selected from —(CH₂)_{c}— and —C(═O)—;
c is 0, 1, 2, or 3; and
B¹ is selected from B¹-5 and B¹-6, or a pharmaceutically acceptable salt or solvate thereof.

12. The compound of any one of the preceding claims, or a pharmaceutically acceptable salt or solvate thereof, wherein
(i) B¹ is B¹-5;
optionally, B¹-5 is B¹-5-B or B¹-5-C: 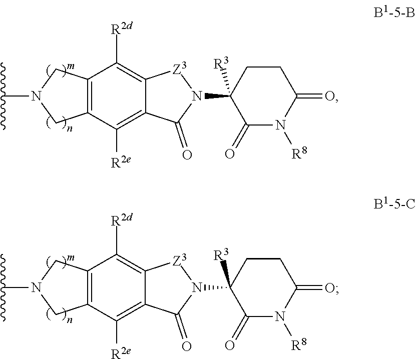
optionally, R^{2d} and R^{2d} are independently selected from hydrogen and halo; and
optionally, Z³ is —C(═O)—; or Z³ is —CR^{6a}R^{6b}—; or
(ii) B¹ is B′-6;
optionally, B¹-6 is B¹-6-B or B¹-6-C: 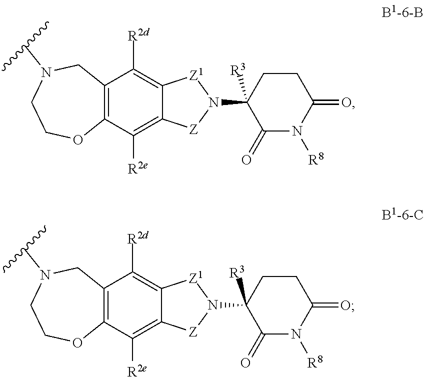
optionally, Z and Z¹ are —C(═O)—; Z is —C(═O)— and Z¹ is —CR^{6a}R^{6b}—; or Z is —CR^{6a}R^{6b}— and Z¹ is —C(═O)—; and
optionally, R^{2d} and R^{2d} are independently selected from hydrogen and halo.

13. The compound of any one of the preceding claims, or a pharmaceutically acceptable salt or solvate thereof, wherein R³ is hydrogen.

14. The compound of any one of the preceding claims, or a pharmaceutically acceptable salt or solvate thereof, wherein R¹ is hydrogen.

15. The compound of any one of the preceding claims, or a pharmaceutically acceptable salt or solvate thereof, wherein B¹ is selected from: 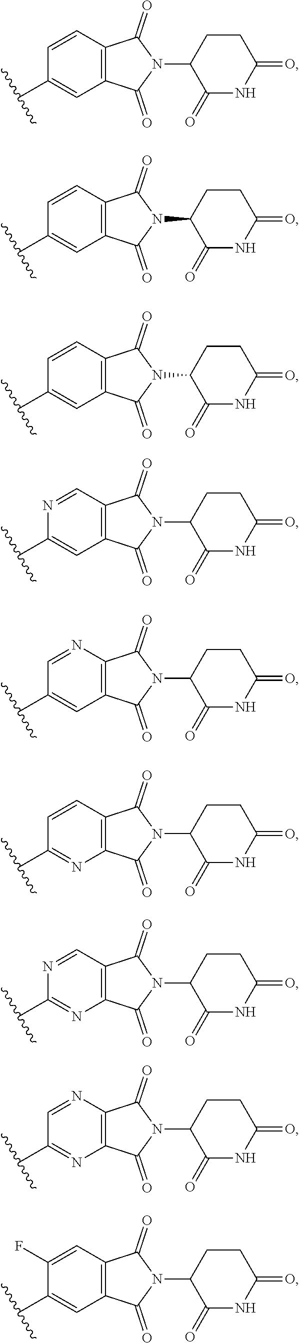 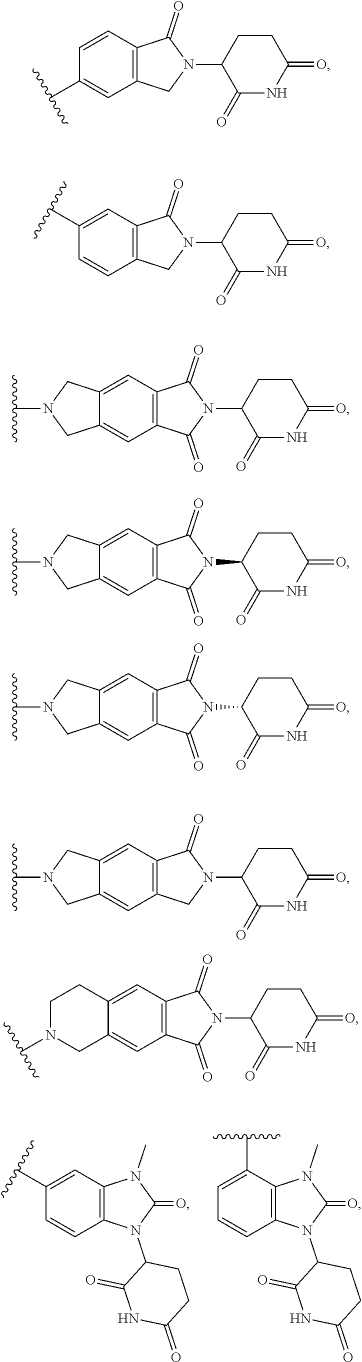 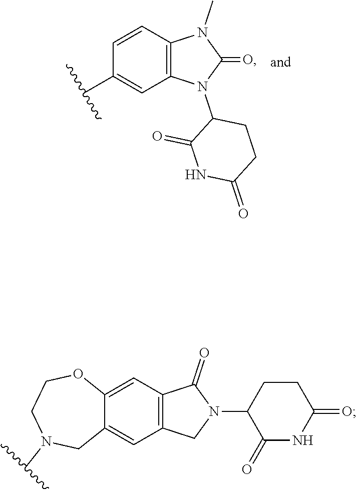 [SMILES: *c1ccc2c(c1)n(C1CCC(=O)NC1=O)c(=O)n2C ; *N1CCOc2cc3c(cc2C1)CN(C1CCC(=O)NC1=O)C3=O]
and
optionally, B¹ is:

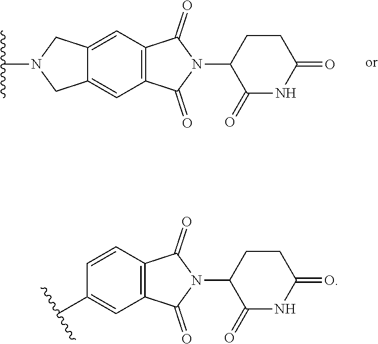 [SMILES: *N1Cc2cc3c(cc2C1)C(=O)N(C1CCC(=O)NC1=O)C3=O ; *c1ccc2c(c1)C(=O)N(C1CCC(=O)NC1=O)C2=O]

16. The compound of any one of the preceding claims, being selected from the compounds described in Table 1, or a pharmaceutically acceptable salt or solvate thereof.

17. A pharmaceutical composition comprising the compound of any one of the preceding claims, or a pharmaceutically acceptable salt or solvate thereof, and a pharmaceutically acceptable carrier.

18. A method of degrading an AR protein in a subject, comprising administering to the subject the compound of any one of the preceding claims or a pharmaceutically acceptable salt or solvate thereof.

19. Use of the compound of any one of the preceding claims or a pharmaceutically acceptable salt or solvate thereof in the manufacture of a medicament for degrading an AR protein in a subject.

20. The compound of any one of the preceding claims or a pharmaceutically acceptable salt or solvate thereof for use in degrading an AR protein in a subject.

21. A method of treating or preventing a disease in a subject in need thereof, comprising administering to the subject the compound of any one of the preceding claims or a pharmaceutically acceptable salt or solvate thereof

22. Use of the compound of any one of the preceding claims or a pharmaceutically acceptable salt or solvate thereof in the manufacture of a medicament for treating or preventing a disease in a subject.

23. The compound of any one of the preceding claims or a pharmaceutically acceptable salt or solvate thereof for use in treating or preventing a disease in a subject.

24. The method, use, or compound for use in any one of the preceding claims, wherein the subject is a mammal.

25. The method, use, or compound for use in any one of the preceding claims, wherein the subject is a human.

26. The method, use, or compound for use in any one of the preceding claims, wherein the disease is associated with degradation of an AR.

27. The method, use, or compound for use in any one of the preceding claims, wherein the disease is cancer, seborrhea, acne, hyperplasia, sebaceous adenoma, hirsutism, alopecia, or hidradenitis suppurativa;
optionally, the disease is a cancer;
optionally, the cancer is selected from the cancers described in Table I;
optionally, the cancer is breast cancer, ovarian cancer, or prostate cancer; and
optionally, the subject is in need of transgender therapy.
